(19) United States
(12) Patent Application Publication
(10) Pub. No.: US 2003/0069276 A1
(43) Pub. Date: Apr. 10, 2003

(54) Amino piperidine derivatives

(76) Inventors: Christopher David Edlin, Balderton (GB); Sally Redshaw, Shillington (GB); Ian Edward David Smith, Willington (GB); Daryl Simon Walter, Knebworth (GB)

Correspondence Address: HOFFMANN-LA ROCHE INC., PATENT LAW DEPARTMENT, 340 KINGSLAND STREET, NUTLEY, NJ, 07110

(21) Appl. No.: 10/104,117

(22) Filed: Mar. 22, 2002

(30) Foreign Application Priority Data
Mar. 30, 2001 (GB) 0108099.3

(51) Int. Cl. C 07D 4 3/02; A61K 31/454

(52) U.S. Cl. 514/336; 546/210

(57) ABSTRACT

The invention comprises novel aminopiperidine derivatives, a process for their manufacture, pharmaceutical compositions and the use of such compounds in medicine. In particular, the compounds of formula I prevent the human immunodeficiency virus (HIV) from entering cells by blocking interaction of the viral envelope protein gp120 with a chemokine receptor on the cell surface. The compounds of this invention may be advantageously used as therapeutic agents for the treatment of diseases mediated by the human immunodeficiency virus (HIV), either alone or in combination with other inhibitors of HIV viral replication or with pharmacoenhancers.

### BACKGROUND OF THE INVENTION

HIV is the causative agent of acquired immunodeficiency syndrome (AIDS), a disease characterised by the destruction of the immune system, particularly of the CD4⁺ T-cell, with attendant susceptibility to opportunistic infections. HIV infection is also associated with a precursor AIDS-related complex (ARC), a syndrome characterised by symptoms such as persistent generalised lymphadenopathy, fever and weight loss.

It has been reported [Liu et al., Cell 86, 367-377 (1996); Samson et al., Nature 382, 722-725 (1996); Dean et al., Science 273, 1856-1862 (1996)] that individuals who are homozygous for a deletion mutation in the CCR5 gene are highly resistant to infection by HIV, and that individuals heterozygous for this mutation have slowed disease progression [Huang et al., Nature Medicine 2, 1240-1243 (1996); Dean et al., Science 273, 1856-1862 (1996)]. Infection by HIV begins with attachment of the virus to a target cell, a process that requires the interaction of gp120 with both CD4 and a chemokine receptor (also termed a coreceptor) on the cell surface. Two important coreceptors for HIV infection are CXCR4 [Feng et al., Science 272, 872-877 (1996); Berson et al J Virol 70, 6288-6295 (1996)] and CCR5 [Alkhatib et al., Science 272, 1955-1958 (1996); Dragic et al., Nature 381, 667-673 (1996); Deng et al., Nature 381, 661-666 (1996)]. It is believed that binding to CD4 causes a conformational change in gp120 which then allows binding to the chemokine receptor [Deng et al., Nature 381, 661-666 (1996)]. Although many chemokine receptors can serve as coreceptors for HIV in vitro, it is believed that the major coreceptor involved in sexual, parenteral and vertical transmission of HIV is the CCR5 receptor [van't Wout et al., J. Clin. Invest. 94, 2060-2067 (1994); Cornelissen, et al J.Virol. 69, 1810-1818 (1995); Veenstra et al., Clin. Infect. Dis. 21, 556-560 (1995)]. Viruses that use CCR5 as coreceptor have been termed R5 viruses, and it is believed that these are the key pathogenic strains of HIV in the majority of patients. Thus, blocking the interaction of HIV with CCR5 should prevent HIV infection of healthy individuals and should slow or halt viral spread and disease progression in infected individuals.

Cyclic amine derivatives are described in WO 99/38514 modulators of chemokine receptor activity.

### SUMMARY OF THE INVENTION

The present invention provides novel aminopiperidine compounds of the general formula:

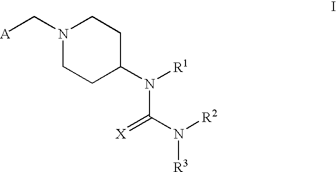

which inhibit entry of HIV into target cells by binding to the CCR5 receptor, optionally without blocking chemokine binding, thereby preventing the interaction of HIV gp120 and CD4 with this receptor, andare consequently useful for the prevention and treatment of HIV-related diseases.

### DETAILED DESCRIPTION OF THE INVENTION

The present invention comprises compounds of the formula:

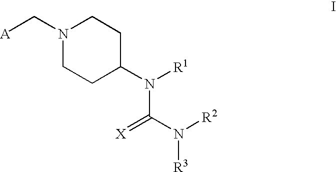

wherein

R¹ is hydrogen, C₁₋₁₂-alkyl, C₃₋₈-cycloalkyl, allyl, substituted C₁₋₄-alkyl, aryl, substituted aryl, heterocyclyl or substituted heterocyclyl;

R² and R³ are independently of each other hydrogen, C₁₋₁₂-alkyl, C₃₋₈-cycloalkyl, allyl, substituted C₁₋₄-alkyl, aryl, substituted aryl, heterocyclyl or substituted heterocyclyl;

X is S or O;

A is selected from the group consisting of:

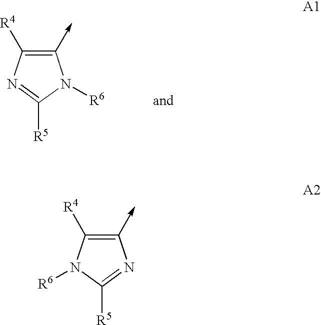

wherein

R⁴ is hydrogen, C₁₋₁₂-alkyl, substituted C₁₋₄-alkyl, C₃₋₈-cycloalkyl, C₁₋₄-alkoxy, CN, COR, CO₂R, CONRR′, NHCOR, aryl, substituted aryl, aryl-C(═O)—, substituted aryl-C(═O)—, aryl-CH(OH)—, substituted aryl-CH(OH)—, heterocyclyl, substituted heterocyclyl, heterocyclyl-C(═O)—, substituted heterocyclyl-C(═O)—, heterocyclyl-CH(OH)—, substituted heterocyclyl-CH(OH)— or NRR′;

R⁵ is hydrogen, C₁₋₁₂-alkyl, substituted C₁₋₄-alkyl, C₃₋₈-cycloalkyl, C₁₋₄-alkoxy, halogen, COR, aryl, substituted aryl, aryl-C(═O)—, substituted aryl-C(═O)—, aryl-CH(OH)—, substituted aryl-CH(OH)—, heterocyclyl, substituted heterocyclyl, heterocyclyl-C(═O)—, substituted heterocyclyl-C(═O)—, heterocyclyl-CH(OH)—, substituted heterocyclyl-CH(OH)— or NRR′;

R⁶ is hydrogen, C₁₋₁₂-alkyl, substituted C₁₋₄-alkyl, C₁₋₄-alkoxy, C₃₋₈-cycloalkyl, COR, CO₂R, CONRR′, NHCOR, SO₂NRR′ or SO₂R;

R and R′ are independently of each other hydrogen, C₁₋₁₂-alkyl, substituted C₁₋₄-alkyl, C₃₋₈-cycloalkyl, aryl, substituted aryl, heterocyclyl or substituted heterocyclyl;

as well as ethers or hydrolyzable esters of compounds of formula I and pharmaceutically acceptable salts thereof.

The term “alkyl” as used herein, and if not specified by the number of carbon atoms, denotes an optionally substituted straight or branched chain hydrocarbon residue containing 1 to 12 carbon atoms, such as methyl, ethyl, n-propyl, isopropyl, n-butyl, sec-butyl, isobutyl, tert.-butyl, pentyl, hexyl, heptyl, octyl, nonyl, decyl, undecyl, dodecyl including their different isomers. The term “C₁₋₁₂-alkyl” denotes a straight or branched chain hydrocarbon residue containing 1 to 12 carbon atoms as defined above. The term “C₁₋₇-alkyl” denotes a straight or branched chain hydrocarbon residue containing 1 to 7 carbon atoms and more preferably the term “C₁₋₄-alkyl” denotes a straight or branched chain hydrocarbon residue containing 1 to 4 carbon atoms.

Suitable substituents for the alkyl group are 1-3 substituents selected from C₃₋₈-cycloalkyl, aryl, heterocyclyl, substituted aryl and substituted heterocyclyl; wherein substituted aryl and substituted heterocyclyl means aryl or heterocyclyl substituted with C₁₋₄-alkoxy, phenyl, phenoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens; in case more than one substituent is attached to the alkyl group, these substituents can be identical or different from each other. Preferred substituents for the alkyl groups are 1-3 substituents selected from C₃₋₈-cycloalkyl, aryl, heterocyclyl, substituted aryl, substituted heterocyclyl and halogen; wherein substituted aryl and substituted heterocyclyl means aryl and heterocyclyl substituted with C₁₋₄-alkoxy, halogen, CN, NO₂, COR, CO₂R, CONRR′, NRR′, NHCOR, SO₂NRR′, C₁₋₄-alkyl or C₁₋₄-alkyl substituted with 1-3 halogens. More preferred substituents for the alkyl groups are 1-3 substituents selected from C₃₋₈-cycloalkyl, phenyl, pyridyl, substituted phenyl and substituted pyridyl, wherein substituted phenyl and substituted pyridyl are substituted with C₁₋₄-alkoxy, halogen, CN, NO₂, COR, CO₂R, CONRR′, NRR′, NHCOR, SO₂NRR′, C₁₋₄-alkyl or C₁₋₄-alkyl substituted with 1-3 halogens.

The substituents for substituted alkyl group are specifically defined below.

Alkyl in R¹ is preferably a straight or branched chain hydrocarbon residue containing 1 to 12 carbon atoms as defined above. Preferred alkyl groups in R¹ are straight or branched chain hydrocarbon residues containing 1 to 7 carbon atoms and, more preferably, the alkyl group in R¹ is methyl, ethyl, propyl, isopropyl, n-butyl, sec-butyl, isobutyl or tert.-butyl.

Alkyl in R² and R³ are, independently of each other, a straight or branched chain hydrocarbon residue containing 1 to 12 carbon atoms, as defined above. Preferred alkyl groups in R² and R³ are straight or branched chain hydrocarbon residues containing 1 to 7 carbon atoms, and more preferred alkyl groups in R² and R³ are methyl, ethyl, propyl, isopropyl, n-butyl, sec-butyl, isobutyl or tert.-butyl.

Alkyl in R⁴, R⁵, R⁶, R and R′ (independently of each other) denotes an optionally substituted straight or branched chain hydrocarbon residue containing 1 to 12 carbon atoms, such as methyl, ethyl, n-propyl, isopropyl, n-butyl, sec-butyl, isobutyl, tert.-butyl, pentyl, hexyl, heptyl, octyl, nonyl, decyl, undecyl, dodecyl including their different isomers. Preferably, alkyl denotes a straight or branched chain hydrocarbon residue containing 1 to 7 carbon atoms and more preferably alkyl denotes a straight or branched chain hydrocarbon residue containing 1 to 4 carbon atoms.

Alkyl in R⁷ and R⁸ are, independently of each other, methyl, ethyl, propyl, isopropyl, n-butyl, sec-butyl, isobutyl or tert.-butyl.

The term “cycloalkyl” as used herein, and if not specified by the number of carbon atoms, denotes a cycloalkyl group containing 3 to 8 carbon atoms, e.g. cyclopropyl, cyclobutyl, cyclopentyl, cyclohexyl, cycloheptyl or cyclooctyl.

Cycloalkyl in R¹ is as defined above, preferably cyclopropyl, cyclobutyl, cyclopentyl or cyclohexyl.

Cycloalkyl in R² and R³ (independently of each other), are as defined above, preferably cyclopropyl, cyclobutyl, cyclopentyl or cyclohexyl.

Cycloalkyl in R⁴, R⁵, R⁶, R and R′ (independently of each other) are as defined above, preferably cyclopropyl, cyclobutyl, cyclopentyl or cyclohexyl.

The term “substituted C₁₋₄-alkyl” as used herein denotes a C₁₋₄-alkyl group which is substituted with 1-3 substituents, preferably 1-2 substituents, more preferably 1 substituent selected from C₃₋₈-cycloalkyl, aryl, heterocyclyl, substituted aryl or substituted heterocyclyl, wherein the substituents in substituted aryl or substituted heterocyclyl are 1, 2, 3 or 4 substituents, preferably 1 or 2 substituents, more preferably 1 substituent selected from C₁₋₄-alkoxy, phenyl, phenoxy, halogen, CN, NO₂, COR, CO₂R, CONRR′, NRR′, NHCOR, SO₂NRR′, SO₂R, C₁₋₄-alkyl and C₁₋₄-alkyl substituted with 1-3 halogens (wherein R and R′ are independently of each other as defined below). Preferably, the term “substituted C₁₋₄-alkyl” as used herein denotes a C₁₋₄-alkyl group substituted with 1-3 substituents, preferably 1-2 substituents, more preferably 1 substituent selected from C₃₋₈-cycloalkyl, aryl, heterocyclyl, substituted aryl and substituted heterocyclyl, wherein substituted aryl and substituted heterocyclyl are aryl or heterocyclyl are substituted with 1, 2, 3 or 4 substituents, preferably 1 or 2 substituents, more preferably 1 substituent selected from C₁₋₄-alkoxy, phenyl, phenoxy, halogen, CN, NO₂, COR, CO₂R, CONRR′, NRR′, NHCOR, SO₂NRR′, C₁₋₄-alkyl or C₁₋₄-alkyl substituted with 1-3 halogens. The term C₁₋₄-alkyl group as used herein denotes a C₁₋₄-alkyl as defined above, preferably a C₁₋₂-alkyl group, which is substituted with the aforementioned substituents; in case more than one substituent is attached to the C₁₋₄-alkyl group, these substituents can be identical or different from each other. Preferred substituents are aryl, heterocyclyl, substituted aryl or substituted heterocyclyl, more preferred substituents are phenyl, pyridyl, substituted phenyl or substituted pyridyl, wherein these substituents are substituted as mentioned above. Examples are cyclopropylmethyl, cyclobutylmethyl, cyclopentylmethyl, cyclohexylmethyl, 2-pyridylmethyl, 2-pyridylethyl, 2-pyridylpropyl, 2-pyridylbutyl, methyl-2-pyridyl-methyl, methyl-2-pyridyl-ethyl, dimethyl-2-pyridyl-methyl, ethyl-2-pyridyl-methyl, methoxy-2-pyridyl-methyl, methoxy-2-pyridyl-ethyl, dimethoxy-2-pyridyl-methyl, fluoro-2-pyridyl-methyl, difluoro-2-pyridyl-methyl, chloro-2-pyridyl-methyl, chloro-2-pyridyl-ethyl, dichloro-2-pyridyl-methyl, dichloro-2-pyridyl-methyl, bromo-2-pyridyl-methyl, dibromo-2-pyridyl-methyl, 3-pyridyl-methyl, 3-pyridyl-ethyl, 3-pyridyl-propyl, 3-pyridyl-butyl, methyl-3-pyridyl-methyl, methyl-3-pyridyl-ethyl, dimethyl-3-pyridyl-methyl, ethyl-3-pyridyl-methyl, methoxy-3-pyridyl-methyl, methoxy-3-pyridyl-ethyl, dimethoxy-3-pyridyl-methyl, fluoro-3-pyridyl-methyl, difluoro-3-pyridyl-methyl, chloro-3-pyridyl-methyl, chloro-3-pyridyl-ethyl, dichloro-3-pyridyl-methyl, dichloro-3-pyridyl-methyl, bromo-3-pyridyl-methyl, dibromo-3-pyridyl-methyl, 4-pyridyl-methyl, 4-pyridyl-ethyl, 4-pyridyl-propyl, 4-pyridyl-butyl, methyl-4-pyridyl-methyl, methyl-4-pyridyl-ethyl, dimethyl-4-pyridyl-methyl, ethyl-4-pyridyl-methyl, methoxy-4-pyridyl-methyl, methoxy-4-pyridyl-ethyl, dimethoxy-4-pyridyl-methyl, fluoro-4-pyridyl-methyl, difluoro-4-pyridyl-methyl, chloro-4-pyridyl-methyl, chloro-4-pyridyl-ethyl, dichloro-4-pyridyl-methyl, dichloro-4-pyridyl-methyl, bromo-4-pyridyl-methyl, dibromo-4-pyridyl-methyl, phenylmethyl (benzyl), phenylethyl, phenylpropyl, phenylbutyl, 2-methylphenylmethyl, 3-methylphenylmethyl, 4-methylphenylmethyl, 2-methylphenylethyl, 3-methylphenylethyl, 4-methylphenylethyl, 2,3-dimethylphenylmethyl, 2,4-dimethylphenylmethyl, 2,5-dimethylphenylmethyl, 2,6-dimethylphenylmethyl, 3,4-dimethylphenylmethyl, 3,5-dimethylphenylmethyl, 3,6-dimethylphenylmethyl, 2-ethylphenylmethyl, 3-ethylphenylmethyl, 4-ethylphenylmethyl, 2,3-diethylphenylmethyl, 2,4-diethylphenylmethyl, 2,5-diethylphenylmethyl, 2,6-diethylphenylmethyl, 3,4-diethylphenylmethyl, 3,5-diethylphenylmethyl, 3,6-diethylphenylmethyl, 2-trifluoromethyl-phenylmethyl, 3-trifluoromethyl-phenylmethyl, 4-trifluoromethyl-phenylmethyl, 2-trifluoromethyl-phenylethyl, 3-trifluoromethyl-phenylethyl, 4-trifluoromethyl-phenylethyl, 2,3-di-trifluoromethyl-phenylmethyl, 2,4-di-trifluoromethyl-phenylmethyl, 2,5-di-trifluoromethyl-phenylmethyl, 2,6-di-trifluoromethyl-phenylmethyl, 3,4-di-trifluoromethyl-phenylmethyl, 3,5-di-trifluoromethyl-phenylmethyl, 3,6-di-trifluoromethyl-phenylmethyl, 2-methoxy-phenylmethyl, 3-methoxy-phenylmethyl, 4-methoxy-phenylmethyl, 2-methoxy-phenylethyl, 3-methoxy-phenylethyl, 4-methoxy-phenylethyl, dimethoxy-phenylmethyl, dimethoxy-phenylethyl, 2,4,6-trimethoxy-phenylmethyl, 2-ethoxy-phenylmethyl, 3-ethoxy-phenylmethyl, 4-ethoxy-phenylmethyl, ethoxy-phenylethyl, diethoxy-phenylmethyl, diethoxy-phenylethyl, 2,4,6-triethoxy-phenylmethyl, 2-fluorophenylmethyl, 3-fluorophenylmethyl, 4-fluorophenylmethyl, 2,3-difluorophenylmethyl, 2,4-difluorophenylmethyl, 2,5-difluorophenylmethyl, 2,6-difluorophenylmethyl, 3,4-difluorophenylmethyl, 3,5-difluorophenylmethyl, 3,6-difluorophenylmethyl, 2-fluorophenylethyl, 3-fluorophenylethyl or 4-fluorophenylethyl, 2-chlorophenylmethyl, 3-chlorophenylmethyl, 4-chlorophenylmethyl, 2,3-dichlorophenylmethyl, 2,4-dichlorophenylmethyl, 2,5-dichlorophenylmethyl, 2,6-dichlorophenylmethyl, 3,4-dichlorophenylmethyl, 3,5-dichlorophenylmethyl, 3,6-dichlorophenylmethyl, 2-chlorophenylethyl, 3-chlorophenylethyl, 4-chlorophenylethyl, 2-bromophenylmethyl, 3-bromophenylmethyl, 4-bromophenylmethyl, 2,3-dibromophenylmethyl, 2,4-dibromophenylmethyl, 2,5-dibromophenylmethyl, 2,6-dibromophenylmethyl, 3,4-dibromophenylmethyl, 3,5-dibromophenylmethyl, 3,6-dibromophenylmethyl, 2-bromophenylethyl, 3-bromophenylethyl or 4-bromophenylethyl. 2-phenyl-phenylmethyl, 3-phenyl-phenylmethyl, 4-phenyl-phenylmethyl, 2-phenoxy-phenylmethyl, 3-phenoxy-phenylmethyl, 4-phenoxy-phenylmethyl, 2-nitro-phenylmethyl, 3-nitro-phenylmethyl, 4-nitro-phenylmethyl, 2-amino-phenylmethyl, 3-amino-phenylmethyl, 4-amino-phenylmethyl, 2-dimethylamino-phenylmethyl, 3-dimethylamino-phenylmethyl, 4-dimethylamino-phenylmethyl, 2-cyano-phenylmethyl, 3-cyano-phenylmethyl, 4-cyano-phenylmethyl, 2-methanesulfonyl-phenylmethyl, 3-methanesulfonyl-phenylmethyl, 4-methanesulfonyl-phenylmethyl, 2-acid methyl ester-phenylmethyl, 3-acid methyl ester-phenylmethyl or 4-acid methyl ester-phenylmethyl.

The term “substituted C₁₋₄-alkyl” for R¹ is as defined above.

For R² and R³ (independently of each other) the term “substituted C₁₋₄-alkyl” as used herein denotes a C₁₋₄-alkyl group which is substituted with 1-3 substituents, preferably 1-2 substituents, more preferably 1 substituent selected from C₃₋₈-cycloalkyl, aryl, heterocyclyl, substituted aryl and substituted heterocyclyl, wherein the substituents in substituted aryl and substituted heterocyclyl are 1, 2, 3 or 4 substituents, preferably 1 or 2 substituents, more preferably 1 substituent selected from C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens. Preferably, the term “substituted C₁₋₄-alkyl” as used herein denotes a C₁₋₄-alkyl group substituted with 1-3 substituents, preferably 1-2 substituents, more preferably 1 substituent selected from C₃₋₈-cycloalkyl, aryl, heterocyclyl, substituted aryl and substituted heterocyclyl, wherein the substituents in substituted aryl and substituted heterocyclyl are 1, 2, 3 or 4 substituents, preferably 1 or 2 substituents, more preferably1 substituent selected from C₁₋₄-alkoxy, halogen, CN, NO₂, COR, CO₂R, CONRR′, NRR′, NHCOR, SO₂NRR′, SO₂R, C₁₋₄-alkyl and C₁₋₄-alkyl substituted with 1-3 halogens (wherein R and R′ are, independently of each other, hydrogen or C₁₋₄-alkyl). The term C₁₋₄-alkyl group as used herein denotes a C₁₋₄-alkyl as defined above, preferably a C₁₋₂-alkyl group, which is substituted with the aforementioned substituents; in case more than one substituent is attached to the C₁₋₄-alkyl group, these substituents can be identical or different from each other. Preferred substituents are aryl, heterocyclyl, substituted aryl or substituted heterocyclyl, more preferably phenyl, pyridyl, substituted phenyl or substituted pyridyl, wherein these substituents are substituted as mentioned above. Examples are 2-pyridylmethyl, 2-pyridylethyl, 2-pyridylpropyl, 2-pyridylbutyl, methyl-2-pyridyl-methyl, methyl-2-pyridyl-ethyl, dimethyl-2-pyridyl-methyl, ethyl-2-pyridyl-methyl, methoxy-2-pyridyl-methyl, methoxy-2-pyridyl-ethyl, dimethoxy-2-pyridyl-methyl, fluoro-2-pyridyl-methyl, difluoro-2-pyridyl-methyl, chloro-2-pyridyl-methyl, chloro-2-pyridyl-ethyl, dichloro-2-pyridyl-methyl, dichloro-2-pyridyl-methyl, bromo-2-pyridyl-methyl, dibromo-2-pyridyl-methyl, 3-pyridyl-methyl, 3-pyridyl-ethyl, 3-pyridyl-propyl, 3-pyridyl-butyl, methyl-3-pyridyl-methyl, methyl-3-pyridyl-ethyl, dimethyl-3-pyridyl-methyl, ethyl-3-pyridyl-methyl, methoxy-3-pyridyl-methyl, methoxy-3-pyridyl-ethyl, dimethoxy-3-pyridyl-methyl, fluoro-3-pyridyl-methyl, difluoro-3-pyridyl-methyl, chloro-3-pyridyl-methyl, chloro-3-pyridyl-ethyl, dichloro-3-pyridyl-methyl, dichloro-3-pyridyl-methyl, bromo-3-pyridyl-methyl, dibromo-3-pyridyl-methyl, 4-pyridyl-methyl, 4-pyridyl-ethyl, 4-pyridyl-propyl, 4-pyridyl-butyl, methyl-4-pyridyl-methyl, methyl-4-pyridyl-ethyl, dimethyl-4-pyridyl-methyl, ethyl-4-pyridyl-methyl, methoxy-4-pyridyl-methyl, methoxy-4-pyridyl-ethyl, dimethoxy-4-pyridyl-methyl, fluoro-4-pyridyl-methyl, difluoro-4-pyridyl-methyl, chloro-4-pyridyl-methyl, chloro-4-pyridyl-ethyl, dichloro-4-pyridyl-methyl, dichloro-4-pyridyl-methyl, bromo-4-pyridyl-methyl, dibromo-4-pyridyl-methyl, phenylmethyl (benzyl), phenylethyl, phenylpropyl, phenylbutyl, 2-methylphenylmethyl, 3-methylphenylmethyl, 4-methylphenylmethyl, 2-methylphenylethyl, 3-methylphenylethyl, 4-methylphenylethyl, 2,3-dimethylphenylmethyl, 2,4-dimethylphenylmethyl, 2,5-dimethylphenylmethyl, 2,6-dimethylphenylmethyl, 3,4-dimethylphenylmethyl, 3,5-dimethylphenylmethyl, 3,6-dimethylphenylmethyl, 2-ethylphenylmethyl, 3-ethylphenylmethyl, 4-ethylphenylmethyl, 2,3-diethylphenylmethyl, 2,4-diethylphenylmethyl, 2,5-diethylphenylmethyl, 2,6-diethylphenylmethyl, 3,4-diethylphenylmethyl, 3,5-diethylphenylmethyl, 3,6-diethylphenylmethyl, 2-trifluoromethyl-phenylmethyl, 3-trifluoromethyl-phenylmethyl, 4-trifluoromethyl-phenylmethyl, 2-trifluoromethyl-phenylethyl, 2,3-di-trifluoromethyl-phenylmethyl, 2,4-di-trifluoromethyl-phenylmethyl, 2,5-di-trifluoromethyl-phenylmethyl, 2,6-di-trifluoromethyl-phenylmethyl, 3,4-di-trifluoromethyl-phenylmethyl, 3,5-di-trifluoromethyl-phenylmethyl, 3,6-di-trifluoromethyl-phenylmethyl, 2-methoxy-phenylmethyl, 3-methoxy-phenylmethyl, 4-methoxy-phenylmethyl, 2-methoxy-phenylethyl, 3-methoxy-phenylethyl, 4-methoxy-phenylethyl, dimethoxy-phenylmethyl, dimethoxy-phenylethyl, 2,4,6-trimethoxy-phenylmethyl, 2-ethoxy-phenylmethyl, 3-ethoxy-phenylmethyl, 4-ethoxy-phenylmethyl, ethoxy-phenylethyl, diethoxy-phenylmethyl, diethoxy-phenylethyl, 2,4,6-triethoxy-phenylmethyl, 2-fluorophenylmethyl, 3-fluorophenylmethyl, 4-fluorophenylmethyl, 2,3-difluorophenylmethyl, 2,4-difluorophenylmethyl, 2,5-difluorophenylmethyl, 2,6-difluorophenylmethyl, 3,4-difluorophenylmethyl, 3,5-difluorophenylmethyl, 3,6-difluorophenylmethyl, 2-fluorophenylethyl, 3-fluorophenylethyl or 4-fluorophenylethyl, 2-chlorophenylmethyl, 3-chlorophenylmethyl, 4-chlorophenylmethyl, 2,3-dichlorophenylmethyl, 2,4-dichlorophenylmethyl, 2,5-dichlorophenylmethyl, 2,6-dichlorophenylmethyl, 3,4-dichlorophenylmethyl, 3,5-dichlorophenylmethyl, 3,6-dichlorophenylmethyl, 2-chlorophenylethyl, 3-chlorophenylethyl, 4-chlorophenylethyl, 2-bromophenylmethyl, 3-bromophenylmethyl, 4-bromophenylmethyl, 2,3-dibromophenylmethyl, 2,4-dibromophenylmethyl, 2,5-dibromophenylmethyl, 2,6-dibromophenylmethyl, 3,4-dibromophenylmethyl, 3,5-dibromophenylmethyl, 3,6-dibromophenylmethyl, 2-bromophenylethyl, 3-bromophenylethyl or 4-bromophenylethyl. 2-phenyl-phenylmethyl, 3-phenyl-phenylmethyl, 4-phenyl-phenylmethyl, 2-phenoxy-phenylmethyl, 3-phenoxy-phenylmethyl, 4-phenoxy-phenylmethyl, 2-nitro-phenylmethyl, 3-nitro-phenylmethyl, 4-nitro-phenylmethyl, 2-amino-phenylmethyl, 3-amino-phenylmethyl, 4-amino-phenylmethyl, 2-dimethylamino-phenylmethyl, 3-dimethylamino-phenylmethyl, 4-dimethylamino-phenylmethyl, 2-cyano-phenylmethyl, 3-cyano-phenylmethyl, 4-cyano-phenylmethyl, 2-methanesulfonyl-phenylmethyl, 3-methanesulfonyl-phenylmethyl, 4-methanesulfonyl-phenylmethyl, 2-acid methyl ester-phenylmethyl, 3-acid methyl ester-phenylmethyl or 4-acid methyl ester-phenylmethyl.

The term “substituted C₁₋₄-alkyl” for R⁴, R⁵ or R⁶ are as defined for these substituents R² and R³ (see above).

The term “substituted C₁₋₄-alkyl” for or R and R′ (independently of each other) as used herein denotes a C₁₋₄-alkyl group which is substituted with 1-3 substituents, preferably 1-2 substituents, more preferably 1 substituent selected from C₃₋₈-cycloalkyl, aryl, heterocyclyl, substituted aryl and substituted heterocyclyl, wherein the substituents in substituted aryl and substituted heterocyclyl are 1, 2, 3 or 4 substituents, preferably 1 or 2 substituents, more preferably 1 substituent selected from C₃₋₈-cycloalkyl, aryl, heterocyclyl, substituted aryl and substituted heterocyclyl; wherein substituted aryl and substituted heterocyclyl means aryl or heterocyclyl substituted with C₁₋₄-alkoxy, halogen, CN, NO₂, COR⁷, CO₂R⁷, CONR⁷R⁸, NR⁷R⁸, NHCOR⁷, SO₂NR⁷R⁸, SO₂R⁷, C₁₋₄-alkyl or C₁₋₄-alkyl substituted with 1-3 halogens (wherein R⁷ and R⁸ are independently of each other hydrogen or C₁₋₄-alkyl). Preferably, the term “substituted C₁₋₄-alkyl” as used herein denotes a C₁₋₄-alkyl group substituted with 1-3 substituents, preferably 1-2 substituents, more preferably 1 substituent selected from C₃₋₈-cycloalkyl, aryl, heterocyclyl, substituted aryl and substituted heterocyclyl, wherein the substituents in substituted aryl and substituted heterocyclyl are 1, 2, 3 or 4 substituents, preferably 1 or 2 substituents, more preferably 1 substituent selected from C₁₋₄-alkoxy, halogen, CN, NO₂, COR⁷, CO₂R⁷, CONR⁷R⁸, NR⁷R⁸, NHCOR⁷, SO₂NR⁷R⁸, SO₂R⁷, C₁₋₄-alkyl and C₁₋₄-alkyl substituted with 1-3 halogens (wherein R⁷ and R⁸ are independently of each other hydrogen or C₁₋₄-alkyl). The term C₁₋₄-alkyl group as used herein denotes a C₁₋₄-alkyl as defined above, preferably a C₁₋₂-alkyl group, which is substituted with the aforementioned substituents; in case more than one substituent is attached to the C₁₋₄-alkyl group, these substituents can be identical or different from each other. Preferred substituents are aryl, heterocyclyl, substituted aryl or substituted heterocyclyl, more preferred substituents are phenyl, pyridyl, substituted phenyl or substituted pyridyl, wherein these substituents are substituted as mentioned above. Examples are cyclopropylmethyl, cyclobutylmethyl, cyclopentylpropyl, cyclohexylbutyl, 2-pyridylmethyl, 2-pyridylethyl, 2-pyridylpropyl, 2-pyridylbutyl, methyl-2-pyridyl-methyl, methyl-2-pyridyl-ethyl, dimethyl-2-pyridyl-methyl, ethyl-2-pyridyl-methyl, methoxy-2-pyridyl-methyl, methoxy-2-pyridyl-ethyl, dimethoxy-2-pyridyl-methyl, fluoro-2-pyridyl-methyl, difluoro-2-pyridyl-methyl, chloro-2-pyridyl-methyl, chloro-2-pyridyl-ethyl, dichloro-2-pyridyl-methyl, dichloro-2-pyridyl-methyl, bromo-2-pyridyl-methyl, dibromo-2-pyridyl-methyl, 3-pyridyl-methyl, 3-pyridyl-ethyl, 3-pyridyl-propyl, 3-pyridyl-butyl, methyl-3-pyridyl-methyl, methyl-3-pyridyl-ethyl, dimethyl-3-pyridyl-methyl, ethyl-3-pyridyl-methyl, methoxy-3-pyridyl-methyl, methoxy-3-pyridyl-ethyl, dimethoxy-3-pyridyl-methyl, fluoro-3-pyridyl-methyl, difluoro-3-pyridyl-methyl, chloro-3-pyridyl-methyl, chloro-3-pyridyl-ethyl, dichloro-3-pyridyl-methyl, dichloro-3-pyridyl-methyl, bromo-3-pyridyl-methyl, dibromo-3-pyridyl-methyl, 4-pyridyl-methyl, 4-pyridyl-ethyl, 4-pyridyl-propyl, 4-pyridyl-butyl, methyl-4-pyridyl-methyl, methyl-4-pyridyl-ethyl, dimethyl-4-pyridyl-methyl, ethyl-4-pyridyl-methyl, methoxy-4-pyridyl-methyl, methoxy-4-pyridyl-ethyl, dimethoxy-4-pyridyl-methyl, fluoro-4-pyridyl-methyl, difluoro-4-pyridyl-methyl, chloro-4-pyridyl-methyl, chloro-4-pyridyl-ethyl, dichloro-4-pyridyl-methyl, dichloro-4-pyridyl-methyl, bromo-4-pyridyl-methyl, dibromo-4-pyridyl-methyl, phenylmethyl (benzyl), phenylethyl, phenylpropyl, phenylbutyl, 2-methylphenylmethyl, 3-methylphenylmethyl, 4-methylphenylmethyl, 2-methylphenylethyl, 3-methylphenylethyl, 4-methylphenylethyl, 2,3-dimethylphenylmethyl, 2,4-dimethylphenylmethyl, 2,5-dimethylphenylmethyl, 2,6-dimethylphenylmethyl, 3,4-dimethylphenylmethyl, 3,5-dimethylphenylmethyl, 3,6-dimethylphenylmethyl, 2-ethylphenylmethyl, 3-ethylphenylmethyl, 4-ethylphenylmethyl, 2,3-diethylphenylmethyl, 2,4-diethylphenylmethyl, 2,5-diethylphenylmethyl, 2,6-diethylphenylmethyl, 3,4-diethylphenylmethyl, 3,5-diethylphenylmethyl, 3,6-diethylphenylmethyl, 2-trifluoromethyl-phenylmethyl, 3-trifluoromethyl-phenylmethyl, 4-trifluoromethyl-phenylmethyl, 2-trifluoromethyl-phenylethyl, 2,3-di-trifluoromethyl-phenylmethyl, 2,4-di-trifluoromethyl-phenylmethyl, 2,5-di-trifluoromethyl-phenylmethyl, 2,6-di-trifluoromethyl-phenylmethyl, 3,4-di-trifluoromethyl-phenylmethyl, 3,5-di-trifluoromethyl-phenylmethyl, 3,6-di-trifluoromethyl-phenylmethyl, 2-methoxy-phenylmethyl, 3-methoxy-phenylmethyl, 4-methoxy-phenylmethyl, 2-methoxy-phenylethyl, 3-methoxy-phenylethyl, 4-methoxy-phenylethyl, dimethoxy-phenylmethyl, dimethoxy-phenylethyl, 2,4,6-trimethoxy-phenylmethyl, 2-ethoxy-phenylmethyl, 3-ethoxy-phenylmethyl, 4-ethoxy-phenylmethyl, ethoxy-phenylethyl, diethoxy-phenylmethyl, diethoxy-phenylethyl, 2,4,6-triethoxy-phenylmethyl, 2-fluorophenylmethyl, 3-fluorophenylmethyl, 4-fluorophenylmethyl, 2,3-difluorophenylmethyl, 2,4-difluorophenylmethyl, 2,5-difluorophenylmethyl, 2,6-difluorophenylmethyl, 3,4-difluorophenylmethyl, 3,5-difluorophenylmethyl, 3,6-difluorophenylmethyl, 2-fluorophenylethyl, 3-fluorophenylethyl or 4-fluorophenylethyl, 2-chlorophenylmethyl, 3-chlorophenylmethyl, 4-chlorophenylmethyl, 2,3-dichlorophenylmethyl, 2,4-dichlorophenylmethyl, 2,5-dichlorophenylmethyl, 2,6-dichlorophenylmethyl, 3,4-dichlorophenylmethyl, 3,5-dichlorophenylmethyl, 3,6-dichlorophenylmethyl, 2-chlorophenylethyl, 3-chlorophenylethyl, 4-chlorophenylethyl, 2-bromophenylmethyl, 3-bromophenylmethyl, 4-bromophenylmethyl, 2,3-dibromophenylmethyl, 2,4-dibromophenylmethyl, 2,5-dibromophenylmethyl, 2,6-dibromophenylmethyl, 3,4-dibromophenylmethyl, 3,5-dibromophenylmethyl, 3,6-dibromophenylmethyl, 2-bromophenylethyl, 3-bromophenylethyl or 4-bromophenylethyl. 2-phenyl-phenylmethyl, 3-phenyl-phenylmethyl, 4-phenyl-phenylmethyl, 2-phenoxy-phenylmethyl, 3-phenoxy-phenylmethyl, 4-phenoxy-phenylmethyl, 2-nitro-phenylmethyl, 3-nitro-phenylmethyl, 4-nitro-phenylmethyl, 2-amino-phenylmethyl, 3-amino-phenylmethyl, 4-amino-phenylmethyl, 2-dimethylamino-phenylmethyl, 3-dimethylamino-phenylmethyl, 4-dimethylamino-phenylmethyl, 2-cyano-phenylmethyl, 3-cyano-phenylmethyl, 4-cyano-phenylmethyl, 2-methanesulfonyl-phenylmethyl, 3-methanesulfonyl-phenylmethyl, 4-methanesulfonyl-phenylmethyl, 2-acid methyl ester-phenylmethyl, 3-acid methyl ester-phenylmethyl or 4-acid methyl ester-phenylmethyl.

The term “alkoxy” as used herein, and if not specified by the number of carbon atoms, denotes a straight or branched chain alkyl-oxy group wherein the “alkyl” portion is as defined above such as methoxy, ethoxy, n-propyloxy, isopropyloxy, n-butyloxy, isobutyloxy, tert.-butyloxy, pentyloxy, hexyloxy, heptyloxy including their different isomers. More preferred alkoxy groups within the invention are methoxy, ethoxy, n-propyloxy, isopropyloxy, n-butyloxy, isobutyloxy or tert.-butyloxy.

The terms “COR, CO₂R, CONRR′, NRR′, NHCOR, SO₂NRR′, SO₂R” within the invention, R and R′ are, independently of each other, hydrogen, C₁₋₁₂-alkyl, substituted C₁₋₄-alkyl, C₃₋₈-cycloalkyl, aryl, substituted aryl, heterocyclyl and substituted heterocyclyl, wherein substituted C₁₋₄-alkyl means alkyl substituted with 1-3 substituents selected from C₃₋₈-cycloalkyl, aryl, heterocyclyl, substituted aryl and substituted heterocyclyl; wherein substituted aryl and substituted heterocyclyl means aryl or heterocyclyl substituted with C₁₋₄-alkoxy, halogen, CN, NO₂, COR⁷, CO₂R⁷, CONR⁷R⁸, NR⁷R⁸, NHCOR⁷, SO₂NR⁷R⁸, SO₂R⁷, C₁₋₄-alkyl or C₁₋₄-alkyl substituted with 1-3 halogens, and wherein substituted aryl are substituted with 1-5 substituents and substituted heterocyclyl are substituted with 1-4 substituents, these substituents selected from C₁₋₄-alkoxy, halogen, CN, NO₂, COR⁷, CO₂R⁷, CONR⁷R⁸, NR⁷R⁸, NHCOR⁷, SO₂NR⁷R⁸, SO₂R⁷, C₁₋₄-alkyl and C₁₋₄-alkyl substituted with 1-3 halogens (R⁷ and R⁸ are independently of each other hydrogen or C₁₋₄-alkyl). Preferably, R and/or R′ are independently of each other hydrogen, C₁₋₁₂-alkyl or aryl, more preferable hydrogen, methyl, ethyl, n-propyl, isopropyl, n-butyl, sec-butyl, isobutyl, tert.-butyl or phenyl. Examples for the terms “COR, CO₂R, CONRR′, NRR′, NHCOR, SO₂NRR′, SO₂R” are SO₂H, SO₂CH₃, SO₂C₂H₅, carboxylic acid methyl ester, carboxylic acid ethyl ester, amino, methylamino, dimethylamino or phenylamino.

The term “aryl” as used herein denotes a phenyl and naphthyl, both optionally benz-fused to an optionally substituted saturated, partially unsaturated or aromatic monocyclic, bicyclic or tricyclic heterocycle or carbocycle e.g. to cyclohexyl or cyclopentyl.

Aryl in R¹ is as defined above and is, most preferably phenyl.

Aryl in R² and R³ are, independently of each other, as defined above and are most preferably phenyl.

Aryl in R⁴, R⁵ or R and R′ (independently of each other) are as defined above, most preferably phenyl.

The term “aryl-C(═O)—,” as used herein for R⁴ or R⁵ denotes an aryl group as defined above (e.g. phenyl and naphthyl) attached to a keto function —C(═O)—. Preferred example is benzoyl.

The term “aryl-CH(OH)—” as used herein for R⁴ or R⁵ denotes an aryl group such as a phenyl or naphthyl group, preferably a phenyl group, attached to a hydroxy-methyl group. Preferred aryl-CH(OH)— is phenyl-CH(OH)—.

The term “substituted aryl” as used herein denotes substituted phenyl and naphthyl, both optionally benz-fused to an optionally substituted saturated, partially unsaturated or aromatic monocyclic, bicyclic or tricyclic heterocycle or carbocycle e.g. to cyclohexyl or cyclopentyl. Suitable substituents for aryl can be selected from 1, 2, 3, 4 or 5 substituents, or 1, 2, 3 or 4 substituent, preferably 1, 2 or 3 substituents, more preferably 1 or 2 substituents, and most preferably 1 substituent, wherein these substituents are selected from C₁₋₄-alkoxy, halogen, CN, NO₂, COR, CO₂R, CONRR′, NRR′, SO₂R, NHCOR, SO₂NRR′, C₁₋₄-alkyl and C₁₋₄-alkyl substituted with 1-3 halogens; in case more than one substituent is attached to the aryl group, these substituents can be identical or different from each other. Preferred substituents for aryl are selected from C₁₋₄-alkoxy, halogen, CN, NO₂, COR, CO₂R, CONRR′, NRR′, NHCOR, SO₂NRR′, C₁₋₄-alkyl and C₁₋₄-alkyl substituted with 1-3 halogens (wherein R and R′ are independently of each other as defined below). More preferably, substituents for aryl are selected from C₁₋₄-alkoxy, halogen, C₁₋₄-alkyl and C₁₋₄-alkyl substituted with 1-3 halogens. Examples of substituted aryl groups are 2-methyl-phenyl, 3-methyl-phenyl, 4-methyl-phenyl, 2,3-dimethylphenyl, 2,4-dimethylphenyl, 2,5-dimethylphenyl, 2,6-dimethylphenyl, 3,4-dimethylphenyl, 3,5-dimethylphenyl, 3,6-dimethylphenyl, 2-methoxy-phenyl, 3-methoxy-phenyl, 4-methoxy-phenyl, 2,3-dimethoxy-phenyl, 2,4-dimethoxy-phenyl, 2,5-dimethoxy-phenyl, 2,6-dimethoxy-phenyl, 3,4-dimethoxy-phenyl, 3,5-dimethoxy-phenyl, 3,6-dimethoxy-phenyl, 2-fluorophenyl, 3-fluorophenyl, 4-fluorophenyl, 2,3-difluorophenyl, 2,4-difluorophenyl, 2,5-difluorophenyl, 2,6-difluorophenyl, 3,4-difluorophenyl, 3,5-difluorophenyl, 3,6-difluorophenyl, 2-chlorophenyl, 3-chlorophenyl, 4-chlorophenyl, 2,3-dichlorophenyl, 2,4-dichlorophenyl, 2,5-dichlorophenyl, 2,6-dichlorophenyl, 3,4-dichlorophenyl, 3,5-dichlorophenyl, 3,6-dichlorophenyl, 2-bromophenyl, 3-bromophenyl, 4-bromophenyl, 2,3-dibromophenyl, 2,4-dibromophenyl, 2,5-dibromophenyl, 2,6-dibromophenyl, 3,4-dibromophenyl, 3,5-dibromophenyl, 3,6-dibromophenyl, 2-trifluoromethyl-phenyl, 3-trifluoromethyl-phenyl, 4-trifluoromethyl-phenyl, 2,3-di-trifluoromethyl-phenyl, 2,4-di-trifluoromethyl-phenyl, 2,5-di-trifluoromethyl-phenyl, 2,6-di-trifluoromethyl-phenyl, 3,4-di-trifluoromethyl-phenyl, 3,5-di-trifluoromethyl-phenyl, 3,6-di-trifluoromethyl-phenyl, 2-amino-phenyl, 3-amino-phenyl, 4-amino-phenyl, 2,3-di-amino-phenyl, 2,4-di-amino-phenyl, 2,5-di-amino-phenyl, 2,6-di-amino-phenyl, 3,4-di-amino-phenyl, 3,5-di-amino-phenyl, 3,6-di-amino-phenyl, 2-dimethylamino-phenyl, 3-dimethylamino-phenyl, 4-dimethylamino-phenyl, 2,3-di-dimethylamino-phenyl, 2,4-di-dimethylamino-phenyl, 2,5-di-dimethylamino-phenyl, 2,6-di-dimethylamino-phenyl, 3,4-di-dimethylamino-phenyl, 3,5-di-dimethylamino-phenyl, 3,6-di-dimethylamino-phenyl, 2-nitro-phenyl, 3-nitro-phenyl, 4-nitro-phenyl, 2,3-di-nitro-phenyl, 2,4-di-nitro-phenyl, 2,5-di-nitro-phenyl, 2,6-di-nitro-phenyl, 3,4-di-nitro-phenyl, 3,5-di-nitro-phenyl, 3,6-di-nitro-phenyl, 2-cyano-phenyl, 3-cyano-phenyl, 4-cyano-phenyl, 2,3-di-cyano-phenyl, 2,4-di-cyano-phenyl, 2,5-di-cyano-phenyl, 2,6-di-cyano-phenyl, 3,4-di-cyano-phenyl, 3,5-di-cyano-phenyl, 3,6-di-cyano-phenyl, 2-carboxylic acid-phenyl, 3-carboxylic acid-phenyl, 4-carboxylic acid-phenyl, 2,3-di-carboxylic acid-phenyl, 2,4-di-carboxylic acid-phenyl, 2,5-di-carboxylic acid-phenyl, 2,6-di-carboxylic acid-phenyl, 3,4-di-carboxylic acid-phenyl, 3,5-di-carboxylic acid-phenyl, 3,6-di-carboxylic acid-phenyl, 2-carboxylic acid methyl ester-phenyl, 3-carboxylic acid methyl ester-phenyl, 4-carboxylic acid methyl ester-phenyl, 2,3-di-carboxylic acid methyl ester-phenyl, 2,4-di-carboxylic acid methyl ester-phenyl, 2,5-di-carboxylic acid methyl ester-phenyl, 2,6-di-carboxylic acid methyl ester-phenyl, 3,4-di-carboxylic acid methyl ester-phenyl, 3,5-di-carboxylic acid methyl ester-phenyl or 3,6-di-carboxylic acid methyl ester-phenyl.

Substituted aryl for R¹, R² and R³ (independently of each other), R⁴, R⁵, R and R′ (independently of each other) are as defined above.

The term “substituted aryl-C(═O)—” as used herein for R⁴ or R⁵ denotes a substituted aryl group as defined above, attached to a keto function —C(═O)—. Suitable substituents for substituted aryl-C(═O)— can be selected from 1, 2, 3, 4 or 5 substituents, or 1, 2, 3 or 4 substituent, preferably 1, 2 or 3 substituents, more preferably 1 or 2 substituents, and most preferably 1 substituent, wherein these substituents are selected from C₁₋₄-alkoxy, halogen, CN, NO₂, COR, CO₂R, CONRR′, NRR′, SO₂R, NHCOR, SO₂NRR′, C₁₋₄-alkyl and C₁₋₄-alkyl substituted with 1-3 halogens; in case more than one substituent is attached to the aryl group, these substituents can be identical or different from each other. Preferred substituents for aryl are selected from C₁₋₄-alkoxy, halogen, CN, NO₂, COR, CO₂R, CONRR′, NRR′, NHCOR, SO₂NRR′, C₁₋₄-alkyl and C₁₋₄-alkyl substituted with 1-3 halogens (wherein R and R′ are independently of each other as defined below). More preferably, substituents for substituted aryl-C(═O)— are selected from C₁₋₄-alkoxy, halogen, C₁₋₄-alkyl and C₁₋₄-alkyl substituted with 1-3 halogens.

The term “substituted aryl-CH(OH)—” as used herein for R⁴ or R⁵ denotes a substituted phenyl group or a substituted naphthyl group, preferably a substituted phenyl group, attached to a hydroxy-methyl group. Suitable substituents for substituted aryl-CH(OH)— can be selected from 1, 2, 3, 4 or 5 substituents, or 1, 2, 3 or 4 substituent, preferably 1, 2 or 3 substituents, more preferably 1 or 2 substituents, and most preferably 1 substituent, wherein these substituents are selected from C₁₋₄-alkoxy, halogen, CN, NO₂, COR, CO₂R, CONRR′, NRR′, SO₂R, NHCOR, SO₂NRR′, C₁₋₄-alkyl and C₁₋₄-alkyl substituted with 1-3 halogens; in case more than one substituent is attached to the aryl group, these substituents can be identical or different from each other. Preferred substituents for aryl are selected from C₁₋₄-alkoxy, halogen, CN, NO₂, COR, CO₂R, CONRR′, NRR′, NHCOR, SO₂NRR′, C₁₋₄-alkyl and C₁₋₄-alkyl substituted with 1-3 halogens (wherein R and R′ are independently of each other as defined below). More preferably, substituents for substituted aryl-CH(OH)— are selected from C₁₋₄-alkoxy, halogen, C₁₋₄-alkyl and C₁₋₄-alkyl substituted with 1-3 halogens. Examples are the aforementioned substituted aryl groups attached to a hydroxy-methyl group, such as 2-methyl-phenyl-hydroxymethyl, 3-methyl-phenyl-hydroxymethyl, 4-methyl-phenyl-hydroxymethyl, 2,3-dimethylphenyl-hydroxymethyl, 2,4-dimethylphenyl-hydroxymethyl, 2,5-dimethylphenyl-hydroxymethyl, 2,6-dimethylphenyl-hydroxymethyl, 3,4-dimethylphenyl-hydroxymethyl, 3,5-dimethylphenyl-hydroxymethyl, 3,6-dimethylphenyl-hydroxymethyl, 2-methoxy-phenyl-hydroxymethyl, 3-methoxy-phenyl-hydroxymethyl, 4-methoxy-phenyl-hydroxymethyl, 2,3-dimethoxy-phenyl-hydroxymethyl, 2,4-dimethoxy-phenyl-hydroxymethyl, 2,5-dimethoxy-phenyl-hydroxymethyl, 2,6-dimethoxy-phenyl-hydroxymethyl, 3,4-dimethoxy-phenyl-hydroxymethyl, 3,5-dimethoxy-phenyl-hydroxymethyl, 3,6-dimethoxy-phenyl-hydroxymethyl.

The term “heterocyclyl” as used herein denotes an aromatic or non-aromatic monocyclic or bicyclic heterocyclic system which contains 1, 2, 3 or 4 hetero atoms, preferably 1, 2 or 3 hetero atoms, with the hetero atoms being selected from nitrogen, oxygen and sulfur. Examples of heterocyclyl are 2-furyl, 3-furyl, 1-pyrrolyl, 2-pyrrolyl, 2-pyridyl, 3-pyridyl, 4-pyridyl, 1-indolyl, 2-indolyl or 3-indolyl, pyridazin-3-yl, pyridazin-4-yl, thiophen-2-yl, thiophen-3-yl, [1,3,4]thiadiazol-2-yl, [1,3,4]thiadiazol-5-yl, or tetrahydro-pyran-4-yl, piperidin-2-yl, piperidin-3-yl, piperidin-4-yl, 1H-imidazol-2-yl, 1H-imidazol-4-yl, 1H-imidazol-5-yl, pyrrolidin-1-yl, pyrrolidin-2-yl, pyrrolidin-3-yl, pyrrolidin-4-yl or pyrrolidin-5-yl.

Heterocyclyl for R¹ is as defined above and is, preferably, 2-pyridyl, 3-pyridyl or 4-pyridyl.

Heterocyclyl for R² and R³ (independently of each other), R⁴, R⁵ or R and R′ (independently of each other) are as defined above. Examples are 2-furyl, 3-furyl, 1-pyrrolyl, 2-pyrrolyl, 2-pyridyl, 3-pyridyl, 4-pyridyl, 1-indolyl, 2-indolyl or 3-indolyl, pyridazin-3-yl, pyridazin-4-yl, thiophen-2-yl, thiophen-3-yl, [1,3,4]thiadiazol-2-yl or tetrahydro-pyran-4-yl, piperidin-2-yl, piperidin-3-yl, piperidin-4-yl, 1H-imidazol-2-yl, 1H-imidazol-4-yl, 1H-imidazol-5-yl, pyrrolidin-1-yl, pyrrolidin-2-yl, pyrrolidin-3-yl, pyrrolidin-4-yl or pyrrolidin-5-yl.

The term “heterocyclyl-C(═O)—,” as used herein for R⁴ or R⁵ denotes a heterocyclyl group such as defined above (e.g. 2-furyl, 3-furyl, 1-pyrrolyl, 2-pyrrolyl, 2-pyridyl, 3-pyridyl, 4-pyridyl, 1-indolyl, 2-indolyl or 3-indolyl, pyridazin-3-yl, pyridazin-4-yl, thiophen-2-yl, thiophen-3-yl, [1,3,4]thiadiazol-2-yl, [1,3,4]thiadiazol-5-yl, or tetrahydro-pyran-4-yl, piperidin-2-yl, piperidin-3-yl, piperidin-4-yl, 1H-imidazol-2-yl, 1H-imidazol-4-yl, 1H-imidazol-5-yl, pyrrolidin-1-yl, pyrrolidin-2-yl, pyrrolidin-3-yl, pyrrolidin-4-yl or pyrrolidin-5-yl) attached to a keto function —C(═O)—.

The term “heterocyclyl-CH(OH)—” as used herein for R⁴ and R⁵ denotes a heterocyclyl group such as defined above (e.g. 2-furyl, 3-furyl, 1-pyrrolyl, 2-pyrrolyl, 2-pyridyl, 3-pyridyl, 4-pyridyl, 1-indolyl, 2-indolyl or 3-indolyl, pyridazin-3-yl, pyridazin-4-yl, thiophen-2-yl, thiophen-3-yl, [1,3,4]thiadiazol-2-yl, [1,3,4]thiadiazol-5-yl, or tetrahydro-pyran-4-yl, piperidin-2-yl, piperidin-3-yl, piperidin-4-yl, 1H-imidazol-2-yl, 1H-imidazol-4-yl, 1H-imidazol-5-yl, pyrrolidin-1-yl, pyrrolidin-2-yl, pyrrolidin-3-yl, pyrrolidin-4-yl or pyrrolidin-5-yl) attached to a hydroxy-methyl group.

The term “substituted heterocyclyl” as used herein denotes substituted aromatic or non-aromatic monocyclic or bicyclic heterocyclic systems which contain one or more hetero atoms selected from nitrogen, oxygen and sulfur, such as 2-furyl, 3-furyl, 1-pyrrolyl, 2-pyrrolyl, 2-pyridyl, 3-pyridyl, 4-pyridyl, 1-indolyl, 2-indolyl or 3-indolyl, [1,3,4]thiadiazol-2-yl, [1,3,4]thiadiazol-5-yl, or piperidin-4-yl, pyridazin-3-yl, pyridazin-4-yl, thiophen-2-yl, thiophen-3-yl, tetrahydro-pyran-4yl, piperidin-4-yl, 1H-imidazol-2-yl, 1H-imidazol-4-yl, 1H-imidazol-5-yl, pyrrolidin-1-yl, pyrrolidin-2-yl, pyrrolidin-3-yl, pyrrolidin-4-yl, pyrrolidin-5-yl. Suitable substituents for heterocyclyl can be selected from 1, 2, 3 or 4 substituents, preferably 1, 2 or 3 substituents, more preferably 1 or 2 substituents, and most preferably 1 substituent, wherein these substituents are selected from C₁₋₄-alkoxy, halogen, CN, NO₂, COR, CO₂R, CONRR′, NRR′, SO₂R, NHCOR, SO₂NRR′, C₁₋₄-alkyl and C₁₋₄-alkyl substituted with 1-3 halogens (wherein R and R′ are as defined below); in case more than one substituent is attached to the heterocyclyl group, these substituents can be identical or different from each other. Preferred substituents for heterocyclyl are selected from C₁₋₄-alkoxy, halogen, CN, NO₂, COR, CO₂R, CONRR′, NRR′, NHCOR, SO₂NRR′, C₁₋₄-alkyl and C₁₋₄-alkyl substituted with 1-3 halogens. More preferable substituents for heterocyclyl are selected from C₁₋₄-alkoxy, COR, halogen, C₁₋₄-alkyl and C₁₋₄-alkyl substituted with 1-3 halogens, more preferred substituents for heterocyclyl are selected from C₁₋₄-alkoxy, halogen, C₁₋₄-alkyl and C₁₋₄-alkyl substituted with 1-3 halogens. Examples of substituted heterocyclyl groups are 2-methyl-pyridyl, 3-methyl-pyridyl, 4-methyl-pyridyl, 2,3-dimethylpyridyl, 2,4-dimethylpyridyl, 2,5-dimethylpyridyl, 2,6-dimethylpyridyl, 3,4-dimethylpyridyl, 3,5-dimethylpyridyl, 3,6-dimethylpyridyl, 2-methoxy-pyridyl, 3-methoxy-pyridyl, 4-methoxy-pyridyl, 2,3-dimethoxy-pyridyl, 2,4-dimethoxy-pyridyl, 2,5-dimethoxy-pyridyl, 2,6-dimethoxy-pyridyl, 3,4-dimethoxy-pyridyl, 3,5-dimethoxy-pyridyl, 3,6-dimethoxy-pyridyl, 2-fluoro-pyridyl, 3-fluoro-pyridyl, 4-fluoro-pyridyl, 2,3-difluoro-pyridyl, 2,4-difluoro-pyridyl, 2,5-difluoro-pyridyl, 2,6-difluoro-pyridyl, 3,4-difluoro-pyridyl, 3,5-difluoro-pyridyl, 3,6-difluoro-pyridyl, 2-chloro-pyridyl, 3-chloro-pyridyl, 4-chloro-pyridyl, 2,3-dichloro-pyridyl, 2,4-dichloro-pyridyl, 2,5-dichloro-pyridyl, 2,6-dichloro-pyridyl, 3,4-dichloro-pyridyl, 3,5-dichloro-pyridyl, 3,6-dichloro-pyridyl, 2-bromo-pyridyl, 3-bromo-pyridyl, 4-bromo-pyridyl, 2,3-dibromo-pyridyl, 2,4-dibromo-pyridyl, 2,5-dibromo-pyridyl, 2,6-dibromo-pyridyl, 3,4-dibromo-pyridyl, 3,5-dibromo-pyridyl, 3,6-dibromo-pyridyl, 2-trifluoromethyl-pyridyl, 3-trifluoromethyl-pyridyl, 4-trifluoromethyl-pyridyl, 2,3-di-trifluoromethyl-pyridyl, 2,4-di-trifluoromethyl-pyridyl, 2,5-di-trifluoromethyl-pyridyl, 2,6-di-trifluoromethyl-pyridyl, 3,4-di-trifluoromethyl-pyridyl, 3,5-di-trifluoromethyl-pyridyl, 3,6-di-trifluoromethyl-pyridyl, 5-methyl-[1,3,4]thiadiazol-2-yl, 2-methyl-[1,3,4]thiadiazol-5-yl, 5-ethyl-[1,3,4]thiadiazol-2-yl, 2-ethyl-[1,3,4]thiadiazol-5-yl, 1-formyl-piperidin-4-yl, 2-formyl-piperidin-4-yl or 3-formyl-piperidin-4-yl. For all the cited examples for “heterocyclyl” these substituents can be at any chemically possible position. For example methylpyridyl means that the methyl substituent may be attached in the 3, 4, 5 or 6 position of a 2-pyridyl or in the 2, 4, 5 or 6 position of a 3-pyridyl or in the 2, 3, 5 or 6 position of a 4-pyridyl.

Substituted heterocyclyl in R¹ is as defined above, preferably 2-pyridyl, 3-pyridyl or 4-pyridyl, substituted with these substituents as defined above.

Substituted heterocyclyl for R² and R³ (independently of each other), R and R′ (independently of each other), R⁴ and R⁵ are as defined above.

The term “substituted heterocyclyl-CH(OH)—” as used herein for R⁴ or R⁵ denotes a substituted heterocyclyl group such as defined above attached to a hydroxy-methyl group. Suitable substituents for substituted heterocyclyl-CH(OH)— can be selected from 1, 2, 3 or 4 substituents, preferably 1, 2 or 3 substituents, more preferably 1 or 2 substituents, and most preferably 1 substituent, wherein these substituents are selected from C₁₋₄-alkoxy, halogen, CN, NO₂, COR, CO₂R, CONRR′, NRR′, SO₂R, NHCOR, SO₂NRR′, C₁₋₄-alkyl and C₁₋₄-alkyl substituted with 1-3 halogens; in case more than one substituent is attached to the heterocyclyl group, these substituents can be identical or different from each other. Preferred substituents for heterocyclyl are selected from C₁₋₄-alkoxy, halogen, CN, NO₂, COR, CO₂R, CONRR′, NRR′, NHCOR, SO₂NRR′, C₁₋₄-alkyl and C₁₋₄-alkyl substituted with 1-3 halogens (wherein R and R′ are independently of each other as defined below). More preferably, substituents for substituted heterocyclyl —C(═O)— are selected from C₁₋₄-alkoxy, halogen, C₁₋₄-alkyl and C₁₋₄-alkyl substituted with 1-3 halogens.

The term “substituted heterocyclyl-C(═O)—” as used herein for R⁴ or R⁵ denotes a substituted heterocyclyl group such as defined above attached to a keto function —C(═O)—. Suitable substituents for substituted heterocyclyl-C(═O)— can be selected from 1, 2, 3 or 4 substituents, preferably 1, 2 or 3 substituents, more preferably 1 or 2 substituents, and most preferably 1 substituent, wherein these substituents are selected from C₁₋₄-alkoxy, halogen, CN, NO₂, COR, CO₂R, CONRR′, NRR′, SO₂R, NHCOR, SO₂NRR′, C₁₋₄-alkyl and C₁₋₄-alkyl substituted with 1-3 halogens; in case more than one substituent is attached to the heterocyclyl group, these substituents can be identical or different from each other. Preferred substituents for heterocyclyl are selected from C₁₋₄-alkoxy, halogen, CN, NO₂, COR, CO₂R, CONRR′, NRR′, NHCOR, SO₂NRR′, C₁₋₄-alkyl and C₁₋₄-alkyl substituted with 1-3 halogens (wherein R and R′ are independently of each other as defined below). More preferably, substituents for substituted heterocyclyl-C(═O)— are selected from C₁₋₄-alkoxy, halogen, C₁₋₄-alkyl and C₁₋₄-alkyl substituted with 1-3 halogens.

The term halogen stands for fluorine, chlorine, bromine and iodine.

The term “X” represents S and O, preferably O.

The compounds of the instant invention may contain an olefinic double bond, this can have the (E) or (Z) configuration. All such isomeric forms of these compounds are embraced by the present invention. The independent syntheses of these compounds or their chromatograpic separations may be achieved as known in the art by appropriate modification of the methodology disclosed herein.

Any functional (i.e. reactive) group present in any of the compounds of the invention may be protected with a protecting group which is known per se, for example, as described in “Protective Groups in Organic Synthesis”, 2nd Ed., T. W. Greene and P. G. M. Wuts, John Wiley & Sons, New York, N.Y., 1991. Groups which are to be protected are for example “hydroxy groups”, “carboxylic acid groups” “amino groups” and “ketone groups”. The term “hydroxy protecting group” includes protecting groups which are usually used to replace the proton of the hydroxy group. The term “carboxylic acid protecting group” includes protecting groups which are usually used to replace the proton of the carboxyl group. The term “amino protecting group” as used herein includes protecting groups that are usually used to replace one proton or both protons of the amino group. Such groups are often employed in peptide chemistry. The term “ketone protecting group” includes protecting groups known in the art such as ketals or thioketals.

Compounds of formula I which are acidic can form pharmaceutically acceptable salts with bases such as alkali metal hydroxides (e.g. sodium hydroxide and potassium hydroxide), alkaline earth metal hydroxides (e.g. calcium hydroxide, barium hydroxide and magnesium hydroxide), and with organic bases (e.g. N-ethyl piperidine, dibenzylamine, and the like). Those compounds of formula (I) which are basic can form pharmaceutically acceptable salts with inorganic acids such as hydrohalic acids (e.g. hydrochloric acid and hydrobromic acid), sulphuric acid, nitric acid and phosphoric acid, and the like, and with organic acids (e.g. with acetic acid, tartaric acid, succinic acid, fumaric acid, maleic acid, malic acid, salicylic acid, citric acid, methanesulphonic acid and p-toluene sulphonic acid, and the like). The formation and isolation of such salts can be carried out according to methods known in the art.

A preferred embodiment of the invention are novel compounds of formula I

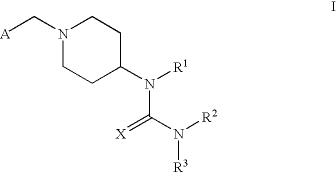

wherein

R¹ is hydrogen, C₁₋₁₂-alkyl, C₃₋₈-cycloalkyl, allyl, substituted C₁₋₄-alkyl, aryl, substituted aryl, heterocyclyl or substituted heterocyclyl,

wherein substituted C₁₋₄-alkyl means alkyl substituted with 1-3 substituents selected from C₃₋₈-cycloalkyl, aryl, heterocyclyl, substituted aryl and substituted heterocyclyl;

wherein substituted aryl and substituted heterocyclyl means aryl and heterocyclyl substituted with C₁₋₄-alkoxy, phenyl, phenoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens, and

wherein substituted aryl means aryl substituted with 1-5 substituents and substituted heterocyclyl means heterocyclyl substituted with 1-4 substituents and these substituents are selected from C₁₋₄-alkoxy, halogen, CN, NO₂, COR, CO₂R, CONRR′, NRR′, SO₂R, NHCOR, SO₂NRR′, C₁₋₄-alkyl and C₁₋₄-alkyl substituted with 1-3 halogens;

R² and R³ are independently of each other hydrogen, C₁₋₁₂-alkyl, C₃₋₈-cycloalkyl, allyl, substituted C₁₋₄-alkyl, aryl, substituted aryl, heterocyclyl or substituted heterocyclyl, wherein substituted C₁₋₄-alkyl means alkyl substituted with 1-3 substituents selected from C₃₋₈-cycloalkyl, aryl, heterocyclyl, substituted aryl and substituted heterocyclyl;

wherein substituted aryl and substituted heterocyclyl means aryl and heterocyclyl substituted with C₁₋₄-alkoxy, halogen, CN, NO₂, COR, CO₂R, CONRR′, NRR′, SO₂R, NHCOR, SO₂NRR′, C₁₋₄-alkyl or C₁₋₄-alkyl substituted with 1-3 halogens, and

wherein substituted aryl means aryl substituted with 1-5 substituents and substituted heterocyclyl means heterocyclyl substituted with 1-4 substituents and these substituents are selected from C₁₋₄-alkoxy, halogen, CN, NO₂, COR, CO₂R, CONRR′, NRR′, SO₂R, NHCOR, SO₂NRR′, C₁₋₄-alkyl and C₁₋₄-alkyl substituted with 1-3 halogens;

X is S or O;

A is selected from the group consisting of:

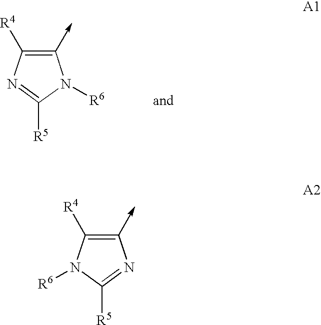

wherein

R⁴ is hydrogen, C₁₋₁₂-alkyl, substituted C₁₋₄-alkyl, C₃₋₈-cycloalkyl, C₁₋₄-alkoxy, CN, COR, CO₂R, CONRR′, NHCOR, aryl, substituted aryl, aryl-C(═O)—, substituted aryl-C(═O)—, aryl-CH(OH)—, substituted aryl-CH(OH)—, heterocyclyl, substituted heterocyclyl, heterocyclyl-C(═O)—, substituted heterocyclyl-C(═O)—, heterocyclyl-CH(OH)—, substituted heterocyclyl-CH(OH)— or NRR′,

wherein substituted C₁₋₄-alkyl means alkyl substituted with 1-3 substituents selected from C₃₋₈-cycloalkyl, aryl, heterocyclyl, substituted aryl and substituted heterocyclyl;

wherein substituted aryl and substituted heterocyclyl means aryl and heterocyclyl substituted with C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens, and

wherein substituted aryl, substituted aryl-C(═O)— or substituted aryl-CH(OH)— are substituted with 1-5 substituents selected from C₁₋₄-alkoxy, halogen, CN, NO₂, COR, CO₂R, CONRR′, NRR′, NHCOR, SO₂NRR′, SO₂R, C₁₋₄-alkyl and C₁₋₄-alkyl substituted with 1-3 halogens, and

wherein substituted heterocyclyl, substituted heterocyclyl-C(═O)— or substituted heterocyclyl-CH(OH)— are substituted with 1-4 substituents selected from C₁₋₄-alkoxy, halogen, CN, NO₂, COR, CO₂R, CONRR′, NRR′, NHCOR, SO₂NRR′, SO₂R, C₁₋₄-alkyl and C₁₋₄-alkyl substituted with 1-3 halogens;

R⁵ is hydrogen, C₁₋₁₂-alkyl, substituted C₁₋₄-alkyl, C₃₋₈-cycloalkyl, C₁₋₄-alkoxy, halogen, COR, aryl, substituted aryl, aryl-C(═O)—, substituted aryl-C(═O)—, aryl-CH(OH)—, substituted aryl-CH(OH)—, heterocyclyl, substituted heterocyclyl, heterocyclyl-C(═O)—, substituted heterocyclyl-C(═O)—, heterocyclyl-CH(OH)—, substituted heterocyclyl-CH(OH)— or NRR′,

from C₃₋₈-cycloalkyl, aryl, heterocyclyl, substituted aryl and substituted heterocyclyl;

wherein substituted aryl and substituted heterocyclyl means aryl and heterocyclyl substituted with C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens, and

wherein substituted aryl, substituted aryl-C(═O)— or substituted aryl-CH(OH)— are substituted with 1-5 substituents selected from C₁₋₄-alkoxy, halogen, CN, NO₂, COR, CO₂R, CONRR′, NRR′, NHCOR, SO₂NRR′, SO₂R, C₁₋₄-alkyl and C₁₋₄-alkyl substituted with 1-3 halogens, and

wherein substituted heterocyclyl, substituted heterocyclyl-C(═O)— or substituted heterocyclyl-CH(OH)— are substituted with 1-4 substituents selected from C₁₋₄-alkoxy, halogen, CN, NO₂, COR, CO₂R, CONRR′, NRR′, NHCOR, SO₂NRR′, SO₂R, C₁₋₄-alkyl and C₁₋₄-alkyl substituted with 1-3 halogens;

R⁶ is hydrogen, C₁₋₁₂-alkyl, substituted C₁₋₄-alkyl, C₁₋₄-alkoxy, C₃₋₈-cycloalkyl, COR, CO₂R, CONRR′, NHCOR, SO₂NRR′, SO₂R,

wherein substituted C₁₋₄-alkyl means alkyl substituted with 1-3 substituents selected from C₃₋₈-cycloalkyl, aryl, heterocyclyl, substituted aryl and substituted heterocyclyl;

wherein substituted aryl and substituted heterocyclyl means aryl and heterocyclyl substituted with C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens;

R and R′ are independently of each other hydrogen, C₁₋₁₂-alkyl, substituted C₁₋₄-alkyl, C₃₋₈-cycloalkyl, aryl, substituted aryl, heterocyclyl and substituted heterocyclyl,

wherein substituted C₁₋₄-alkyl means alkyl substituted with 1-3 substituents selected from C₃₋₈-cycloalkyl, aryl, heterocyclyl, substituted aryl and substituted heterocyclyl;

wherein substituted aryl and substituted heterocyclyl means aryl and heterocyclyl substituted with C₁₋₄-alkoxy, halogen, CN, NO₂, COR⁷, CO₂R⁷, CONR⁷R⁸, NR⁷R⁸, NHCOR⁷, SO₂NR⁷R⁸, SO₂R⁷, C₁₋₄-alkyl or C₁₋₄-alkyl substituted with 1-3 halogens, and

wherein substituted aryl are substituted with 1-5 substituents and substituted heterocyclyl are substituted with 1-4 substituents, these substituents selected from C₁₋₄-alkoxy, halogen, CN, NO₂, COR⁷, CO₂R⁷, CONR⁷R⁸, NR⁷R⁸, NHCOR⁷, SO₂NR⁷R⁸, SO₂R⁷, C₁₋₄-alkyl and C₁₋₄-alkyl substituted with 1-3 halogens;

R⁷ and R⁸ are independently of each other hydrogen or C₁₋₄-alkyl; as well as ethers or hydrolyzable esters of compounds of formula I and pharmaceutically acceptable salts thereof.

Other preferred embodiments of the invention are novel compounds of formula I wherein

R¹ is hydrogen, C₁₋₁₂-alkyl, C₃₋₈-cycloalkyl, allyl, substituted C₁₋₄-alkyl, aryl, substituted aryl or heterocyclyl,

wherein substituted C₁₋₄-alkyl means alkyl substituted with 1-3 substituents selected from C₃₋₈-cycloalkyl, aryl, heterocyclyl, substituted aryl and substituted heterocyclyl;

wherein substituted aryl and substituted heterocyclyl means aryl and heterocyclyl substituted with C₁₋₄-alkoxy, phenyl, phenoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens, and

wherein substituted aryl means aryl substituted with 1-5 substituents selected from C₁₋₄-alkoxy, halogen, CN, NO₂, COR, CO₂R, CONRR′, NRR′, SO₂R, NHCOR, SO₂NRR′, C₁₋₄-alkyl and C₁₋₄-alkyl substituted with 1-3 halogens, preferably wherein

R¹ is hydrogen, C₁₋₁₂-alkyl, C₃₋₈-cycloalkyl, allyl, substituted C₁₋₄-alkyl, phenyl, substituted phenyl or pyridyl,

wherein substituted C₁₋₄-alkyl means alkyl substituted with 1-3 substituents selected from C₃₋₈-cycloalkyl, phenyl, pyridyl, substituted phenyl and substituted pyridyl;

wherein substituted phenyl and substituted pyridyl are substituted with C₁₋₄-alkoxy, phenyl, phenoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens, and

wherein substituted phenyl is substituted with 1-5 substituents selected from C₁₋₄-alkoxy, halogen, CN, NO₂, COR, CO₂R, CONRR′, NRR′, SO₂R, NHCOR, SO₂NRR′, C₁₋₄-alkyl and C₁₋₄-alkyl substituted with 1-3 halogens, more preferably wherein

R¹ is hydrogen, C₁₋₁₂-alkyl, C₃₋₈-cycloalkyl, allyl, substituted C₁₋₄-alkyl, phenyl, substituted phenyl or pyridyl,

wherein substituted C₁₋₄-alkyl means alkyl substituted with 1-3 substituents selected from C₃₋₈-cycloalkyl, phenyl, pyridyl and substituted phenyl; wherein substituted phenyl is substituted with C₁₋₄-alkoxy, phenyl, phenoxy, halogen, CN, NO₂, CO₂R, NRR′, SO₂R, C₁₋₄-alkyl or C₁₋₄-alkyl substituted with 1-3 halogens, and

wherein substituted phenyl is substituted with 1-5 substituents selected from C₁₋₄-alkoxy, halogen, C₁₋₄-alkyl and C₁₋₄-alkyl substituted with 1-3 halogens, most preferably wherein

R¹ is hydrogen, C₁₋₁₂-alkyl, C₃₋₈-cycloalkyl, allyl, substituted C₁₋₄-alkyl, phenyl, substituted phenyl or pyridyl,

wherein substituted C₁₋₄-alkyl means alkyl substituted with 1-3 substituents selected from C₃₋₈-cycloalkyl, phenyl, pyridyl and substituted phenyl; wherein substituted phenyl is substituted with C₁₋₄-alkoxy, phenyl, phenoxy, chlorine, CN, NO₂, CO₂R, NRR′, SO₂R, C₁₋₄-alkyl or C₁₋₄-alkyl substituted with 1-3 fluorines, and

wherein substituted phenyl is substituted with 1-5 substituents selected from C₁₋₄-alkoxy, chlorine, C₁₋₄-alkyl and C₁₋₄-alkyl substituted with 1-3 fluorines;

R² and R³are independently of each other hydrogen, C₁₋₁₂-alkyl, C₃₋₈-cycloalkyl, substituted C₁₋₄-alkyl, aryl, substituted aryl, heterocyclyl or substituted heterocyclyl, wherein substituted C₁₋₄-alkyl means alkyl substituted with 1-3 substituents selected from C₃₋₈-cycloalkyl, aryl, heterocyclyl, substituted aryl and substituted heterocyclyl; wherein substituted aryl and substituted heterocyclyl means aryl and heterocyclyl substituted with C₁₋₄-alkoxy, halogen, CN, NO₂, COR, CO₂R, CONRR′, NRR′, SO₂R, NHCOR, SO₂NRR′, C₁₋₄-alkyl or C₁₋₄-alkyl substituted with 1-3 halogens, and

wherein substituted aryl means aryl substituted with 1-5 substituents and substituted heterocyclyl means heterocyclyl substituted with 1-4 substituents and these substituents are selected from C₁₋₄-alkoxy, halogen, CN, NO₂, COR, CO₂R, CONRR′, NRR′, SO₂R, NHCOR, SO₂NRR′, C₁₋₄-alkyl and C₁₋₄-alkyl substituted with 1-3 halogens, preferably wherein

R² and R³ are independently of each other hydrogen, C₁₋₁₂-alkyl, C₃₋₈-cycloalkyl, substituted C₁₋₄-alkyl, phenyl, substituted phenyl, heterocyclyl or substituted heterocyclyl,

wherein substituted C₁₋₄-alkyl means alkyl substituted with 1-3 substituents selected from C₃₋₈-cycloalkyl, phenyl, pyridyl, substituted phenyl and substituted pyridyl, wherein substituted phenyl or substituted pyridyl are substituted with C₁₋₄-alkoxy, halogen, CN, NO₂, COR, CO₂R, CONRR′, NRR′, SO₂R, NHCOR, SO₂NRR′, C₁₋₄-alkyl or C₁₋₄-alkyl substituted with 1-3 halogens, and

wherein substituted phenyl is substituted with 1-5 substituents and substituted heterocyclyl means heterocyclyl substituted with 1-4 substituents and these substituents are selected from C₁₋₄-alkoxy, halogen, CN, NO₂, COR, CO₂R, CONRR′, NRR′, SO₂R, NHCOR, SO₂NRR′, C₁₋₄-alkyl and C₁₋₄-alkyl substituted with 1-3 halogens, more preferably wherein

R² and R³ are independently of each other hydrogen, C₁₋₁₂-alkyl, C₃₋₈-cycloalkyl, substituted C₁₋₄-alkyl, phenyl, substituted phenyl, heterocyclyl or substituted heterocyclyl,

wherein substituted C₁₋₄-alkyl means alkyl substituted with 1-3 substituents selected from phenyl, pyridyl and substituted phenyl, wherein substituted phenyl is substituted with C₁₋₄-alkoxy, halogen, NO₂, C₁₋₄-alkyl or C₁₋₄-alkyl substituted with 1-3 halogens, and

wherein substituted phenyl is substituted with 1-5 substituents and substituted heterocyclyl means heterocyclyl substituted with 1-4 substituents and these substituents are selected from C₁₋₄-alkoxy, halogen, CN, NO₂, CO₂R, NRR′, C₁₋₄-alkyl and C₁₋₄-alkyl substituted with 1-3 halogens, most preferably wherein

R² and R³ are independently of each other hydrogen, C₁₋₁₂-alkyl, C₃₋₈-cycloalkyl, substituted C₁₋₄-alkyl, phenyl, substituted phenyl, heterocyclyl or substituted heterocyclyl,

wherein substituted C₁₋₄-alkyl means alkyl substituted with 1-3 substituents selected from phenyl, pyridyl and substituted phenyl; wherein substituted phenyl is substituted with NO₂, and

wherein substituted phenyl is substituted with 1-5 substituents and substituted heterocyclyl means heterocyclyl substituted with 1-4 substituents and these substituents are selected from C₁₋₄-alkoxy, fluorine, chlorine, CN, NO₂, CO₂R, NRR′, C₁₋₄-alkyl and C₁₋₄-alkyl substituted with 1-3 fluorines;

X is S or O,

preferably wherein

X is O;

A is selected from the group consisting of:

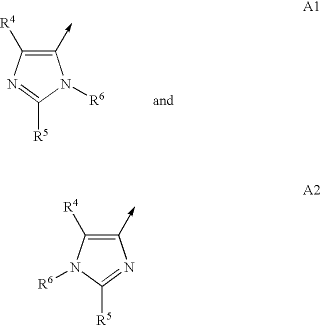

wherein

R⁴ is hydrogen, C₁₋₁₂-alkyl, CO₂R or aryl, preferably wherein

R⁴ is hydrogen, C₁₋₁₂-alkyl, CO₂R or phenyl;

R⁵ is hydrogen, C₁₋₁₂-alkyl, substituted C₁₋₄-alkyl, halogen, aryl, substituted aryl, aryl-C(═O)—, aryl-CH(OH)— or NRR′,

wherein substituted C₁₋₄-alkyl means alkyl substituted with 1-3 substituents selected from C₃₋₈-cycloalkyl, aryl, heterocyclyl, substituted aryl and substituted heterocyclyl;

wherein substituted aryl and substituted heterocyclyl means aryl and heterocyclyl substituted with C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens, and wherein substituted aryl means aryl substituted with 1-5 substituents selected from C₁₋₄-alkoxy, halogen, CN, NO₂, COR, CO₂R, CONRR′, NRR′, NHCOR, SO₂NRR′, SO₂R, C₁₋₄-alkyl and C₁₋₄-alkyl substituted with 1-3 halogens, preferably wherein

R⁵ is hydrogen, C₁₋₁₂-alkyl, substituted C₁₋₄-alkyl, halogen, phenyl, substituted phenyl, phenyl-C(═O)—, phenyl-CH(OH)— or NRR′,

wherein substituted C₁₋₄-alkyl means alkyl substituted with 1-3 substituents selected from C₃₋₈-cycloalkyl, phenyl, heterocyclyl, substituted phenyl and substituted heterocyclyl; wherein substituted phenyl and substituted heterocyclyl are substituted with C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens, and

wherein substituted phenyl is substituted with 1-5 substituents selected from C₁₋₄-alkoxy, halogen, CN, NO₂, COR, CO₂R, CONRR′, NRR′, NHCOR, SO₂NRR′, SO₂R, C₁₋₄-alkyl and C₁₋₄-alkyl substituted with 1-3 halogens, more preferably wherein

R⁵ is hydrogen, C₁₋₁₂-alkyl, substituted C₁₋₄-alkyl, halogen, phenyl, substituted phenyl, phenyl-C(═O)—, phenyl-CH(OH)— or NRR′,

wherein substituted C₁₋₄-alkyl means alkyl substituted with 1-3 substituents selected from phenyl and substituted phenyl; wherein substituted phenyl is substituted with C₁₋₄-alkoxy, halogen, C₁₋₄-alkyl or C₁₋₄-alkyl substituted with 1-3 halogens, and

wherein substituted phenyl is substituted with 1-5 substituents selected from C₁₋₄-alkoxy, halogen, C₁₋₄-alkyl and C₁₋₄-alkyl substituted with 1-3 halogens, most preferably wherein

R⁵ is hydrogen, C₁₋₁₂-alkyl, substituted C₁₋₄-alkyl, halogen, phenyl, substituted phenyl, phenyl-C(═O)—, phenyl-CH(OH)— or NRR′,

wherein substituted C₁₋₄-alkyl means alkyl substituted with 1-3 substituents selected from phenyl, and

wherein substituted phenyl is substituted with 1-5 substituents selected from C₁₋₄-alkoxy, chlorine, C₁₋₄-alkyl and C₁₋₄-alkyl substituted with 1-3 fluorines;

R⁶ is hydrogen, C₁₋₁₂-alkyl or substituted C₁₋₄-alkyl,

wherein substituted C₁₋₄-alkyl means alkyl substituted with 1-3 substituents selected from C₃₋₈-cycloalkyl, aryl, heterocyclyl, substituted aryl and substituted heterocyclyl;

wherein substituted aryl and substituted heterocyclyl means aryl and heterocyclyl substituted with C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens, preferably wherein

R⁶ is hydrogen, C₁₋₁₂-alkyl or substituted C₁₋₄-alkyl,

wherein substituted C₁₋₄-alkyl means alkyl substituted with 1-3 substituents selected from C₃₋₈-cycloalkyl, phenyl, heterocyclyl, substituted phenyl and substituted heterocyclyl; wherein substituted phenyl or substituted heterocyclyl are substituted with C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens, more preferably wherein

R⁶ is hydrogen, C₁₋₁₂-alkyl or substituted C₁₋₄-alkyl,

wherein substituted C₁₋₄-alkyl means alkyl substituted with 1-3 substituents selected from phenyl and substituted phenyl; wherein substituted phenyl is substituted with C₁₋₄-alkoxy, halogen, C₁₋₄-alkyl or C₁₋₄-alkyl substituted with 1-3 halogens, most preferably wherein

R⁶ is hydrogen, C₁₋₁₂-alkyl or substituted C₁₋₄-alkyl,

wherein substituted C₁₋₄-alkyl means alkyl substituted with 1-3 substituents selected from phenyl;

R and R′ are independently of each other hydrogen or C₁₋₁₂-alkyl.

Other preferred embodiments of the invention are novel compounds of formula I wherein

R¹ is hydrogen, C₁₋₇-alkyl, C₃₋₆-cycloalkyl, allyl, substituted C₁₋₂-alkyl, phenyl, substituted phenyl or pyridyl,

wherein substituted C₁₋₂-alkyl means alkyl substituted with 1-3 substituents selected from C₃₋₆-cycloalkyl, phenyl, pyridyl and substituted phenyl; wherein substituted phenyl is substituted with C₁₋₂-alkoxy, phenyl, phenoxy, chlorine, CN, NO₂, CO₂R, NRR′, SO₂R, C₁₋₂-alkyl or C₁₋₂-alkyl substituted with 1-3 fluorines, and

wherein substituted phenyl is substituted with 1-5 substituents selected from C₁₋₂-alkoxy, chlorine, C₁₋₂-alkyl and C₁₋₂-alkyl substituted with 1-3 fluorines, preferably wherein

R¹ is hydrogen, C₁₋₄-alkyl, C₃₋₆-cycloalkyl, allyl, substituted C₁-alkyl, phenyl, substituted phenyl or pyridyl,

wherein substituted C₁-alkyl means alkyl substituted with 1-3 substituents selected from C₃₋₆-cycloalkyl, phenyl, pyridyl and substituted phenyl; wherein substituted phenyl is substituted with C₁-alkoxy, phenyl, phenoxy, chlorine, CN, NO₂, CO₂R, NRR′, SO₂R, C₁-alkyl or C₁-alkyl substituted with 1-3 fluorines, and

wherein substituted phenyl is substituted with 1-5 substituents selected from C₁-alkoxy, chlorine, C₁-alkyl and C₁-alkyl substituted with 1-3 fluorines;

R² and R³are independently of each other hydrogen, C₁₋₇-alkyl, C₃₋₆-cycloalkyl, substituted C₁₋₂-alkyl, phenyl, substituted phenyl, heterocyclyl or substituted heterocyclyl,

wherein substituted C₁₋₂-alkyl means alkyl substituted with 1-3 substituents selected from phenyl, pyridyl and substituted phenyl; wherein substituted phenyl is substituted with NO₂, and

wherein substituted phenyl is substituted with 1-5 substituents and substituted heterocyclyl means heterocyclyl substituted with 1-4 substituents and these substituents are selected from C₁₋₂-alkoxy, fluorine, chlorine, CN, NO₂, CO₂R, NRR′, C₁₋₂-alkyl and C₁₋₂-alkyl substituted with 1-3 fluorines, preferably wherein

R² and R³ are independently of each other hydrogen, C₁₋₄-alkyl, C₃₋₆-cycloalkyl, substituted C₁-alkyl, phenyl, substituted phenyl, heterocyclyl or substituted heterocyclyl,

wherein substituted C₁-alkyl means alkyl substituted with 1-3 substituents selected from phenyl, pyridyl and substituted phenyl; wherein substituted phenyl is substituted with NO₂, and

wherein substituted phenyl is substituted with 1-5 substituents and substituted heterocyclyl means heterocyclyl substituted with 1-4 substituents and these substituents are selected from C₁-alkoxy, fluorine, chlorine, CN, NO₂, CO₂R, NRR′, C₁-alkyl and C₁-alkyl substituted with 1-3 fluorines;

X is S or O;

A is selected from the group consisting of:

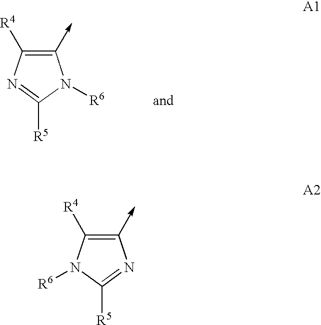

wherein

R⁴ is hydrogen, C₁₋₇-alkyl, CO₂R or phenyl;

R⁵ is hydrogen, C₁₋₇-alkyl, substituted C₁₋₂-alkyl, halogen, phenyl, substituted phenyl, phenyl-C(═O)—, phenyl-CH(OH)— or NRR′,

wherein substituted C₁₋₂-alkyl means alkyl substituted with 1-3 substituents selected from phenyl, and

wherein substituted phenyl is substituted with 1-5 substituents selected from C₁₋₂-alkoxy, chlorine, C₁₋₂-alkyl and C₁₋₂-alkyl substituted with 1-3 fluorines, preferably wherein

R⁵ is hydrogen, C₁₋₄-alkyl, substituted C₁-alkyl, halogen, phenyl, substituted phenyl, phenyl-C(═O)—, phenyl-CH(OH)— or NRR′,

wherein substituted C₁-alkyl means alkyl substituted with 1-3 substituents selected from phenyl, and

wherein substituted phenyl is substituted with 1-5 substituents selected from C₁-alkoxy, chlorine, C₁-alkyl and C₁-alkyl substituted with 1-3 fluorines;

R⁶ is hydrogen, C₁₋₇-alkyl or substituted C₁₋₂-alkyl,

wherein substituted C₁₋₂-alkyl means alkyl substituted with 1-3 substituents selected from phenyl, preferably wherein

R⁶ is hydrogen, C₁₋₅-alkyl or substituted C₁-alkyl,

wherein substituted C₁-alkyl means alkyl substituted with 1-3 substituents selected from phenyl;

R and R′ are independently of each other hydrogen or C₁₋₇-alkyl, preferably wherein

R and R′ are independently of each other hydrogen or C₁₋₄-alkyl.

Another preferred embodiment of the invention are novel compounds of formula I wherein

X is O, or

wherein

A is A1, or

wherein

A is A2.

More preferred embodiments of compounds of formula I, as well as ethers or hydrolyzable esters of compounds of formula I and pharmaceutically acceptable salts thereof, are listed in table 1:

TABLE 1

| STRUCTURE | SYSTEMATIC NAME |
|---|---|
| 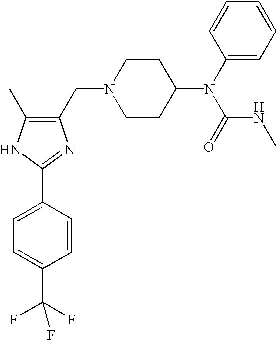 | 1-[1-[[2-[4-(Trifluoromethyl)phenyl]-5-methyl-1H-imida- zol-4-yl]methyl]-4-piperidinyl]-3-methyl-1-phenylurea |
| 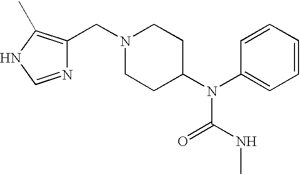 | 3-Methyl-1-[1-[(5-methyl-1H-imidazol-4-yl)meth- yl]-4-piperidinyl]-1-phenylurea |
| 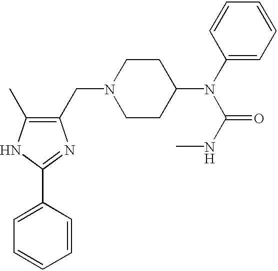 | 3-Methyl-1-[1-[(5-methyl-2-phenyl-1H-imidazol-4-yl)meth- yl]-4-piperidinyl]-1-phenylurea |
| 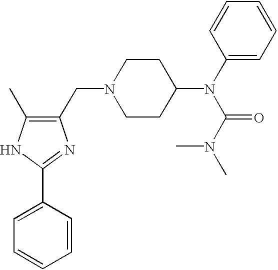 | 1,1-Dimethyl-3-[1-[(5-methyl-2-phenyl-1H-imidazol-4-yl)meth- yl]-4-piperidinyl]-3-phenylurea |
| 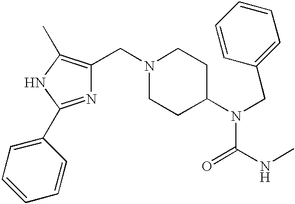 | 1-Benzyl-3-methyl-1-[1-[(5-methyl-2-phenyl-1H-imida- zol-4-yl)methyl]-4-piperidinyl]urea |
| 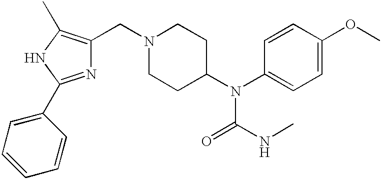 | 1-(4-Methoxyphenyl)-3-methyl-1-[1-[(5-methyl-2-phenyl-1H-imi- dazol-4-yl)methyl]-4-piperidinyl]urea |
| 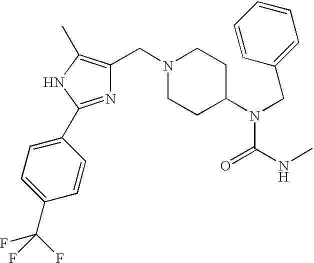 | 1-Benzyl-3-methyl-1-[1-[[5-methyl-2-[4-(tri- fluoromethyl)phenyl]-1H-imidazol-4-yl]methyl]-4-piperi- dinyl]urea |
| 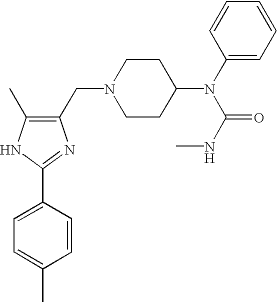 | 3-Methyl-1-[1-[[5-methyl-2-(4-methylphenyl)-1H-imida- zol-4-yl]methyl]-4-piperidinyl]-1-phenylurea |
| 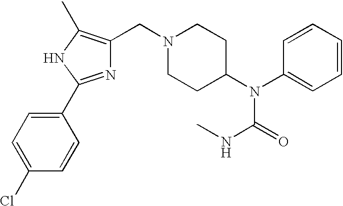 | 1-[1-[[2-(4-Chlorophenyl)-5-methyl-1H-imidazol-4-yl]meth- yl]-4-piperidinyl]-3-methyl-1-phenylurea |
| 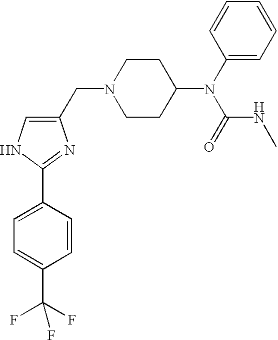 | 3-Methyl-1-phenyl-1-[1-[[2-[4-(tri- fluoromethyl)phenyl]-1H-imidazol-4-yl]methyl]-4-piperidinyl]urea |
| 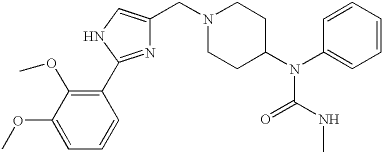 | 1-[1-[[2-(2,3-Dimethoxyphenyl)-1H-imidazol-4-yl]meth- yl]-4-piperidinyl]-3-methyl-1-phenylurea |
| 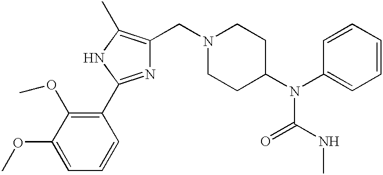 | 1-[1-[[2-(2,3-Dimethoxyphenyl)-5-methyl-1H-imidazol-4-yl]meth- yl]-4-piperidinyl]-3-methyl-1-phenylurea |
| 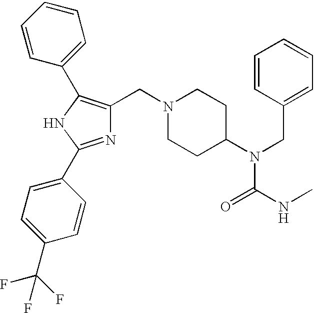 | 1-Benzyl-3-methyl-1-[1-[[5-phenyl-2-[4-(tri- fluoromethyl)phenyl]-1H-imidazol-4-yl]methyl]-4-piperi- dinyl]urea |
| 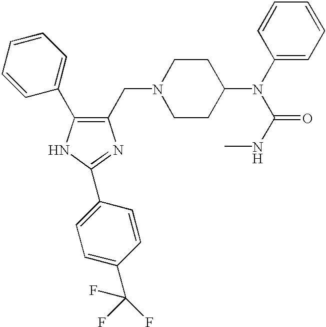 | 3-Methyl-1-phenyl-1-[1-[[5-phenyl-2-[4-(tri- fluoromethyl)phenyl]-1H-imidazol-4-yl]methyl]-4-piperidinyl]urea |
| 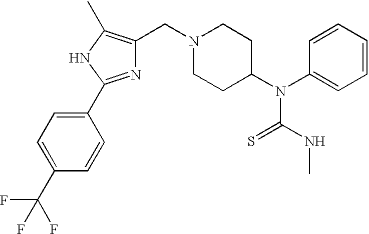 | 3-Methyl-1-[1-[[5-methyl-2-[4-(tri- fluoromethyl)phenyl]-1H-imidazol-4-yl]methyl]-4-piperi- dinyl]-1-phenylthiourea |
| 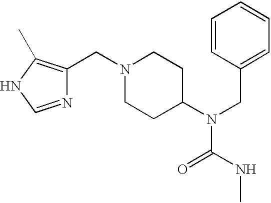 | 1-Benzyl-3-methyl-1-[1-[(5-methyl-1H-imidazol-4-yl)meth- yl]-4-piperidinyl]urea |
| 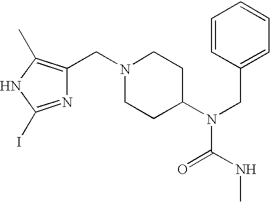 | 1-Benzyl-1-[1-[(2-iodo-5-methyl-1H-imidazol-4-yl)meth- yl]-4-piperidinyl]-3-methylurea |
| 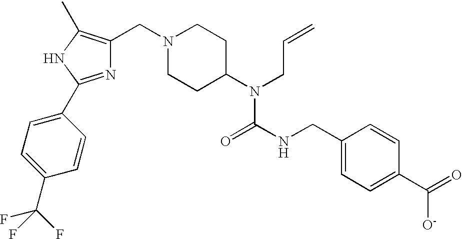 | 1-Allyl-1-[1-[[5-methyl-2-[4-(trifluoromethyl)phenyl]-1H-imida- zol-4-yl]methyl]-4-piperidinyl]-3-(4-nitrobenzyl)urea |
| 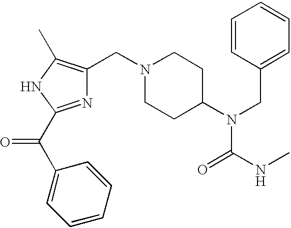 | 1-[1-[(2-Benzoyl-5-methyl-1H-imidazol-4-yl)methyl]-4-piperi- dinyl]-1-benzyl-3-methylurea |
| 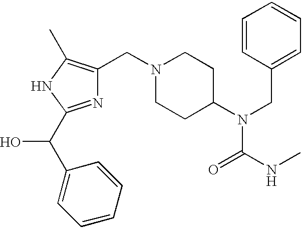 | 1-Benzyl-1-[1-[[2-[(RS)-(hydroxy)(phenyl)methyl]-5-meth- yl-1H-imidazol-4-yl]methyl]-4-piperidinyl]-3-methyl- urea |
| 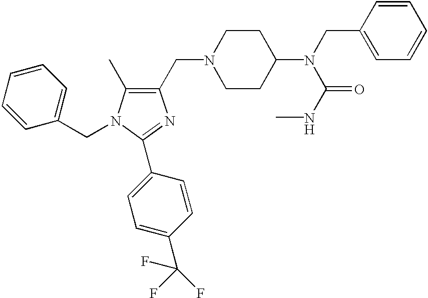 | 1-Benzyl-1-[1-[[1-benzyl-5-methyl-2-[4-(tri- fluoromethyl)phenyl]-1H-imidazol-4-yl]methyl]-4-piperi- dinyl]-3-methylurea |
| 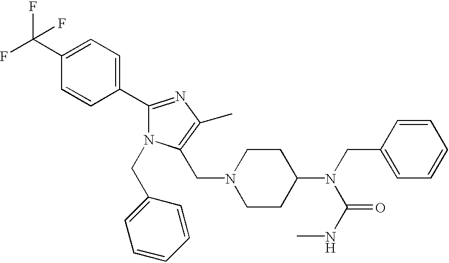 | 1-Benzyl-1-[1-[[3-benzyl-5-methyl-2-[4-(tri- fluoromethyl)phenyl]-3H-imidazol-4-yl]methyl]-4-piperi- dinyl]-3-methylurea |
| 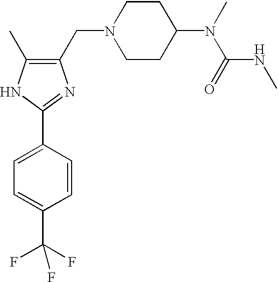 | 1-[1-[[2-[4-(Trifluoromethyl)phenyl]-5-methyl-1H-imida- zol-4-yl]methyl]-4-piperidinyl]-1,3-dimethylurea |
| 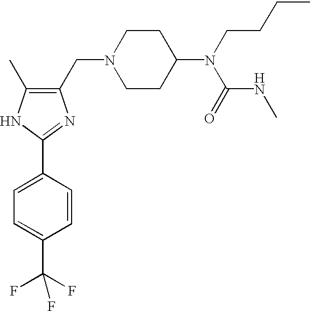 | 1-Butyl-1-[1-[[2-[4-(trifluoromethyl)phenyl]-5-methyl-1H-imida- zol-4-yl]methyl]-4-piperidinyl]-3-methylurea |
| 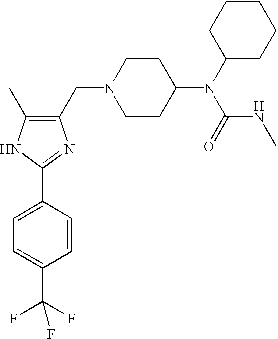 | 1-Cyclohexyl-1-[1-[[2-[4-(trifluoromethyl)phenyl]-5-meth- yl-1H-imidazol-4-yl]methyl]-4-piperidinyl]-3-methyl- urea |
| 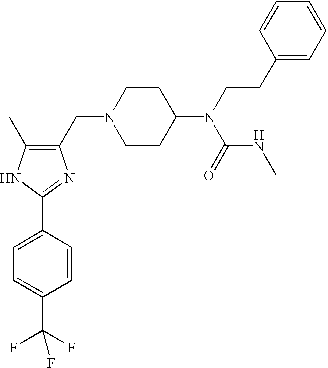 | 1-[1-[[2-[4-(Trifluoromethyl)phenyl]-5-methyl-1H-imida- zol-4-yl]methyl]-4-piperidinyl]-3-methyl-1-(2-phen- ethyl)urea |
| 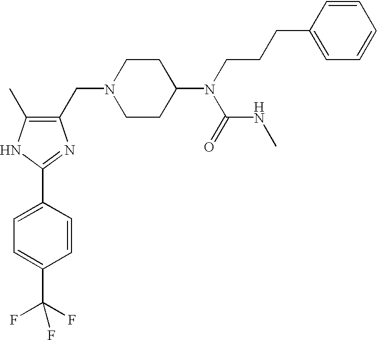 | 1-[1-[[2-[4-(Trifluoromethyl)phenyl]-5-methyl-1H-imida- zol-4-yl]methyl]-4-piperidinyl]-3-methyl-1-(3-phenyl- propyl)urea |
| 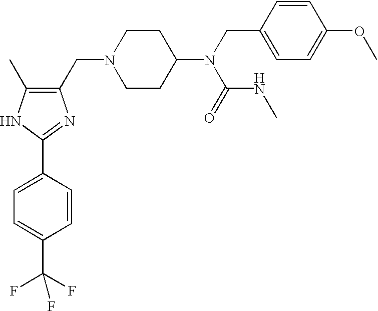 | 1-[1-[[2-[4-(Trifluoromethyl)phenyl]-5-methyl-1H-imida- zol-4-yl]methyl]-4-piperidinyl]-1-(4-methoxy- benzyl)-3-methylurea |
| 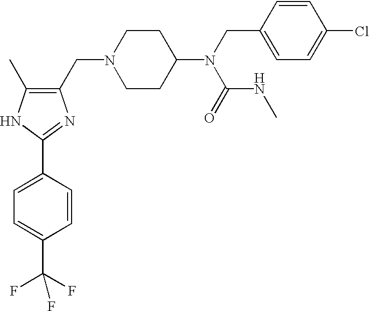 | 1-(4-Chlorobenzyl)-1-[1-[[2-[4-(tri- fluoromethyl)phenyl]-5-methyl-1H-imidazol-4-yl]meth- yl]-4-piperidinyl]-3-methylurea |
| 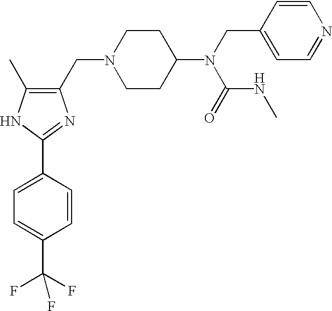 | 1-[1-[[2-[4-(Trifluoromethyl)phenyl]-5-methyl-1H-imida- zol-4-yl]methyl]-4-piperidinyl]-3-methyl-1-[(4-pyri- dyl)methyl]urea |
| 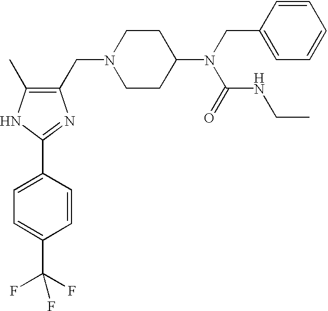 | 1-Benzyl-3-ethyl-1-[1-[[2-[4-(trifluoromethyl)phenyl]-5-meth- yl-1H-imidazol-4-yl]methyl]-4-piperidinyl]urea |
| 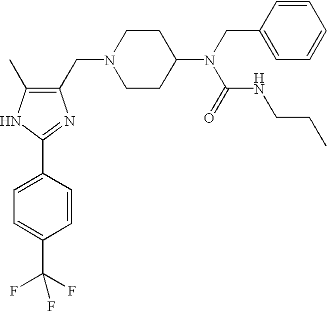 | 1-Benzyl-1-[1-[[2-[4-(trifluoromethyl)phenyl]-5-methyl-1H-imida- zol-4-yl]methyl]-4-piperidinyl]-3-propylurea |
| 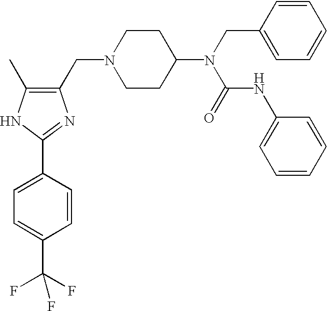 | 1-Benzyl-1-[1-[[2-[4-(trifluoromethyl)phenyl]-5-methyl-1H-imida- zol-4-yl]methyl]-4-piperidinyl]-3-phenylurea |
| 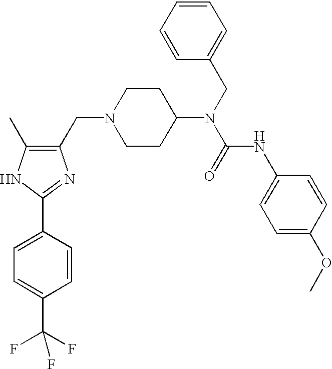 | 1-Benzyl-1-[1-[[2-[4-trifluoromethyl-phenyl]-5-methyl-1H-imida- zol-4-yl]methyl]-4-piperidinyl]-3-(4-methoxy- phenyl)urea |
| 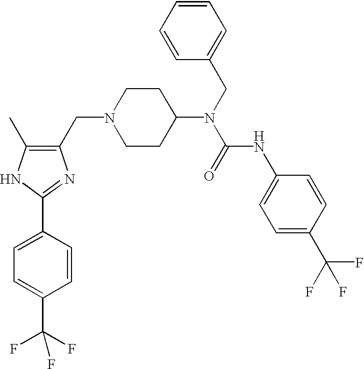 | 1-Benzyl-3-[4-(trifluoromethyl)phenyl]1-[1-[[2-[4-(trifluoro- methyl)phenyl-5-methyl-1H-imidazol-4-yl]meth- yl]-4-piperidinyl]urea |
| 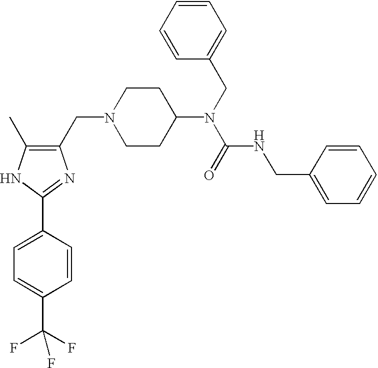 | 1,3-Dibenzyl-1-[1-[[2-[4-(trifluoromethyl)phenyl]-5-meth- yl-1H-imidazol-4-yl]methyl]-4-piperidinyl]urea |
| 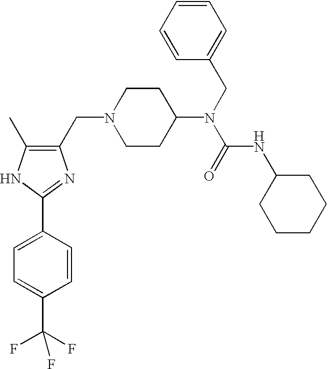 | 1-Benzyl-3-cyclohexyl-1-[1-[[2-[4-(trifluoro- methyl)phenyl]-5-methyl-1H-imidazol-4-yl]meth- yl]-4-piperidinyl]urea |
| 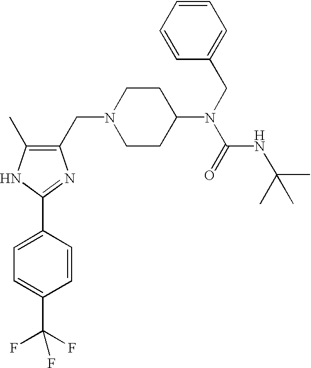 | 1-Benzyl-3-tert.-butyl-1-[1-[[2-[4-(trifluoro- methyl)phenyl]-5-methyl-1H-imidazol-4-yl]meth- yl]-4-piperidinyl]urea |
| 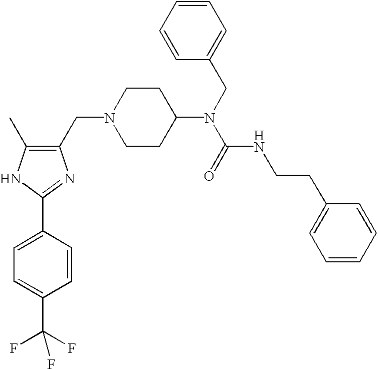 | 1-Benzyl-1-[1-[[2-[4-(trifluoromethyl)phenyl]-5-methyl-1H-imida- zol-4-yl]methyl]-4-piperidinyl]-3-(2-phenyl- ethyl)urea |
| 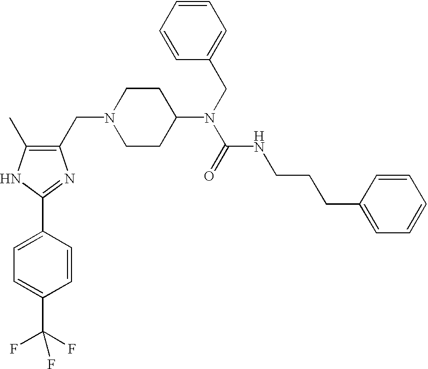 | 1-Benzyl-1-[1-[[2-[4-(trifluoromethyl)phenyl]-5-methyl-1H-imida- zol-4-yl]methyl]-4-piperidinyl]-3-(3-phenyl- propyl)urea |
| 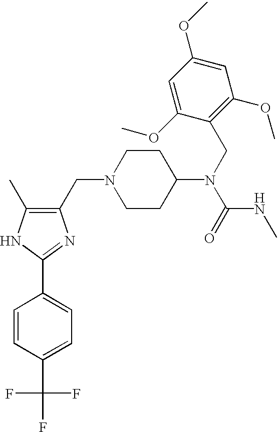 | 1-[1-[[2-[4-(Trifluoromethyl)phenyl]-5-methyl-1H-imida- zol-4-yl]methyl]-4-piperidinyl]-1-(2,4,6-tri- methoxybenzyl)-3-methylurea |
| 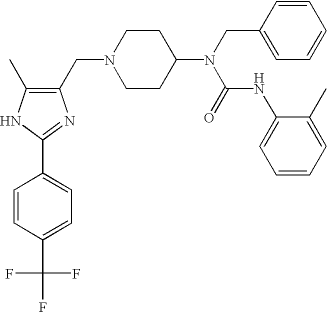 | 1-Benzyl-1-[1-[[2-[4-(trifluoromethyl)phenyl]-5-methyl-1H-imida- zol-4-yl]methyl]-4-piperidinyl]-3-(2-methylphenyl)urea |
| 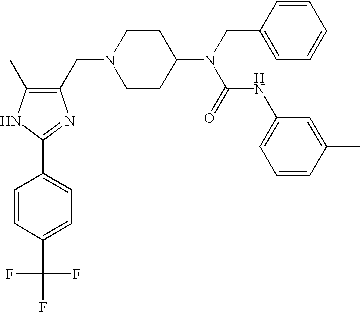 | 1-Benzyl-1-[1-[[2-[4-(trifluoromethyl)phenyl]-5-methyl-1H-imida- zol-4-yl]methyl]-4-piperidinyl]-3-(3-methyl- phenyl)urea |
| 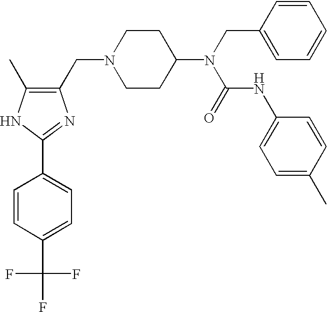 | 1-Benzyl-1-[1-[[2-[4-(trifluoromethyl)phenyl]-5-methyl-1H-imida- zol-4-yl]methyl]-4-piperidinyl]-3-(4-methyl- phenyl)urea |
| 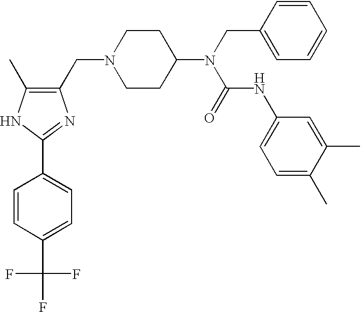 | 1-Benzyl-1-[1-[[2-[4-(trifluoromethyl)phenyl]-5-methyl-1H-imida- zol-4-yl]methyl]-4-piperidinyl]-3-(3,4-dimethyl- phenyl)urea |
| 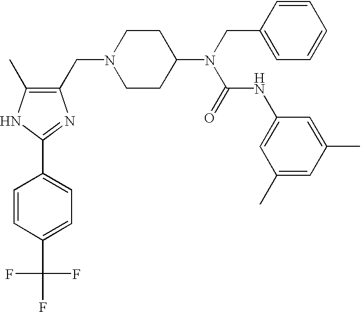 | 1-Benzyl-1-[1-[[2-[4-(trifluoromethyl)phenyl]-5-methyl-1H-imida- zol-4-yl]methyl]-4-piperidinyl]-3-(3,5-dimethyl- phenyl)urea |
| 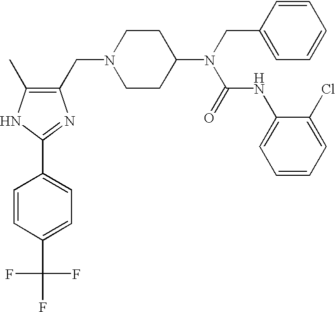 | 1-Benzyl-3-(2-chlorophenyl)-1-[1-[[2-[4-(trifluoro- methyl)phenyl]-5-methyl-1H-imidazol-4-yl]meth- yl]-4-piperidinyl]urea |
| 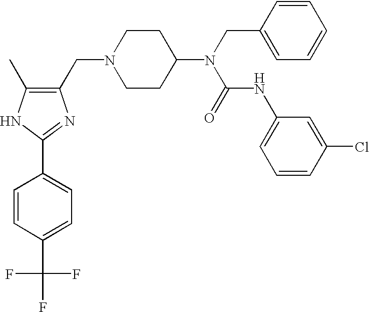 | 1-Benzyl-3-(3-chlorophenyl)-1-[1-[[2-[4-(trifluoro- methyl)phenyl]-5-methyl-1H-imidazol-4-yl]meth- yl]-4-piperidinyl]urea |
| 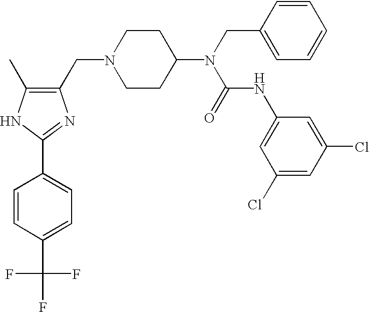 | 1-Benzyl-3-(3,5-dichlorophenyl)-1-[1-[[2-[4-(tri- fluoromethyl)phenyl]-5-methyl-1H-imidazol-4-yl]meth- yl]-4-piperidinyl]urea |
| 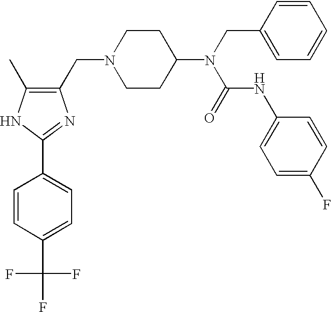 | 1-Benzyl-3-(4-fluorophenyl)-1-[1-[[2-[4-(trifluoro- methyl)phenyl]-5-methyl-1H-imidazol-4-yl]meth- yl]-4-piperidinyl]urea |
| 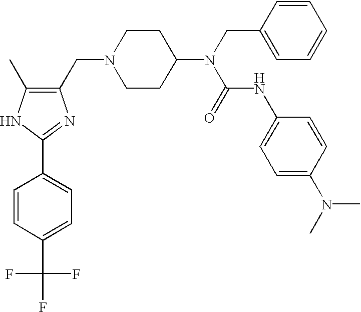 | 1-Benzyl-1-[1-[[2-[4-(trifluoromethyl)phenyl]-5-methyl-1H-imida- zol-4-yl]methyl]-4-piperidinyl]-3-[4-(dimethyl- amino)phenyl]urea |
| 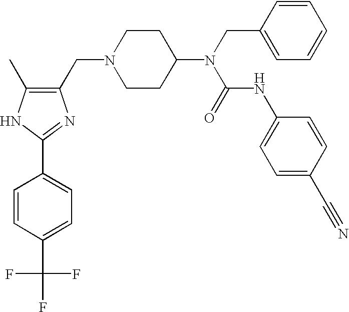 | 1-Benzyl-3-(4-cyanophenyl)-1-[1-[[2-[4-(trifluoro- methyl)phenyl]-5-methyl-1H-imidazol-4-yl]meth- yl]-4-piperidinyl]urea |
| 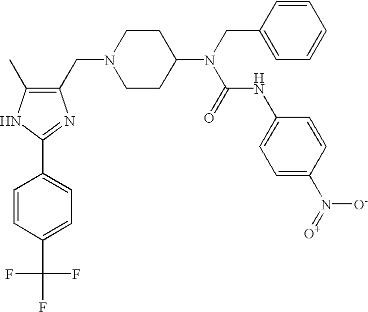 | 1-Benzyl-1-[1-[[2-[4-(trifluoromethyl)phenyl]-5-methyl-1H-imida- zol-4-yl]methyl]-4-piperidinyl]-3-(4-nitro- phenyl)urea |
| 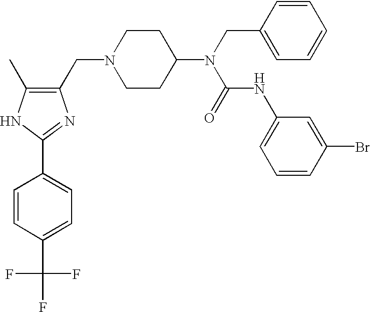 | 1-Benzyl-3-(3-bromophenyl)-1-[1-[[2-[4-(trifluoro- methyl)phenyl]-5-methyl-1H-imidazol-4-yl]meth- yl]-4-piperidinyl]urea |
| 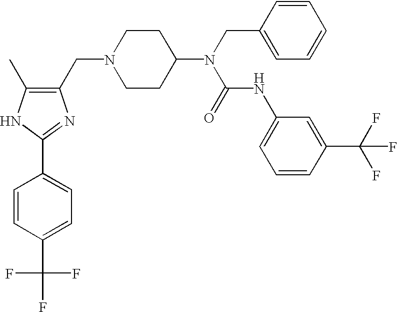 | 1-Benzyl-3-[3-(trifluoromethyl)phenyl]-1-[1-[[2-[4-(trifluoro- methyl)phenyl]-5-methyl-1H-imidazol-4-yl]meth- yl]-4-piperidinyl]urea |
| 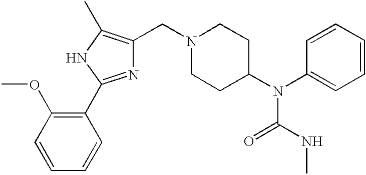 | 1-[1-[[2-(2-Methoxyphenyl)-5-methyl-1H-imidazol-4-yl]meth- yl]-4-piperidinyl]-3-methyl-1-phenylurea |
| 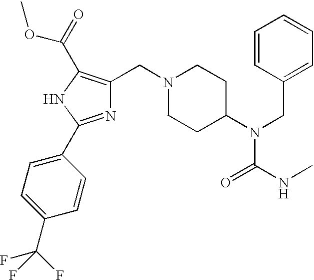 | Methyl 5-[[4-(1-benzyl-3-methyl- ureido)piperidino]methyl]-2-[4-(tri- fluoromethyl)phenyl]-3H-imidazole-4-carboxylate |
| 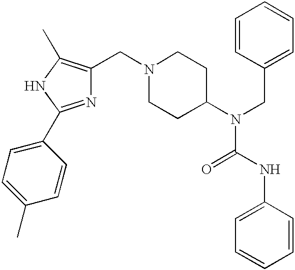 | 1-Benzyl-1-[1-[5-methyl-2-(4-methyl- phenyl)-1H-imidazol-4-ylmethyl]-4-piperi- dinyl]-3-phenylurea |
| 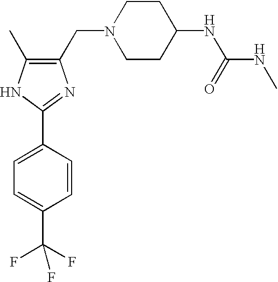 | 1-Methyl-3-{1-[5-methyl-2-(4-trifluoromethyl-phenyl)-1H-imida- zol-4-ylmethyl]-piperidin-4-yl}-urea |
| 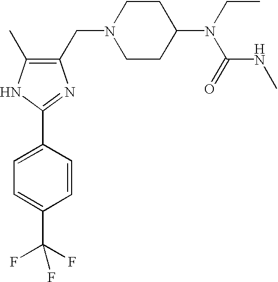 | 1-Ethyl-3-methyl-1-{1-[5-methyl-2-(4-trifluoromethyl-phenyl)-1H-imi- dazol-4-ylmethyl]-piperidin-4-yl}-urea |
| 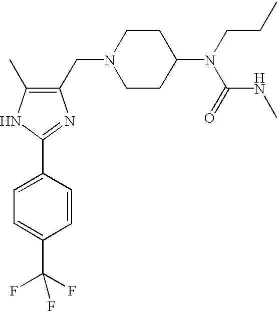 | 3-Methyl-1-{1-[5-methyl-2-(4-trifluoromethyl-phenyl)-1H-imida- zol-4-ylmethyl]-piperidin-4-yl}-1-propyl-urea |
| 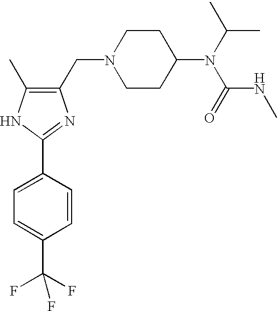 | 1-Iospropyl-3-methyl-1-{1-[5-methyl-2-(4-tri- fluoromethyl-phenyl)-1H-imidazol-4-ylmethyl]-piperi- din-4-yl}-urea |
| 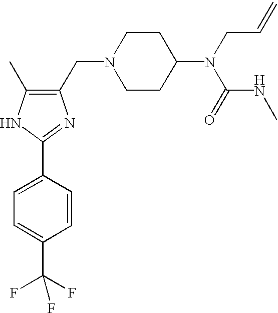 | 1-Allyl-3-methyl-1-{1-[5-methyl-2-(4-trifluoromethyl-phe- nyl)-1H-imidazol-4-ylmethyl]-piperidin-4-yl}-urea |
| 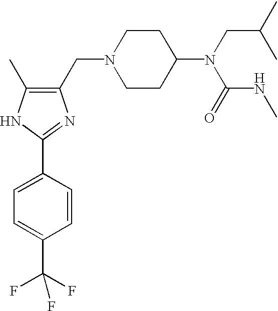 | 1-Isobutyl-3-methyl-1-{1-[5-methyl-2-(4-tri- fluoromethyl-phenyl)-1H-imidazol-4-ylmethyl]-piperi- din-4-yl}-urea |
| 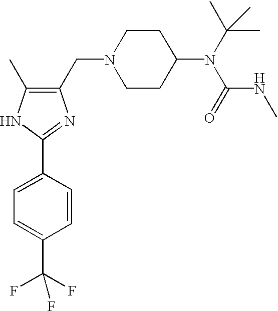 | 1-tert.-butyl-3-methyl-1-{1-[5-methyl-2-(4-trifluoro- methyl-phenyl)-1H-imidazol-4-ylmethyl]-piperi- 4-yl}-urea |
| 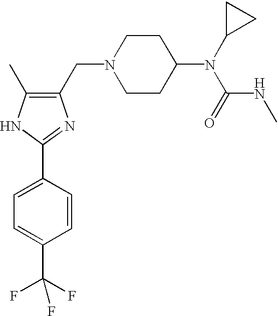 | 1-Cyclopropyl-3-methyl-1-{1-[5-methyl-2-(4-tri- fluoromethyl-phenyl)-1H-imidazol-4-ylmethyl]-piperi- din-4-yl}-urea |
| 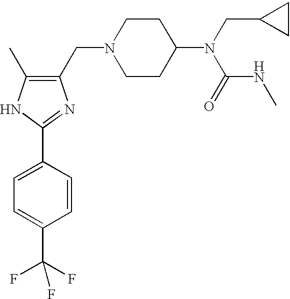 | 1-Cyclopropylmethyl-3-methyl-1-{1-[5-methyl-2-(4-tri- fluoromethyl-phenyl)-1H-imidazol-4-ylmethyl]-piperi- din-4-yl}-urea |
| 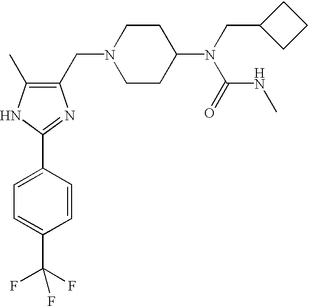 | 1-Cyclobutylmethyl-3-methyl-1-{1-[5-methyl-2-(4-tri- fluoromethyl-phenyl)-1H-imidazol-4-ylmethyl]-piperi- din-4-yl}-urea |
| 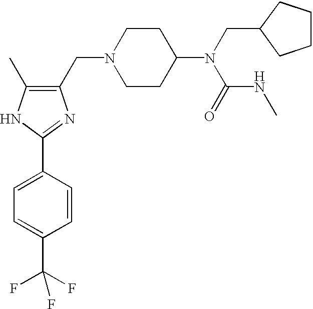 | 1-Cyclopentylmethyl-3-methyl-1-{1-[5-methyl-2-(4-tri- fluoromethyl-phenyl)-1H-imidazol-4-ylmethyl]-piperi- din-4-yl}-urea |
| 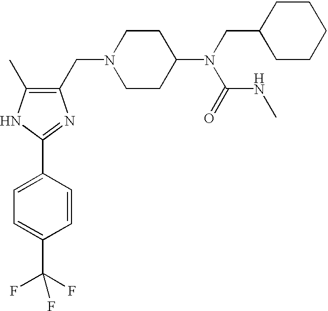 | 1-Cyclohexylmethyl-3-methyl-1-{1-[5-methyl-2-(4-tri- fluoromethyl-phenyl)-1H-imidazol-4-ylmethyl]-piperi- din-4-yl}-urea |
| 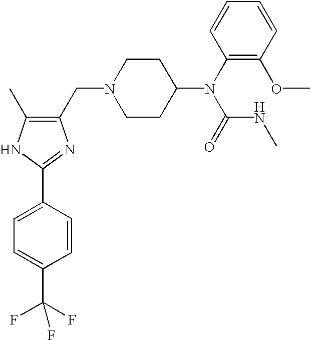 | 1-(2-Methoxy-phenyl)-3-methyl-1-{1-[5-methyl-2-(4-tri- fluoromethyl-phenyl)-1H-imidazol-4-ylmethyl]-piperi- din-4-yl}-urea |
| 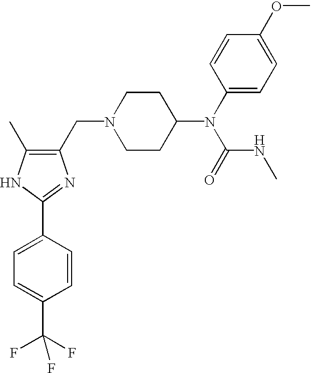 | 1-(4-Methoxy-phenyl)-3-methyl-1-{1-[5-methyl-2-(4-tri- fluoromethyl-phenyl)-1H-imidazol-4-ylmethyl]-piperi- din-4-yl}-urea |
| 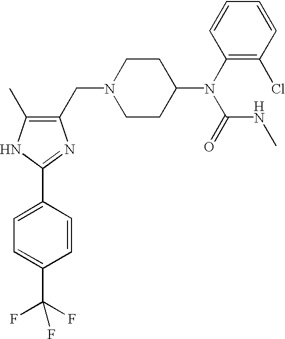 | 1-(2-Chloro-phenyl)-3-methyl-1-{1-[5-methyl-2-(4-tri- fluoromethyl-phenyl)-1H-imidazol-4-ylmethyl]-piperidin-4-yl}-urea |
| 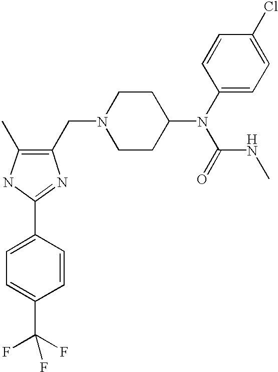 | 1-(4-Chloro-phenyl)-3-methyl-1-{1-[5-methyl-2-(4-tri- fluoromethyl-phenyl)-1H-imidazol-4-ylmethyl]-piperi- din-4-yl}-urea |
| 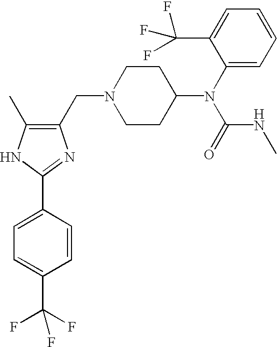 | 3-Methyl-1-{1-[5-methyl-2-(4-trifluoromethyl-phenyl)-1H-imida- zol-4-ylmethyl]-piperidin-4-yl}-1-(2-tri- fluoromethyl-phenyl)-urea |
| 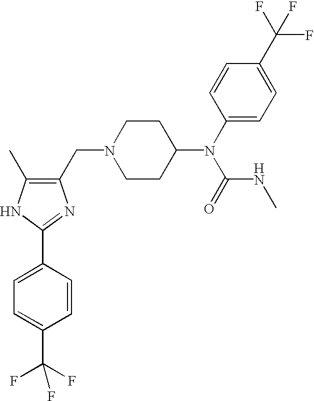 | 3-Methyl-1-{1-[5-methyl-2-(4-trifluoromethyl-phenyl)-1H-imida- zol-4-ylmethyl]-piperidin-4-yl}-1-(4-tri- fluoromethyl-phenyl)-urea |
| 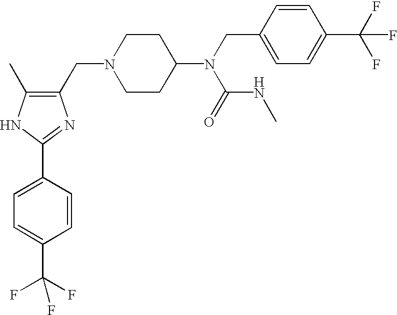 | 3-Methyl-1-{1-[5-methyl-2-(4-trifluoromethyl-phenyl)-1H-imida- zol-4-ylmethyl]-piperidin-4-yl}-1-(4-tri- fluoromethyl-benzyl)-urea |
| 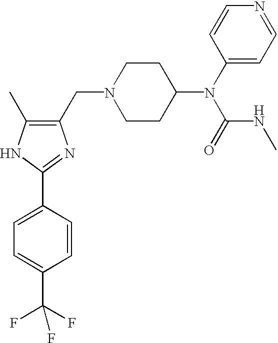 | 3-Methyl-1-{1-[5-methyl-2-(4-trifluoromethyl-phenyl)-1H-imida- zol-4-ylmethyl]-piperidin-4-yl}-1-pyridin-4-yl-urea |
| 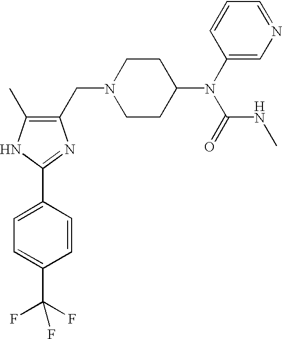 | 3-Methyl-1-{1-[5-methyl-2-(4-trifluoromethyl-phenyl)-1H-imida- zol-4-ylmethyl]-piperidin-4-yl}-1-pyridin-3-yl-urea |
| 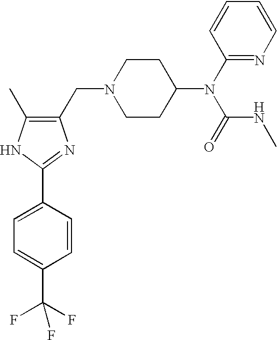 | 3-Methyl-1-{1-[5-methyl-2-(4-trifluoromethyl-phenyl)-1H-imida- zol-4-ylmethyl]-piperidin-4-yl}-1-pyridin-3-ylmethyl-urea |
| 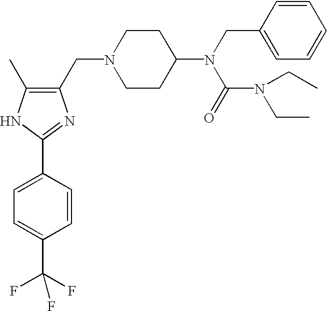 | 1-Benzyl-3,3-diethyl-1-{1-[5-methyl-2-(4-trifluoro- methyl-phenyl)-1H-imidazol-4-ylmethyl]-piperi- din-4-yl}-urea |
| 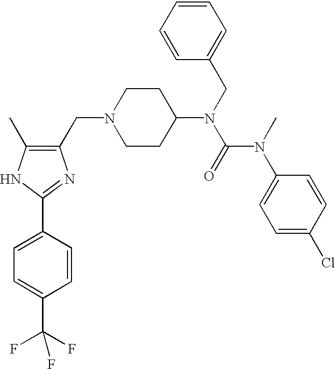 | 1-Benzyl-3-(4-chloro-phenyl)-3-methyl-1-[1-[5-methyl-2-(4-tri- fluoromethyl-phenyl)-1H-imidazol-4-ylmethyl]-piperi- din-4-yl}-urea |
| 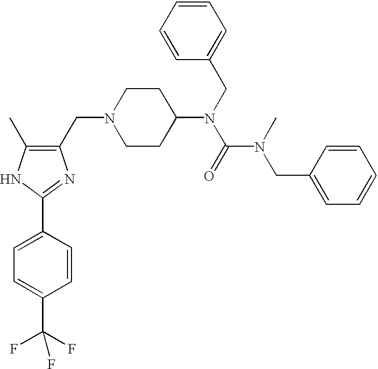 | 1,3-Dibenzyl-3-methyl-1-{1-[5-methyl-2-(4-trifluoro- methyl-phenyl)-1H-imidazol-4-ylmethyl]-piperi- din-4-yl}-urea |
| 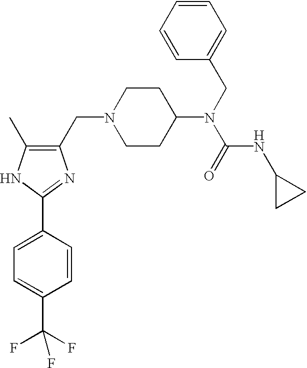 | 1-Benzyl-3-cyclopropyl-1-{1-[5-methyl-2-(4-trifluoro- methyl-phenyl)-1H-imidazol-4-ylmethyl]-piperidin-4-yl}-urea |
| 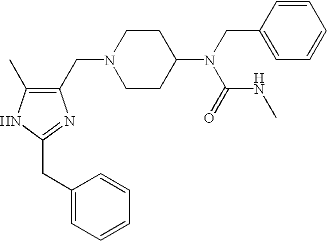 | 1-Benzyl-1-[1-(2-benzyl-5-methyl-1H-imidazol-4-ylmeth- yl)-piperidin-4-yl]-3-methyl-urea |
| 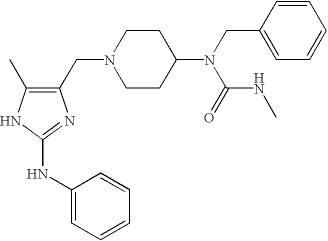 | 1-Benzyl-3-methyl-1-[1-(5-methyl-2-phenylamino-1H-imida- zol-4-ylmethyl)-piperidin-4-yl]-urea |
| 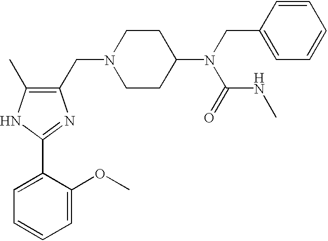 | 1-Benzyl-1-{1-[2-(2-methoxy-phenyl)-5-methyl-1H-imida- zol-4-ylmethyl]-piperidin-4-yl}-3-methyl-urea |
| 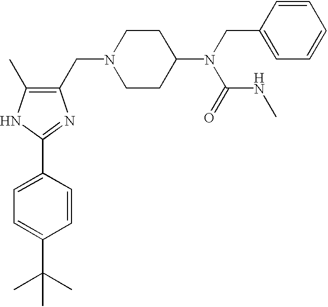 | 1-Benzyl-1-{1-[2-(4-tert.-butyl-phenyl)-5-methyl-1H-imida- zol-4-ylmethyl]-piperidin-4-yl}-3-methyl-urea |
| 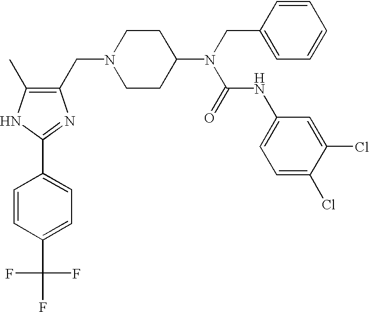 | 1-Benzyl-3-(3,4-dichloro-phenyl)-1-{1-[5-methyl-2-(4-tri- fluoromethyl-phenyl)-1H-imidazol-4-ylmethyl]-piperi- din-4-yl}-urea |
| 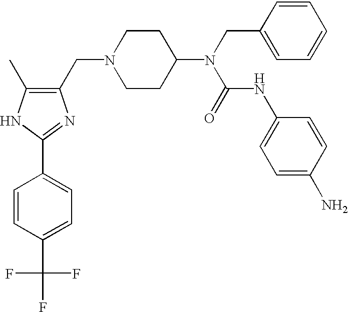 | 3-(4-Amino-phenyl)-1-benzyl-1-{1-[5-methyl-2-(4-tri- fluoromethyl-phenyl)-1H-imidazol-4-ylmethyl]-piperi- din-4-yl}-urea |
| 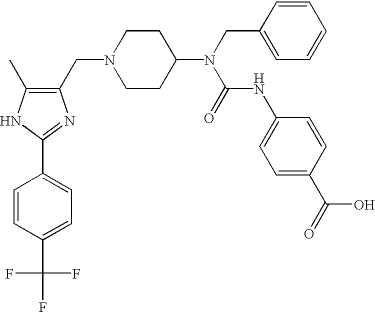 | 4-(3-Benzyl-3-{1-[5-methyl-2-(4-trifluoromethyl-phen- yl)-1H-imidazol-4-ylmethyl]-piperidin-4-yl}-ureido)-benzoic acid |
| 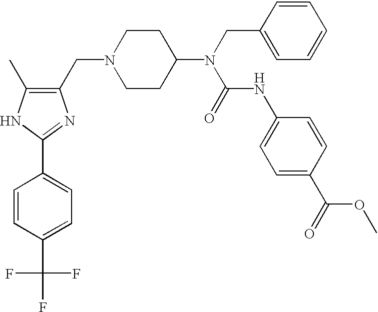 | 4-(3-Benzyl-3-{1-[5-methyl-2-(4-trifluoromethyl-phenyl)-1H-imida- zol-4-ylmethyl]-piperidin-4-yl}-ureido)-benzoic acid methyl ester |
| 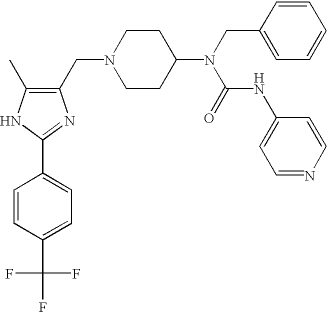 | 1-Benzyl-1-{1-[5-methyl-2-(4-trifluoromethyl-phenyl)-1H-imida- zol-4-ylmethyl]-piperidin-4-yl}-3-pyridin-4-yl-urea |
| 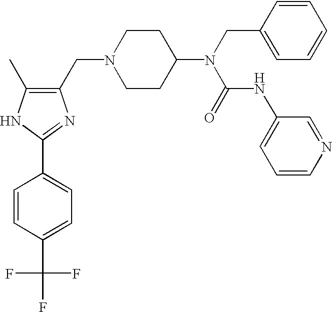 | 1-Benzyl-1-{1-[5-methyl-2-(4-trifluoromethyl-phenyl)-1H-imida- zol-4-ylmethyl]-piperidin-4-yl}-3-pyridin-3-yl-urea |
| 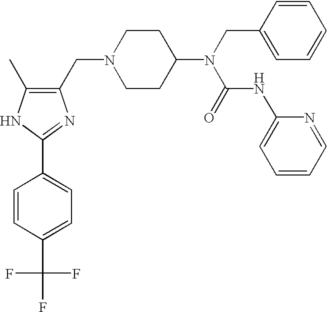 | 1-Benzyl-1-{1-[5-methyl-2-(4-trifluoromethyl-phenyl)-1H-imida- zol-4-ylmethyl]-piperidin-4-yl}-3-pyridin-2-yl-urea |
| 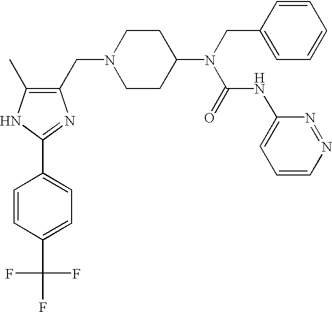 | 1-Benzyl-1-{1-[5-methyl-2-(4-trifluoromethyl-phenyl)-1H-imida- zol-4-ylmethyl]-piperidin-4-yl}-3-pyridazin-3-yl-urea |
| 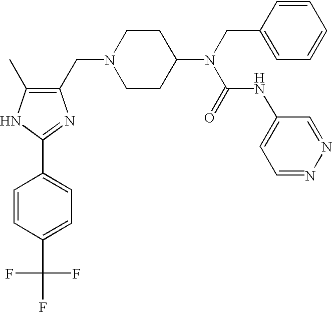 | 1-Benzyl-1-{1-[5-methyl-2-(4-trifluoromethyl-phenyl)-1H-imida- zol-4-ylmethyl]-piperidin-4-yl}-3-pyridazin-4-yl-urea |
| 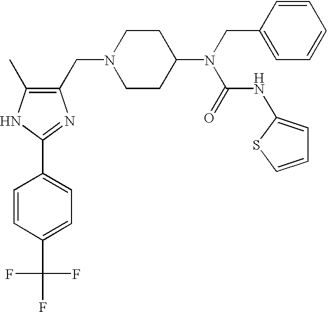 | 1-Benzyl-1-{1-[5-methyl-2-(4-trifluoromethyl-phenyl)-1H-imida- zol-4-ylmethyl]-piperidin-4-yl}-3-thiophen-2-yl-urea |
| 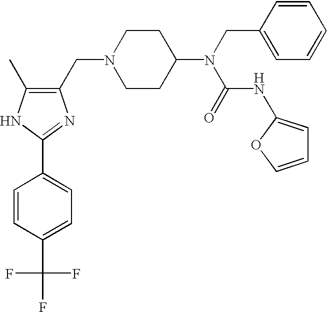 | 1-Benzyl-3-furan-2-yl-1-{1-[5-methyl-2-(4-tri- fluoromethyl-phenyl)-1H-imidazol-4-ylmethyl]-piperi- din-4-yl}-urea |
| 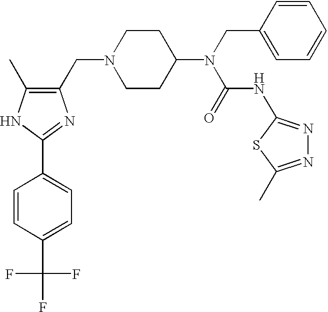 | 1-Benzyl-3-(5-methyl-[1,3,4]thiadiazol-2-yl)-1-{1-[5-meth- yl-2-(4-trifluoromethyl-phenyl)-1H-imidazol-4-ylmeth- yl]-piperidin-4-yl}-urea |
| 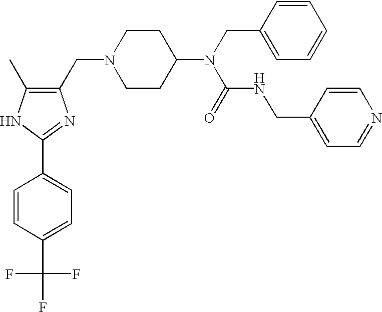 | 1-Benzyl-1-{1-[5-methyl-2-(4-trifluoromethyl-phenyl)-1H-imida- zol-4-ylmethyl]-piperidin-4-yl}-3-pyridin-4-ylmethyl-urea |
| 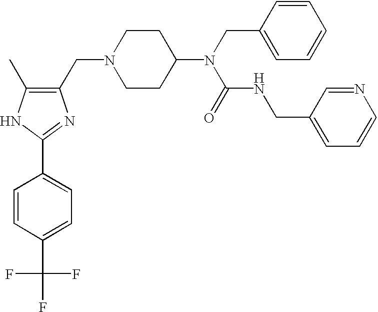 | 1-Benzyl-1-{1-[5-methyl-2-(4-trifluoromethyl-phenyl)-1H-imida- zol-4-ylmethyl]-piperidin-4-yl}-3-pyridin-3-ylmethyl-urea |
| 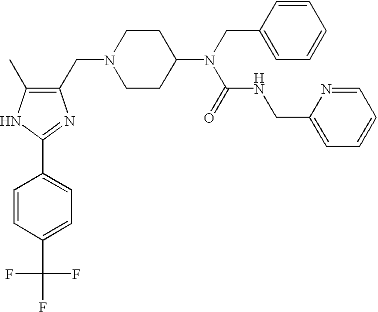 | 1-Benzyl-1-{1-[5-methyl-2-(4-trifluoromethyl-phenyl)-1H-imida- zol-4-ylmethyl]-piperidin-4-yl}-3-pyridin-2-ylmethyl-urea |
| 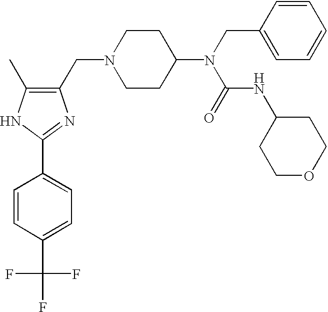 | 1-Benzyl-1-{1-[5-methyl-2-(4-trifluoromethyl-phenyl)-1H-imida- zol-4-ylmethyl]-piperidin-4-yl}-3-(tetrahydro-pyran-4-yl)-urea |
| 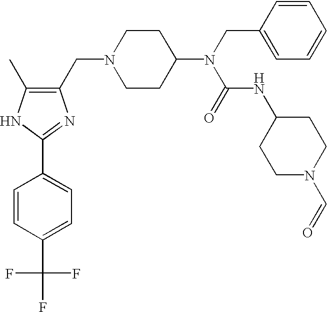 | 1-Benzyl-3-(1-formyl-piperidin-4-yl)-1-{1-[5-methyl-2-(4-tri- fluoromethyl-phenyl)-1H-imidazol-4-ylmethyl]-piperi- din-4-yl}-urea |
| 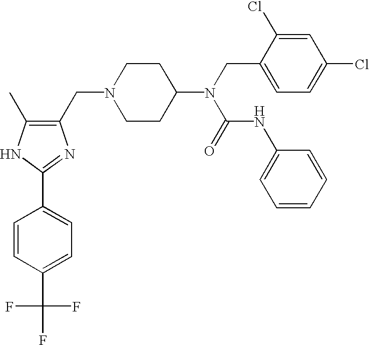 | 1-(2,4-Dichloro-benzyl)-1-{1-[5-methyl-2-(4-tri- fluoromethyl-phenyl)-1H-imidazol-4-ylmethyl]-piperi- din-4-yl}-3-phenyl-urea |
| 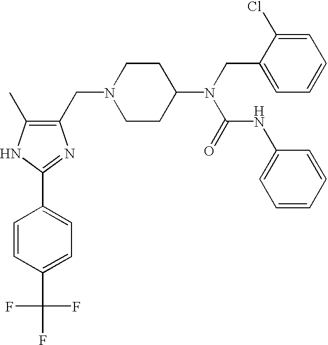 | 1-(2-Chloro-benzyl)-1-{1-[5-methyl-2-(4-tri- fluoromethyl-phenyl)-1H-imidazol-4-ylmethyl]-piperi- din-4-yl}-3-phenyl-urea |
| 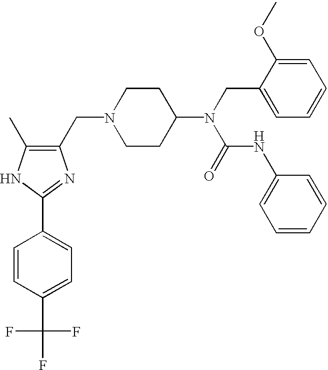 | 1-(2-Methoxy-benzyl)-1-{1-[5-methyl-2-(4-tri- fluoromethyl-phenyl)-1H-imidazol-4-ylmethyl]-piperi- din-4-yl}-3-phenyl-urea |
| 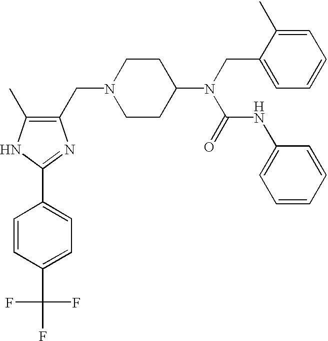 | 1-(2-Methyl-benzyl)-1-{1-[5-methyl-2-(4-tri- fluoromethyl-phenyl)-1H-imidazol-4-ylmethyl]-piperi- din-4-yl}-3-phenyl-urea |
| 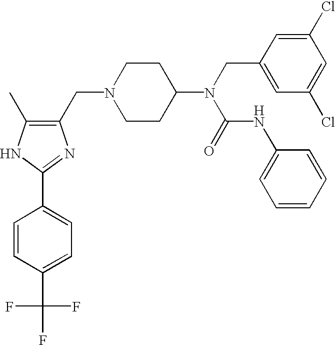 | 1-(3,5-Dichloro-benzyl)-1-{1-[5-methyl-2-(4-tri- fluoromethyl-phenyl)-1H-imidazol-4-ylmethyl]-piperi- din-4-yl}-3-phenyl-urea |
| 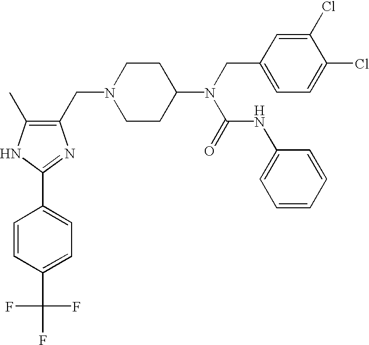 | 1-(3,4-Dichloro-benzyl)-1-{1-[5-methyl-2-(4-tri- fluoromethyl-phenyl)-1H-imidazol-4-ylmethyl]-piperi- din-4-yl}-3-phenyl-urea |
| 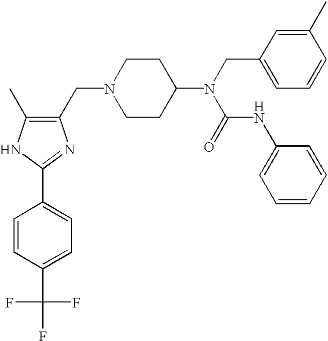 | 1-(3-Methyl-benzyl)-1-{1-[5-methyl-2-(4-tri- fluoromethyl-phenyl)-1H-imidazol-4-ylmethyl]-piperi- din-4-yl}-3-phenyl-urea |
| 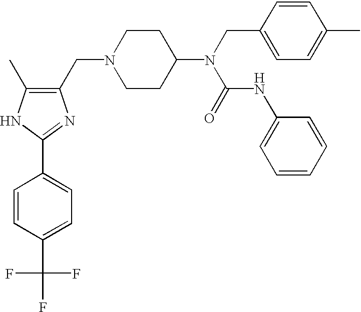 | 1-(4-Methyl-benzyl)-1-{1-[5-methyl-2-(4-trifluoro- methyl-phenyl)-1H-imidazol-4-ylmethyl]-piperi- din-4-yl}-3-phenyl-urea |
| 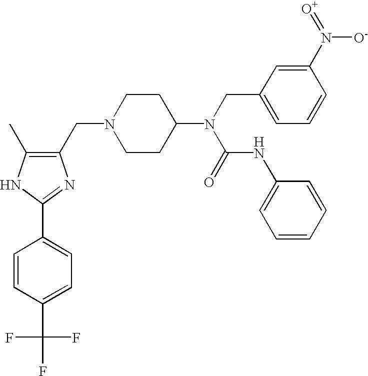 | 1-{1-[5-Methyl-2-(4-trifluoromethyl-phenyl)-1H-imida- zol-4-ylmethyl]-piperidin-4-yl}-1-(3-nitro-benzyl)-3-phenyl-urea |
| 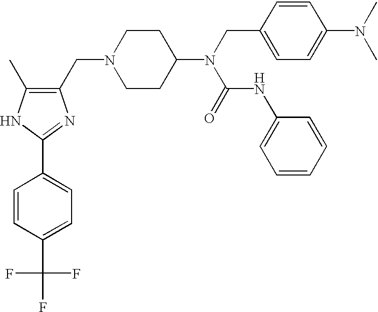 | 1-(4-Dimethylamino-benzyl-1-{1-[5-methyl-2-(4-tri- fluoromethyl-phenyl)-1H-imidazol-4-ylmethyl]-piperi- din-4-yl}-3-phenyl-urea |
| 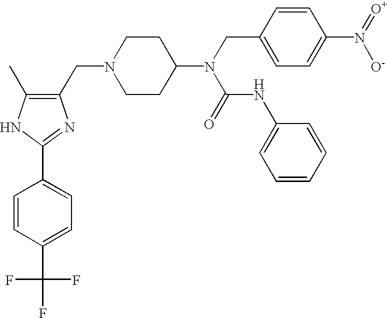 | 1-{1-[5-Methyl-2-(4-trifluoromethyl-phenyl)-1H-imida- zol-4-ylmethyl]-piperidin-4-yl}-1-(4-nitro-benzyl)-3-phenyl-urea |
| 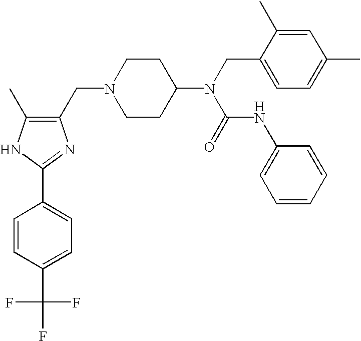 | 1-(2,4-Dimethyl-benzyl)-1-{1-[5-methyl-2-(4-tri- fluoromethyl-phenyl)-1H-imidazol-4-ylmethyl]-piperi- din-4-yl}-3-phenyl-urea |
| 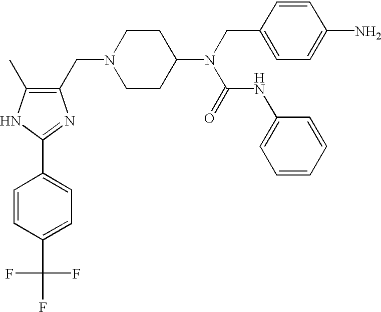 | 1-(4-Amino-benzyl)-1-{1-[5-methyl-2-(4-tri- fluoromethyl-phenyl)-1H-imidazol-4-ylmethyl]-piperi- din-4-yl}-3-phenyl-urea |
| 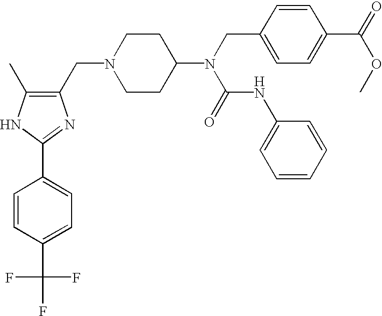 | 4-(1-{1-[5-Methyl-2-(4-trifluoromethyl-phenyl)-1H-imida- zol-4-ylmethyl]-piperidin-4-yl}-3-phenyl-ureido- methyl)-benzoic acid methyl ester |
| 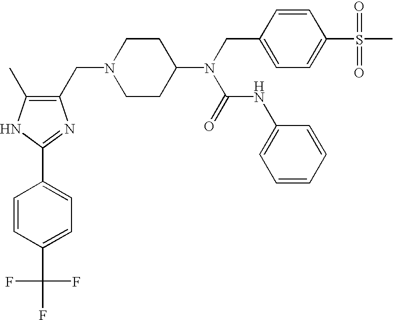 | 1-(4-Methanesulfonyl-benzyl)-1-{1-[5-methyl-2-(4-tri- fluoromethyl-phenyl)-1H-imidazol-4-ylmethyl]-piperi- din-4-yl}-3-phenyl-urea |
| 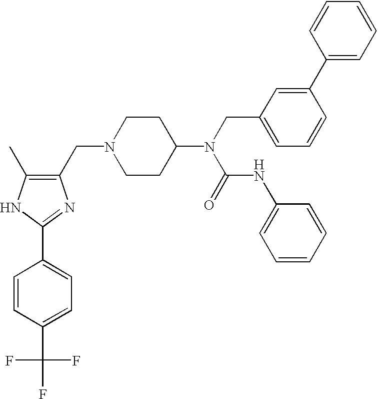 | 1-Biphenyl-3-ylmethyl-1-{1-[5-methyl-2-(4-tri- fluoromethyl-phenyl)-1H-imidazol-4-ylmethyl]-piperi- din-4-yl}-3-phenyl-urea |
| 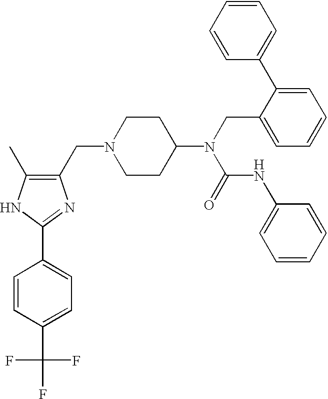 | 1-Biphenyl-2-ylmethyl-1-{1-[5-methyl-2-(4-tri- fluoromethyl-phenyl)-1H-imidazol-4-ylmethyl]-piperi- din-4-yl}-3-phenyl-urea |
| 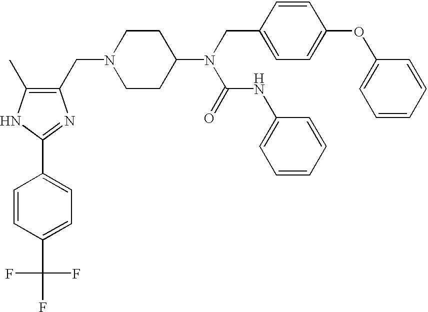 | 1-{1-[5-Methyl-2-(4-trifluoromethyl-phenyl)-1H-imida- zol-4-ylmethyl]-piperidin-4-yl}-1-(4-phenoxy-benzyl)-3-phenyl-urea |
| 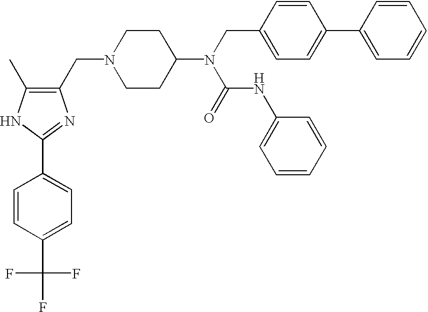 | 1-Biphenyl-4-ylmethyl-1-{1-[5-methyl-2-(4-tri- fluoromethyl-phenyl)-1H-imidaozl-4-ylmethyl]-piperi- din-4-yl}-3-phenyl-urea |
| 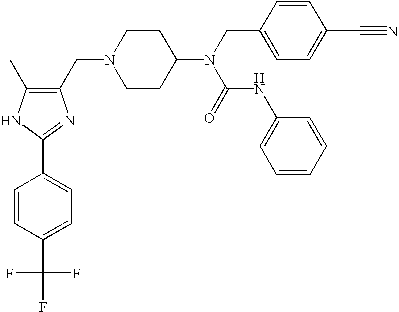 | 1-(4-Cyano-benzyl)-1-{1-[5-methyl-2-(4-tri- fluoromethyl-phenyl)-1H-imidazol-4-ylmethyl]-piperi- din-4-yl}-3-phenyl-urea |
| 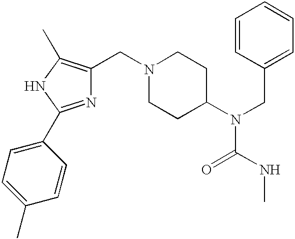 | 1-Benzyl-3-methyl-1-[1-(5-methyl-2-p-tolyl-1H-imi- dazol-4-ylmethyl)-piperidin-4-yl]-urea |
| 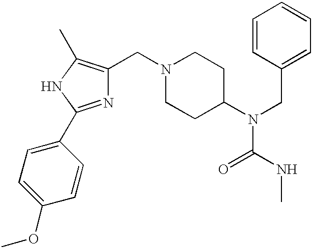 | 1-Benzyl-1-{1-[2-(4-methoxy-phenyl)-5-meth- yl-1H-imidazol-4-ylmethyl]-piperidin-4-yl}-3-methyl-urea |
| 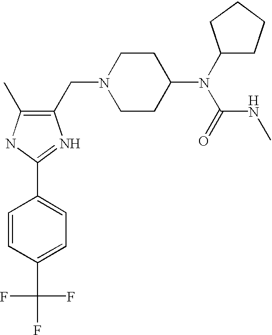 | 1-Cyclopentyl-3-methyl-1-{1-[5-methyl-2-(4-tri- fluoromethyl-phenyl)-1H-imidazol-4-ylmethyl]-piperidin-4-yl}-urea |
| 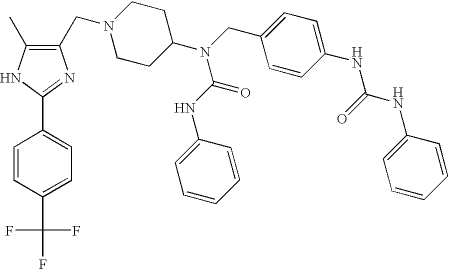 | 1-{1-[5-Methyl-2-(4-trifluoromethyl-phenyl)-1H-imida- zol-4-ylmethyl]-piperi- din-4-yl}-3-phenyl-1-[4-(3-phenyl-ureido)-benzyl]-urea |
| 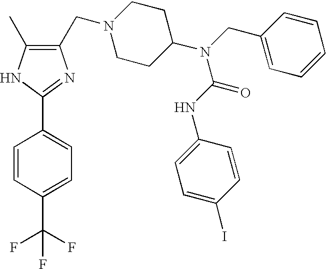 | 1-Benzyl-3-(4-iodo-phenyl-1-{1-[5-methyl-2-(4-tri- fluoromethyl-phenyl)-1H-imidazol-4-ylmethyl]-piperi- din-4-yl}-urea |

Chemokines and their receptors are potent activators and chemoattractants for leukocyte subpopulations and some non-hemopoietic cells. Whilst more studies are needed to delineate in more detail which chemokines and receptors are important in different diseases, they have been implicated in autoimmune disease [Arimilli et al Immunol. Rev. 177, 43-51 (2000)], diseases such as allergy, psoriasis, atherosclerosis, and malaria [Murdoch et al., Blood 95, 3032-3043 (2000)], multiple sclerosis [Zhang et al., Mult. Scler. 6, 3-13 (2000)], renal disease [Wada et al., Clin. Exp. Nephrol. 4, 273-280 (2000)], as well as in allograft rejection [Hancock et al., Curr. Opin. Immunol. 12, 511-516. (2000)].

CCR5, specifically, is believed to be the major coreceptor involved in sexual, parenteral and vertical transmission of HIV [van't Wout et al., J. Clin. Invest. 94, 2060-2067 (1994); Cornelissen, et al J. Virol. 69, 1810-1818 (1995); Veenstra et al., Clin. Infect. Dis. 21, 556-560 (1995)]. CCR5, specifically, may also have an etiological role in colitis [Ajuebor et al., J. Immunol. 166, 552-558 (2001)], multiple sclerosis [Simpson et al., J. Neuroimmunol. 108, 192-200 (2000)], diabetes [Cameron et al., J. Immunol. 165, 1102-1110 (2000)] and Alzheimer's disease [Xia and Hyman, Journal of Neurovirology 5, 32-41 (1999)].

The aminopiperidine derivatives provided by the present invention are useful in the treatment of the human or animal body. They can be used as medicaments, especially for treating viral diseases (HIV, HCV, and HBV infection), immune mediated conditions or diseases, bacterial diseases, parasitic diseases, inflammatory diseases, hyperproliferative vascular diseases, as anti-depressants, for the treatment of tumors, and cancer and to prevent allograft rejection. Especially, the present aminopiperidine derivatives are therapeutically active substances in the prevention and treatment of infection by the human immunodeficiency virus (HIV) and can be used as medicaments for the treatment of such diseases.

In particular, compounds of the present invention, and pharmaceutical compositions containing the same, are useful as chemotherapeutic agents, inhibitors of viral replication and modulators of the immune system. They can be used for the treatment of diseases mediated by retroviruses such as the human immunodeficiency virus (HIV), either alone or in combination with other inhibitors of HIV replication such as protease inhibitors, reverse transcriptase inhibitors and fusion inhibitors or with pharmacoenhancers such as cytochrome P450 inhibitors.

The aminopiperidine derivatives provided by the present invention can be used alone, or in combination with other therapeutically active agents, for example, an immunosuppressant, a chemotherapeutic agent, an anti-viral agent, an antibiotic, an anti-parasitic agent, an anti-inflammatory agent, an anti-fungal agent and/or an anti-vascular hyperproliferation agent.

Compounds, whenever prepared by the processes of the present invention are also an object of the present invention.

Assay Method:

Resonance Energy Transfer Assay (RET):

The activity of the compounds was determined using a fusion assay developed on the basis of the principle of resonance energy transfer, using HeLa cells stably transfected with gp120/gp41 from the macrophage-tropic primary isolate HIV-1JRFL and PM1 cells as previously described (Litwin, V et al (1996) “Human immunodeficiency virus type I membrane fusion mediated by a laboratory-adapted strain and a primary isolate analyzed by resonance energy transfer” J Virol 70(9), 6437-6441). The following minor modifications were applied: the assay buffer used comprised PBS/15% FCS (filtered through a 0.2 uM filter); cells were not washed three times in PBS before reading; all compounds were tested in a final concentration of 1% DMSO, and the monoclonal antibody Leu3a (330 ng/mL) was added to each plate, as a positive control (for 100% inhibition of cell fusion).

gp120-sCD4-CCR5 Binding Assay:

The gp120-sCD4-CCR5 binding assay was carried out as previously described (Dragic, T., A. Trkola, et al. (2000). “A binding pocket for a small molecule inhibitor of HIV-1 entry within the transmembrane helices of CCR5.” Proc Natl Acad Sci USA 97: 5639-44.) with the following minor modifications: the cell line used for these experiments was a CHO-K1 cell line stably transfected with the human CCR5 gene; the gp120-CD4 complex comprised recombinant biotinylated gp120 (JRFL strain) and soluble recombinant CD4; and all compounds were tested in a final concentration of 1% DMSO.

All reagents and cell lines were obtained from Progenics Pharmaceuticals Inc, Tarrytown, N.Y., USA, and are commercially available or can be prepared according to the methods described and the information given in the papers above.

In the assay, compounds of the formulas I range in activity from an IC₅₀ of about 0.5 to about 1500 nM, with preferred compounds having a range of activity from about 0.5 to about 750 nM, more preferably about 0.5 to 300 nM, and most preferably about 0.5 to 50 nM.

| | | FACS IC₅₀ |
|---|---|---|
| Structure | Name | (μM) |
| 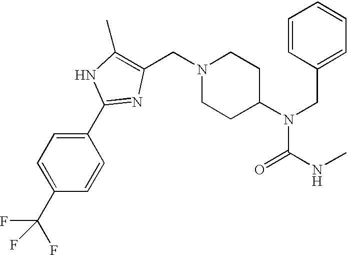 | 1-Benzyl-3-methyl-1-[1-[[5-methyl-2-[4- (trifluoromethyl)phenyl]-1H-imidazol-4- yl]methyl]-4-piperidinyl]urea | 0.11 |
| 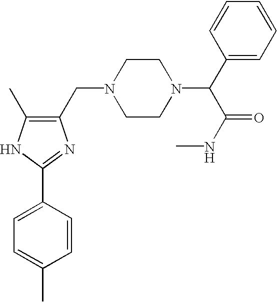 | 3-Methyl-1-[1-[[5-methyl-2-(4- methylphenyl)-1H-imidazol-4-yl]methyl]-4- piperidinyl]-1-phenylurea | 0.18 |
| 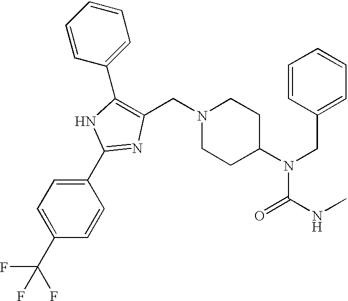 | 1-Benzyl-3-methyl-1-[1-[[5-phenyl-2-[4- (trifluoromethyl)phenyl]-1H-imidazol-4- yl]methyl]-4-piperidinyl]urea | 19.1 |
| 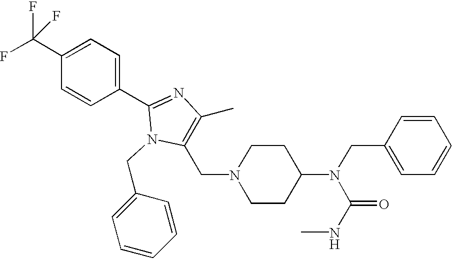 | 1-Benzyl-1-[1-[[3-benzyl-5-methyl-2-[4- (trifluoromethyl)phenyl]-3H-imidazol-4- yl]methyl]-4-piperidinyl]-3-methylurea | 1.1 |
| 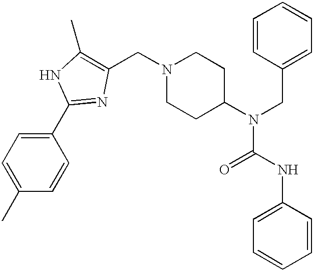 | 1-Benzyl-1-[1-[5-methyl-2-(4- methylphenyl)-1H-imidazol-4-ylmethyl]-4- piperidinyl]-3-phenylurea | 0.03 |
| 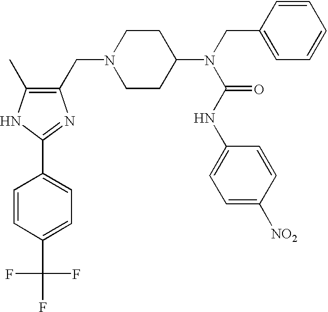 | 1-Benzyl-1-[1-[[2-[4- (trifluoromethyl)phenyl]-5-methyl-1H- imidazol-4-yl]methyl]-4-piperidinyl]-3-(4- nitrophenyl)urea | 0.45 |
| 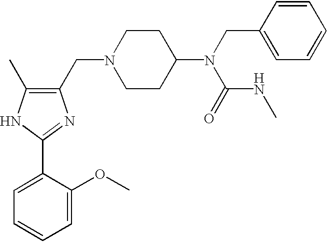 | 1-Benzyl-1-{1-[2-(2-methoxy-phenyl)-5- methyl-1H-imidazol-4-ylmethyl]-piperidin- 4-yl}-3-methyl-urea | 9.6 |

The aminopiperidine derivatives provided by the present invention, as well as their pharmaceutically useable salts, can be used as medicaments in the form of pharmaceutical preparations. The pharmaceutical preparations can be administered enterally, either orally, e.g. in the form of tablets, coated tablets, dragées, hard and soft gelatine capsules, solutions, emulsions, syrups, or suspensions, or rectally, e.g. in the form of suppositories. They can also be administered parenterally (intramuscularly, intravenously, or subcutaneously), e.g. in the form of injection solutions, or nasally, e.g. in the form of nasal sprays.

For the manufacture of pharmaceutical preparations, the aminopiperidine derivatives, as well as their pharmaceutically useable salts, can be formulated with a therapeutically inert, inorganic or organic excipient for the production of tablets, coated tablets, dragées, hard and soft gelatine capsules, solutions, emulsions or suspensions.

Suitable excipients for tablets, coated tablets, dragées, and hard gelatin capsules are, for example, lactose, corn starch and derivatives thereof, talc, and stearic acid or its salts.

Suitable excipients for soft gelatine capsules are, for example, vegetable oils, waxes, fats, semi-solid and liquid polyols.

Suitable excipients for injection solutions are, for example, water, saline, alcohols, polyols, glycerine or vegetable oils.

Suitable excipients for suppositories are, for example, natural and hardened oils, waxes, fats, semi-liquid or liquid polyols.

Suitable excipients for solutions and syrups for enteral use are, for example, water, polyols, saccharose, invert sugar and glucose.

The pharmaceutical preparations of the present invention may also be provided as sustained release formulations or other appropriate formulations.

The pharmaceutical preparations can also contain preservatives, solubilizers, stabilizers, wetting agents, emulsifiers, sweeteners, colorants, flavourants, salts for adjustment of the osmotic pressure, buffers, masking agents or antioxidants.

The pharmaceutical preparations may also contain other therapeutically active agents known in the art.

The aminopiperidine derivatives provided by the present invention are useful in the treatment of immune mediated conditions and diseases, viral diseases, bacterial diseases, parasitic diseases, inflammatory diseases, hyperproliferative vascular diseases, allograft rejection, tumors, and cancers.

The dosage can vary within wide limits and will, of course, be adjusted to the individual requirements in each particular case. For oral administration, a daily dosage of between about 0.01 and about 100 mg/kg body weight per day should be appropriate in monotherapy and/or in combination therapy. A typical preparation will contain from about 5% to about 95% active compound (w/w). The daily dosage can be administered as a single dosage or in divided dosages, typically between 1 and 5 dosages per day.

The aminopiperidine derivatives provided by the present invention or the medicaments thereof may be used in monotherapy or combination therapy, i.e. the treatment may be in conjunction with the administration of one or more additional therapeutically active substance(s). When the treatment is combination therapy, such administration may be concurrent or sequential with respect to that of the aminopiperidine derivatives of the present invention. Concurrent administration, as used herein thus includes administration of the agents at the same time or at different times.

It will be understood that references herein to treatment extend to prophylaxis as well as to treatment of existing conditions. Treatment of a disease or condition, as used herein, also includes preventing, inhibiting, regressing, reversing, alleviating or relieving the disease or condition, or the clinical symptoms thereof. The term “subject” as used herein refers to animals, including humans and other mammals.

The compounds of the present invention can be prepared as shown in the following schemes:

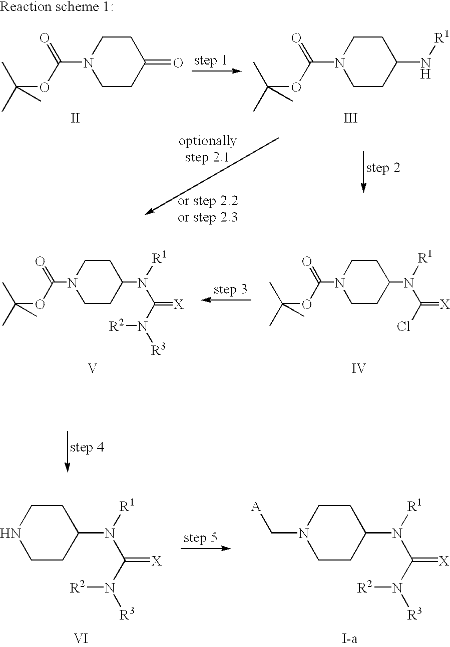

wherein R¹, R², R³, X and A are as defined for compounds of formula I.

Also part of the present invention is the preparation of compounds of formula I-a

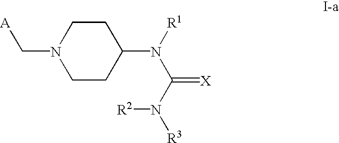

which process comprises

reacting a compound of formula VI

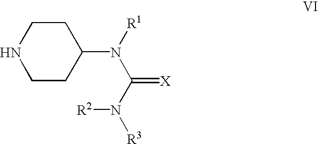

with a carboxaldehyde of formula A-CHO,

wherein A are as defined in formula I and subsequently reducing the reaction product with a reducing agent; or with a methylene halide of formula A-CH₂Hal,

wherein R¹, R², R³, A and X are as defined in formula I and Hal is Cl, Br or I.

The reaction represents step 5 of reaction scheme 1 and is described in more detail below.

In reaction scheme 1, step 1 is the reaction of an N-protected piperidone derivative of formula II (commercially available) with an amine of formula R¹NH₂, wherein R¹ is as defined for compounds of formula I (commercially available or synthesised according to known methods from textbooks on organic chemistry e.g. from J. March (1992), “Advanced Organic Chemistry: Reactions, Mechanisms and Structure”, 4^{th} ed. John Wiley and Sons) in the presence of an appropriate reducing agent and, optionally, an appropriate acid to obtain aminopiperidine derivative of formula III as described in the literature, for example in Ryder et al., Bioorg Med Chem Lett, 9, 2453-8 (1999), or Abdel-Magid et al., J Org Chem, 61, 3849-62 (1996).

Appropriate reducing agents for the reaction are known from the art and are, for example, lithium aluminium hydride, sodium borohydride, sodium cyanoborohydride or diusobutylaluminium hydride, and, preferably, sodium triacetoxyborohydride, and appropriate acids are carboxylic acids such as acetic acid or mineral acids such as hydrochloric acid. The reaction is carried out in an inert organic solvent such as an ether (e.g. tetrahydrofuran, diethyl ether, dibutyl ether or dioxane), a halogenated hydrocarbon (e.g. dichloromethane or trichloromethane), a hydrocarbon (e.g. cyclohexane, methyl cyclohexane, decaline, benzene, toluene, o-xylene, m-xylene or p-xylene), or a mixture of the aforementioned solvents, preferably dichoromethane at a reaction temperature from 0° C. to the boiling temperature of the reaction mixture, most preferably at ambient temperature.

The reaction can also be carried out under a hydrogen atmosphere in the presence of an appropriate catalyst (for example, a palladium catalyst such as palladium on charcoal). This reaction is carried out in an organic solvent, preferably at ambient temperature.

Alternatively, the imine can be pre-formed and subsequently reduced using a reducing agent such as sodium triacetoxyborohydride or under a hydrogen atmosphere in the presence of an appropriate catalyst as described above.

In reaction scheme 1, the N-tert.-butoxycarbonyl protecting group of the derivative of formula II can be replaced by other known N-protecting groups, for example those known from ‘Protecting groups in organic synthesis’ 3rd Ed. T. W. Greene, P. G. M. Wuts; Wiley-Interscience, New York 1999.

In step 2 of reaction scheme 1, an aminopiperidine derivative of formula III is converted to the corresponding piperidinecarbamoyl chloride or piperidinethiocarbamoyl chloride derivative of formula IV as, for example, described in Tsai et al., Biorg Med Chem, 7, 29-38 (1999). The reaction to obtain the piperidinecarbamoyl chloride is conveniently carried out with diphosgene, triphosgene or, preferably, phosgene, and the reaction to obtain the piperidinethiocarbamoyl chloride is carried out with dithiophosgene, trithiophosgene or thiophosgene in the presence of a base such as potassium carbonate, sodium carbonate, magnesium carbonate, calcium carbonate, potassium hydrogen carbonate, sodium hydrogen carbonate, magnesium hydrogen carbonate or calcium hydrogen carbonate, preferably sodium hydrogen carbonate. The reaction is carried out at a reaction temperature from −20° C. to the boiling temperature of the reaction mixture, preferably at a reaction temperature between −10° C. and 60° C., most preferably at 0° C. Appropriate solvents for the reaction are inert organic solvents such as ethers (e.g. tetrahydrofuran, diethyl ether, dibutyl ether or dioxane), halogenated hydrocarbons (e.g. dichloromethane or trichloromethane), hydrocarbons (e.g. cyclohexane, methyl cyclohexane, decaline, benzene, toluene, o-xylene, m-xylene or p-xylene) or mixtures of the aforementioned solvents, preferably a mixture of dichloromethane and saturated aqueous sodium hydrogen carbonate.

In step 3 of reaction scheme 1, a piperidinecarbamoyl chloride derivative of formula IV is reacted with HNR²R³, wherein R² and R³ are as defined for compounds of formula I, to obtain a piperidinylurea derivative of formula V. The reaction is carried out using methods similar to those described in for example, Richard C. Larock; Comprehensive Organic Transformations: a guide to functional group preparations, 2nd Edition, 1999, John Wiley and Sons, Inc., New York or J. March (1992), “Advanced Organic Chemistry: Reactions, Mechanisms and Structure”, 4^{th} ed. John Wiley and Sons, for example by combining the reagents in an appropriate solvent at a reaction temperature from −20° C. to the boiling temperature of the reaction mixture, preferably at a reaction temperature between −10° C. and 60° C., most preferably at 0° C. Appropriate solvents for the reaction are ethers (e.g. tetrahydrofuran, diethyl ether, dibutyl ether or dioxane), hydrocarbons (e.g. cyclohexane, methyl cyclohexane, decaline, benzene, toluene, o-xylene, m-xylene or p-xylene), halogenated hydrocarbons (e.g. dichloromethane or trichloromethane), polar aprotic solvents (e.g. dimethylsulfoxide, N,N-dimethylacetamide or N,N-dimethylformamide) or a mixture of the aforementioned solvents. Preferred solvents for the reaction are the aforementioned ethers, most preferably tetrahydrofuran.

Optionally, steps 2 and 3 of reaction scheme 1 can be replaced by step 2.1 of the reaction scheme, by following the reaction conditions described in step 1 of reaction scheme 7 (synthesis via isocyanate and isothiocyanate derivatives). The preferred solvent for this reaction is dichloromethane and the reaction is preferably carried out at ambient temperature.Alternatively, derivative V can be obtained either by reacting derivative III with a suitably activated carbamate (step 2.2), or by converting derivative III into an activated carbamate derivative and reacting this with an appropriate amine (step 2.3). The reactions may be carried out as described in the literature, for example in Lagu et al., J Med Chem, 42, 4794-803 (1999), Rodriguez et al., J Med Chem, 27, 1222-1225 (1984), Sen et al., IzvAkad Nauk SSSR, Ser Khim, 3, 548-51 (1993), Corriu et al., J Organomet Chem, 419, 9-26 (1991), and Takatari et al., J Med Chem, 32, 56-64 (1989).

In step 4 of reaction scheme 1, the protecting group of the piperidinylurea derivative of formula V is cleaved in the presence of trifluoroacetic acid to obtain the deprotected piperidinylurea derivative of formula VI. Alternatively, the reaction can be carried out with other acids as described in ‘Protecting groups in organic synthesis’ 3^{rd} Ed. T. W. Greene, P. G. M. Wuts; Wiley-Interscience, New York 1999 (examples of other acids are: hydrochloric acid, acetyl chloride/methanol, p-toluene sulphonic acid, sulphuric acid, trimethylsilyl iodide, trimethylsilyltrifluoromethanesulphonate, methanesulphonic acid, boron trifluoride diethyl etherate, cerium ammonium nitrate). The reaction is conveniently carried out in an organic solvent such as an ether (e.g. tetrahydrofuran, diethyl ether, dibutyl ether or dioxane), a hydrocarbon (e.g. cyclohexane, methyl cyclohexane, decaline, benzene, toluene, o-xylene, m-xylene or p-xylene), a halogenated hydrocarbon (e.g. dichloromethane or trichloromethane) or a mixture of the aforementioned solvents. Preferred solvents for the reaction are the aforementioned halogenated hydrocarbons; the most preferred solvent is dichloromethane. The reaction is carried out at a reaction temperature from −20° C. to the boiling temperature of the reaction mixture, preferably at a reaction temperature between −10° C. and 60° C., most preferably between 0° C. and 60° C.

In step 5 of reaction scheme 1, the deprotected piperidinyl urea derivative of formula VI is reacted with a carboxaldehyde of formula A-CHO, wherein A is as defined for compounds of formula I (commercially available or synthesised according to known methods from textbooks on organic chemistry e.g. from J. March (1992), “Advanced Organic Chemistry: Reactions, Mechanisms and Structure”, 4^{th} ed. John Wiley and Sons), and subsequently reduced with an appropriate reducing agent, to obtain the 1-substituted piperidinyl urea of formula I-a. Appropriate reducing agents for the reaction are known from the art and are, for example, lithium aluminium hydride, sodium cyanoborohydride or diisobutylaluminium hydride, and, preferably, sodium triacetoxyborohydride. The reaction is carried out in an inert organic solvent such as an ether (e.g. tetrahydrofuran, diethyl ether, dibutyl ether or dioxane), a halogenated hydrocarbon (e.g. dichloromethane or trichloromethane), a hydrocarbon (e.g. cyclohexane, methyl cyclohexane, decaline, benzene, toluene, o-xylene, m-xylene or p-xylene), or a mixture of the aforementioned solvents, preferably dichloromethane, at a reaction temperature from 0° C. to the boiling temperature of the reaction mixture, preferably at ambient temperature.

The reaction can also be carried out under a hydrogen atmosphere in the presence of an appropriate catalyst (for example a palladium catalyst such as palladium on charcoal). This reaction is carried out in an organic solvent, preferably at ambient temperature.

Alternatively, the imine can be pre-formed and subsequently reduced using a reducing agent such sodium triacetoxyborohydride or under a hydrogen atmosphere in the presence of an appropriate catalyst as described above.

An alternative method of carrying out step 5 of reaction scheme 1 is to react a deprotected piperidinyl urea derivative of formula VI with a halo compound of formula A-CH₂Hal wherein A is as defined for compounds of formula I and Hal is chlorine, bromine or iodine, preferably chlorine to obtain a 1-substituted piperidinyl urea of formula I-a. Compounds of formula A-CH₂Hal are commercially available or can be synthesized according to methods known in the art, for example via conversion of an alcohol to the corresponding chloride with e.g. thionyl chloride or according to other methods known from textbooks on organic chemistry e.g. from J. March (1992), “Advanced Organic Chemistry: Reactions, Mechanisms and Structure”, 4^{th} ed. John Wiley and Sons), The reaction is optionally carried out in the presence of an appropriate base and in an appropriate solvent. Appropriate bases are, for example, potassium carbonate, sodium carbonate, magnesium carbonate, calcium carbonate, potassium hydroxide, sodium hydroxide, magnesium hydroxide, calcium hydroxide or N(C₁₋₄-alkyl)₃, wherein different or the same C₁₋₄-alkyl groups are attached to the N-atom. Examples of the aforementioned amines are N(CH₃)₃, N(C₂H₅)₃, N(isoC₃H₇)₃ and, preferably, N(C₂H₅)(isoC₃H₇)₂. The reaction is carried out in an appropriate inert organic solvent such as an ether (e.g. tetrahydrofuran, diethyl ether, dibutyl ether or dioxane), a halogenated hydrocarbon (e.g. dichloromethane or trichloromethane), a hydrocarbon (e.g. cyclohexane, methyl cyclohexane, decaline, benzene, toluene, o-xylene, m-xylene or p-xylene) or a mixture of the aforementioned solvents, preferably dicholoromethane, at a reaction temperature from 0° C. to the boiling temperature of the reaction mixture, preferably at ambient temperature.

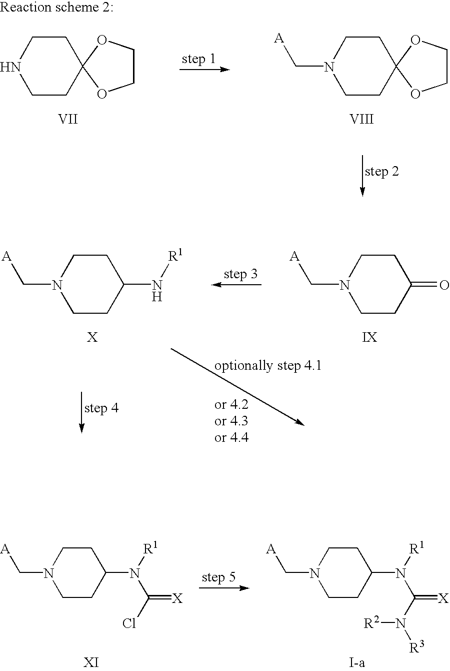

wherein R¹, R², R³, X and A are as defined for compounds of formula I.

In accordance with the present invention, the preparation of compounds of formula I-a

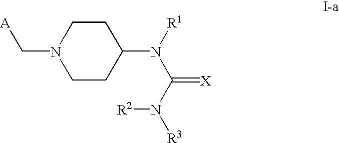

which process comprises

reacting a compound of formula X

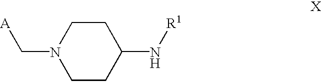

with phosgene or thiophosgene of formula X═CCl₂, to obtain compound of formula XI

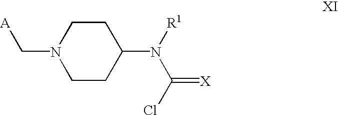

and subsequently reacting compound of formula XI with HNR²R³; or

with a compound of formula XXIV,

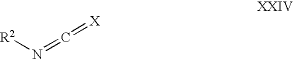

and further reacting the compound of formula I-b

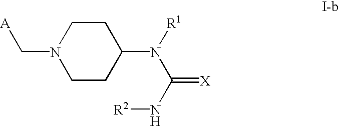

obtained with R³-Hal,

wherein R¹, R², R³, A and X are as defined for compounds of formula I and Hal is chlorine or bromine.

The reaction represents step 4 and 5 of reaction scheme 2 or step 1 of reaction scheme 7 and is described in more detail below.

In reaction scheme 2, step 1 is carried out in the same manner as that described for step 5 of reaction scheme 1 in that a protected piperidinone of formula VII (commercially available) is reacted with a carboxaldehyde of formula A-CHO, wherein A is as defined for compounds of formula I, and subsequently reduced with an appropriate reducing agent, to obtain a 1-substituted piperidine derivative of formula VIII. The compounds of formula A-CHO are commercially available or can be synthesised according to other known methods from textbooks on organic chemistry e.g. from J. March (1992), “Advanced Organic Chemistry: Reactions, Mechanisms and Structure”, 4^{th} ed. John Wiley and Sons).

In step 1 of reaction scheme 2, the protected piperidinyl derivative of formula VII is reacted with a carboxaldehyde of formula A-CHO, wherein A is as defined for compounds of formula I (commercially available or synthesised according to known methods from textbooks on organic chemistry e.g. from J. March (1992), “Advanced Organic Chemistry: Reactions, Mechanisms and Structure”, 4^{th} ed. John Wiley and Sons), and subsequently reduced with an appropriate reducing agent, to obtain the substituted piperidinyl of formula VIII. Appropriate reducing agents for the reaction are known from the art and are for example lithium aluminium hydride, sodium cyanoborohydride or diusobutylaluminium hydride, and, preferably, sodium triacetoxyborohydride. The reaction is carried out in an inert organic solvent such as an ether (e.g. tetrahydrofuran, diethyl ether, dibutyl ether or dioxane), a halogenated hydrocarbons (e.g. dichloromethane or trichloromethane), a hydrocarbon (e.g. cyclohexane, methyl cyclohexane, decaline, benzene, toluene, o-xylene, m-xylene or p-xylene), or a mixture of the aforementioned solvents, preferably dichloromethane, at a reaction temperature from 0° C. to the boiling temperature of the reaction mixture, preferably at ambient temperature.

The reaction can also be carried out under hydrogen atmosphere in the presence of an appropriate catalyst (for example a palladium catalyst such as palladium on charcoal). This reaction is carried out in an organic solvent, preferably at ambient temperature.

Alternatively, the imine can be pre-formed and subsequently reduced using a reducing agent such as sodium triacetoxyborohydride or under a hydrogen atmosphere in the presence of an appropriate catalyst or under transfer hydrogenation conditions such as ammonium formate or cyclohexadiene in the presence of a palladium catalyst as described above.

An alternative method of carrying out step 1 of reaction scheme 2 is to react a protected piperidinyl derivative of formula VII with a halo compound of formula A-CH₂Hal wherein A is as defined for compounds of formula I and Hal is chlorine, bromine or iodine, preferably chlorine to obtain a 1-substituted piperidinyl of formula VIII. Compounds of formula A-CH₂Hal are commercially available or can be synthesized according to methods known in the art, for example via conversion of an alcohol to the corresponding chloride with e.g. thionyl chloride or according to other methods known from textbooks on organic chemistry e.g. from J. March (1992), “Advanced Organic Chemistry: Reactions, Mechanisms and Structure”, 4^{th} ed. John Wiley and Sons), The reaction is optionally carried out in the presence of an appropriate base and in an appropriate solvent. Appropriate bases are, for example, potassium carbonate, sodium carbonate, magnesium carbonate, calcium carbonate, potassium hydroxide, sodium hydroxide, magnesium hydroxide, calcium hydroxide or N(C₁₋₄-alkyl)₃, wherein different or the same C₁₋₄-alkyl groups are attached to the N-atom. Examples of the aforementioned amines are N(CH₃)₃, N(C₂H₅)₃ or N(isoC₃H₇)₃. The reaction is carried out in an appropriate inert organic solvent such as an ether (e.g. tetrahydrofuran, diethyl ether, dibutyl ether or dioxane), a halogenated hydrocarbon (e.g. dichloromethane or trichloromethane), a hydrocarbon (e.g. cyclohexane, methyl cyclohexane, decaline, benzene, toluene, o-xylene, m-xylene or p-xylene) or a mixture of the aforementioned solvents, preferably dicholoromethane, at a reaction temperature from 0° C. to the boiling temperature of the reaction mixture, preferably at ambient temperature.

In step 2 of reaction scheme 2, the protected ketone function of the compound of formula VIII is deprotected in the presence of an appropriate acid to obtain thel-substituted-piperidin-4-one of formula IX. Appropriate acids for the deprotection reaction are mineral acids, tosic acid, and Lewis acids, as described for example in ‘Protecting groups in organic synthesis’ 3^{rd} Ed. T. W. Greene, P. G. M. Wuts; Wiley-Interscience, New York 1999. Examples of suitable acids are, pyridinium tosylate, acetic acid, perchloric acid, bromodimethylborane, trimethylsilyl iodide, titanium(IV) chloride, 2,3-dichloro-5,6-dicyano-1,4-benzoquinone, samarium(III) chloride, sodium iodide/cesium(III) chloride), preferably mineral acids, most preferably hydrochloric acid. The reaction is carried out in water or in an inert organic solvent such as an ether (e.g. tetrahydrofuran, diethyl ether, dibutyl ether or dioxane), a halogenated hydrocarbon (e.g. dichloromethane or trichloromethane), a hydrocarbon (e.g. cyclohexane, methyl cyclohexane, decaline, benzene, toluene, o-xylene, m-xylene or p-xylene), an alcohol (e.g. methanol, ethanol, propanol, butanol, octanol or cyclohexanol), a polar aprotic solvent (e.g. dimethylsulfoxide N,N-dimethylacetamide or N,N-dimethylformamide) or a mixture of the aforementioned organic solvents. The reaction temperature is preferably between −20° C. and the boiling temperature of the reaction mixture, preferably between 50° C. and 150° C. and most preferably between 80° C. and 120° C.

In step 3 of reaction scheme 2, the reaction is carried out in the same manner as described for the first step of reaction scheme 1 in that a 1-substituted-piperidinone of formula IX is reacted with an amine of formula R¹NH₂, wherein R¹ is as defined for compounds of formula I, in the presence of an appropriate reducing agent and an appropriate acid to obtain an aminopiperidine derivative of formula X. The amines of formula R¹NH₂ are commercially available or can be synthesised according to known methods from textbooks on organic chemistry e.g. from J. March (1992), “Advanced Organic Chemistry: Reactions, Mechanisms and Structure”, 4^{th} ed. John Wiley and Sons). Alternatively, as in step 5 of reaction scheme 1, the imine can be pre-formed and subsequently reduced using a reducing agent such as sodium triacetoxyborohydride or under a hydrogen atmosphere in the presence of an appropriate catalyst as described above.

In step 4 of reaction scheme 2, an aminopiperidine derivative of formula X is converted to the corresponding piperidinecarbamoyl chloride derivative of formula XI as for example described in Tsai et al., Biorg Med Chem, 7, 29-38 (1999). The reaction is carried out as described for step 2 in reaction scheme 1.

In step 5 of reaction scheme 2, a piperidinecarbamoyl chloride derivative of formula XI is reacted with HNR²R³, wherein R² and R³ are as defined for compounds of formula I, to obtain piperidine compound of formula I-a. The reaction is carried out as described for step 3 in reaction scheme 1. Optionally, steps 4 and 5 of reaction scheme 2 can be replaced by step 4.1 of the reaction scheme, by following the reaction conditions described in step 1 of reaction scheme 7 (synthesis via isocyanate and isothiocyanate derivatives). The preferred solvent for this reaction is dichloromethane and the reaction is preferably carried out at ambient temperature. Alternatively, derivative I-a can be obtained either by reacting derivative III with a suitably activated carbamate (step 4.2), or by converting derivative III into an activated carbamate derivative and reacting this with an appropriate amine (step 4.3). The reactions may be carried out as described in the literature, for example in Lagu et al., J Med Chem, 1999, 42, 4794-803; Rodriguez et al., J Med Chem, 27, 1222-1225, (1984); Sen et al., IzvAkad Nauk SSSR, Ser Khim, 3, 548-51, (1993); Corriu et al., J Organomet Chem, 1991, 419, 9-26; Takatari et al., J Med Chem, 32, 56-64, (1989). Alternatively, compound of formula Ib may be obtained by reacting a suitable carbamoyl chloride, prepared according to the French patent FR2234293, and a compound of formula X (step 4.4).

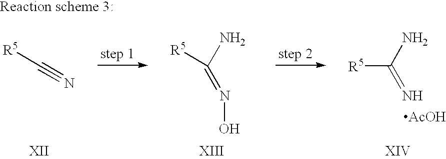

wherein R⁵ is as defined for compounds of formula I.

In reaction scheme 3, step 1 is the reaction of a nitrile derivative of formula XII (commercially available or synthesized according to known methods in textbooks on organic chemistry, for example J. March (1992), “Advanced Organic Chemistry: Reactions, Mechanisms and Structure”, 4^{th} ed. John Wiley and Sons) with hydroxylamine hydrochloride and an appropriate base to obtain an amidoxime of formula XIII as, for example, described in Judkins et al., Syn Com, 26, 4351-67,(1996). Appropriate bases for the reaction are potassium carbonate, sodium carbonate, potassium hydrogen carbonate, sodium hydrogen carbonate, magnesium carbonate, calcium carbonate, potassium hydroxide, sodium hydroxide, magnesium hydroxide, calcium hydroxide and alkoxides, preferably sodium carbonate, and most preferably potassium tert.-butoxide The reaction is conveniently carried out in water or an organic solvent such as an ether (e.g. tetrahydrofuran, diethyl ether, dibutyl ether or dioxane), a halogenated hydrocarbon (e.g. dichloromethane or trichloromethane), a hydrocarbon (e.g. cyclohexane, methyl cyclohexane, decaline, benzene, toluene, o-xylene, m-xylene or p-xylene, an alcohol (e.g. methanol, ethanol, propanol, butanol, octanol or cyclohexanol), a polar aprotic solvent (e.g. dimethylsulfoxide, N,N-dimethylacetamide or N,N-dimethylformamide), or a mixture of the aforementioned organic solvents, preferably the aforementioned alcohols and most preferably methanol or ethanol. The reaction temperature is preferably between −20° C. to the boiling temperature of the reaction mixture, preferably between 30° C. and 150° C. and most preferably between 50° C. and 130° C.

In step 2 of reaction scheme 3, the amidoxime of formula XIII is converted to the corresponding amidine acetate of formula XIV as, for example, described in Judkins et al., Syn Com, 26, 4351-67, (1996). The amidoxime is dissolved in an alcoholic solvent or a carboxylic acid, preferably acetic acid and reacted with acetic anhydride or, optionally carboxylic acids, under reductive conditions for example in the presence of a palladium catalyst (e.g. palladium on charcoal) under a hydrogen atmosphere, or under transfer hydrogenation conditions for example ammonium formate or cyclohexadiene and a palladium catalyst (e.g. palladium on charcoal) or other reducing agents known in the art. Different reaction conditions, for example using tin(II) chloride and hydrogen chloride would lead to the corresponding amidine hydrochlorides. Alternatively, the amidines of formula XIV can be prepared by reduction of the corresponding nitro and nitroso compounds as, for example described in J. March (1992), “Advanced Organic Chemistry: Reactions, Mechanisms and Structure”, 4^{th} ed. John Wiley and Sons. The reaction is preferably carried out at a reaction temperature between −20° C. and the boiling temperature of the reaction mixture, preferably between 0° C. and 70° C. and most preferably at ambient temperature.

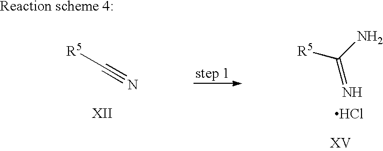

wherein R⁵ is as defined for compounds of formula I.

In reaction scheme 4, a nitrile derivative of formula XII (commercially available or synthesized according to known methods in textbooks on organic chemistry, for example J. March (1992), “Advanced Organic Chemistry: Reactions, Mechanisms and Structure”, 4^{th} ed. John Wiley and Sons) is reacted with ammonium chloride in the presence of an appropriate base as, for example, described in Moss et al., JACS, 107, 2743-8, (1985) to obtain an amidine hydrochloride of formula XV. Appropriate bases for the reaction are alkoxides, preferably methoxide, most preferably sodium methoxide. The reaction is conveniently carried out in an inert organic solvent such as a halogenated hydrocarbon (e.g. dichloromethane or trichloromethane), a hydrocarbon (e.g. cyclohexane, methyl cyclohexane, decaline, benzene, toluene, o-xylene, m-xylene or p-xylene), alcohols (e.g. methanol, ethanol, propanol, butanol, octanol or cyclohexanol), or a mixture of the aforementioned inert organic solvents, preferably the aforementioned alcohols and most preferably methanol. The reaction is preferably carried out at a reaction temperature between −20° C. and the boiling temperature of the reaction mixture, preferably between 0° C. and 70° C. and most preferably at ambient temperature.

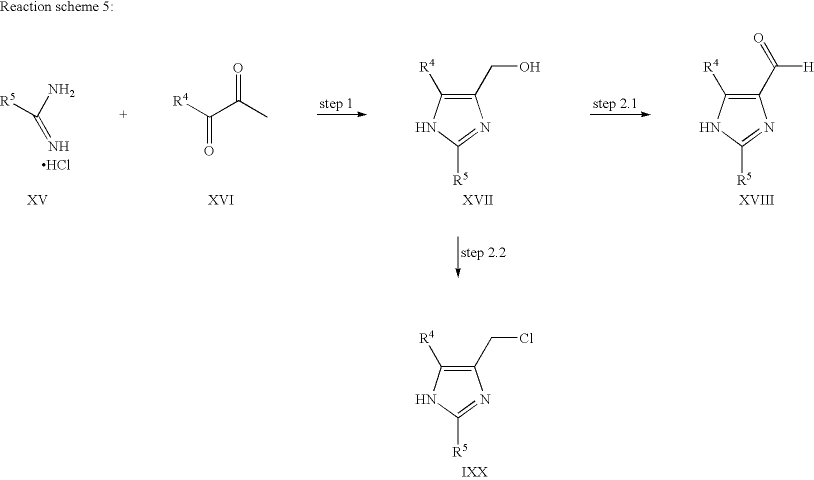

wherein R⁵ is as defined for compounds of formula I and R⁴ is hydrogen, C₁₋₁₂-alkyl, substituted C₁₋₄-alkyl, C₃₋₈-cycloalkyl, C₁₋₄-alkoxy, aryl, substituted aryl, heterocyclyl or substituted heterocyclyl, wherein substituted C₁₋₄-alkyl means alkyl substituted with 1-3 substituents selected from aryl, heterocyclyl, substituted aryl and substituted heterocyclyl; wherein substituted aryl and substituted heterocyclyl means aryl or heterocyclyl substituted with C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens, or substituted heterocyclyl are substituted with 1-4 substituents selected from C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens.

In reaction scheme 5, step 1 is the reaction of an amidine hydrochloride of formula XV or an amidine acetate of formula XIV with a dione derivative of formula XVI (commercially available or synthesized according to known methods in textbooks on organic chemistry, for example J. March (1992), “Advanced Organic Chemistry: Reactions, Mechanisms and Structure”, 4^{th} ed. John Wiley and Sons) in the presence of an appropriate base, followed by reaction with an appropriate acid to obtain a substituted imidazole compound of formula XVII as described in the literature, for example in U.S. Pat. No. 4,126,444 or McNab et al., JCS. Perkin Trans 1, 15, 2203-2210, (1993). The reaction is conveniently carried out, firstly, at a reaction temperature from −20° C. to 50° C., preferably 0° C. and subsequently (for the acidic reaction) at a reaction temperature between 50° C. and the boiling temperature of the reaction mixture, preferably at the boiling temperature of the reaction mixture. Appropriate bases for the reaction are, for example, potassium carbonate, sodium carbonate, potassium hydrogen carbonate, sodium hydrogen carbonate, magnesium carbonate, calcium carbonate, caesium carbonate, potassium hydroxide, sodium hydroxide, magnesium hydroxide, calcium hydroxide, preferably sodium hydroxide. Appropriate acids for the subsequent reaction are mineral acids (e.g. hydrochloric acid, sulphuric acid, and perchloric acid), carboxylic acids (e.g. acetic acid), and p-toluenesulphonic acid, preferably hydrochloric acid. Further, the reaction is carried out in water or an organic solvent such as an alcohol (e.g. methanol, ethanol, propanol, butanol, octanol or cyclohexanol), a polar aprotic solvent (e.g. dimethylsulfoxide, N,N-dimethylacetamide or N,N-dimethylformamide), water or a mixture of the aforementioned organic solvents, preferably water.

In step 2.1 of reaction scheme 5, the hydroxy-methyl group of the substituted imidazole compound of formula XVII is oxidized with an appropriate oxidizing agent to obtain the corresponding aldehyde imidazole compound of formula XVIII. The reaction is carried out according to any known method of oxidation of a benzylic alcohol to the corresponding benzylic aldehyde, for example Swern (oxalyl chloride and dimethyl sulphoxide), Dess-Martin periodinane, tetrapropyl ammonium perruthernate or pyridinium chlorochromate. The reaction is conveniently carried out with manganese dioxide as oxidizing agent in a non-oxidizable organic solvent such as an ether (e.g. tetrahydrofuran, diethyl ether, dibutyl ether or dioxane), a halogenated hydrocarbon (e.g. dichloromethane or trichloromethane), a hydrocarbon (e.g. cyclohexane, methyl cyclohexane, decaline, benzene, toluene, o-xylene, m-xylene or p-xylene or a mixture of the aforementioned organic solvents, preferably 1,4-dioxane. The reaction temperature is preferably between −78° C. and the boiling temperature of the reaction mixture, preferably between 50° C. and 140° C. and most preferably between 60° C. and 120° C.

In step 2.2 of reaction scheme 5, a hydroxymethyl-substituted imidazole compound of formula XVII is treated with an appropriate chlorinating agent to obtain the corresponding chloromethyl-substituted imidazole compound of formula IXX. The reaction is carried out according to known methods for converting a hydroxymethyl group into the corresponding chloromethyl group, for example by treatment with chlorinating agents such as thionyl chloride, oxalyl chloride, phosphorus trichloride, phosphorus pentachloride, and triphenyl phosphine/carbon tetrachloride, preferably thionyl chloride. The reaction is optionally carried out in an inert organic solvent such as an ether (e.g. tetrahydrofuran, diethyl ether, dibutyl ether or dioxane), a halogenated hydrocarbon (e.g. dichloromethane or trichloromethane), a hydrocarbon (e.g. cyclohexane, methyl cyclohexane, decaline, benzene, toluene, o-xylene, m-xylene or p-xylene), or a mixture of the aforementioned organic solvents, preferably with no added solvent. The reaction temperature is preferably between 78° C. and the boiling temperature of the reaction mixture, preferably between 50° C. and 140° C. and most preferably between 60° C. and 120° C.

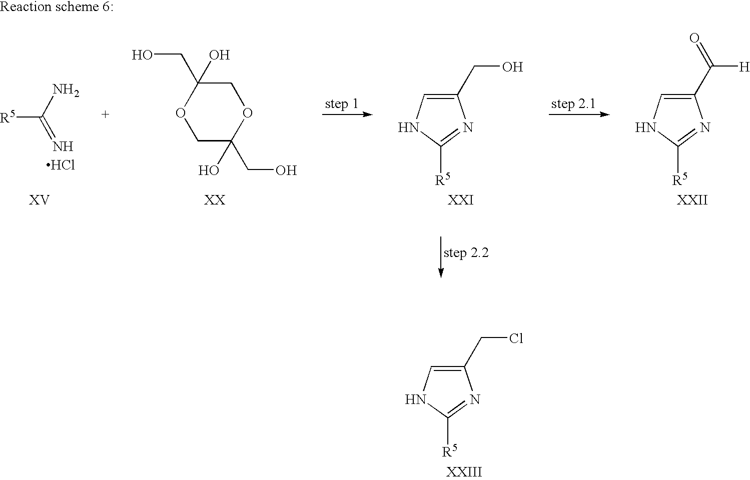

wherein R⁵ is as defined for compounds of formula I.

In reaction scheme 6, step 1 is the reaction of an amidine hydrochloride of formula XV or an amidine acetate of formula XIV with 1,3-dihydroxyacetone dimer of formula XX to obtain an imidazole compound of formula XXI, as described, for example, in Thurkaufet al., J Med Chem, 38, 2251-2255, (1995). The reaction is carried out in the presence of liquid ammonia or an ammonia solution, preferably 0.880 ammonia solution at a reaction temperature between −80° C. and the boiling temperature of the reaction mixture, preferably between 70° C. and 90° C., and most preferably at 80° C.

In step 2.1 of reaction scheme 6, the hydroxymethyl group of a substituted imidazole compound of formula XXI is oxidized with an appropriate oxidizing agent to obtain the corresponding aldehyde imidazole compound of formula XXII. The reaction is carried out as described for step 2.1 in reaction scheme 5.

In step 2.2 of reaction scheme 6, the hydroxymethyl group of a substituted imidazole compound of formula XXI is converted to the corresponding chloromethyl group by treatment with an appropriate chlorinating agent to obtain the corresponding chloromethyl-imidazole compound of formula XXIII. The reaction is carried out as described for step 2.2 in reaction scheme 5.

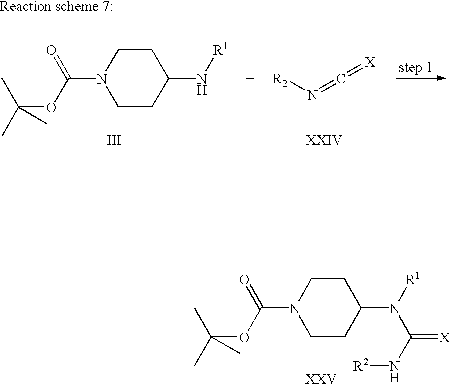

wherein R¹, R² and X are as defined for compounds of formula I.

In reaction scheme 7, an aminopiperidine derivative of formula III is reacted with an isothiocyanate or isocyanate of formula XXIV (commercially available or synthesized according to known methods in textbooks on organic chemistry, for example J. March (1992), “Advanced Organic Chemistry: Reactions, Mechanisms and Structure”, 4^{th} ed. John Wiley and Sons) to give a piperidinyl thiourea or a piperidinyl urea derivative of formula XXV. Appropriate solvents for the reaction are organic solvents such as ethers (e.g. tetrahydrofuran, diethyl ether, dibutyl ether or dioxane), halogenated hydrocarbons (e.g. dichloromethane or trichloromethane), hydrocarbons (e.g. cyclohexane, methyl cyclohexane, decaline, benzene, toluene, o-xylene, m-xylene or p-xylene), alcohols (e.g. methanol, ethanol, propanol, butanol, octanol or cyclohexanol), or a mixture of the aforementioned organic solvents, preferably dichloromethane or a mixture of toluene and ethanol. The reaction is carried out at a reaction temperature from −20° C. to the boiling temperature of the reaction mixture, preferably at a reaction temperature between 0° C. and 110° C., most preferably at ambient temperature for dichloromethane and between 60° C. and 100° C. for toluene/ethanol.

An alternative method for the synthesis of a piperidinyl thiourea or a piperidinyl urea derivative of formula XXV is the reaction of an aminopiperidine derivative of formula III with a suitably activated thiocarbamate or carbamate.

Optionally, the NHR²-function of a piperidinyl thiourea or a piperidinyl urea derivative of formula XXV may be reacted with R³-Hal, wherein R³ is as defined for compounds of formula I and Hal is chlorine or bromine, according to methods known in the art, for example Hoffmann-alkylation, to obtain a piperidine compound of formula V. This reaction is known from textbooks on organic chemistry for example J. March (1992), “Advanced Organic Chemistry: Reactions, Mechanisms and Structure”, 4^{th} ed. John Wiley and Sons.

Piperidinyl thiourea or piperidinyl urea derivatives of formula XXV are subsequently deprotected as described in step 4 of reaction scheme 1

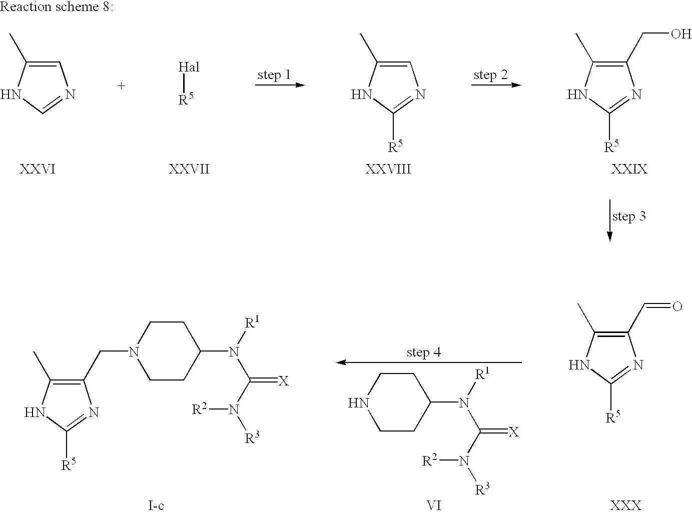

wherein R¹, R², R³ and X are as defined for compounds of formula I, and R⁵ is C₁₋₁₂-alkyl, substituted C₁₋₄-alkyl, C₃₋₈-cycloalkyl, aryl, substituted aryl, heterocyclyl or substituted heterocyclyl, wherein substituted C₁₋₄-alkyl means alkyl substituted with 1-3 substituents selected from aryl, heterocyclyl, substituted aryl and substituted heterocyclyl; wherein substituted aryl and substituted heterocyclyl means aryl or heterocyclyl substituted with C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens; and wherein substituted aryl means aryl substituted with 1-5 substituents selected from C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens, and wherein substituted heterocyclyl is substituted with 1-4 substituents selected from C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens and wherein Hal is fluorine, chlorine, bromine or iodine.

In reaction scheme 8, step 1 is the reaction of a substituted imidazole derivative of formula XXVI with a chloride derivative of formula XXVII in an appropriate solvent followed by reaction with an appropriate base, to obtain a substituted imidazolyl phenyl methanone derivative of formula XXVIII as, for example described in Bastiaansen et al., Synthesis, 675-6, (1978). The reaction of the substituted imidazole derivative of formula XXVI with the chloride derivative of formula XXVII is carried out under an inert atmosphere such as a nitrogen or argon atmosphere in the presence of a base such as pyridine or a tertiary amine (e.g. trimethylamine, triethylamine, and tripropylamine). Optionally, an inert organic solvent such as a halogenated hydrocarbon (e.g. dichloromethane or trichloromethane), a hydrocarbon (e.g. cyclohexane, methyl cyclohexane, decaline, benzene, toluene, o-xylene, m-xylene or p-xylene), or a mixture of the aforementioned mentioned solvents may be used. Preferably, the reaction is carried out using a mixture of pyridine and triethylamine as the solvent. This part of the reaction is conveniently carried out at a reaction temperature from −20° C. to 70° C., preferably at ambient temperature. Appropriate bases for the second part of the reaction are potassium carbonate, sodium carbonate, potassium hydrogen carbonate, sodium hydrogen carbonate, magnesium carbonate, calcium carbonate, cesium carbonate, potassium hydroxide, sodium hydroxide, magnesium hydroxide, and calcium hydroxide, preferably sodium hydroxide. An appropriate solvent is water. This part of the reaction is carried out at a reaction temperature between 50° C. and the boiling temperature of the reaction mixture, preferably at the boiling temperature of the reaction mixture.

The reaction may be carried out as described above or according to Gompper et al., Chem Ber, 92, 550 (1959) or Hlasta et al., Bioorg Med Chem Lett, 7, 89-94, (1997).

In step 2 of reaction scheme 8, a substituted imidazolyl derivative of formula XXVIII is reacted with formaldehyde or paraformaldehyde in the presence of an appropriate base to obtain the corresponding substituted imidazolyl methanol compound of formula XXIX, as for example described in Watson et al., Syn Com, 22, 2971-7, (1992). Appropriate bases for the reaction are potassium carbonate, sodium carbonate, potassium hydrogen carbonate, sodium hydrogen carbonate, magnesium carbonate, calcium carbonate, cesium carbonate, potassium hydroxide, sodium hydroxide, magnesium hydroxide, and calcium hydroxide, preferably sodium hydroxide. The reaction is preferably carried out at a reaction temperature between −20° C. and the boiling temperature of the reaction mixture, preferably between 0° C. and 100° C. and most preferably at a reaction temperature between 30° C. and 70° C. Further, the reaction is carried out in water or an organic solvent such as an ether (e.g. tetrahydrofuran, diethyl ether, dibutyl ether or dioxane), a halogenated hydrocarbon (e.g. dichloromethane or trichloromethane), a hydrocarbon (e.g. cyclohexane, methyl cyclohexane, decaline, benzene, toluene, o-xylene, m-xylene or p-xylene), pyridine, an alcohol (e.g. methanol, ethanol, propanol, butanol, octanol or cyclohexanol) or a mixture of the aforementioned solvents, preferably water and ethanol.

In step 3 of reaction scheme 8, a substituted imidazole methanol compound of formula XXIX is oxidized with an appropriate oxidizing agent to obtain the corresponding imidazole aldehyde compound of formula XXX. The reaction is carried out as described for step 2.1 in reaction scheme 5.

In step 4 of reaction scheme 8, an imidazole aldehyde compound of formula XXX is reacted with a piperidine derivative of formula VI (synthesized as described in reaction scheme 1 or by deprotection of compound XXV from reaction scheme 7) to obtain a piperidinylurea of formula I-c. The reaction is carried out as described for step 5 in reaction scheme 1.

If R⁵ in a compound of formula I-c is an optionally substituted phenyl-carbonyl group the carbonyl group may be reduced with an appropriate reducing agent to the corresponding phenylhydroxymethyl group as, for example, described in Ooi & Suschitzy, J Chem Soc, 2871(1982). Appropriate reducing agents are sodium borohydride, lithium aluminium hydride, di-isobutyl aluminium hydride, alane (preparation in situ according to methods known in the art), or other hydride reducing reagents known in the art, preferably sodium borohydride. The reaction is carried out at a reaction temperature between −78° C. and the boiling temperature of the reaction mixture, preferably between 0° C. and 70° C., and most preferably at ambient temperature. Further, the reaction is carried out in an organic solvent such as an ether (e.g. tetrahydrofuran, diethyl ether, dibutyl ether or dioxane), a halogenated hydrocarbon (e.g. dichloromethane or trichloromethane), a hydrocarbon (e.g. cyclohexane, methyl cyclohexane, decaline, benzene, toluene, o-xylene, m-xylene or p-xylene), pyridine, an alcohol (e.g. methanol, ethanol, isopropanol, butanol, octanol or cyclohexanol), a polar aprotic solvents (e.g. dimethylsulfoxide, N,N-dimethylacetamide or N,N-dimethylformamide), or a mixture of the aforementioned organic solvents, preferably isopropyl alcohol.

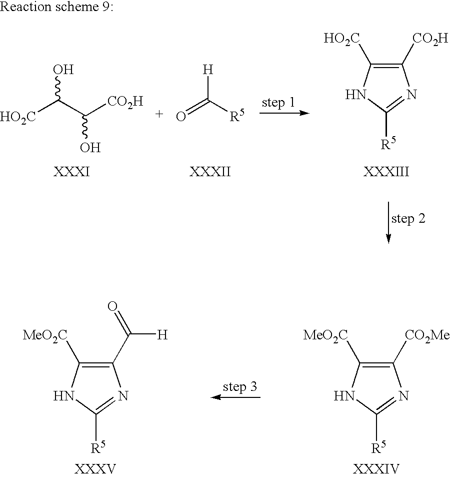

wherein R⁵ is C₁₋₁₂-alkyl, substituted C₁₋₄-alkyl, C₃₋₈-cycloalkyl, aryl, substituted aryl, heterocyclyl, or substituted heterocyclyl, wherein substituted C₁₋₄-alkyl means alkyl substituted with 1-3 substituents selected from aryl, heterocyclyl, substituted aryl and substituted heterocyclyl; wherein substituted aryl and substituted heterocyclyl means aryl or heterocyclyl substituted with C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens,;and wherein substituted aryl means aryl substituted with 1-5 substituents selected from C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens; and wherein substituted heterocyclyl means heterocyclyl substituted with 1-4 substituents selected from C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens.

In reaction scheme 9, step 1 is the reaction of racemic tartaric acid of formula XXXI (commercially available) with concentrated nitric acid, followed by fuming nitric acid and sulfuric acid at a reaction temperature from 10° C. to 60° C., preferably at a reaction temperature from 20° C. to 50° C. The reaction mixture is subsequently cooled to a temperature from −20° C. to 0° C., preferably −10° C., to obtain a solid intermediate which is reacted with a substituted aldehyde derivative of formula XXXII (commercially available or synthesised according to methods known in the art) at a pH of 6 to 8, preferably 7, in the presence of ammonia solution, preferably concentrated ammonia solution, to obtain a phenyl-substituted imidazole derivative of formula XXXIII. The reaction temperature is preferably in the range of −20° C. to 20° C., more preferably in the range of −10° C. to 10° C. This type of reaction is described by MacKinnon et al in Tetrahedron, 54, 9837-48, (1998).

In step 2 of reaction scheme 9, the dicarboxylic acid derivative of formula XXXIII is esterified using a lower alcohol, for example methanol, in the presence of an appropriate mineral acid, to obtain the corresponding diester of formula XXXIV. The esterification reaction is carried out according to methods known from textbooks on organic chemistry e.g. from J. March (1992), “Advanced Organic Chemistry: Reactions, Mechanisms and Structure”, 4^{th} ed. John Wiley and Sons. Appropriate acids for the esterification reaction are mineral acids (e.g. hydrochloric acid and sulphuric acid), and p-toluenesulphonic acid, preferably sulphuric acid. The reaction is carried out at a reaction temperature between ambient temperature to the boiling temperature of the reaction mixture, preferably at the boiling temperature of the reaction mixture, optionally in the presence of an organic solvent such as an ether (e.g. tetrahydrofuran, diethyl ether, dibutyl ether or dioxane) or a hydrocarbon (e.g. cyclohexane, methyl cyclohexane, decaline, benzene, toluene, o-xylene, m-xylene or p-xylene).

In step 3 of reaction scheme 9, the diester of formula XXXIV is treated with an appropriate reducing agent to obtain the corresponding formyl imidazole compound of formula XXXV. Appropriate reducing agents for the reaction are known from the art and are for example diisobutylaluminiumhydride. The reaction is carried out in the presence of sodium hydride in an inert organic solvent such as an ether (e.g. tetrahydrofuran, diethyl ether, dibutyl ether or dioxane), a hydrocarbon (e.g. cyclohexane, methyl cyclohexane, decaline, toluene, o-xylene, m-xylene or p-xylene) or a halogenated aromatic hydrocarbon, at a reaction temperature between −78° C. and the boiling temperature of the reaction mixture, preferably starting at a reaction temperature between 50° C. and the boiling temperature of the reaction mixture (after the addition of sodium hydride) and at a temperature between −78° C. and 0° C. for the addition of the reducing agent. This type of reaction is known in the art and is, for example, carried out as described in WO 9119715.

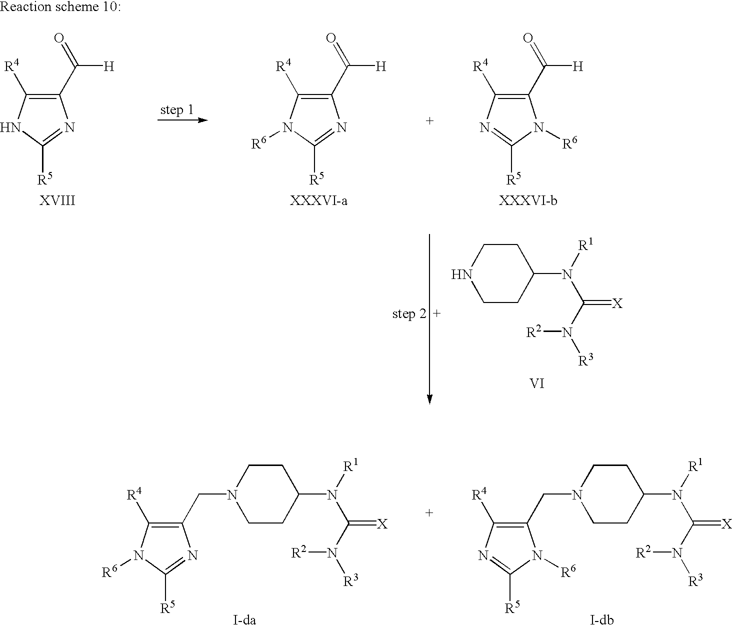

wherein R¹, R², R³, R⁴, R⁵ and X are as defined for compounds of formula I, and wherein R⁶ is C₁₋₁₂-alkyl, substituted C₁₋₄-alkyl, C₃₋₈-cycloalkyl, COR, CO₂R; wherein substituted C₁₋₄-alkyl means alkyl substituted with 1-3 substituents selected from C₃₋₈-cycloalkyl, aryl, heterocyclyl, substituted aryl and substituted heterocyclyl; wherein substituted aryl and substituted heterocyclyl means aryl or heterocyclyl substituted with C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens; and wherein substituted aryl are substituted with 1-5 substituents and substituted heterocyclyl are substituted with 1-4 substituents, these substituents selected from C₁₋₄-alkoxy, halogen, CN, NO₂, COR⁷, CO₂R⁷, CONR⁷R⁸, NR⁷R⁸, NHCOR⁷, SO₂NR⁷R⁸, SO₂R⁷, C₁₋₄-alkyl or C₁₋₄-alkyl substituted with 1-3 halogens.

In reaction scheme 10, step 1 is the reaction of an imidazole compound of formula XVIII with R⁶-Hal, wherein R⁶ is as defined above and Hal is Cl, Br, F or I (commercially available or synthesised according to known methods from textbooks on organic chemistry e.g. from J. March (1992), “Advanced Organic Chemistry: Reactions, Mechanisms and Structure”, 4^{th} ed. John Wiley and Sons) in the presence of an appropriate base to obtain a mixture of the corresponding N-alkylated or arylated imidazole. Appropriate bases for the reaction are known from the art and are for example tertiary amines, carbonates (e.g.sodium carbonate, magnesium carbonate, calcium carbonate or cesium carbonate), alkyl lithiums (e.g. methyl lithium or ethyl lithium), metal hydrides (e.g. sodium hydride, lithium hydride or calcium hydride), preferably sodium hydride. The reaction is carried out in an inert organic solvent such as a polar aprotic solvents (e.g. dimethylsulfoxide, N,N-dimethylacetamide or N,N-dimethylformamide, an ether (e.g. tetrahydrofuran, diethyl ether, dibutyl ether or dioxane), a chlorinated hydrocarbon (e.g. dichloromethane or trichloromethane), a hydrocarbon (e.g. cyclohexane, methyl cyclohexane, decaline, benzene, toluene, o-xylene, m-xylene or p-xylene), or mixtures of the aforementioned solvents, preferably dimethyl formamide. The reaction is carried out at a reaction temperature from −20° C. to the boiling temperature of the reaction mixture, preferably at ambient temperature.

In step 2 of reaction scheme 10, the substituted imidazole derivative of formula XXXVI-a and XXXVI-b is reacted with a piperidine derivative of formula VI and subsequently reduced with an appropriate reducing agent to obtain the substituted piperidinyl derivatives of formula I-da and I-db. Appropriate reducing agents for the reaction are known from the art and are, for example, sodium cyanoborohydride or diisobutylaluminium hydride, preferably sodium triacetoxyborohydride. The reaction is carried out in an inert organic solvent such as an ether (e.g. tetrahydrofuran, diethyl ether, dibutyl ether or dioxane), a halogenated hydrocarbon (e.g. dichloromethane or trichloromethane), a hydrocarbon (e.g. cyclohexane, methyl cyclohexane, decaline, benzene, toluene, o-xylene, m-xylene or p-xylene), or a mixture of the aforementioned solvents, preferably dichloromethane, at a reaction temperature from 0° C. to the boiling temperature of the reaction mixture, preferably at ambient temperature.

The reaction can also be carried out under hydrogen atmosphere in the presence of an appropriate catalyst (for example a palladium catalyst such as palladium on charcoal). This reaction is carried out in an organic solvent, preferably at ambient temperature.

Alternatively, the imine can be pre-formed and subsequently reduced using a reducing agent such as sodium triacetoxyborohydride or under a hydrogen atmosphere in the presence of an appropriate catalyst as described above.

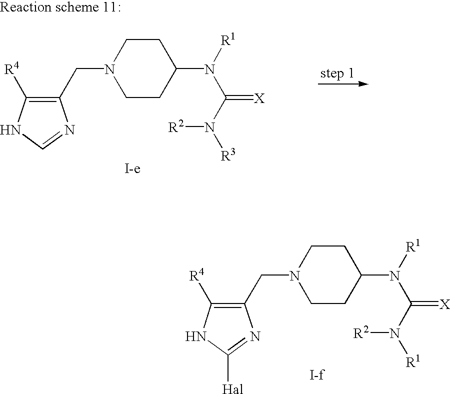

wherein R¹, R², R³, R⁴, and X are as defined for compounds of formula I and Hal is chlorine, bromine or iodine.

In step 1 of reaction scheme 11, an imidazole derivative of formula I-e (commercially available or synthesized according to the methods described before) is treated with chlorine, bromine or iodine, preferably iodine, in the presence of an appropriate base to obtain the corresponding iodo-imidazole derivative of formula I-f. Appropriate bases for the reaction are known from the art and are, for example, carbonates (e.g. sodium carbonate, magnesium carbonate, potassium carbonate or cesium carbonate), hydrogen carbonates (e.g. sodium hydrogen carbonate or potassium hydrogen carbonate), hydroxides (e.g. sodium hydroxide, potassium hydroxide, calcium hydroxide or barium hydroxide), preferably sodium hydroxide. The reaction is carried out in an inert organic solvent such as a polar aprotic solvents (e.g. dimethylsulfoxide, N,N-dimethylacetamide or N,N-dimethylformamide, an ether (e.g. tetrahydrofuran, diethyl ether, dibutyl ether or dioxane), a chlorinated hydrocarbon (e.g. dichloromethane or trichloromethane), hydrocarbons (e.g. cyclohexane, methyl cyclohexane, decaline, benzene, toluene, o-xylene, m-xylene or p-xylene), an alcohol (e.g. methanol, ethanol, propanol, butanol, octanol or cyclohexanol), or a mixture of the aforementioned solvents, preferably a mixture of dichloromethane and water. The reaction is carried out at a reaction temperature from −20° C. to the boiling temperature of the reaction mixture, preferably at ambient temperature.

The following examples illustrate the present invention:

In the following examples the abbreviations used have the following significations:

min minute(s)

h hour(s)

d day(s)

DMAW 120 denotes asolvent mixture containing dichloromethane, methanol, acetic acid and water in the ratio 120:15:3:2 respectively

DMAW 240 denotes a solvent mixture containing dichloromethane, methanol, acetic acid and water in the ratio 240:24:32:21 respectively

All temperatures are given in degrees Celsius (° C.).

Mass spectra were recorded under electron impact conditions on a THERMOQUEST MAT95 S with a source temperature of 200° C. or under electrospray ionization spectra conditions, on either a THERMOQUEST SSQ 7000 [Solvent 0.085% TFA in 90% Acetonitrile/water; flow rate 100 microliters/min; capillary 250° C.; spray voltage 5 KV; sheath gas 80 psi], or an LC-MS system (liquid chromatograph coupled to mass spectrum) THERMOQUEST TSQ 7000 ELECTROSPRAY or MICROMASS PLATFORM ELECTROSPRAY [Solvent 0.1% TFA in water or 0.085% TFA in 90% acetonitrile/water or 0.085% TFA in acetonitrile]. With regard to the known starting materials, some of these may be purchased from commercial suppliers. Catalogue numbers for commercially available starting materials are provided. Other known starting materials and their analogues can be prepared by methods well known in the art. Examples of compounds available from commercial suppliers, and citations to the synthesis of other compounds and their analogues are provided in the following:

Compounds, whenever prepared by the processes of the present invention are also an object of the present invention.

### EXAMPLES ACCORDING to REACTION SCHEME 1

### Reaction Scheme 1, Step 1

### 4-Phenylamino-piperidine-1-carboxylic Acid Tert.-butyl Ester

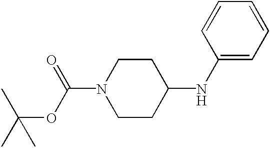

A solution of N-tert-butoxycarbonyl-4-piperidone (Lancaster 13361, 7 g) and aniline (Aldrich 24228-4, 3.3 g) in dichloromethane (200 ml) was treated with sodium triacetoxyborohydride (Aldrich 31639, 10.4 g) followed by acetic acid (2.1 g) and the mixture stirred for 2 h at ambient temperature. 1M Aqueous sodium hydroxide solution (100 ml) was added, followed by diethyl ether (200 ml) and the mixture stirred vigorously for 5 min. The organic phase was separated, washed with water (100 ml), followed by brine (100 ml), dried (anhydrous magnesium sulphate), filtered and evaporated to give the title compound as a white solid (9.5 g, 98%). Mass spectrum 277 [M+H]⁺.

The following compounds were produced in a manner analogous to that described above, by replacing aniline with the appropriate amine

| Systematic name | Structure | m/z [M + H]⁺ |
|---|---|---|
| 4-Benzylamino-piperidine-1- carboxylic acid tert.-butyl ester | 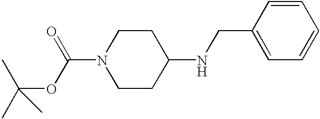 | 291 |
| 4-(4-Methoxy-phenylamino)- piperidine-1-carboxylic acid tert.- butyl ester | 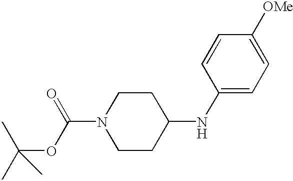 | 307 |
| 4-Allylamino-piperidine-1- carboxylic acid tert.-butyl ester | 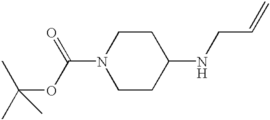 | 241 |

### Reaction Scheme 1, Step 2

### 4-Phenylaminocarbamoylchloride-piperidine-1-carboxylic Acid Tert.-butyl Ester

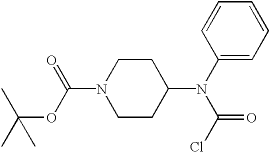

To a rapidly stirring, ice-cold solution of 4-phenylamino-piperidine-1-carboxylic acid tert.-butyl ester (5 g) in dichloromethane (500 ml) and saturated aqueous sodium hydrogen carbonate solution (400 ml) was added 20% phosgene in toluene (Fluka 79380, 50 ml). After 1 h the organic phase was separated, dried (anhydrous magnesium carbonate), filtered and evaporated to give the title compound as a pale yellow solid (6.2 g, 100%). Mass spectrum 339 [M+H]⁺.

The following compounds were produced in a manner analogous to that described above by replacing the 4-phenylamino-piperidine-1-carboxylic acid tert.-butyl ester with an appropriate amine:

| Name | Structure | m/z [M + H]⁺ |
|---|---|---|
| 4-Benzylcarbamylchloride-piperidine- 1-carboxylic acid tert.-butyl ester | 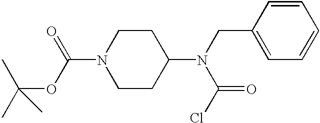 | 353 |
| 4-(4-Methoxy- phenylcarbamylchloride)-piperidine- 1-carboxylic acid tert.-butyl ester | 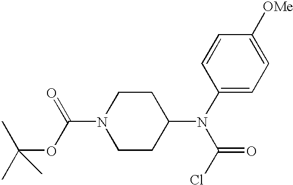 | 369 |
| 4-Allylcarbamyl chloride-piperidine- 1-carboxylic acid tert.-butyl ester | 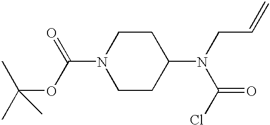 | 303 |

### Reaction Scheme 1, Step 3

### 4-(3-Methyl-1-phenyl-ureido)-piperidine-1-carboxylic Acid Tert.-butyl Ester

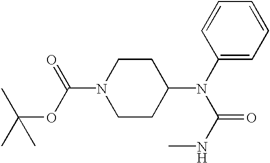

To an ice-cold solution of methylamine (Fluka 65590, 33% in ethanol, 2.5 ml) in ethanol (30 ml) was added, slowly, a solution of 4-phenylaminocarbamoyl chloride-piperidine-1-carboxylic acid tert.-butyl ester (3 g) in tetrahydrofuran (10 ml) and the mixture allowed to stir for 1 h. The volatile solvents were removed under reduced pressure and the residue partitioned between dichloromethane (40 ml) and water (30 ml). The organic layer was separated, dried (anhydrous magnesium sulphate), filtered and evaporated. The residue was recyrstallized from toluene to give the title compound as a white, crystalline solid (2.1 g, 71%). Mass spectrum 334 [M+H]⁺.

The following compounds were produced in a manner analogous to that described above, by replacing methylamine with the appropriate amine and the 4-phenylaminocarbamoyl chloride-piperidine-1-carboxylic acid tert.-butyl ester with the appropriate carbamoyl chloride:

| Systematic name | Structure | m/z [M + H]⁺ |
|---|---|---|
| 4-(1-Benzyl-3-methyl-ureido)- piperidine-1-carboxylic acid tert.- butyl ester | 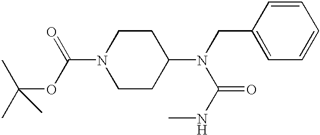 | 348 |
| 4-[1-(4-Methoxy-phenyl)-3-methyl- ureido]-piperidine-1-carboxylic acid tert.-butyl ester | 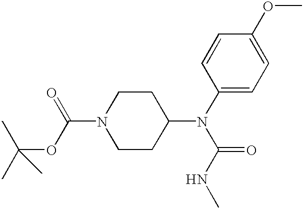 | 364 |
| 4-(3,3-Dimethyl-1-phenyl-ureido)- piperidine-1-carboxylic acid tert.- butyl ester | 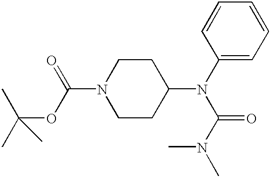 | 348 |
| 4-[1-Allyl-3-(4-nitro-benzyl)- ureido]-piperidine-1-carboxylic acid tert.-butyl ester | 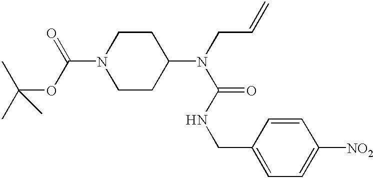 | 419 |

### Reaction Scheme 1, Step 4

### 3-Methyl-1-phenyl-1-piperidin-4-yl-urea

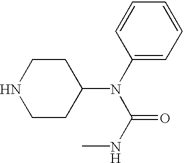

A solution of 4-(3-methyl-1-phenyl-ureido)-piperidine-1-carboxylic acid tert.-butyl ester (15.2 g) in dichloromethane (80 ml) was treated with trifluoroacetic acid (20 ml) and the mixture stirred at ambient temperature for 1 h. The mixture was evaporated and the residue partioned between 2M aqueous sodium hydoxide solution (100 ml) and dichloromethane (200 ml). The organic phase was separated, dried (anhydrous magnesium sulphate), filtered and evaporated to give the title compound as a white solid (10.1 g, 95%). Mass spectrum 234 [M+H]⁺.

The following compounds were produced in a manner analogous to that described above by replacing the 4-(3-methyl-1-phenyl-ureido)-piperidine-1-carboxylic acid tert.-butyl ester with the appropriate tert-butoxycarbonyl derivative:

| Systematic name | Structure | m/z [M + H]⁺ |
|---|---|---|
| 1-Benzyl-3-methyl-1-piperidin-4-yl-urea | 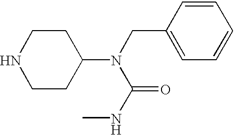 | 248 |
| 1-(4-Methoxy-phenyl)-3-methyl-1- piperidin-4-yl-urea | 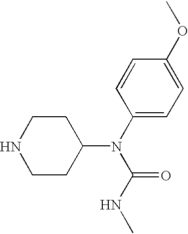 | 264 |
| 1-Allyl-3-(4-nitro-benzyl)-1- piperidin-4-yl-urea | 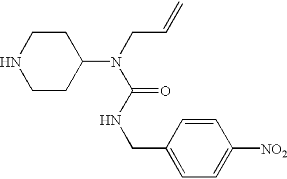 | 389 |
| 1,1-Dimethyl-3-phenyl-3-piperidin-4- yl-urea | 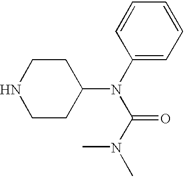 | 248 |
| 1-Benzyl-1-piperidin-4-yl-3-pyridin- 2-yl-urea | 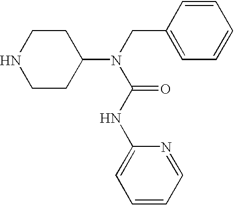 | 311 |

### Reaction Scheme 1, Step 5

### 3-Methyl-1-{1-[5-methyl-2-(4-trifluoromethyl-phenyl)-1H-imidazol-4-ylmethyl]-piperidin-4-yl}-1-phenyl-urea

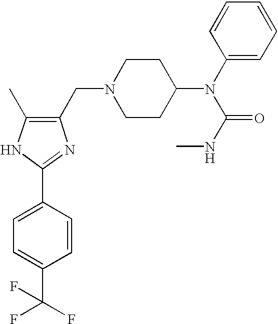

A mixture of 3-methyl-1-phenyl-1-piperidin-4-yl-urea (55 mg) and 5-methyl-2-(4-trifluoromethyl-phenyl)-1H-imidazole-4-carbaldehyde (60 mg) in dichloromethane (10 ml) was treated with sodium triacetoxyborohydride (Aldrich 31639-3, 70 mg) and stirred at ambient temperature for 2 h. Ethyl acetate (40 ml) was added, followed by saturated aqueous sodium hydrogen carbonate (20 ml), the organic layer was separated, dried (anhydrous magnesium sulphate), filtered and evaporated. The residue was purified by flash chromatography on silica gel eluting with DMAW 240. The resulting acetate salt was partitioned between dichloromethane (10 ml) and 2M aqueous sodium hydroxide solution (10 ml). The organic phase was separated, dried (anhydrous magnesium sulphate), filtered and evaporated to leave the title compound as a white solid (30 mg, 26%). Mass spectrum 472 [M+H]⁺.

The following compounds were produced in a manner analogous to that described above, by using the appropriate aldehyde prepared as described in reaction schemes 5, 6, 8 or 9 in place of 5-methyl-2-(4-trifluoromethyl-phenyl)-1H-imidazole-4-carbaldehyde and the appropriate amine prepared as described in reaction schemes 1 or 7 in place of 3-methyl-1-phenyl-1-piperidin-4-yl-urea.

| Systematic name | Structure | m/z [M + H]⁺ |
|---|---|---|
| 3-Methyl-1-[1-[(5-methyl-1H-imidazol- 4-yl)methyl]-4-piperidinyl]-1- phenylurea | 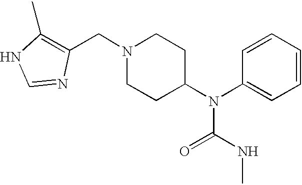 | 328 |
| 3-Methyl-1-[1-[(5-methyl-2-phenyl-1H- imidazol-4-yl)methyl]-4-piperidinyl]-1- phenylurea | 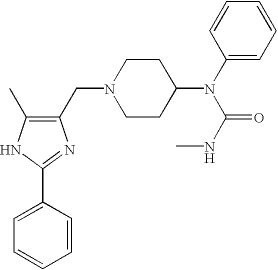 | 404 |
| 1,1-Dimethyl-3-[1-[(5-methyl-2-phenyl- 1H-imidazol-4-yl)methyl]-4- piperidinyl]-3-phenylurea | 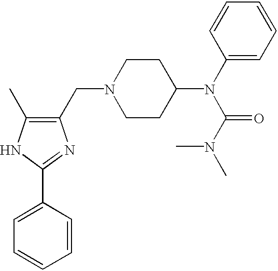 | 418 |
| 1-Benzyl-3-methyl-1-[1-[(5-methyl-2- phenyl-1H-imidazol-4-yl)methyl]-4- piperidinyl]urea | 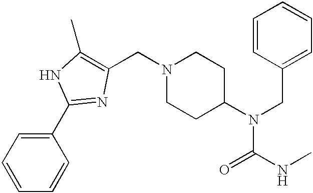 | 418 |
| 1-(4-Methoxyphenyl)-3-methyl-1-[1- [(5-methyl-2-phenyl-1H-imidazol-4- yl)methyl]-4-piperidinyl]urea | 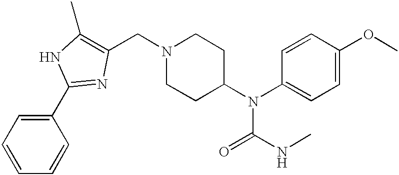 | 434 |
| 1-Benzyl-3-methyl-1-[1-[[5-methyl-2- [4-(trifluoromethyl)phenyl]-1H- imidazol-4-yl]methyl]-4- piperidinyl]urea | 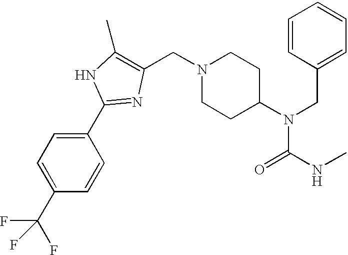 | 486 |
| 3-Methyl-1-[1-[[5-methyl-2-(4- methylphenyl)-1H-imidazol-4- yl]methyl]-4-piperidinyl]-1-phenylurea | 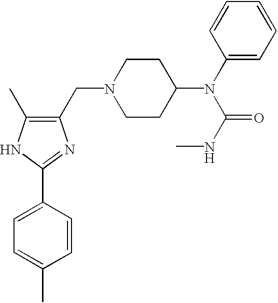 | 418 |
| 1-[1-[[2-(4-Chlorophenyl)-5-methyl- 1H-imidazol-4-yl]methyl]-4- piperidinyl]-3-methyl-1-phenylurea | 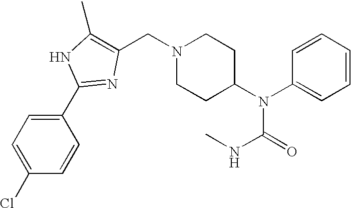 | 439 |
| 3-Methyl-1-phenyl-1-[1-[[2-[4- (trifluoromethyl)phenyl]-1H-imidazol- 4-yl]methyl]-4-piperidinyl]urea | 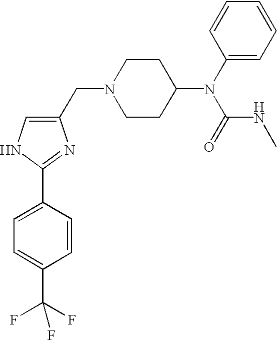 | 458 |
| 1-[1-[[2-(2,3-Dimethoxyphenyl)-1H- imidazol-4-yl]methyl]-4-piperidinyl]-3- methyl-1-phenylurea | 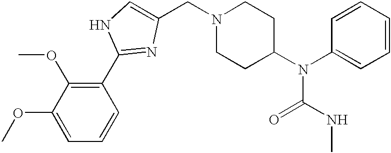 | 450 |
| 1-[1-[[2-(2,3-Dimethoxyphenyl)-5- methyl-1H-imidazol-4-yl]methyl]-4- piperidinyl]-3-methyl-1-phenylurea | 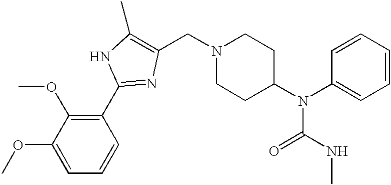 | 464 |
| 1-Benzyl-3-methyl-1-[1-[[5-phenyl-2- [4-(trifluoromethyl)phenyl]-1H- imidazol-4-yl]methyl]-4- piperidinyl]urea | 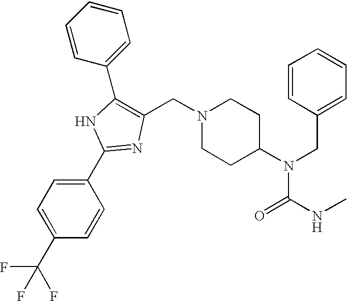 | 548 |
| 3-Methyl-1-phenyl-1-[1-[[5-phenyl-2- [4-(trifluoromethyl)phenyl]-1H- imidazol-4-yl]methyl]-4- piperidinyl]urea | 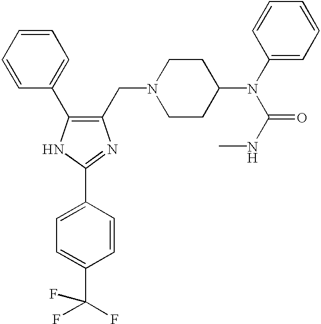 | 534 |
| 1-Benzyl-3-methyl-1-[1-[(5-methyl-1H- imidazol-4-yl)methyl]-4- piperidinyl]urea | 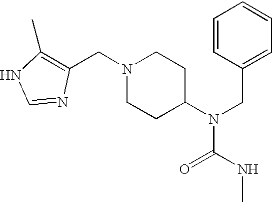 | 342 |
| 1-Allyl-1-[1-[[5-methyl-2-[4- (trifluoromethyl)phenyl]-1H-imidazol- 4-yl]methyl]-4-piperidinyl]-3-(4- nitrobenzyl)urea | 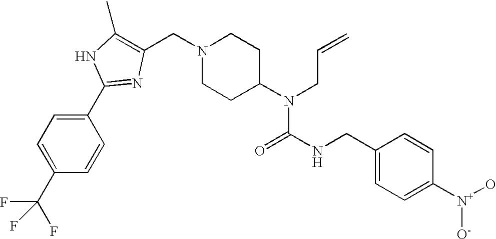 | 557 |
| 1-[1-[(2-Benzoyl-5-methyl-1H- imidazol-4-yl)methyl]-4-piperidinyl]-1- benzyl-3-methylurea | 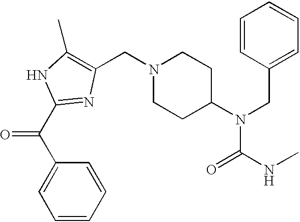 | 446 |
| 1-Benzyl-3-methyl-1-[1-(5-methyl-2-p- tolyl-1H-imidazol-4-ylmethyl)- piperidin-4-yl]-urea | 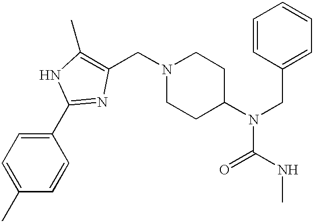 | 432 |
| 1-Benzyl-1-{1-[2-(4-methoxy-phenyl)-5- methyl-1H-imidazol-4-ylmethyl]- piperidin-4-yl}-3-methyl-urea | 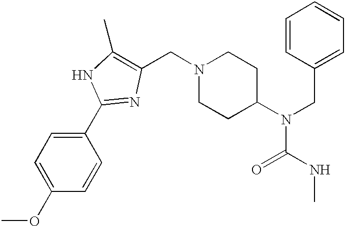 | 448 |
| 1-Benzyl-1-{1-[5-methyl-2-(4- trifluoromethyl-phenyl)-1H-imidazol-4- ylmethyl]-piperidin-4-yl}-3-pyridin-2- yl-urea | 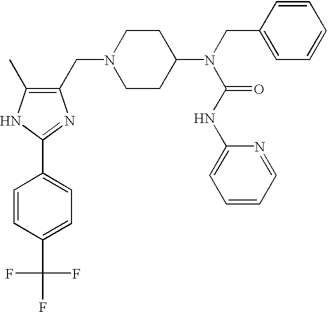 | 549.1 |

### Alternative Method of Reaction Scheme 1 Step 5: Alkylation via a Chloromethylimidazole Intermediate

### 1-[1-[[2-[4-(Trifluoromethyl)phenyl]-5-methyl-1H-imidazol-4-yl]methyl]-4-piperidinyl]-3-methyl-1-phenylurea

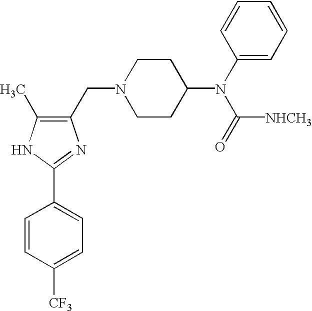

2-[4′-(Trifluromethyl)phenyl]-4-methylimidazole-5-methanol (770 mg) was treated cautiously with of thionyl chloride (5 ml) and the resulting solution heated at 70° C. for 15 min, then cooled and evaporated. The residue was re-evaporated twice with toluene (10 ml). The resulting viscous oil was dissolved in dichloromethane (30 ml), cooled in an ice/water bath and then treated with 4-(3′-methyl-1′-phenylureido)piperidine (700 ml) followed by dropwise treatment with a solution of ethyldiisopropylamine (2 ml) in dichloromethane (5 ml). After 1 h, the mixture was treated with saturated aqueous sodium hydrogen carbonate solution (30 ml). The organic solution was separated, dried (anhydrous magnesium sulfate), filtered and evaporated. The residue was subjected to flash chromatography using a gradient elution [dichloromethane/methanol (97:3) to dichloromethane/methanol/acetic acid/water (240:24:3:2)]. Product-containing fractions were combined and evaporated. The residue was evaporated twice with toluene (20 ml) and then dissolved in dichloromethane (40 ml). The solution was washed with 2M aqueous sodium hydroxide (40 ml), dried (anhydrous magnesium sulfate), filtered and concentrated in vacuo to about 5 ml. Hexane (30 ml) was added carefully to precipitate the 1-[1-[[2-[4-(trifluoromethyl)phenyl]-5-methyl-1H-imidazol-4-yl]methyl]-4-piperidinyl]-3-methyl-1-phenylurea as a white solid (330 mg, 23%). Mass spectrum 472 (M+H)⁺.

### EXAMPLES ACCORDING TO REACTION SCHEME 2

### Reaction Scheme 2, Step 1

### 8-[5-Methyl-2-(4-trifluoromethyl-phenyl)-1H-imidazol-4-ylmethyl]-1,4-dioxa-8-aza-spiro[4.5]decane

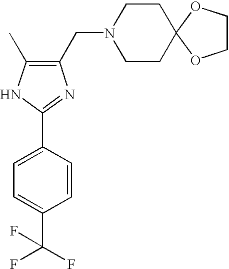

A mixture of 5-methyl-2-(4-trifluoromethyl-phenyl)-1H-imidazole-4-carbaldehyde (1.6 g) and 4-piperidone ethylene ketal (Avocado, 0.9 g) in dichloromethane (60 ml) was treated with sodium triacetoxyborohydide (Aldrich, 1.86 g) and allowed to stir at ambient temperature for 12 h. 2M aqueous sodium hydroxide solution (50 ml) was added and the mixture stirred vigorously for 5 min. The organic phase was separated, washed with water (50 ml), dried (anhydrous magnesium sulphate), filtered and the solvent removed under reduced pressure. The residue was subject to flash chromatography on silica gel using a gradient elution (dichloromethane/methanol 100:0 to 98:2). This gave the title compound as a white solid (1.21 g, 50%). Mass spectrum 382 [M+H]⁺.

### Reaction Scheme 2, Step 2

### N-Benzyl-1-[[2-[4-(Trifluoromethyl)phenyl]-5-methyl-1H-imidazol-4-yl]methyl]-4-piperidinone

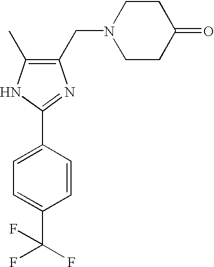

A mixture of 8-[5-methyl-2-(4-trifluoromethyl-phenyl)-1H-imidazol-4-ylmethyl]-1,4-dioxa-8-aza-spiro[4.5]decane (16.4 g) and 6M hydrochloric acid (200 ml) was heated to 90° C. for 30 min, cooled and neutralised with 8M aqueous sodium hydroxide solution. The mixture was extracted with dichloromethane (2×250 ml), and the organic extracts were combined, dried (anhydrous magnesium sulphate), filtered and evaporated. The residue was subjected to flash chromatography on silica gel using a gradient elution (DMAW 240 to DMAW 120) and the resultant acetate salt was partitioned between dichloromethane (100 ml) and 2M aqueous sodium hydroxide (75 ml). The organic layer was separated, dried (anhydrous magnesium sulphate), filtered and evaporated under reduced pressure to give the title compound as a white solid (9.65 g, 66%). Mass spectrum 438 [M+H]⁺.

### Reaction Scheme 2, Step 3

### N-Benzyl-1-[[2-[4-(trifluoromethyl)phenyl]-5-methyl-1H-imidazol-4-yl]methyl]-4-piperidinamine

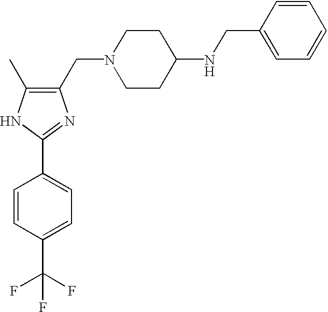

A solution of 1-[[2-[4-(trifluoromethyl)phenyl]-5-methyl-1H-imidazol-4-yl]methyl]-4-piperidinone (570 mg) in dichloromethane (10 ml) was treated with benzylamine (164 mg) followed by sodium triacetoxyborohydride (488 mg) and a solution of acetic acid (92 mg) in dichloromethane (5 ml) and stirred at ambient temperature for 1 h. The mixture was diluted with dichloromethane (40 ml), washed with 1M aqueous sodium hydroxide solution (10 ml), water (2×40 ml) and brine (30 ml). The organic layer was dried (MgSO4), filtered and removed under reduced pressure to give the title compound as a white solid (645 mg, 99%). Mass spectrum 429 [M+H]⁺.

| Systematic Name | Structure | m/z [M + H]⁺ |
|---|---|---|
| 1-[[2-[4- (Trifluoromethyl)phenyl]-5- methyl-1H-imidazol-4-yl]methyl]- N-(2,4,6-trimethoxybenzyl)-4- piperidinamine | 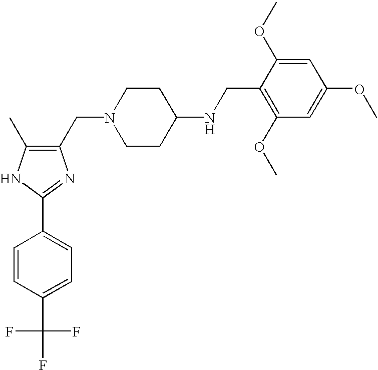 | 519 |
| 1-[[2-[4- (Trifluoromethyl)phenyl]-5- methyl-1H-imidazol-4-yl]methyl]- N-methyl-4-piperidinamine | 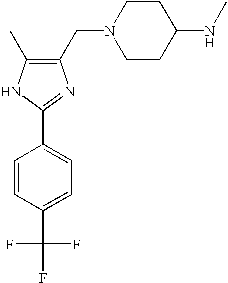 | 353 |
| N-Ethyl-1-[[5-methyl-2-[4- (trifluoromethyl)phenyl]-5- methyl-1H-imidazol-4-yl]methyl]- 4-piperidinamine | 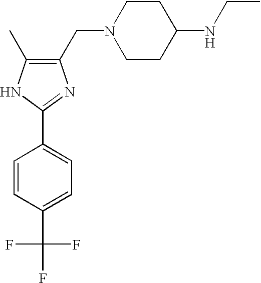 | 367 |
| 1-[[2-[4- (Trifluoromethyl)phenyl]-5- methyl-1H-imidazol-4-yl]methyl]- N-propyl-4-piperidinamine | 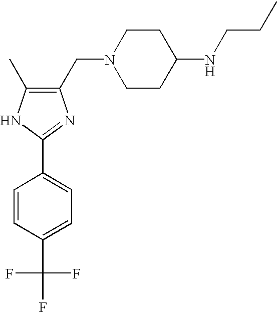 | 381 |
| 1-[[2-[4- (Trifluoromethyl)phenyl]-5- methyl-1H-imidazol-4-yl]methyl]- N-isopropyl-4-piperidinamine | 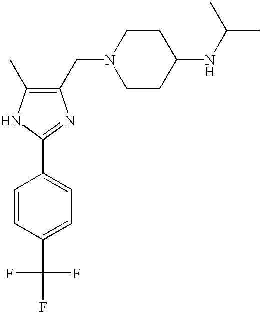 | 381 |
| N-Allyl-1-[[2-[4- (trifluoromethyl)phenyl]-5- methyl-1H-imidazol-4-yl]methyl]- 4-piperidinamine | 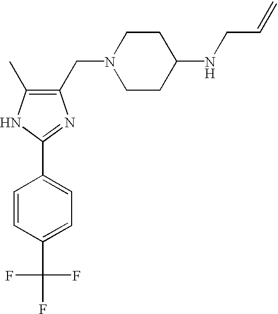 | 379 |
| N-Butyl-1-[[2-[4- (trifluoromethyl)phenyl]-5- methyl-1H-imidazol-4-yl]methyl]- 4-piperidinamine | 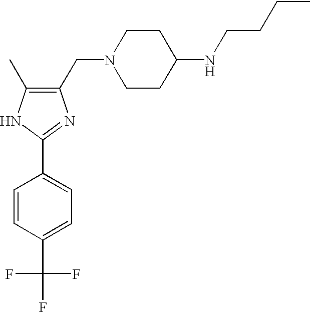 | 395 |
| N-Cyclopropyl-1-[[2-[4- (trifluoromethyl)phenyl]-5- methyl-1H-imidazol-4-yl]methyl]- 4-piperidinamine | 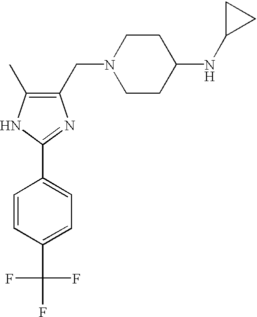 | 379 |
| N-(Cyclopropylmethyl)-1-[[2-[4- (trifluoromethyl)phenyl]-5- methyl-1H-imidazol-4-yl]methyl]- 4-piperidinamine | 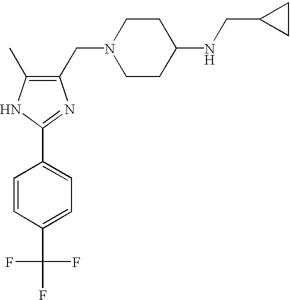 | 393 |
| N-Cyclopentyl-1-[[2-[4- (trifluoromethyl)phenyl]-5- methyl-1H-imidazol-4-yl]methyl]- N-piperidinamine | 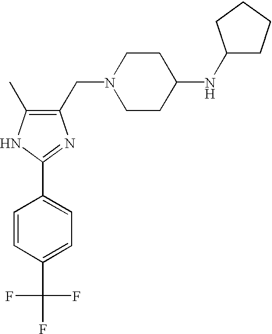 | 407 |
| N-Cyclohexyl-1-[[2-[4- (trifluoromethyl)phenyl]-5- methyl-1H-imidazol-4-yl]methyl]- 4-piperidinamine | 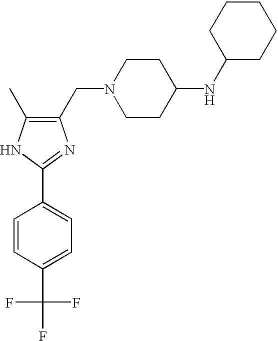 | 421 |
| N-(Cyclohexylmethyl)-1-[[2-[4- (trifluoromethyl)phenyl]-5- methyl-1H-imidazol-4-yl]methyl]- 4-piperidinamine | 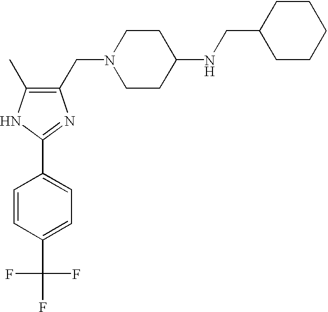 | 435 |
| 1-[[2-[4- (Trifluoromethyl)phenyl]-5- methyl-1H-imidazol-4-yl]methyl]- N-(2-phenylethyl)-4- piperidinamine | 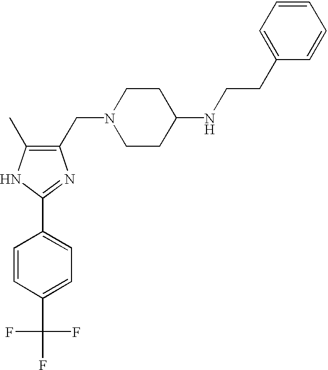 | 443 |
| 1-[[2-[4- (Trifluoromethyl)phenyl]-5- methyl-1H-imidazol-4-yl]methyl]- N-(3-phenylpropyl)-4- piperidinamine | 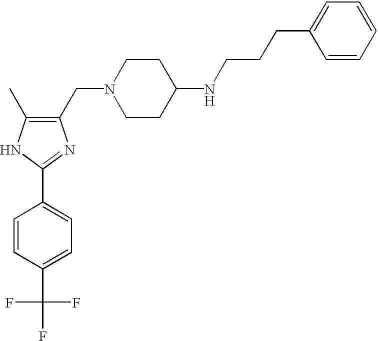 | 457 |
| 1-[[2-[4- (Trifluoromethyl)phenyl]-5- methyl-1H-imidazol-4-yl]methyl]- N-(4-methoxyphenyl)-4- piperidinamine | 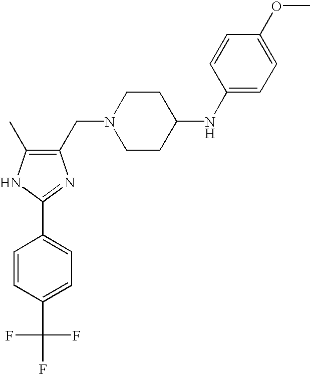 | 445 |
| 1-[[2-[4- (Trifluoromethyl)phenyl]-5- methyl-1H-imidazol-4-yl]methyl]- N-(4-methoxybenzyl)-4- piperidinamine | 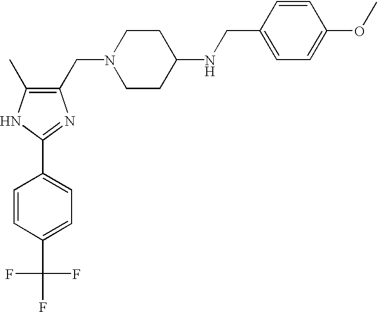 | 459 |
| N-(4-Chlorobenzyl)-1-[[2-[4- (trifluoromethyl)phenyl]-5- methyl-1H-imidazol-4-yl]methyl]- 4-piperidinamine | 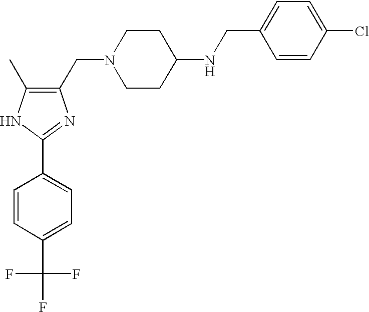 | 464 |
| N-[1-[[2-[4- (Trifluoromethyl)phenyl]-5- methyl-1H-imidazol-4-yl]methyl]- 4-piperidinyl]-4- pyridinemethylamine | 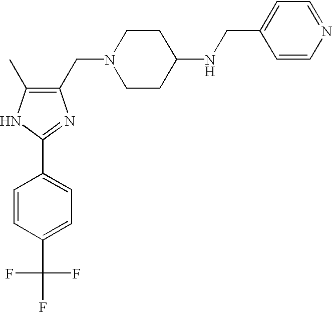 | 430 |
| N-[1-[[2-[4- (Trifluoromethyl)phenyl]-5- methyl-1H-imidazol-4-yl]methyl]- 4-piperidinyl]-3- pyridinemethylamine | 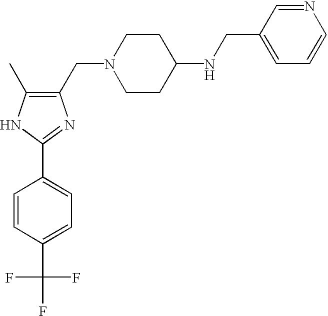 | 430 |
| N-Cyclobutyl-1-[[2-[4- (trifluoromethyl)phenyl]-5- methyl-1H-imidazol-4-yl]methyl]- 4-piperidinamine | 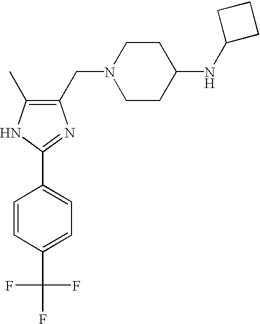 | 393 |
| N-(2,4-Dichlorobenzyl)-4-[[2-[4- (trifluoromethyl)phenyl]-5- methyl-1H-imidazol-4-yl]methyl]- 4-piperidinamine | 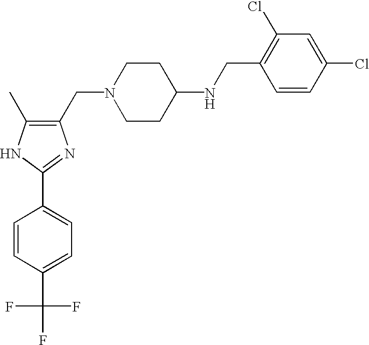 | 498 |
| N-(2-Chlorobenzyl)-4-[[2-[4- (trifluoromethyl)phenyl]-5- methyl-1H-imidazol-4-yl]methyl]- 4-piperidinamine | 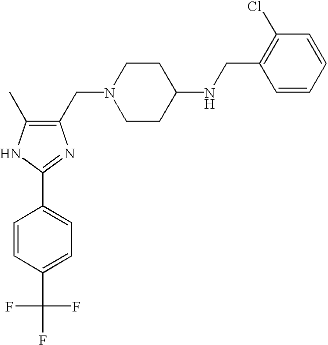 | 464 |
| 4-[[2-[4- (Trifluoromethyl)phenyl]-5- methyl-1H-imidazol-4-yl]methyl]- N-(2-methoxybenzyl)-4- piperidinamine | 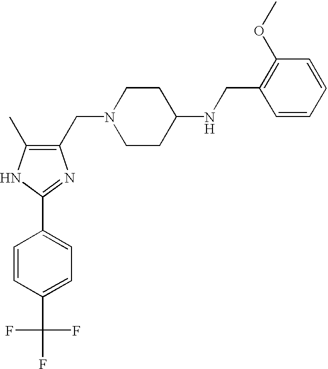 | 459 |
| 4-[[2-[4- (Trifluoromethyl)phenyl]-5- methyl-1H-imidazol-4-yl]methyl]- N-(2-methylbenzyl)-4- piperidinamine | 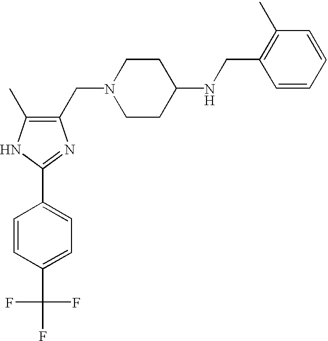 | 443 |
| N-(3,5-Dichlorobenzyl)-4-[[2-[4- (trifluoromethyl)phenyl]-5- methyl-1H-imidazol-4-yl]methyl]- 4-piperidinamine | 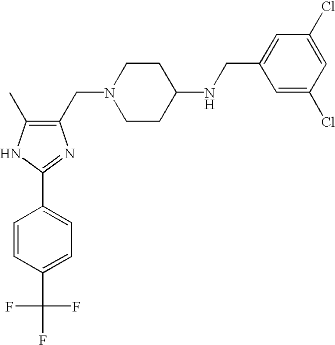 | 498 |
| N-(3,4-Dichlorobenzyl)-4-[[2-[4- (trifluoromethyl)phenyl]-5- methyl-1H-imidazol-4-yl]methyl]- 4-piperidinamine | 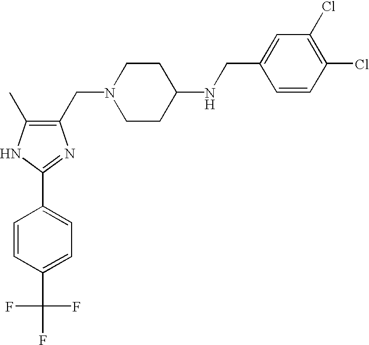 | 498 |
| 4-[[2-[4- (Trifluoromethyl)phenyl]-5- methyl-1H-imidazol-4-yl]methyl]- N-(3-methylbenzyl)-4- piperidinamine | 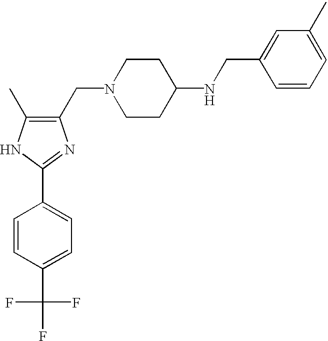 | 443 |
| 4-[[2-[4- (Trifluoromethyl)phenyl]-5- methyl-1H-imidazol-4-yl]methyl]- N-(3-nitrobenzyl)-4- piperidinamine | 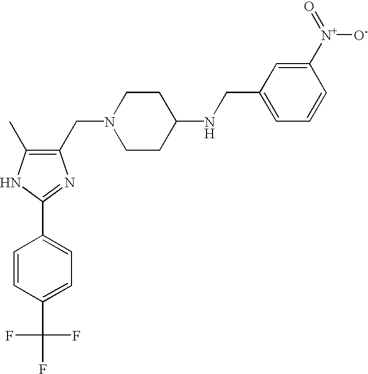 | 474 |
| 4-[[2-[4- (Trifluoromethyl)phenyl]-5- methyl-1H-imidazol-4-yl]methyl]- N-[4-(dimethylamino)benzyl]-4- piperidinamine | 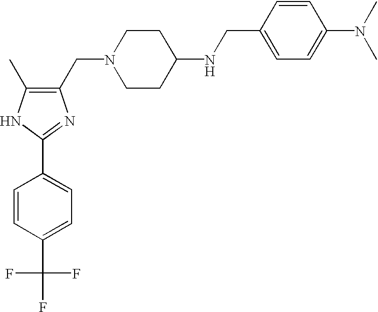 | 472 |
| 4-[[2-[4- (Trifluoromethyl)phenyl]-5- methyl-1H-imidazol-4-yl]methyl]- N-(4-nitrobenzyl)-4- piperidinamine | 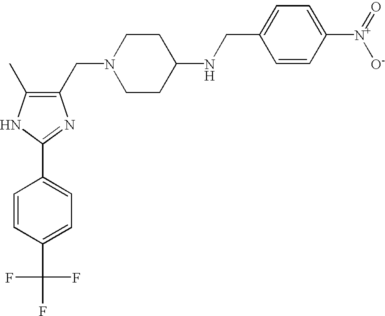 | 474 |
| N-(4-Aminobenzyl)-4-[[2-[4- (trifluoromethyl)phenyl]-5- methyl-1H-imidazol-4-yl]methyl]- 4-piperidinamine | 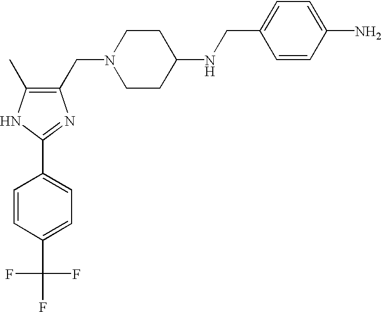 | 444 |
| Methyl 4-[[1-[[2-[4- (trifluoromethyl)phenyl]-5- methyl-1H-imidazol-4-yl]methyl]- piperidinyl]aminomethyl]benzoate | 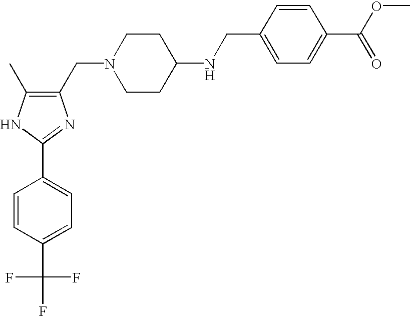 | 487 |
| 4-[[2-[4-(Trifluoromethyl)phenyl]-5- methyl-1H-imidazol-4-yl]methyl]- N-[4-(methanesulfonyl)benzyl]-4- piperidinamine | 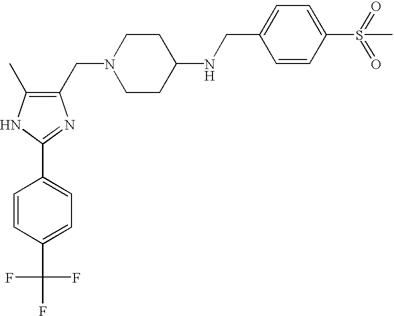 | 507 |
| N-[(3-Biphenylyl)methyl]-4-[[2- [4-(trifluoromethyl)phenyl]-5- methyl-1H-imidazol-4-yl]methyl]- 4-piperidinamine | 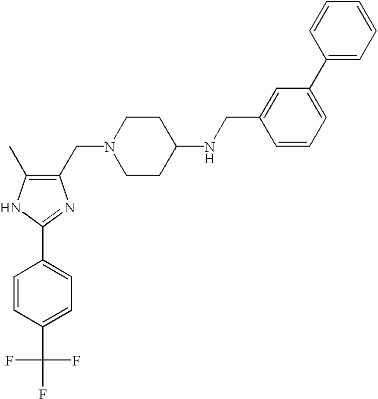 | 505 |
| 4-[[2-[4- (Trifluoromethyl)phenyl]-5- methyl-1H-imidazol-4-yl]methyl]- N-(4-phenoxybenzyl)-4- piperidinamine | 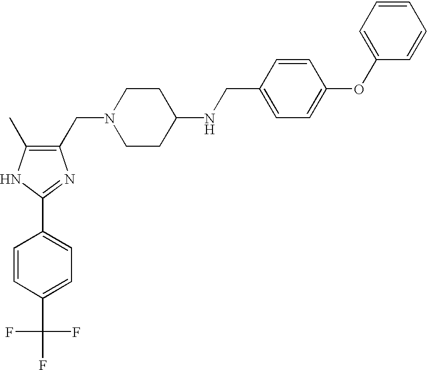 | 521 |
| N-[(4-Biphenylyl)methyl]-4-[[2- [4-(trifluoromethyl)phenyl]-5- methyl-1H-imidazol-4-yl]methyl]- 4-piperidinamine | 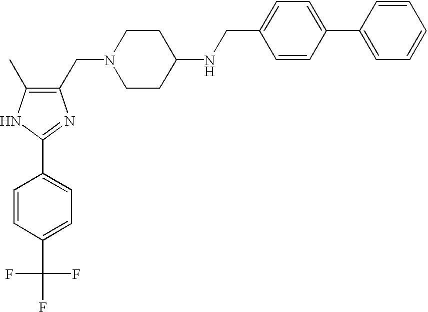 | 505 |
| 4-[[1-[[2-[4- (Trifluoromethyl)phenyl]-5- methyl-1H-imidazol-4-yl]methyl]-4- piperidinyl]aminomethyl]benzonitrile | 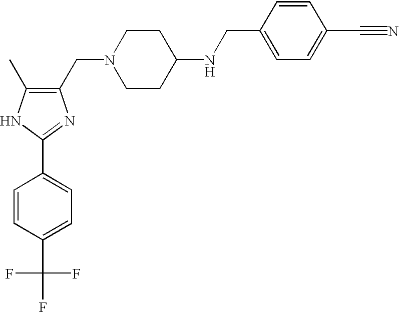 | 454 |
| Isobutyl-{1-[5-methyl-2-(4- trifluoromethyl-phenyl)-1H- imidazol-4-ylmethyl]-piperidin-4- yl}-amine | 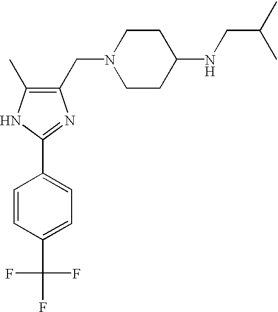 | 395 |
| {1-[5-Methyl-2-(4- trifluoromethyl-phenyl)-1H- imidazol-4-ylmethyl]-piperidin-4- yl}-(4-trifluoromethyl-benzyl)- amine | 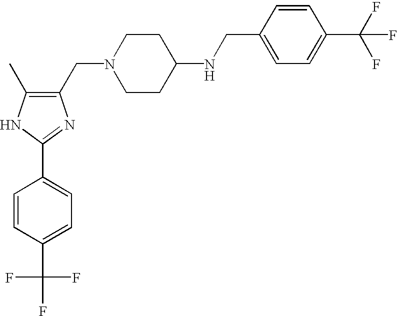 | 497 |
| 1-[4-({1-[5-Methyl-2-(4- trifluoromethyl-phenyl)-1H- imidazol-4-ylmethyl]-piperidin-4- ylamino}-methyl)-phenyl]-3- phenyl-urea | 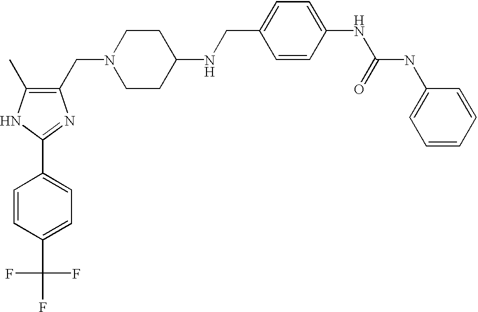 | 563 |

### Reaction Scheme 2, Step 3.1

### 1-Benzyl-3-[4-(trifluoromethyl)phenyl]1-[1-[[2-[4-(trifluoromethyl)phenyl-5-methyl-1H-imidazol-4-yl]methyl]-4-piperidinyl]urea

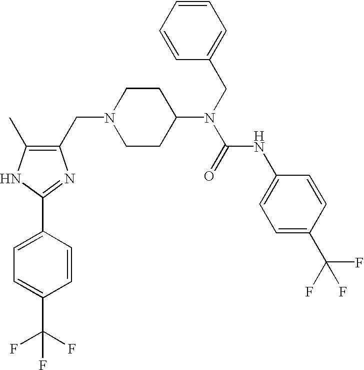

N-Benzyl-1-[[2-[4-(trifluoromethyl)phenyl]-5-methyl-1H-imidazol-4-yl]methyl]-4-piperidinamine (64 mg) was dissolved in dichloromethane (1 ml) and treated with a solution of 4-(trifluoromethyl)phenyl isocyanate (Lancaster Synthesis 12576, 31 mg) in dichloromethane (1 ml). The mixture was stirred at ambient temperature for 18 h and then evaporated. Flash chromatography using a gradient elution [dichloromethane/methanol (95:5) to dichloromethane/methanol (90:10)] afforded, upon evaporation of the product-containing fractions, 1-benzyl-3-[4-(trifluoromethyl)phenyl]1-[1-[[2-[4-(trifluoromethyl)phenyl]-5-methyl-1H-imidazol-4-yl]methyl]-4-piperidinyl]urea as a white solid (69 mg, 75%) Mass spectrum 616 (M+H)⁺.

### Reaction Scheme 2, Step 4.1

### 1,3-Dibenzyl-1-[1-[[2-[4-(trifluoromethyl)phenyl]-5-methyl-1H-imidazol-4-yl]methyl]-4-piperidinyl]urea

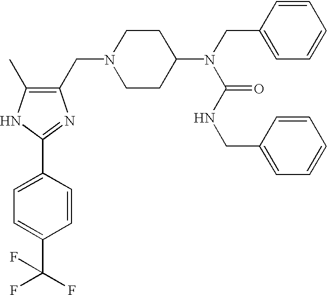

A solution of benzyl-{1-[5-methyl-2-(4-trifluoromethyl-phenyl)-1H-imidazol-4-ylmethyl]-piperidin-4-yl}-amine (64 mg) in dichloromethane (1 ml) was added to a solution of benzylisocyanate (20 mg) in dichloromethane (2 ml) and the mixture was stirred at room temperature for 2 h. The reaction mixture was loaded directly onto a pre-packed silica gel flash chromatography column and eluted with 20% methanol in dichloromethane. This gave the title compound as a white solid (59 mg, 72%). Mass spectrum 548 [M+H]⁺.

The following compounds were produced in a manner analogous to that described above by using the appropriate isocyanate and the appropriately substituted aminopiperidine:

| Systematic name | Structure | m/z [M + H]⁺ |
|---|---|---|
| 1-[1-[[2-[4-(Trifluoromethyl)phenyl]-5- methyl-1H-imidazol-4-yl]methyl]-4- piperidinyl]-1,3-dimethylurea | 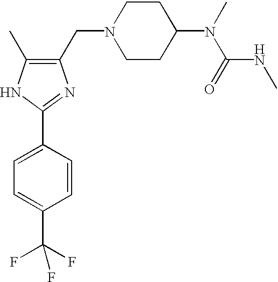 | 410 |
| 1-Butyl-1-[1-[[2-[4- (trifluoromethyl)phenyl]-5-methyl-1H- imidazol-4-yl]methyl]-4-piperidinyl]-3- methylurea | 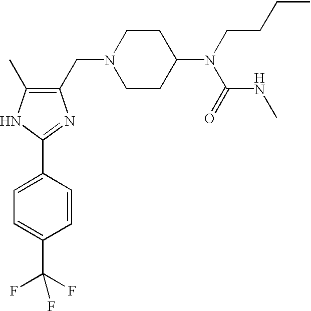 | 452 |
| 1-Cyclohexyl-1-[1-[[2-[4- (trifluoromethyl)phenyl]-5-methyl-1H- imidazol-4-yl]methyl]-4-piperidinyl]-3- methylurea | 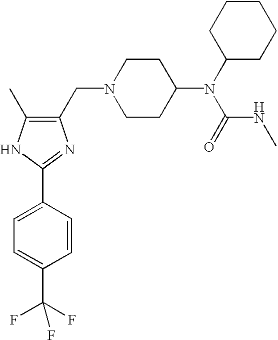 | 478 |
| 1-[1-[[2-[4-(Trifluoromethyl)phenyl]-5- methyl-1H-imidazol-4-yl]methyl]-4- piperidinyl]-3-methyl-1-(2- phenethyl)urea | 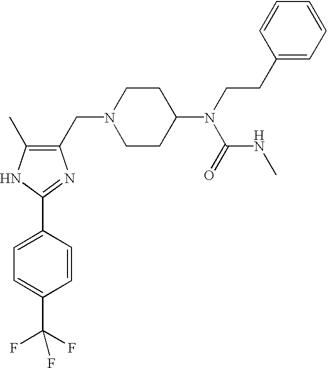 | 500 |
| 1-[1-[[2-[4-(Trifluoromethyl)phenyl]-5- methyl-1H-imidazol-4-yl]methyl]-4- piperidinyl]-3-methyl-1-(3- phenylpropyl)urea | 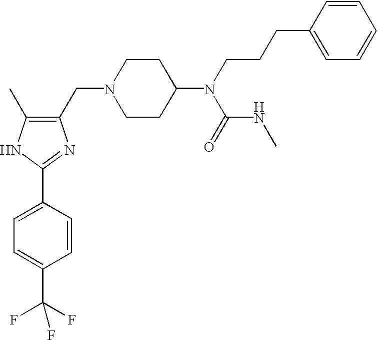 | 514 |
| 1-[1-[[2-[4-(Trifluoromethyl)phenyl]-5- methyl-1H-imidazol-4-yl]methyl]-4- piperidinyl]-1-(4-methoxybenzyl)-3- methylurea | 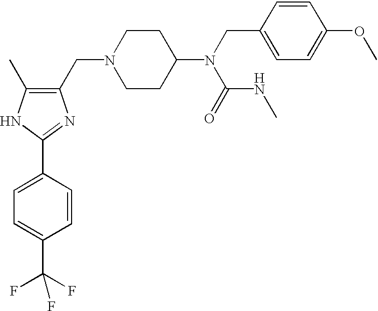 | 516 |
| 1-(4-Chlorobenzyl)-1-[1-[[2-[4- (trifluoromethyl)phenyl]-5-methyl-1H- imidazol-4-yl]methyl]-4-piperidinyl]-3- methylurea | 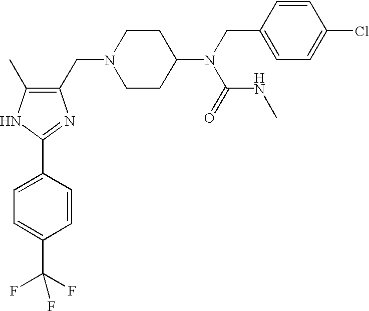 | 521 |
| 1-[1-[[2-[4-(Trifluoromethyl)phenyl]-5- methyl-1H-imidazol-4-yl]methyl]-4- piperidinyl]-3-methyl-1-[(4- pyridyl)methyl]urea | 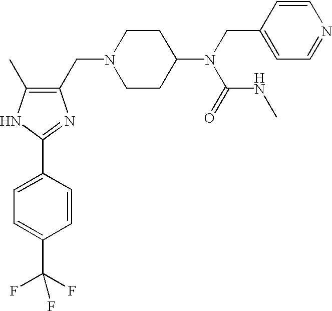 | 487 |
| 1-Benzyl-3-ethyl-1-[1-[[2-[4- (trifluoromethyl)phenyl]-5-methyl-1H- imidazol-4-yl]methyl]-4- piperidinyl]urea | 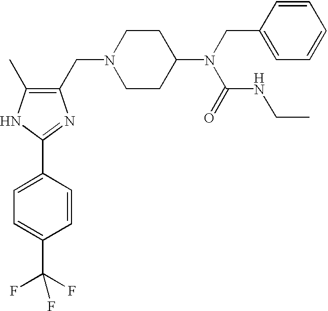 | 500 |
| 1-Benzyl-1-[1-[[2-[4- (trifluoromethyl)phenyl]-5-methyl-1H- imidazol-4-yl]methyl]-4-piperidinyl]-3- propylurea | 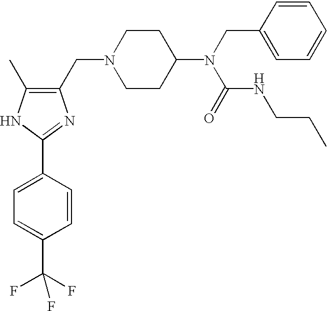 | 514 |
| 1-Benzyl-1-[1-[[2-[4- (trifluoromethyl)phenyl]-5-methyl-1H- imidazol-4-yl]methyl]-4-piperidinyl]-3- phenylurea | 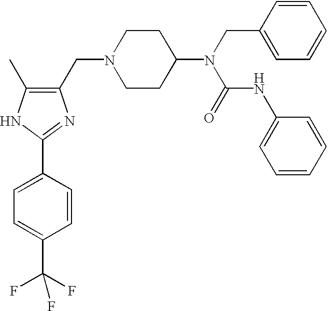 | 548 |
| 1-Benzyl-1-[1-[[2-[4-trifluoromethyl- phenyl]-5-methyl-1H-imidazol-4- yl]methyl]-4-piperidinyl]-3-(4- methoxyphenyl)urea | 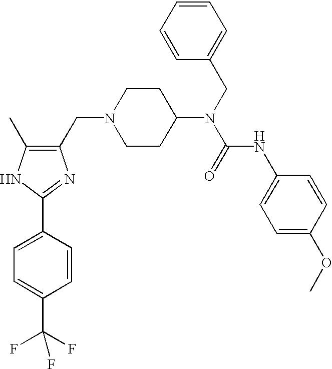 | 578 |
| 1-Benzyl-3-[4- (trifluoromethyl)phenyl]1-[1-[[2-[4- (trifluoromethyl)phenyl-5-methyl-1H- imidazol-4-yl]methyl]-4- piperidinyl]urea | 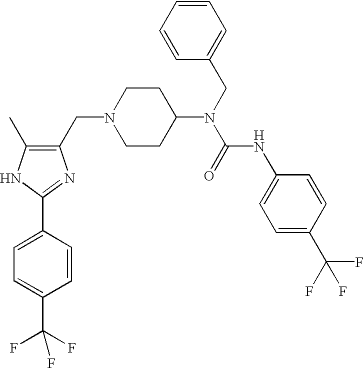 | 616 |
| 1-Benzyl-3-cyclohexyl-1-[1-[[2-[4- (trifluoromethyl)phenyl]-5-methyl-1H- imidazol-4-yl]methyl]-4- piperidinyl]urea | 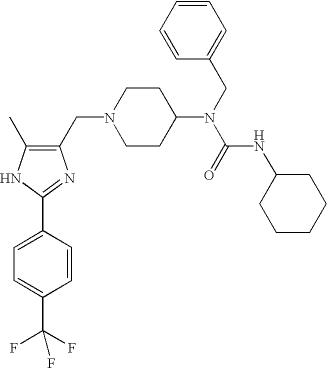 | 554 |
| 1-Benzyl-3-tert.-butyl-1-[1-[[2-[4- (trifluoromethyl)phenyl]-5-methyl-1H- imidazol-4-yl]methyl]-4- piperidinyl]urea | 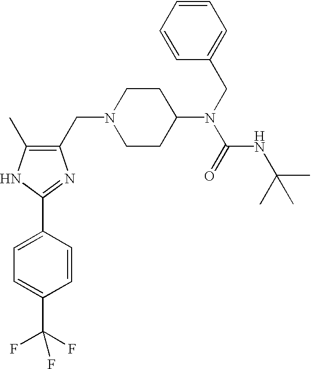 | 528 |
| 1-Benzyl-1-[1-[[2-[4- (trifluoromethyl)phenyl]-5-methyl-1H- imidazol-4-yl]methyl]-4-piperidinyl]-3- (2-phenylethyl)urea | 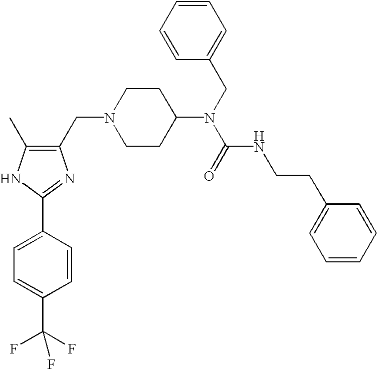 | 576 |
| 1-Cyclopropylmethyl-3-methyl-1-{1-[5- methyl-2-(4-trifluoromethyl-phenyl)- 1H-imidazol-4-ylmethyl]-piperidin-4- yl}-urea | 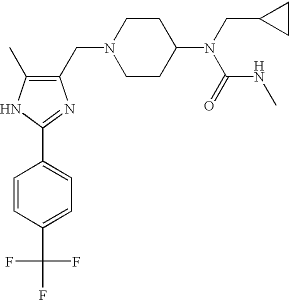 | 450.1 |
| 1-Cyclopentyl-3-methyl-1-{1-[5-methyl- 2-(4-trifluoromethyl-phenyl)-1H- imidazol-4-ylmethyl]-piperidin-4-yl}- urea | 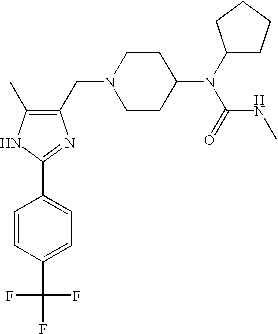 | 464.1 |
| 1-Cyclohexylmethyl-3-methyl-1-{1-[5- methyl-2-(4-trifluoromethyl-phenyl)- 1H-imidazol-4-ylmethyl]-piperidin-4- yl}-urea | 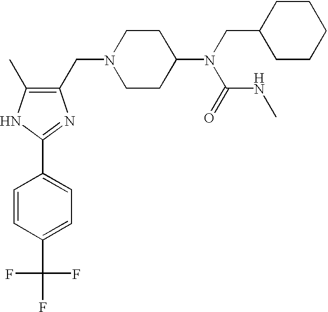 | 492.1 |
| 3-Methyl-1-{1-[5-methyl-2-(4- trifluoromethyl-phenyl)-1H-imidazol-4- ylmethyl]-piperidin-4-yl}-1-(4- trifluoromethyl-benzyl)-urea | 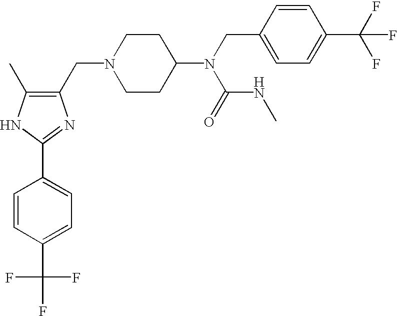 | 554.2 |
| 3-Methyl-1-{1-[5-methyl-2-(4- trifluoromethyl-phenyl)-1H-imidazol-4- ylmethyl]-piperidin-4-yl}-1-pyridin-3- ylmethyl-urea | 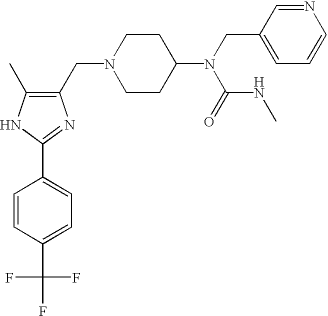 | 487.1 |
| 1-(2,4-Dichloro-benzyl)-1-{1-[5-methyl- 2-(4-trifluoromethyl-phenyl)-1H- imidazol-4-ylmethyl]-piperidin-4-yl}-3- phenyl-urea | 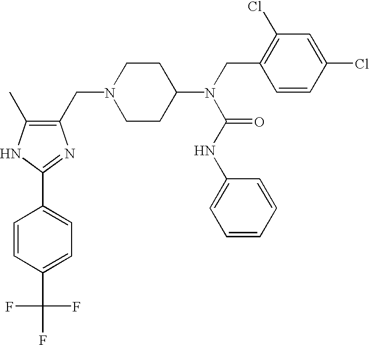 | 616.1 |
| 1-(2-Chloro-benzyl)-1-{1-[5-methyl-2- (4-trifluoromethyl-phenyl)-1H- imidazol-4-ylmethyl]-piperidin-4-yl}-3- phenyl-urea | 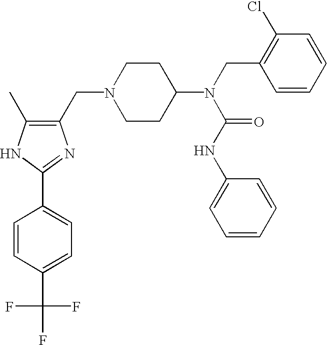 | 582.1 |
| 1-(2-Methoxy-benzyl)-1-{1-[5-methyl-2- (4-trifluoromethyl-phenyl)-1H- imidazol-4-ylmethyl]-piperidin-4-yl}-3- phenyl-urea | 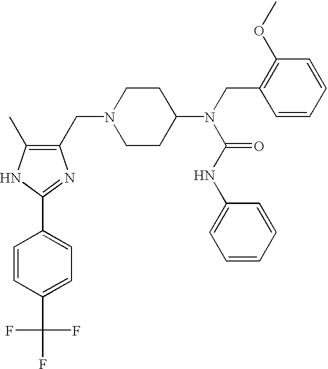 | 578.2 |
| 1-(2-Methyl-benzyl)-1-{1-[5-methyl-2- (4-trifluoromethyl-phenyl)-1H- imidazol-4-ylmethyl]-piperidin-4-yl}-3- phenyl-urea | 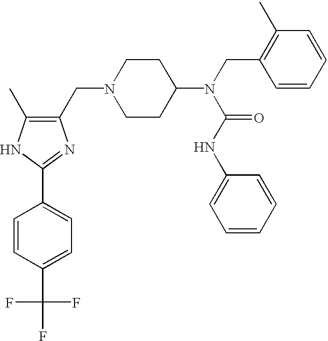 | 562.2 |
| 1-(3,5-Dichloro-benzyl)-1-{1-[5-methyl- 2-(4-trifluoromethyl-phenyl)-1H- imidazol-4-ylmethyl]-piperidin-4-yl}-3- phenyl-urea | 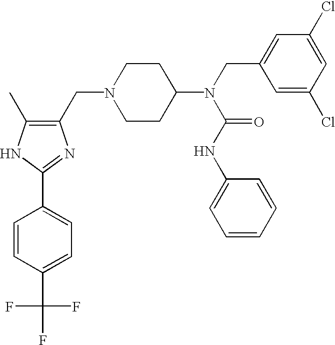 | 616.1 |
| 1-(3,4-Dichloro-benzyl)-1-{1-[5-methyl- 2-(4-trifluoromethyl-phenyl)-1H- imidazol-4-ylmethyl]-piperidin-4-yl}-3- phenyl-urea | 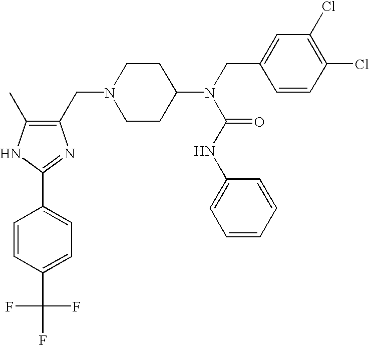 | 616.1 |
| 1-(3-Methyl-benzyl)-1-{1-[5-methyl-2- (4-trifluoromethyl-phenyl)-1H- imidazol-4-ylmethyl]-piperidin-4-yl}-3- phenyl-urea | 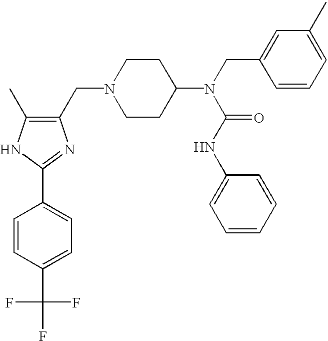 | 562.2 |
| 1-{1-[5-Methyl-2-(4-trifluoromethyl- phenyl)-1H-imidazol-4-ylmethyl]- piperidin-4-yl}-1-(3-nitro-benzyl)-3- phenyl-urea | 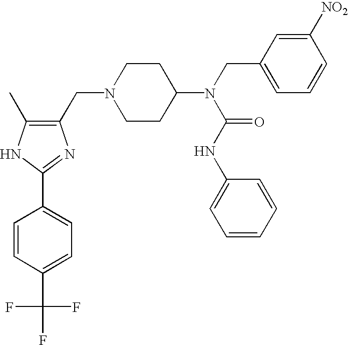 | 593.1 |
| 1-(4-Dimethylamino-benzyl)-1-{1-[5- methyl-2-(4-trifluoromethyl-phenyl)- 1H-imidazol-4-ylmethyl]-piperidin-4- yl}-3-phenyl-urea | 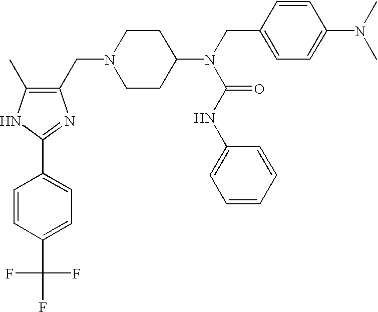 | 591.2 |
| 1-{1-[5-Methyl-2-(4-trifluoromethyl- phenyl)-1H-imidazol-4-ylmethyl]- piperidin-4-yl}-1-(4-nitro-benzyl)-3- phenyl-urea | 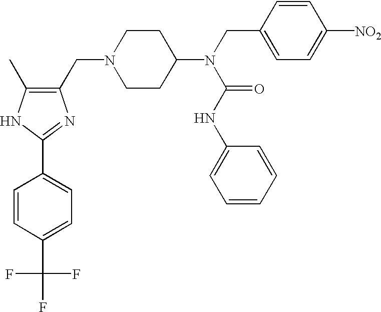 | 593.1 |
| 1-{1-[5-Methyl-2-(4-trifluoromethyl- phenyl)-1H-imidazol-4-ylmethyl]- piperidin-4-yl}-3-phenyl-1-[4-(3- phenyl-ureido)-benzyl]-urea | 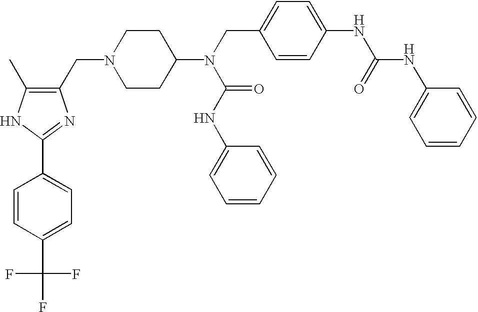 | 682.2 |
| 4-(1-{1-[5-Methyl-2-(4-trifluoromethyl- phenyl)-1H-imidazol-4-ylmethyl]- piperidin-4-yl}-3-phenyl-ureidomethyl)- benzoic acid methyl ester | 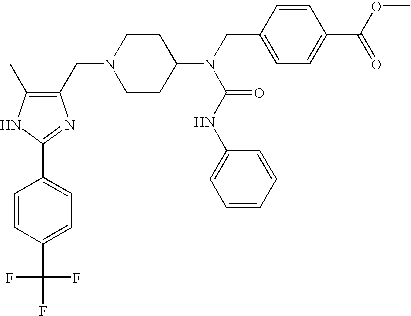 | 606.2 |
| 1-(4-Methanesulfonyl-benzyl)-1-{1-[5- methyl-2-(4-trifluoromethyl-phenyl)- 1H-imidazol-4-ylmethyl]-piperidin-4- yl}-3-phenyl-urea | 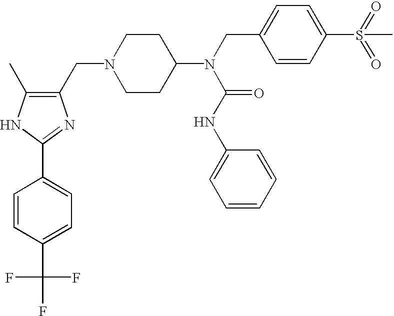 | 626.1 |
| 1-Biphenyl-3-ylmethyl-1-{1-[5-methyl- 2-(4-trifluoromethyl-phenyl)-1H- imidazol-4-ylmethyl]-piperidin-4-yl}-3- phenyl-urea | 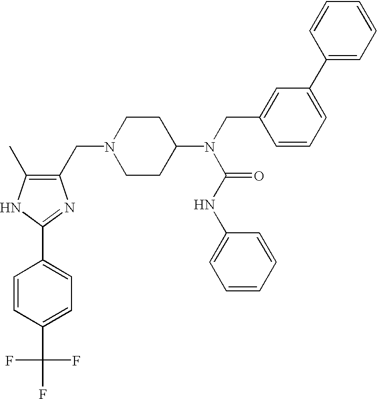 | 624.2 |
| RO-33-8371/000 | 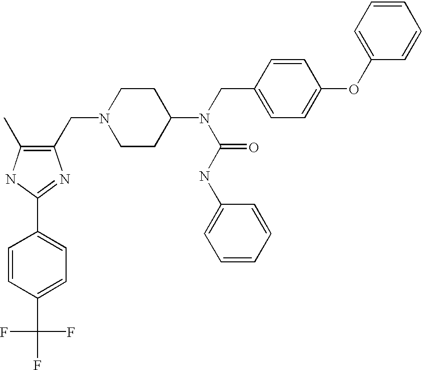 | 640.2 |
| 1-Biphenyl-4-ylmethyl-1-{1-[5-methyl- 2-(4-trifluoromethyl-phenyl)-1H- imidazol-4-ylmethyl]-piperidin-4-yl}-3- phenyl-urea | 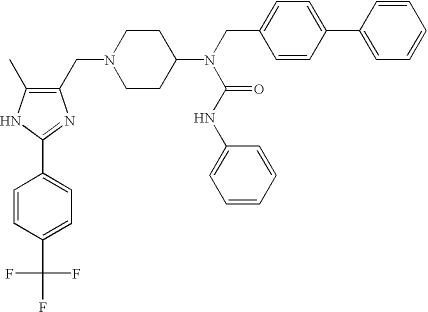 | 624.2 |
| 1-(4-Cyano-benzyl)-1-{1-[5-methyl-2- (4-trifluoromethyl-phenyl)-1H- imidazol-4-ylmethyl]-piperidin-4-yl}-3- phenyl-urea | 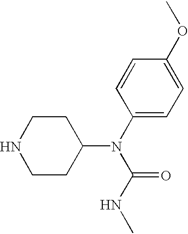 | 573.2 |
| 1-Benzyl-3-(4-iodo-phenyl)-1-{1-[5- methyl-2-(4-trifluoromethyl-phenyl)- 1H-imidazol-4-ylmethyl}-piperidin-4- yl}-urea | 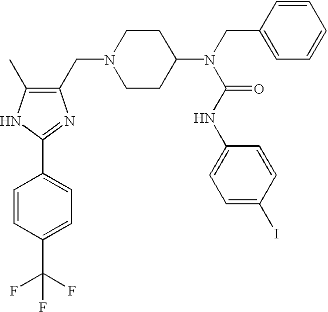 | 674.0 |
| 3-Methyl-1-{1-[5-methyl-2-(4- trifluoromethyl-phenyl)-1H-imidazol-4- ylmethyl]-piperidin-4-yl}-1-propyl-urea | 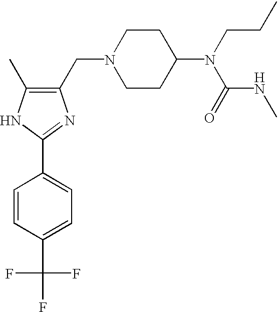 | 438.1 |
| 1-Isopropyl-3-methyl-1-{1-[5-methyl-2- (4-trifluoromethyl-phenyl)-1H- imidazol-4-ylmethyl]-piperidin-4-yl}- urea | 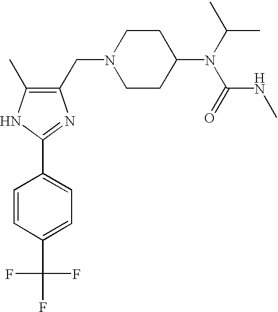 | 438.1 |
| 1-Isobutyl-3-methyl-1-{1-[5-methyl-2- (4-trifluoromethyl-phenyl)-1H- imidazol-4-ylmethyl]-piperidin-4-yl}- urea | 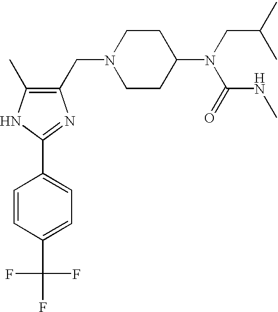 | 452.1 |
| 1-Cyclopropyl-3-methyl-1-{1-[5-methyl- 2-(4-trifluoromethyl-phenyl)-1H- imidazol-4-ylmethyl]-piperidin-4-yl}- urea | 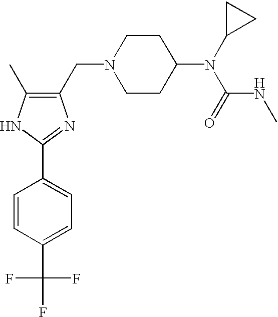 | 436.1 |
| 1-Benzyl-3-(3,4-dichloro-phenyl)-1-{1- [5-methyl-2-(4-trifluoromethyl-phenyl)- 1H-imidazol-4-ylmethyl]-piperidin-4- yl}-urea | 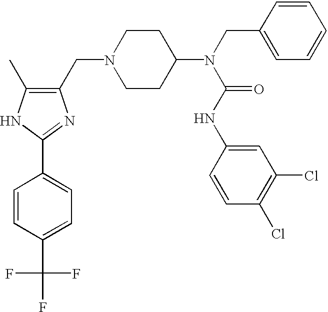 | 616.0 |
| 4-(3-Benzyl-3-{1-[5-methyl-2-(4- trifluoromethyl-phenyl)-1H-imidazol-4- ylmethyl]-piperidin-4-yl}-ureido)- benzoic acid methyl ester | 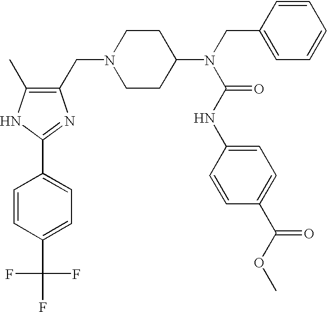 | 606.1 |

### Reaction Scheme 2, Step 4.4

### 1-Benzyl-3-(4-chloro-phenyl)-3-methyl-1-{1-[5-methyl-2-(4-trifluoromethyl-phenyl)-1H-imidazol-4-ylmethyl]-piperidin-4-yl}-urea

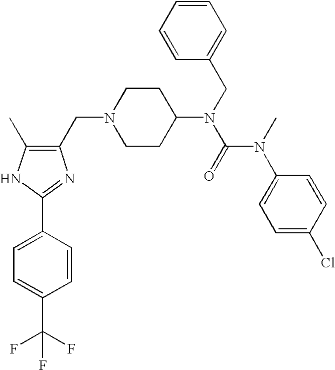

A solution of p-chloro-N-methylbenzylamine (282 mg) in dichloromethane (10 ml) was treated with pyridine (0.96 ml) followed by a solution of 20% phosgene in toluene (3.1 ml) and stirred at ambient temperature for 16 h. The mixture was quenched by the addition of saturated sodium hydrogen carbonate (10 ml), and the organic layer was en separated, dried (anhydrous magnesium sulphate), filtered and evaporated under reduced pressure. The residue was dissolved in dichloromethane (10 ml) and a solution of N-benzyl-1-[[2-[4-(trifluoromethyl)phenyl]-5-methyl-1H-imidazol-4-yl]methyl]-4-piperidinamine (657 mg) in dichloromethane (10 ml) was added followed by more pyridine (0.96 ml) and the mixture stirred for a further 16 h. The mixture was diluted with dichloromethane (40 ml) followed by brine (2×20 ml). The organic layer was separated, dried (anhydrous magnesium sulphate), filtered and evaporated under reduced pressure. The residue was purified by flash chromotography eluting with 10% methanol in dichloromethane to give the title compound (512 mg, 56%). Mass spectrum 597 [M+H]⁺.

| Systematic name | Structure | m/z [M + H]+ |
|---|---|---|
| 1,3-Dibenzyl-3-methyl-1-{1-[5-methyl- 2-(4-trifluoromethyl-phenyl)-1H- imidazol-4-ylmethyl]-piperidin-4-yl}-urea | 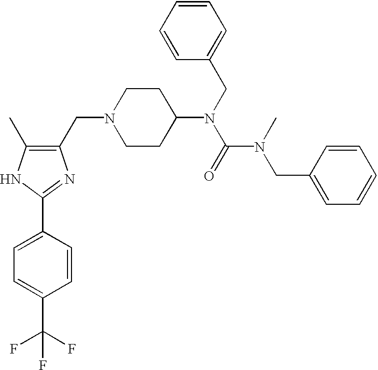 | 576 |
| 1-Benzyl-3-cyclopropyl-1-{1-[5-methyl- 2-(4-trifluoromethyl-phenyl)-1H- imidazol-4-ylmethyl]-piperidin-4-yl}-urea | 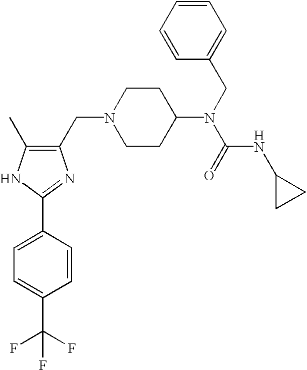 | 512 |
| 1-Benzyl-1-[1-[[2-[4- (trifluoromethyl)phenyl]-5-methyl-1H- imidazol-4-yl]methyl]-4-piperidinyl]-3- (3-phenylpropyl)urea | 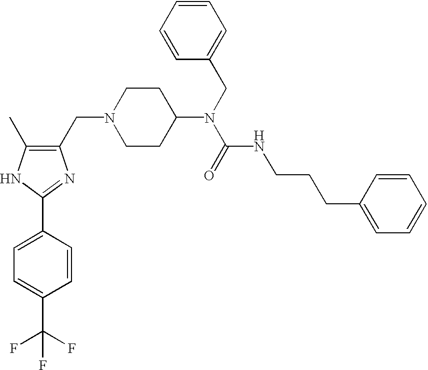 | 590 |

### EXAMPLES ACCORDING TO REACTION SCHEME 3

### Reaction Scheme 3, Step 1

### 4-Trifluoromethylphenyl-amidoxime

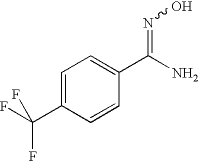

A solution of 4-trifluoromethyl benzonitrile (Avocado 14514, 15 g) in toluene (200 ml) was treated with methanol (15 ml) followed by hydroxylamine hydrochloride (2.25 g) and potassium tert-butoxide (3.52 g). The mixture was heated to 80° C. and treated with further portions of hydroxylamine hydrochloride (1.07 g) and potassium tert-butoxide (3.52 g) after 2, 4 and 6 h. The mixture was stirred for 16 h, and then cooled. The solvents were evaporated and the residue partitioned between water (100 ml) and dichloromethane (200 ml). The aqueous layer was extracted with two further portions of dichloromethane (2×200 ml). The organic solutions were combined, dried (anhydrous magnesium sulphate), filtered and evaporated to give the title compound as a white solid (16.7 g, 93%). Mass spectrum, 215 [M+H]⁺.

The following compounds were produced in a manner analogous to that described above by using the appropriately substituted benzonitrile in place of 4-trifluoromethyl benzonitrile:

| Systematic name | Structure | m/z [M + H]⁺ |
|---|---|---|
| N-Hydroxy-4-methyl-benzamidine | 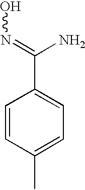 | 151 |
| 4-tert.-butyl-N-hydroxy-benzamidine | 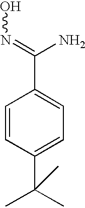 | 193 |
| N-Hydroxy-2,3-dimethoxy-benzamidine | 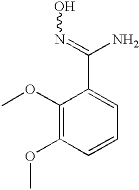 | 197 |
| N-Hydroxy-4-methoxy-benzamidine | 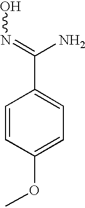 | 167 |
| N-Hydroxy-2-methoxy-benzamidine | 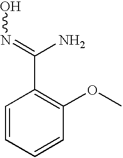 | 167 |
| 4-Dimethylamino-N-hydroxy-benzamidine | 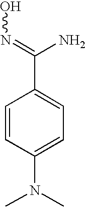 | 151 |
| 3-Chloro-N-hydroxy-benzamidine | 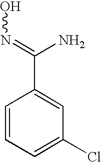 | 171 |
| 2-Chloro-N-hydroxy-benzamidine | 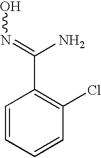 | 171 |

### Reaction Scheme 3, Step 2

### 4-Trifluoromethylphenyl Amidine Acetate

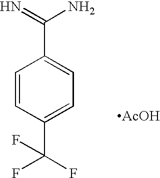

A solution of 4-trifluoromethyl amidoxime (16.7 g) in acetic acid (400 ml) was treated with acetic anhydride (11.6 ml). After 15 min, 10% palladium on charcoal (Fluka, 2.5 g) was added and the mixture was shaken under an atmosphere of hydrogen for 2 h. The mixture was filtered through Hyflo, evaporated, and then azeotroped twice with toluene. The resulting white solid was triturated with hexane to yield the title compound as a white solid (19.1 g, 94%). Mass spectrum 189 [M+H]⁺.

The following compounds were produced in a manner analogous to that described above by replacing the 4-trifluoromethyl amidoxime with the appropriate amidoxime:

| Systematic name | Structure | m/z [M + H]⁺ |
|---|---|---|
| 4-Methyl-benzamidine acetate | 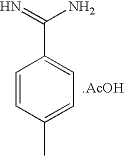 | 135 |
| 4-tertButyl-benzamidine acetate | 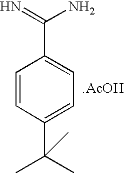 | 177 |
| 2,3-Dimethoxy-benzamidine acetate | 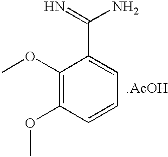 | 181 |
| 4-Methoxy-benzamidine acetate | 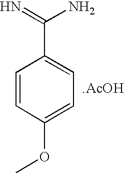 | 151 |
| 2-Methoxy-benzamidine acetate | 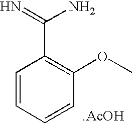 | 151 |
| 4-Dimethylamino-benzamidine acetate | 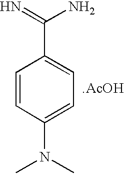 | 135 |
| 3-Chloro-benzamidine acetate | 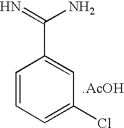 | 155 |
| 2-Chloro-benzamidine acetate | 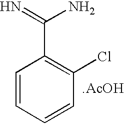 | 155 |

### EXAMPLE FROM REACTION SCHEME 4 OF THE PROCESS

### 4-(Trifluoromethyl)benzamidine Hydrochloride

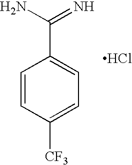

A solution of 4-(trifluoromethyl)benzonitrile (Avocado 14514, 15 g) in anhydrous methanol (90 ml) was treated with sodium methoxide (0.50 g) and the resulting solution stirred for 4 d at ambient temperature. After this time, ammonium chloride (4.7 g) was added and the mixture stirred for a further day. The mixture was subsequently evaporated and the residual white solid triturated in diethyl ether, filtered and dried to afford of 4-(trifluoromethyl)benzamidine hydrochloride as a white solid (14.2 g, 72%). Mass spectrum 188 [M]⁺.

### EXAMPLES ACCORDING TO REACTION SCHEME 5

### Reaction Scheme 5, Step 1

### [5-Methyl-2-(4-trifluoromethyl-phenyl)-1H-imidazol-4-yl]-methanol

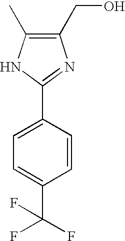

A suspension of 4-trifluoromethyl benzamidine acetate (20 g) and 2, 3-butanedione (8 g) in water (40 ml) was treated with 2M aqueous sodium hydroxide solution until pH8 was reached. The mixture was cooled in an ice bath and stirred for 2 h, the resultant solid was then collected by filtration and washed with water. The wet solid was treated with 4M aqueous hydrochloric acid (150 ml) and heated under reflux for 4 h then cooled in an ice bath and the pH adjusted to pH9 with 8M aqueous sodium hydroxide solution. The resultant solid was collected by filtration, washed sequentially with water and 50% aqueous ethanol and dried to give the title compound as a white solid (16.9 g, 82%). Mass spectrum 257 [M+H]⁺.

The following compounds were produced in a manner analogous to that described above by using the appropriate amidine acetate or hydrochloride prepared as described in reaction scheme 3 or reaction scheme 4 in place of the 4-trifluoromethyl benzamidine acetate

| Systematic name | Structure | m/z [M + H]⁺ |
|---|---|---|
| (5-Methyl-2-p-tolyl-1H-imidazol-4-yl)-methanol | 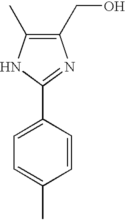 | 203 |
| [2-(4-tert.-butyl-phenyl)-5-methyl-1H-imidazol-4- yl]-methanol | 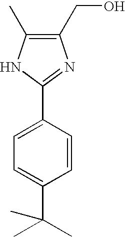 | 245 |
| [2-(2,3-Dimethoxy-phenyl)-5-methyl-1H-imidazol- 4-yl]-methanol | 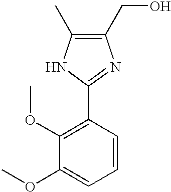 | 249 |
| [2-(4-Methoxy-phenyl)-5-methyl-1H-imidazol-4- yl]-methanol | 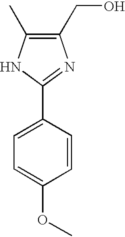 | 219 |
| [2-(2-Methoxy-phenyl)-5-methyl-1H-imidazol-4- yl]-methanol | 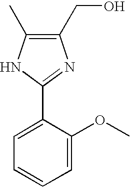 | 219 |
| [2-(4-Dimethylamino-phenyl)-5-methyl-1H- imidazol-4-yl]-methanol | 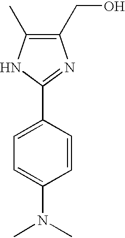 | 232 |
| [2-(3-Chloro-phenyl)-5-methyl-1H-imidazol-4-yl]- methanol | 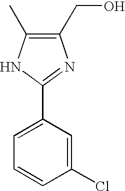 | 223 |
| [2-(2-Chloro-phenyl)-5-methyl-1H-imidazol-4-yl]- methanol | 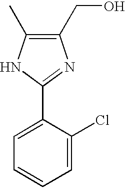 | 223 |
| (5-Methyl-2-phenyl-1H-imidazol-4-yl)-methanol | 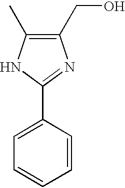 | 189 |

### Reaction Scheme 5, Step 2.1

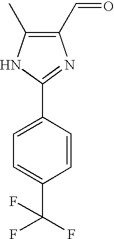

### 5-Methyl-2-(4-trifluoromethyl-phenyl)-1H-imidazole-4-carbaldehyde

A mixture of [5-methyl-2-(4-trifluoromethyl-phenyl)-1H-imidazol-4-yl]-methanol (1.2 g) and manganese dioxide (4 g) in 1,4-dioxane (50 ml) was heated at reflux for 1.5 h. The hot mixture was filtered through Hyflo and the filtered solids washed with hot 1,4-dioxane. The solvent was removed under reduced pressure and the residue was recrystallized from cyclohexane/ethyl acetate to yield the title compound as a pale yellow solid (0.6 g, 50%). Mass spectrum 255 [M+H]⁺.

The following compounds were synthesised in a manner analogous to that described above by using the appropriate hydroxymethyl imidazole, prepared as described in reaction scheme 5, step 1, in place of the [5-methyl-2-(4-trifluoromethyl-phenyl)-1H-imidazol-4-yl]-methanol:

| Systematic name | Structure | m/z [M + H]+ |
|---|---|---|
| 5-Methyl-2-phenyl-1H-imidazole-4- carbaldehyde | 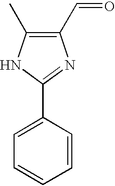 | 187 |
| 2-(2,3-Dimethoxy-phenyl)-5-methyl-1H- imidazole-4-carbaldehyde | 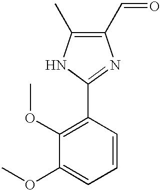 | 247 |

### Reaction Scheme 5, Step 2.2

### 4-Chloromethyl-5-methyl-2-(4-trifluoromethyl-phenyl)-1H-imidazole

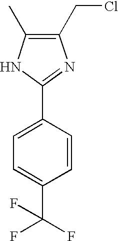

[5-Methyl-2-(4-trifluoromethyl-phenyl)-1H-imidazol-4-yl]-methanol (20 g) was treated with thionyl chloride (250 ml) and heated at 85° C. for 20 min. The thionyl chloride was removed under reduced pressure and the residue azeotroped twice with toluene to give the title compound as a pale yellow solid (14.5 g, 68%). Mass spectrum 274 [M+H]⁺.

The following compounds were synthesised in a manner analogous to that described above by using the appropriate hydroxymethyl imidazole, prepared as described in reaction scheme 5, step 1, in place of the [5-methyl-2-(4-trifluoromethyl-phenyl)-1H-imidazol-4-yl]-methanol:

| Systematic Name | Structure | m/z [M + H]⁺ |
|---|---|---|
| 4-Chloromethyl-5-methyl-2-p-tolyl-1H-imidazole | 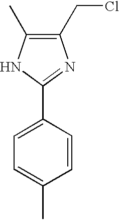 | 221 |
| 2-(4-tert.-butyl-phenyl)-4-chloromethyl-5-methyl- 1H-imidazole | 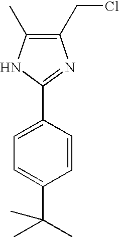 | 263 |
| 4-Chloromethyl-2-(4-methoxy-phenyl)-5-methyl-1H- imidazole | 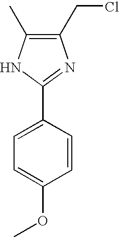 | 237 |
| 4-Chloromethyl-2-(2-methoxy-phenyl)-5-methyl-1H- imidazole | 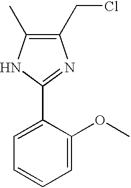 | 237 |
| [4-(4-Chloromethyl-5-methyl-1H-imidazol-2-yl)- phenyl]-dimethyl-amine | 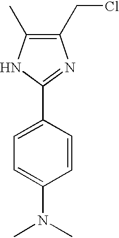 | 250 |
| 4-Chloromethyl-2-(3-chloro-phenyl)-5-methyl-1H- imidazole | 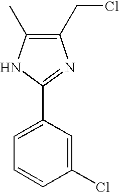 | 242 |
| 4-Chloromethyl-2-(2-chloro-phenyl)-5-methyl-1H- imidazole | 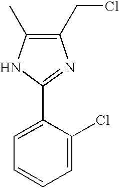 | 242 |

### EXAMPLES ACCORDING TO REACTION SCHEME 6

### Reaction Scheme 6, Step 1

### [2-(4-Trifluoromethyl-phenyl)-1H-imidazol-4-yl]-methanol

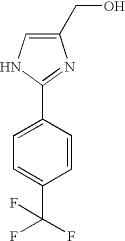

A mixture of 4-trifluoromethylbenzamidine hydrochloride (2.5 g) and 1,3-dihydroxyacetone dimer (Avocado 14189, 2 g) was heated at 80° C. in concentrated ammonia solution (20 ml) for 1 h. The mixture was allowed to cool and the product extracted with ethyl acetate (150 ml). The organic phase was dried (anhydrous magnesium sulphate), filtered and evaporated. The residue was triturated in diethyl ether to give the title compound as a white solid (1.2 g, 44%). Mass spectrum 243 [M+H]⁺.

The following compounds were synthesised using a method analogous to that described above by using the appropriate amidine hydrochloride, prepared as described in reaction scheme 4 or the amidine acetate prepared as described in reaction scheme 3, in place of the 4-trifluoromethylbenzamidine hydrochloride

| Systematic name | Structure | m/z [M + H]⁺ |
|---|---|---|
| [2-(2,3-Dimethoxy-phenyl)-1H-imidazol-4-yl]- methanol | 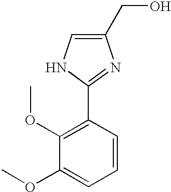 | 235 |

### Reaction Scheme 6, Step 2.2

The following compounds were produced in a manner analogous to that described in reaction scheme 5, step 2.2 by using the appropriate hydroxymethyl imidazole, prepared as described in reaction scheme 6, step1, in place of the [5-methyl-2-(4-trifluoromethyl-phenyl)-1H-imidazol-4-yl]-methanol:

| Systematic name | Structure | m/z [M + H]⁺ |
|---|---|---|
| 4-Chloromethyl-2-(4-trifluoromethyl-phenyl)- 1H-imidazole | 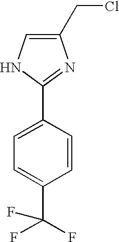 | 261 |
| 4-Chloromethyl-2-(2,3-dimethoxy-phenyl)-1H- imidazole | 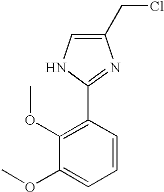 | 253 |

### EXAMPLES ACCORDING TO REACTION SCHEME 7

### Reaction Scheme 7, Step 1

### 4-(3-Methyl-1-phenyl-thioureido)-piperidine-1-carboxylic Acid Tert.-butyl Ester

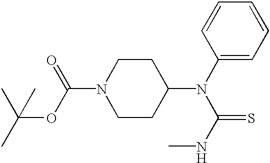

A solution of 4-phenylamino-piperidine-1-carboxylic acid tert.-butyl ester (0.4 g) in a mixture of ethanol (10 ml) and toluene (10 ml) was treated with methylisothiocyanate (Aldrich 11277-1, 0.11 g) and heated to 80° C. for 2 h. The solvents were removed under reduced pressure and the residue was triturated with hexane to give the title compound as a white solid (0.27 g, 53%). Mass spectrum 340 [M+H]⁺.

### 4-(3-Methyl-1-phenyl-thioureido)-piperidine

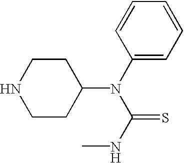

A solution of 4-(3-methyl-1-phenyl-thioureido)-piperidine-1-carboxylic acid tert.-butyl ester (200 mg) in dichloromethane (10 ml) was treated with trifluoroacetic acid (3 ml) and stirred at ambient temperature overnight. The solvents were evaporated and the residue partitioned between dichloromethane (50 ml) and aqueous sodium hydroxide solution (1M, 40 ml). The organic layer was separated, dried (anhydrous magnesium sulphate), filtered and evaporated under reduced pressure to give the title compound as a white solid (80 mg, 56%). Mass spectrum 250 [M+H]⁺.

### 3-Methyl-1-[1-[[5-methyl-2-[4(trifluoromethyl)phenyl-1H-imidazol-4-yl]methyl]-4-piperidinyl]-1-phenylthiourea

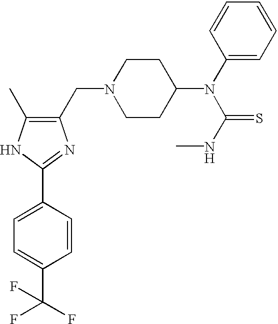

To a mixture of 4-(3-methyl-1-phenyl-thioureido)-piperidine (60 mg) and 5-methyl-2-(4-trifluoromethyl-phenyl)-1H-imidazole-4-carbaldehyde (65 mg) in dichloromethane (10 ml) was added sodium triacetoxy borohydride (75 mg) followed by acetic acid (2 drops) and the mixture stirred at ambient temperature for 4 h. Dichloromethane (50 ml) was added and the mixture washed with 1M aqueous sodium hydroxide solution (50 ml) followed by brine (50 ml). The organic layer was dried (anhydrous magnesium sulphate), filtered and evaporated under reduced pressure. The residue was purified by flash chromatography eluting with 2% methanol in dichloromethane to give the title compound as a white solid (30 mg, 26%). Mass spectrum 488 [M+H]⁺.

### EXAMPLES ACCORDING TO REACTION SCHEME 8

### Reaction Scheme 8, Step 1

### (5-Methyl-1H-imidazol-2-yl)-phenyl-methanone

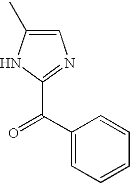

Benzoyl chloride (17 g) was added dropwise to a stirred solution of 4-methylimidazole (Aldrich 19988-5, 5 g) in a mixture of pyridine (5 ml) and triethylamine (17 ml) under an atmosphere of nitrogen and stirring continued for 2 h (mechanical stirring required). 7.5M Aqueous sodium hydroxide solution (6 ml) was added and the mixture heated under reflux for 40 min. The mixture was allowed to cool and diluted with water (40 ml). The resultant precipitate was collected by filtration, washed with water, dried and recrystallized from toluene to give the title compound as a white solid (1.7 g, 15%). Mass spectrum 187 [M+H]⁺.

### Reaction Scheme 8, Step 2

### (5-Methyl-1H-imidazol-2-yl)-phenyl-methanol

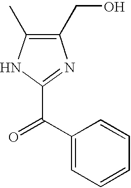

A mixture of 5-methyl-1H-imidazol-2-yl)-phenyl-methanone (1 g), 36% w/w formaldehyde in water (6.4 ml), 2M aqueous sodium hydroxide (2 ml), ethanol (30 ml) and water (15 ml) was heated at 55° C. for 48 h. The volatile organics were removed under reduced pressure and the residue partitioned between dichloromethane (30 ml) and a further portion of water (10 ml). The aqueous layer was re-extracted with dichloromethane (2×20 ml). The combined organic solutions were dried (anhydrous magnesium sulphate ), filtered and evaporated under reduced pressure. Flash chromatography eluting with 5% methanol in dichloromethane gave the title compound as white solid (0.69 g, 60%). Mass spectrum 217 [M+H]⁺.

### Reaction Scheme 8, Step 3

### 2-Benzoyl-5-methyl-1H-imidazole-4-carbaldehyde

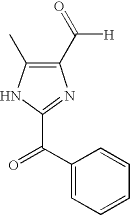

A solution of (5-methyl-1H-imidazol-2-yl)-phenyl-methanol in dichloromethane (25 ml) and 1,4-dioxane (25 ml) was treated with manganese dioxide (2.6 g) and heated at 80° C. for 1 h. The mixture was filtered through celite and the organic solution was dried (anhydrous magnesium sulphate), filtered and evaporated under reduced pressure to give the title compound as a white solid (308 mg, 48%). Mass spectrum 215 [M+H]⁺.

### Reaction Scheme 8, Step 4

This reaction is carried out in a manner analogous to that described in reaction scheme 1 step 5.

### 1-[1-[(2-Benzoyl-5-methyl-1H-imidazol-4-yl)]methyl]-4-piperidinyl]-1-benzyl-3-methylurea

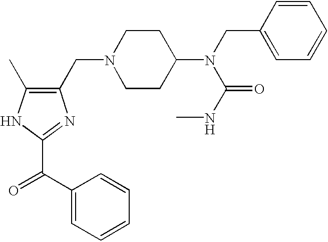

To a mixture of 2-benzoyl-5-methyl-1H-imidazole-4-carboxaldehyde (300 mg) and 1-benzyl-3-methyl-1-piperidin-yl-urea (350 mg) in dichloromethane (25 ml) was added sodium triacetoxy borohydride (420 mg) and the mixture stirred at ambient temperature for 3 h. The mixture was washed with aqueous sodium hydroxide solution (1M, 20 ml), and brine (2×20 ml), dried (anhydrous magnesium sulphate), filtered and the solvents removed under reduced pressure. The residue was purified by flash chromatography eluting with 4% methanol in dichloromethane to give the title compound as white solid (405 mg, 65%). Mass spectrum 446 [M+H]⁺.

### Reaction Scheme 8, Step 5

### 1-Benzyl-1-[1-[[2-[(RS)-(hydroxy)(phenyl)methyl]-5-methyl-1H-imidazol-4-yl]methyl]-4-piperidinyl]-3-methylurea

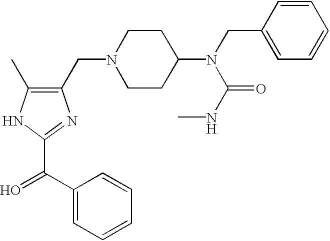

To a solution of 1-benzyl-1-{1-[2-(hydroxy-phenyl-methyl)-5-methyl-1H-imidazol-4-ylmethyl]-piperidin-4-yl}-3-methyl-urea (0.06 g) in isopropyl alcohol (8 ml) was added sodium borohydride (0.03 g) and the mixture stirred at ambient temperature for 1 h. The mixture was then treated with saturated sodium chloride solution (20 ml) and extracted with ethyl acetate (2×20 ml). The combined organic solutions were dried (anhydrous magnesium sulphate), filtered and evaporated. The residue was purified by flash chromatography on silica gel eluting with DMAW 240. The resultant acetate salt was partitioned between dichloromethane (100 ml) and 2M aqueous sodium hydroxide (10 ml) . The organic phase was separated, dried (anhydrous magnesium sulphate), filtered and evaporated to give the title compound as a white solid (33 mg, 54%). Mass spectrum 448 [M+H]⁺.

### Reaction Scheme 9, Step 1

### Methyl 2-[4-(trifluoromethyl)phenyl]-imidazole-4,5-dicarboxylate

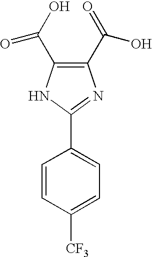

To d-tartaric acid (6.0 g) was added concentrated nitric acid (70%, 7 ml) followed cautiously by fuming nitric acid (100%, 17 ml). Concentrated sulfuric acid (26 ml) was added dropwise ensuring the temperature was kept between 30° C. and 40° C. by the judicious use of an ice/water bath to cool the mixture as required. Upon addition, the mixture was cooled to 0° C. using an ice/water bath. The precipitated solid was filtered off and dried. The dried solid was added to crushed ice (10 g), the mixture cooled to −10° C. and neutralised by the addition of concentrated aqueous ammonia. A further 12 ml of concentrated aqueous ammonia was added followed by 4-(trifluoromethyl)benzaldehyde (Avocado 15276, 6.96 g). The mixture was stirred at 0° C. for 6 h then for 18 h at ambient temperature. The mixture was neutralised with concentrated hydrochloric acid and the precipitated product was filtered, washed with water and dried to give 2-[4-(trifluoromethyl)phenyl]imidazole-4,5-dicarboxylic acid a white solid. (740 mg, 6%). ¹H NMR (400 MHz,DMSO-d₆): δ [ppm] 7.89 (2H, d), 8.36 (2H, d); Mass spectrum 342 [M+H+CH₃CN]⁺.

### Reaction Scheme 9, Step 2

### Dimethyl 2-[4-(trifluoromethyl)phenyl]imidazole-4,5-dicarboxylate

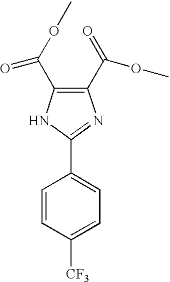

A solution of 2-[4-(trifluoromethyl)phenyl]imidazole-4,5-dicarboxylic acid (600 mg) in methanol (30 ml) was treated with concentrated sulfuric acid (0.5 ml) and the mixture heated at reflux for 5 h then cooled and allowed to stand for 18 h. The solvent was evaporated and the residue partitioned between ethyl acetate (20 ml) and saturated aqueous sodium hydrogen carbonate solution (20 ml). The organic phase was separated, dried (anhydrous magnesium sulfate), filtered and evaporated to give dimethyl 2-[4-(trifluoromethyl)phenyl]imidazole-4,5-dicarboxylate as a white solid (320 mg, 49%). Mass spectrum 329 [M+H]⁺.

### Reaction Scheme 9, Step 3

### Methyl 2-[4-(trifluoromethyl)phenyl]-4-formylimidazole-5-carboxylate

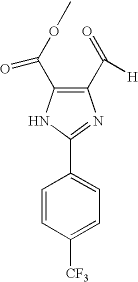

A solution of dimethyl 2-[4-(trifluoromethyl)phenyl]imidazole-4,5-dicarboxylate (300 mg) in tetrahydrofuran (20 ml) was treated cautiously with 60% w/w sodium hydride (44 mg) and the mixture heated at 60° C. for 5 min. The mixture was then cooled to −70° C. using a dry ice/acetone bath and treated dropwise with 1M diisobutylaluminium hydride in dichloromethane (1.1 ml). After 1.5 h, a further 1.1 ml of diisobutylaluminium hydride solution was added dropwise. After a further 2 h, the reaction mixture was treated cautiously with 50% v/v aqueous acetic acid (2 ml) and then allowed to warm to ambient temperature. The mixture was evaporated and the residue partitioned between ethyl acetate (20 ml) and saturated aqueous sodium hydrogen carbonate solution (20 ml). The organic phase was separated, dried (anhydrous magnesium sulfate), filtered and evaporated. The product was purified by flash chromatography using diethyl ether/isohexane (2:1)as eluant to give methyl 2-[4-(trifluoromethyl)phenyl]-4-formylimidazole-5-carboxylateas a white solid (40 mg, 15%). Mass spectrum 299 [M+H]⁺.

### EXAMPLES ACCORDING TO REACTION SCHEME 10

### Reaction Scheme 10, Step 1

### 1-Benzyl-5-methyl-2-(4-trifluoromethyl-phenyl)-1H-imidazole-4-carbaldehyde and 3-benzyl-5-methyl-2-(4-trifluoromethyl-phenyl)-3H-imidazole-4-carbaldehyde

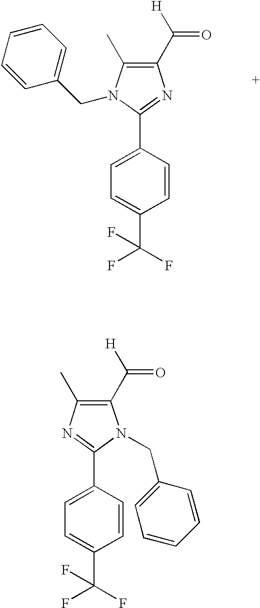

To a suspension of 60% w/w sodium hydride (47 mg) in dimethyl formamide (10 ml) was added a solution of 5-methyl-2-(4-trifluoromethyl-phenyl)-1H-imidazole-4-carbaldehyde (250 mg) in dimethyl formamide (2 ml) and the mixture stirred at ambient temperature for 45 min. Benzyl bromide (16 μl) was added and stirring continued for a further 2 h. The dimethyl formamide was removed under reduced pressure and the residue partitioned between ethyl acetate (50 ml) and water. The organic solution was separated, dried (anhydrous sodium sulphate), filtered and evaporated under reduced pressure to give the title compounds as a 1:1 mixture (280 mg, 84%). This mixture was used directly in the next step. Mass spectrum 345 [M+H]⁺.

### Reaction Scheme 10, Step 2

### 1-Benzyl-1-{1-[1-benzyl-5-methyl-2-(4-trifluoromethyl-phenyl)-1H-imidazol-4-ylmethyl]-piperidin-4-yl}-3-methyl-urea and 1-benzyl-1-{1-[3-benzyl-5-methyl-2-(4-trifluoromethyl-phenyl)-3H-imidazol-4-ylmethyl]-piperidin-4-yl}-3-methyl-urea

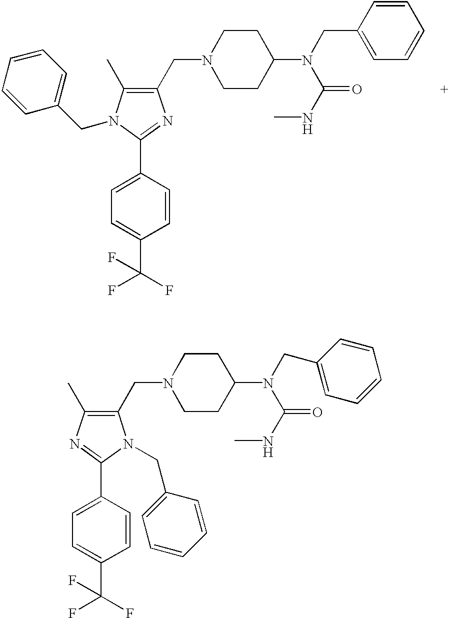

To a mixture of 1-benzyl-5-methyl-2-(4-trifluoromethyl-phenyl)-1H-imidazole-4-carbaldehyde and 3-benzyl-5-methyl-2-(4-trifluoromethyl-phenyl) -3H-imidazole-4-carbaldehyde (80 mg) in dichloromethane (10 ml) was added 1-benzyl-3-methyl-1-piperidin-4-yl-urea (57 mg) followed by sodium triacetoxyborohydride (80 mg) and the mixture was stirred at ambient temperature for 16 h. Saturated aqueous sodium hydrogen carbonate solution (10 ml) was added, the organic layer was then separated, dried (anhydrous sodium sulpahate), filtered and concentrated under reduced pressure. The residue was purified using a preparative liquid chromatography—mass spectroscopy system with a YMC-ODSA C-18 reverse phase column, using a gradient elution over 15 min. At t=0 min A=95%, B=5%, at t=15 min A=5%, B=95% (A=water/0.1% formic acid B=90% methanol/10% water/0.1% formic acid. This gave 1-benzyl-1-{1-[3-benzyl-5-methyl-2-(4-trifluoromethyl-phenyl)-3H-imidazol-4-ylmethyl]-piperidin-4-yl}-3-methyl-urea (Rt=4.08 min, 9 mg, 7%) and 1-benzyl-1-{1-[1-benzyl-5-methyl-2-(4-trifluoromethyl-phenyl)-3H-imidazol-4-ylmethyl]-piperidin-4-yl}-3-methyl-urea (Rt=6.60 min, 14 mg, 11%), both as white solids. Mass spectrum 577 [M+H]⁺.

### EXAMPLES ACCORDING TO REACTION SCHEME 11

### Reaction Scheme 11, Step 1

### 1-Benzyl-1-[1-(2-iodo-5-methyl-1H-imidazol-4-ylmethyl)-piperidin-4-yl]-3-methyl-urea

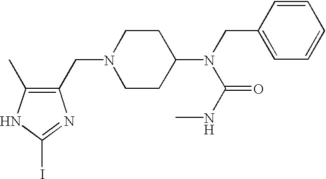

A solution of 1-benzyl-3-methyl-1-[1-(5-methyl-1H-imidazol-4-ylmethyl)-piperidin-4-yl]-urea (200 mg) in a mixture of dichloromethane (20 ml) and water (20 ml) was treated dropwise with a solution of iodine (150 mg) in dichloromethane (10 ml) and stirred at ambient temperature for 15 min. The pH of the mixture was adjusted to 9 by the addition of 2M aqueous sodium hydroxide solution and stirring was continued for 24 h. The organic solution was separated, washed with water (50 ml), dried (anhydrous magnesium sulphate), filtered and concentrated under reduced pressure. The residue was subjected to flash chromatography eluting with DMAW 240 to give the title compound as a white solid (35 mg, 12%). Mass spectrum 468 [M+H]⁺.

Further examples according to reaction schemes 1-11 with coresponding mass data:

| Systematic name | Structure | m/z [M + H]⁺ |
|---|---|---|
| 1-Cyclopentylmethyl-3-methyl-1-{1-[5- methyl-2-(4-trifluoromethyl-phenyl)- 1H-imidazol-4-ylmethyl]-piperidin-4- yl}-urea | 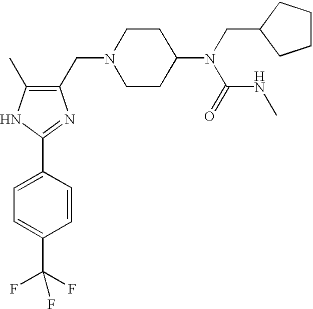 | 465 |
| 1-Cyclohexylmethyl-3-methyl-1-{1-[5- methyl-2-(4-trifluoromethyl-phenyl)- 1H-imidazol-4-ylmethyl]-piperidin-4- yl}-urea | 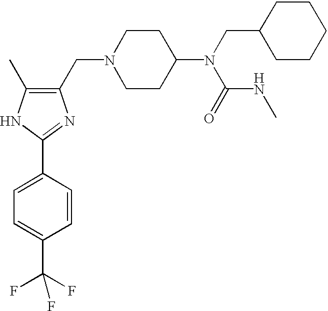 | 493 |
| 1-Benzyl-3-(4-chloro-phenyl)-3-methyl- 1-{1-[5-methyl-2-(4-trifluoromethyl- phenyl)-1H-imidazol-4-ylmethyl]- piperidin-4-yl}-urea | 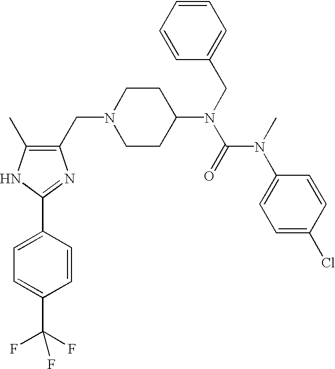 | 597/599 (contains chlorine) |
| 1,3-Dibenzyl-3-methyl-1-{1-[5-methyl- 2-(4-trifluoromethyl-phenyl)-1H- imidazol-4-ylmethyl]-piperidin-4-yl}-urea | 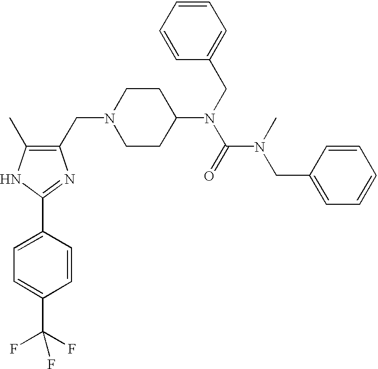 | 577 |
| 1-Benzyl-1-{1-[2-(2-methoxy-phenyl)-5- methyl-1H-imidazol-4-ylmethyl]- piperidin-4-yl}-3-methyl-urea | 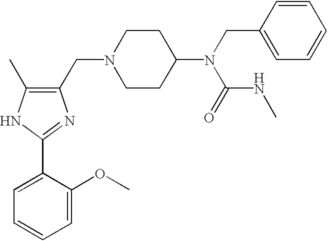 | 449 |
| 4-(3-Benzyl-3-{1-[5-methyl-2-(4- trifluoromethyl-phenyl)-1H-imidazol-4- ylmethyl]-piperidin-4-yl]-ureido)- benzoic acid | 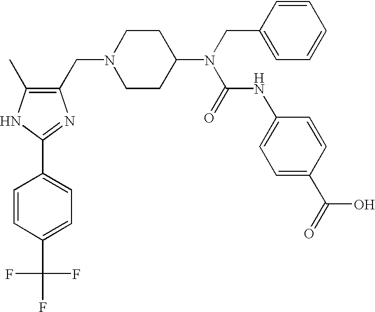 | 593 |
| 1-(4-Methyl-benzyl)-1-{1-[5-methyl-2- (4-trifluoromethyl-phenyl)-1H-imidazol- 4-ylmethyl]-piperidin-4-yl}-3-phenyl-urea | 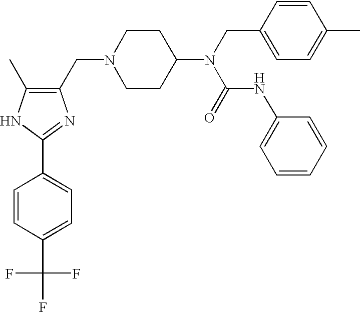 | 563 |
| 1-(2,4-Dimethyl-benzyl)-1-{1-[5-methyl- 2-(4-trifluoromethyl-phenyl)-1H- imidazol-4-ylmethyl]-piperidin-4-yl}-3- phenyl-urea | 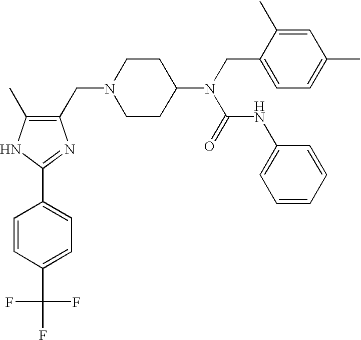 | 577 |

### EXAMPLE I

Tablets of the following composition are produced in a conventional manner:

mg/Tablet

| | |
|---|---|
| Active ingredient (preferabyly a compound as listed in table 1) | 100 |
| Powdered. lactose | 95 |
| White corn starch | 3 |
| Polyvinylpyrrolidone | 8 |
| Na carboxymethylstarch | 10 |
| Magnesium stearate | 2 |
| Tablet weight | 250 |

### EXAMPLE II

Tablets of the following composition are produced in a conventional manner:

mg/Tablet

| | |
|---|---|
| Active ingredient (preferabyly a compound as listed in table 1) | 200 |
| Powdered. lactose | 100 |
| White corn starch | 64 |
| Polyvinylpyrrolidone | 12 |
| Na carboxymethylstarch | 20 |
| Magnesium stearate | 4 |
| Tablet weight | 400 |

### EXAMPLE III

Capsules of the following composition are produced:

mg/Capsule

| | |
|---|---|
| Active ingredient (preferabyly a compound as listed in table 1) | 50 |
| Crystalline, lactose | 60 |
| Microcrystalline cellulose | 34 |
| Talc | 5 |
| Magnesium stearate | 1 |
| Capsule fill weight | 150 |

1. A compound of the formula 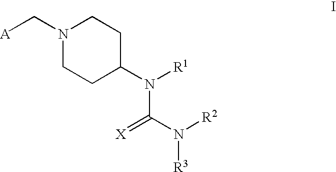
wherein
R¹ is hydrogen, C₁₋₁₂-alkyl, C₃₋₈-cycloalkyl, allyl, substituted C₁₋₄-alkyl, aryl, substituted aryl, heterocyclyl or substituted heterocyclyl;
R² and R³ are independently of each other hydrogen, C₁₋₁₂-alkyl, C₃₋₈-cycloalkyl, allyl, substituted C₁₋₄-alkyl, aryl, substituted aryl, heterocyclyl or substituted heterocyclyl;
X is S or O;
A is selected from the group consisting of: 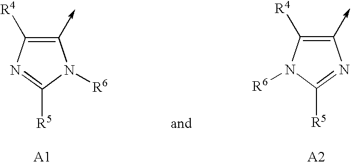
wherein
R⁴ is hydrogen, C₁₋₂-alkyl, substituted C₁₋₄-alkyl, C₃₋₈-cycloalkyl, C₁₋₄-alkoxy, CN, COR, CO₂R, CONRR′, NHCOR, aryl, substituted aryl, aryl-C(═O)—, substituted aryl-C(═O)—, aryl-CH(OH)—, substituted aryl-CH(OH)—, heterocyclyl, substituted heterocyclyl, heterocyclyl-C(═O)—, substituted heterocyclyl-C(═O)—, heterocyclyl-CH(OH)—, substituted heterocyclyl-CH(OH)— or NRR′;
R⁵ is hydrogen, C₁₋₁₂-alkyl, substituted C₁₋₄-alkyl, C₃₋₈-cycloalkyl, C₁₋₄-alkoxy, halogen, COR, aryl, substituted aryl, aryl-C(═O)—, substituted aryl-C(═O)—, aryl-CH(OH)—, substituted aryl-CH(OH)—, heterocyclyl, substituted heterocyclyl, heterocyclyl-C(═O)—, substituted heterocyclyl-C(═O)—, heterocyclyl-CH(OH)—, substituted heterocyclyl-CH(OH)— or NRR′;
R⁶ is hydrogen, C₁₋₁₂-alkyl, substituted C₁₋₄-alkyl, C₁₋₄-alkoxy, C₃₋₈-cycloalkyl, COR, CO₂R, CONRR′, NHCOR, SO₂NRR′ or SO₂R;
R and R′ are independently of each other hydrogen, C₁₋₁₂-alkyl, substituted C₁₋₄-alkyl, C₃₋₈-cycloalkyl, aryl, substituted aryl, heterocyclyl, substituted heterocyclyl, or where R is a substituent in a group CO₂R, R is a group which provides a hydrolyzable pharmaceutically acceptable ester;
and the pharmaceutically acceptable salts thereof.

2. The compound according to claim 1 wherein
R¹ is hydrogen, C₁₋₁₂-alkyl, C₃₋₈-cycloalkyl, allyl, substituted C₁₋₄-alkyl, aryl, substituted aryl, heterocyclyl or substituted heterocyclyl,
wherein substituted C₁₋₄-alkyl means alkyl substituted with 1-3 substituents selected from C₃₋₈-cycloalkyl, aryl, heterocyclyl, substituted aryl and substituted heterocyclyl; wherein substituted aryl and substituted heterocyclyl means aryl and heterocyclyl substituted with C₁₋₄-alkoxy, phenyl, phenoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens, and
wherein substituted aryl means aryl substituted with 1-5 substituents and substituted heterocyclyl means heterocyclyl substituted with 1-4 substituents and these substituents are selected from C₁₋₄-alkoxy, halogen, CN, NO₂, COR, CO₂R, CONRR′, NRR′, SO₂R, NHCOR, SO₂NRR′, C₁₋₄-alkyl and C₁₋₄-alkyl substituted with 1-3 halogens;
R² and R³ are independently of each other hydrogen, C₁₋₁₂-alkyl, C₃₋₈-cycloalkyl, allyl, substituted C₁₋₄-alkyl, aryl, substituted aryl, heterocyclyl or substituted heterocyclyl,
wherein substituted C₁₋₄-alkyl means alkyl substituted with 1-3 substituents selected from C₃₋₈-cycloalkyl, aryl, heterocyclyl, substituted aryl and substituted heterocyclyl; wherein substituted aryl and substituted heterocyclyl means aryl and heterocyclyl substituted with C₁₋₄-alkoxy, halogen, CN, NO₂, COR, CO₂R, CONRR′, NRR′, SO₂R, NHCOR, SO₂NRR′, C₁₋₄-alkyl or C₁₋₄-alkyl substituted with 1-3 halogens, and
wherein substituted aryl means aryl substituted with 1-5 substituents and substituted heterocyclyl means heterocyclyl substituted with 1-4 substituents and these substituents are selected from C₁₋₄-alkoxy, halogen, CN, NO₂, COR, CO₂R, CONRR′, NRR′, SO₂R, NHCOR, SO₂NRR′, C₁₋₄-alkyl and C₁₋₄-alkyl substituted with 1-3 halogens;
X is S or O;
A is selected from the group consisting of: 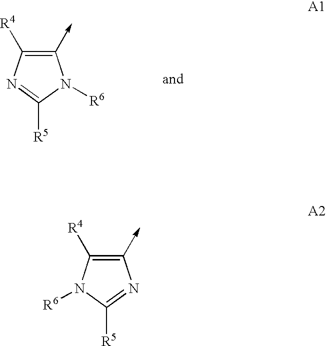
wherein
R⁴ is hydrogen, C₁₋₁₂-alkyl, substituted C₁₋₄-alkyl, C₃₋₈-cycloalkyl, C₁₋₄-alkoxy, CN, COR, CO₂R, CONRR′, NHCOR, aryl, substituted aryl, aryl-C(═O)—, substituted aryl-C(═O)—, aryl-CH(OH)—, substituted aryl-CH(OH)—, heterocyclyl, substituted heterocyclyl, heterocyclyl-C(═O)—, substituted heterocyclyl-C(═O)—, heterocyclyl-CH(OH)—, substituted heterocyclyl-CH(OH)— or NRR′,
wherein substituted C₁₋₄-alkyl means alkyl substituted with 1-3 substituents selected from C₃₋₈-cycloalkyl, aryl, heterocyclyl, substituted aryl and substituted heterocyclyl; wherein substituted aryl and substituted heterocyclyl means aryl and heterocyclyl substituted with C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens, and
wherein substituted aryl, substituted aryl-C(═O)— or substituted aryl-CH(OH)— are substituted with 1-5 substituents selected from C₁₋₄-alkoxy, halogen, CN, NO₂, COR, CO₂R, CONRR′, NRR′, NHCOR, SO₂NRR′, SO₂R, C₁₋₄-alkyl and C₁₋₄-alkyl substituted with 1-3 halogens, and
wherein substituted heterocyclyl, substituted heterocyclyl-C(═O)— or substituted heterocyclyl-CH(OH)— are substituted with 1-4 substituents selected from C₁₋₄-alkoxy, halogen, CN, NO₂, COR, CO₂R, CONRR′, NRR′, NHCOR, SO₂NRR′, SO₂R, C₁₋₄-alkyl and C₁₋₄-alkyl substituted with 1-3 halogens;
R⁵ is hydrogen, C₁₋₁₂-alkyl, substituted C₁₋₄-alkyl, C₃₋₈-cycloalkyl, C₁₋₄-alkoxy, halogen, COR, aryl, substituted aryl, aryl-C(═O)—, substituted aryl-C(═O)—, aryl-CH(OH)—, substituted aryl-CH(OH)—, heterocyclyl, substituted heterocyclyl, heterocyclyl-C(═O)—, substituted heterocyclyl-C(═O)—, heterocyclyl-CH(OH)—, substituted heterocyclyl-CH(OH)— or NRR′,
wherein substituted C₁₋₄-alkyl means alkyl substituted with 1-3 substituents selected from C₃₋₈-cycloalkyl, aryl, heterocyclyl, substituted aryl and substituted heterocyclyl; wherein substituted aryl and substituted heterocyclyl means aryl and heterocyclyl substituted with C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens, and
wherein substituted aryl, substituted aryl-C(═O)— or substituted aryl-CH(OH)— are substituted with 1-5 substituents selected from C₁₋₄-alkoxy, halogen, CN, NO₂, COR, CO₂R, CONRR′, NRR′, NHCOR, SO₂NRR′, SO₂R, C₁₋₄-alkyl and C₁₋₄-alkyl substituted with 1-3 halogens, and
wherein substituted heterocyclyl, substituted heterocyclyl-C(═O)— or substituted heterocyclyl-CH(OH)— are substituted with 1-4 substituents selected from C₁₋₄-alkoxy, halogen, CN, NO₂, COR, CO₂R, CONRR′, NRR′, NHCOR, SO₂NRR′, SO₂R, C₁₋₄-alkyl and C₁₋₄-alkyl substituted with 1-3 halogens;
R⁶ is hydrogen, C₁₋₁₂-alkyl, substituted C₁₋₄-alkyl, C₁₋₄-alkoxy, C₃₋₈-cycloalkyl, COR, CO₂R, CONRR′, NHCOR, SO₂NRR′or SO₂R,
wherein substituted C₁₋₄-alkyl means alkyl substituted with 1-3 substituents selected from C₃₋₈-cycloalkyl, aryl, heterocyclyl, substituted aryl and substituted heterocyclyl; wherein substituted aryl and substituted heterocyclyl means aryl and heterocyclyl substituted with C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens;
R and R′ are independently of each other hydrogen, C₁₋₁₂-alkyl, substituted C₁₋₄-alkyl, C₃₋₈-cycloalkyl, aryl, substituted aryl, heterocyclyl or substituted heterocyclyl,
wherein substituted C₁₋₄-alkyl means alkyl substituted with 1-3 substituents selected from C₃₋₈-cycloalkyl, aryl, heterocyclyl, substituted aryl and substituted heterocyclyl; wherein substituted aryl and substituted heterocyclyl means aryl and heterocyclyl substituted with C₁₋₄-alkoxy, halogen, CN, NO₂, COR⁷, CO₂R⁷, CONR⁷R⁸, NR⁷R⁸, NHCOR⁷, SO₂NR⁷R⁸, SO₂R⁷, C₁₋₄-alkyl or C₁₋₄-alkyl substituted with 1-3 halogens, and
wherein substituted aryl are substituted with 1-5 substituents and substituted heterocyclyl are substituted with 1-4 substituents, these substituents selected from C₁₋₄-alkoxy, halogen, CN, NO₂, COR⁷, CO₂R⁷, CONR⁷R⁸, NR⁷R⁸, NHCOR⁷, SO₂NR⁷R⁸, SO₂R⁷, C₁₋₄-alkyl and C₁₋₄-alkyl substituted with 1-3 halogens;
R⁷ and R⁸ are independently of each other hydrogen or C₁₋₄-alkyl.

3. The compound according to claim 1 wherein
R¹ is hydrogen, C₁₋₁₂-alkyl, C₃₋₈-cycloalkyl, allyl, substituted C₁₋₄-alkyl, aryl, substituted aryl or heterocyclyl,
wherein substituted C₁₋₄-alkyl means alkyl substituted with 1-3 substituents selected from C₃₋₈-cycloalkyl, aryl, heterocyclyl, substituted aryl and substituted heterocyclyl; wherein substituted aryl and substituted heterocyclyl means aryl and heterocyclyl substituted with C₁₋₄-alkoxy, phenyl, phenoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens, and
wherein substituted aryl means aryl substituted with 1-5 substituents selected from C₁₋₄-alkoxy, halogen, CN, NO₂, COR, CO₂R, CONRR′, NRR′, SO₂R, NHCOR, SO₂NRR′, C₁₋₄-alkyl and C₁₋₄-alkyl substituted with 1-3 halogens;
R² and R³ are independently of each other hydrogen, C₁₋₁₂-alkyl, C₃₋₈-cycloalkyl, substituted C₁₋₄-alkyl, aryl, substituted aryl, heterocyclyl or substituted heterocyclyl,
wherein substituted C₁₋₄-alkyl means alkyl substituted with 1-3 substituents selected from C₃₋₈-cycloalkyl, aryl, heterocyclyl, substituted aryl and substituted heterocyclyl; wherein substituted aryl and substituted heterocyclyl means aryl and heterocyclyl substituted with C₁₋₄-alkoxy, halogen, CN, NO₂, COR, CO₂R, CONRR′, NRR′, SO₂R, NHCOR, SO₂NRR′, C₁₋₄-alkyl or C₁₋₄-alkyl substituted with 1-3 halogens, and
wherein substituted aryl means aryl substituted with 1-5 substituents and substituted heterocyclyl means heterocyclyl substituted with 1-4 substituents and these substituents are selected from C₁₋₄-alkoxy, halogen, CN, NO₂, COR, CO₂R, CONRR′, NRR′, SO₂R, NHCOR, SO₂NRR′, C₁₋₄-alkyl and C₁₋₄-alkyl substituted with 1-3 halogens;
X is S or O;
A is selected from the group consisting of: 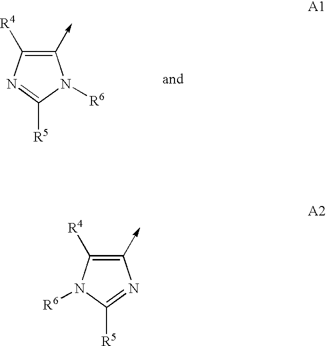
wherein
R⁴ is hydrogen, C₁₋₁₂-alkyl, CO₂R or aryl;
R⁵ is hydrogen, C₁₋₁₂-alkyl, substituted C₁₋₄-alkyl, halogen, aryl, substituted aryl, aryl-C(═O)—, aryl-CH(OH)— or NRR′,
wherein substituted C₁₋₄-alkyl means alkyl substituted with 1-3 substituents selected from C₃₋₈-cycloalkyl, aryl, heterocyclyl, substituted aryl and substituted heterocyclyl; wherein substituted aryl and substituted heterocyclyl means aryl and heterocyclyl substituted with C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens, and
wherein substituted aryl means aryl substituted with 1-5 substituents selected from C₁₋₄-alkoxy, halogen, CN, NO₂, COR, CO₂R, CONRR′, NRR′, NHCOR, SO₂NRR′, SO₂R, C₁₋₄-alkyl and C₁₋₄-alkyl substituted with 1-3 halogens;
R⁶ is hydrogen, C₁₋₁₂-alkyl or substituted C₁₋₄-alkyl,
wherein substituted C₁₋₄-alkyl means alkyl substituted with 1-3 substituents selected from C₃₋₈-cycloalkyl, aryl, heterocyclyl, substituted aryl and substituted heterocyclyl; wherein substituted aryl and substituted heterocyclyl means aryl and heterocyclyl substituted with C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens;
R and R′ are independently of each other hydrogen or C₁₋₁₂-alkyl.

4. The compound according to claim 1 wherein
R¹ is hydrogen, C₁₋₁₂-alkyl, C₃₋₈-cycloalkyl, allyl, substituted C₁₋₄-alkyl, phenyl, substituted phenyl or pyridyl,
wherein substituted C₁₋₄-alkyl means alkyl substituted with 1-3 substituents selected from C₃₋₈-cycloalkyl, phenyl, pyridyl, substituted phenyl and substituted pyridyl; wherein substituted phenyl and substituted pyridyl are substituted with C₁₋₄-alkoxy, phenyl, phenoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens, and
wherein substituted phenyl is substituted with 1-5 substituents selected from C₁₋₄-alkoxy, halogen, CN, NO₂, COR, CO₂R, CONRR′, NRR′, SO₂R, NHCOR, SO₂NRR′, C₁₋₄-alkyl and C₁₋₄-alkyl substituted with 1-3 halogens;
R² and R³ are independently of each other hydrogen, C₁₋₁₂-alkyl, C₃₋₈-cycloalkyl, substituted C₁₋₄-alkyl, phenyl, substituted phenyl, heterocyclyl or substituted heterocyclyl,
wherein substituted C₁₋₄-alkyl means alkyl substituted with 1-3 substituents selected from C₃₋₈-cycloalkyl, phenyl, pyridyl, substituted phenyl and substituted pyridyl; wherein substituted phenyl or substituted pyridyl are substituted with C₁₋₄-alkoxy, halogen, CN, NO₂, COR, CO₂R, CONRR′, NRR′, SO₂R, NHCOR, SO₂NRR′, C₁₋₄-alkyl or C₁₋₄-alkyl substituted with 1-3 halogens, and
wherein substituted phenyl is substituted with 1-5 substituents and substituted heterocyclyl means heterocyclyl substituted with 1-4 substituents and these substituents are selected from C₁₋₄-alkoxy, halogen, CN, NO₂, COR, CO₂R, CONRR′, NRR′, SO₂R, NHCOR, SO₂NRR′, C₁₋₄-alkyl and C₁₋₄-alkyl substituted with 1-3 halogens;
X is S or O;
A is selected from the group consisting of: 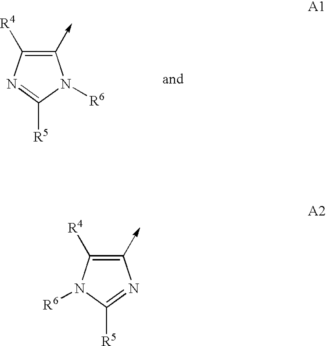
wherein
R⁴ is hydrogen, C₁₋₁₂-alkyl, CO₂R or phenyl;
R⁵ is hydrogen, C₁₋₁₂-alkyl, substituted C₁₋₄-alkyl, halogen, phenyl, substituted phenyl, phenyl-C(═O)—, phenyl-CH(OH)— or NRR′,
wherein substituted C₁₋₄-alkyl means alkyl substituted with 1-3 substituents selected from C₃₋₈-cycloalkyl, phenyl, heterocyclyl, substituted phenyl and substituted heterocyclyl; wherein substituted phenyl and substituted heterocyclyl are substituted with C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens, and
wherein substituted phenyl is substituted with 1-5 substituents selected from C₁₋₄-alkoxy, halogen, CN, NO₂, COR, CO₂R, CONRR′, NRR′, NHCOR, SO₂NRR′, SO₂R, C₁₋₄-alkyl and C₁₋₄-alkyl substituted with 1-3 halogens;
R⁶ is hydrogen, C₁₋₁₂-alkyl or substituted C₁₋₄-alkyl,
wherein substituted C₁₋₄-alkyl means alkyl substituted with 1-3 substituents selected from C₃₋₈-cycloalkyl, phenyl, heterocyclyl, substituted phenyl and substituted heterocyclyl; wherein substituted phenyl or substituted heterocyclyl are substituted with C₁₋₄-alkoxy, halogen, CN, NO₂, COR, CO₂R, CONRR′, NRR′, NHCOR, SO₂NRR′, SO₂R, C₁₋₄-alkyl or C₁₋₄-alkyl substituted with 1-3 halogens;
R and R′ are independently of each other hydrogen or C₁₋₁₂-alkyl.

5. The compound according to claim 1 wherein
R¹ is hydrogen, C₁₋₁₂-alkyl, C₃₋₈-cycloalkyl, allyl, substituted C₁₋₄-alkyl, phenyl, substituted phenyl or pyridyl,
wherein substituted C₁₋₄-alkyl means alkyl substituted with 1-3 substituents selected from C₃₋₈-cycloalkyl, phenyl, pyridyl and substituted phenyl; wherein substituted phenyl is substituted with C₁₋₄-alkoxy, phenyl, phenoxy, halogen, CN, NO₂, CO₂R, NRR′, SO₂R, C₁₋₄-alkyl or C₁₋₄-alkyl substituted with 1-3 halogens, and
wherein substituted phenyl is substituted with 1-5 substituents selected from C₁₋₄-alkoxy, halogen, C₁₋₄-alkyl and C₁₋₄-alkyl substituted with 1-3 halogens; R² and R³ are independently of each other hydrogen, C₁₋₁₂-alkyl, C₃₋₈-cycloalkyl, substituted C₁₋₄-alkyl, phenyl, substituted phenyl, heterocyclyl or substituted heterocyclyl,
wherein substituted C₁₋₄-alkyl means alkyl substituted with 1-3 substituents selected from phenyl, pyridyl and substituted phenyl; wherein substituted phenyl is substituted with C₁₋₄-alkoxy, halogen, NO₂, C₁₋₄-alkyl or C₁₋₄-alkyl substituted with 1-3 halogens, and
wherein substituted phenyl is substituted with 1-5 substituents and substituted heterocyclyl means heterocyclyl substituted with 1-4 substituents and these substituents are selected from C₁₋₄-alkoxy, halogen, CN, NO₂, CO₂R, NRR′, C₁₋₄-alkyl and C₁₋₄-alkyl substituted with 1-3 halogens;
X is S or O;
A is selected from the group consisting of: 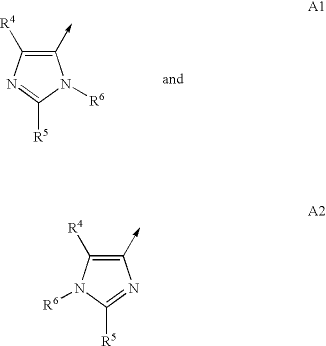
wherein
R⁴ is hydrogen, C₁₋₁₂-alkyl, CO₂R or phenyl;
R⁵ is hydrogen, C₁₋₁₂-alkyl, substituted C₁₋₄-alkyl, halogen, phenyl, substituted phenyl, phenyl-C(═O)—, phenyl-CH(OH)— or NRR′,
wherein substituted C₁₋₄-alkyl means alkyl substituted with 1-3 substituents selected from phenyl and substituted phenyl, wherein substituted phenyl is substituted with C₁₋₄-alkoxy, halogen, C₁₋₄-alkyl or C₁₋₄-alkyl substituted with 1-3 halogens, and
wherein substituted phenyl is substituted with 1-5 substituents selected from C₁₋₄-alkoxy, halogen, C₁₋₄-alkyl and C₁₋₄-alkyl substituted with 1-3 halogens; R⁶ is hydrogen, C₁₋₁₂-alkyl or substituted C₁₋₄-alkyl,
wherein substituted C₁₋₄-alkyl means alkyl substituted with 1-3 substituents selected from phenyl and substituted phenyl; wherein substituted phenyl is substituted with C₁₋₄-alkoxy, halogen, C₁₋₄-alkyl or C₁₋₄-alkyl substituted with 1-3 halogens;
R and R′ are independently of each other hydrogen or C₁₋₁₂-alkyl.

6. The compound according to claim 1 wherein
R¹ is hydrogen, C₁₋₁₂-alkyl, C₃₋₈-cycloalkyl, allyl, substituted C₁₋₄-alkyl, phenyl, substituted phenyl or pyridyl,
wherein substituted C₁₋₄-alkyl means alkyl substituted with 1-3 substituents selected from C₃₋₈-cycloalkyl, phenyl, pyridyl and substituted phenyl, wherein substituted phenyl is substituted with C₁₋₄-alkoxy, phenyl, phenoxy, chlorine, CN, NO₂, CO₂R, NRR′, SO₂R, C₁₋₄-alkyl or C₁₋₄-alkyl substituted with 1-3 fluorines, and
wherein substituted phenyl is substituted with 1-5 substituents selected from C₁₋₄-alkoxy, chlorine, C₁₋₄-alkyl and C₁₋₄-alkyl substituted with 1-3 fluorines; R² and R³ are independently of each other hydrogen, C₁₋₁₂-alkyl, C₃₋₈-cycloalkyl, substituted C₁₋₄-alkyl, phenyl, substituted phenyl, heterocyclyl or substituted heterocyclyl,
wherein substituted C₁₋₄-alkyl means alkyl substituted with 1-3 substituents selected from phenyl, pyridyl and substituted phenyl, wherein substituted phenyl is substituted with NO₂, and
wherein substituted phenyl is substituted with 1-5 substituents and substituted heterocyclyl means heterocyclyl substituted with 1-4 substituents and these substituents are selected from C₁₋₄-alkoxy, fluorine, chlorine, CN, NO₂, CO₂R, NRR′, C₁₋₄-alkyl and C₁₋₄-alkyl substituted with 1-3 fluorines;
X is S or O;
A is selected from the group consisting of: 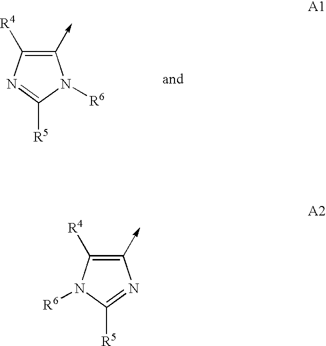
wherein
R⁴ is hydrogen, C₁₋₁₂-alkyl, CO₂R or phenyl;
R⁵ is hydrogen, C₁₋₁₂-alkyl, substituted C₁₋₄-alkyl, halogen, phenyl, substituted phenyl, phenyl-C(═O)—, phenyl-CH(OH)— or NRR′,
wherein substituted C₁₋₄-alkyl means alkyl substituted with 1-3 substituents selected from phenyl, and
wherein substituted phenyl is substituted with 1-5 substituents selected from C₁₋₄-alkoxy, chlorine, C₁₋₄-alkyl and C₁₋₄-alkyl substituted with 1-3 fluorines;
R⁶ is hydrogen, C₁₋₁₂-alkyl or substituted C₁₋₄-alkyl,
wherein substituted C₁₋₄-alkyl means alkyl substituted with 1-3 substituents selected from phenyl;
R and R′ are independently of each other hydrogen or C₁₋₁₂-alkyl.

7. The compound according to claim 1 wherein
R¹ is hydrogen, C₁₋₇-alkyl, C₃₋₆-cycloalkyl, allyl, substituted C₁₋₂-alkyl, phenyl, substituted phenyl or pyridyl,
wherein substituted C₁₋₂-alkyl means alkyl substituted with 1-3 substituents selected from C₃₋₆-cycloalkyl, phenyl, pyridyl and substituted phenyl, wherein substituted phenyl is substituted with C₁₋₂-alkoxy, phenyl, phenoxy, chlorine, CN, NO₂, CO₂R, NRR′, SO₂R, C₁₋₂-alkyl or C₁₋₂-alkyl substituted with 1-3 fluorines, and
wherein substituted phenyl is substituted with 1-5 substituents selected from C₁₋₂-alkoxy, chlorine, C₁₋₂-alkyl and C₁₋₂-alkyl substituted with 1-3 fluorines; R² and R³ are independently of each other hydrogen, C₁₋₇-alkyl, C₃₋₆-cycloalkyl, substituted C₁₋₂-alkyl, phenyl, substituted phenyl, heterocyclyl or substituted heterocyclyl,
wherein substituted C₁₋₂-alkyl means alkyl substituted with 1-3 substituents selected from phenyl, pyridyl and substituted phenyl, wherein substituted phenyl is substituted with NO₂, and
wherein substituted phenyl is substituted with 1-5 substituents and substituted heterocyclyl means heterocyclyl substituted with 1-4 substituents and these substituents are selected from C₁₋₂-alkoxy, fluorine, chlorine, CN, NO₂, CO₂R, NRR′, C₁₋₂-alkyl and C₁₋₂-alkyl substituted with 1-3 fluorines;
X is S or O;
A is selected from the group consisting of: 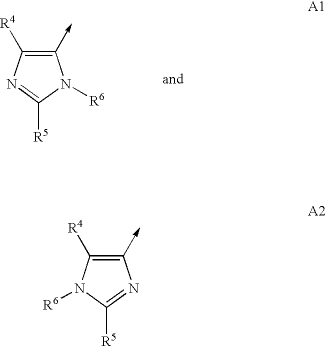
wherein
R⁴ is hydrogen, C₁₋₇-alkyl, CO₂R or phenyl;
R⁵ is hydrogen, C₁₋₇-alkyl, substituted C₁₋₂-alkyl, halogen, phenyl, substituted phenyl, phenyl-C(═O)—, phenyl-CH(OH)— or NRR′,
wherein substituted C₁₋₂-alkyl means alkyl substituted with 1-3 substituents selected from phenyl, and
wherein substituted phenyl is substituted with 1-5 substituents selected from C₁₋₂-alkoxy, chlorine, C₁₋₂-alkyl and C₁₋₂-alkyl substituted with 1-3 fluorines;
R⁶ is hydrogen, C₁₋₇-alkyl or substituted C₁₋₂-alkyl,
wherein substituted C₁₋₂-alkyl means alkyl substituted with 1-3 substituents selected from phenyl;
R and R′ are independently of each other hydrogen or C₁₋₇-alkyl.

8. The compound according to claim 1 wherein
R¹ is hydrogen, C₁₋₄-alkyl, C₃₋₆-cycloalkyl, allyl, substituted C₁-alkyl, phenyl, substituted phenyl or pyridyl,
wherein substituted C₁-alkyl means alkyl substituted with 1-3 substituents selected from C₃₋₆-cycloalkyl, phenyl, pyridyl and substituted phenyl, wherein substituted phenyl is substituted with C₁-alkoxy, phenyl, phenoxy, chlorine, CN, NO₂, CO₂R, NRR′, SO₂R, C₁-alkyl or C₁-alkyl substituted with 1-3 fluorines, and
wherein substituted phenyl is substituted with 1-5 substituents selected from C₁-alkoxy, chlorine, C₁-alkyl and C₁-alkyl substituted with 1-3 fluorines; R² and R³are independently of each other hydrogen, C₁₋₄-alkyl, C₃₋₆-cycloalkyl, substituted C₁-alkyl, phenyl, substituted phenyl, heterocyclyl or substituted heterocyclyl,
wherein substituted C₁-alkyl means alkyl substituted with 1-3 substituents selected from phenyl, pyridyl and substituted phenyl, wherein substituted phenyl is substituted with NO₂, and
wherein substituted phenyl is substituted with 1-5 substituents and substituted heterocyclyl means heterocyclyl substituted with 1-4 substituents and these substituents are selected from C₁-alkoxy, fluorine, chlorine, CN, NO₂, CO₂R, NRR′, C₁-alkyl and C₁-alkyl substituted with 1-3 fluorines;
X is S or O;
A is selected from the group consisting of: 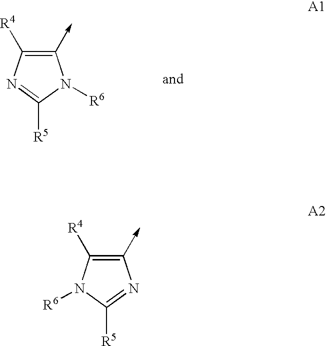
wherein
R⁴ is hydrogen, C₁₋₄-alkyl, CO₂R or phenyl;
R⁵ is hydrogen, C₁₋₄-alkyl, substituted C₁-alkyl, halogen, phenyl, substituted phenyl, phenyl-C(═O)—, phenyl-CH(OH)— or NRR′,
wherein substituted C₁-alkyl means alkyl substituted with 1-3 substituents selected from phenyl, and
wherein substituted phenyl is substituted with 1-5 substituents selected from C₁-alkoxy, chlorine, C₁-alkyl and C₁-alkyl substituted with 1-3 fluorines;
R⁶ is hydrogen, C₁₋₅-alkyl or substituted C₁-alkyl,
wherein substituted C₁-alkyl means alkyl substituted with 1-3 substituents selected from phenyl;
R and R′ are independently of each other hydrogen or C₁₋₄-alkyl.

9. The compound according to claim 1 wherein
X is O.

10. The compound according to claim 1 wherein
A is A1.

11. The compound according to claim 1 wherein
A is A2.

12. A compound selected from the group consisting of:
1-[1-[[2-[4-(Trifluoromethyl)phenyl]-5-methyl-1H-imidazol-4-yl]methyl]-4-piperidinyl]-3-methyl-1-phenylurea,
3-Methyl-1-[1-[(5-methyl-1H-imidazol-4-yl)methyl]-4-piperidinyl]-1-phenylurea,
3-Methyl-1-[1-[(5-methyl-2-phenyl-1H-imidazol-4-yl)methyl]-4-piperidinyl]-1-phenylurea,
1,1-Dimethyl-3-[1-[(5-methyl-2-phenyl-1H-imidazol-4-yl)methyl]-4-piperidinyl]-3-phenylurea,
1-Benzyl-3-methyl-1-[1-[(5-methyl-2-phenyl-1H-imidazol-4-yl)methyl]-4-piperidinyl]urea,
1-(4-Methoxyphenyl)-3-methyl-1-[1-[(5-methyl-2-phenyl-1H-imidazol-4-yl)methyl]-4-piperidinyl]urea,
1-Benzyl-3-methyl-1-[1-[[5-methyl-2-[4-(trifluoromethyl)phenyl]-1H-imidazol-4-yl]methyl]-4-piperidinyl]urea,
3-Methyl-1-[1-[[5-methyl-2-(4-methylphenyl)-1H-imidazol-4-yl]methyl]-4-piperidinyl]-1-phenylurea,
1-[1-[[2-(4-Chlorophenyl)-5-methyl-1H-imidazol-4-yl]methyl]-4-piperidinyl]-3-methyl-1-phenylurea,
3-Methyl-1-phenyl-1-[1-[[2-[4-(trifluoromethyl)phenyl]-1H-imidazol-4-yl]methyl]-4-piperidinyl]urea,
1-[1-[[2-(2,3-Dimethoxyphenyl)-1H-imidazol-4-yl]methyl]-4-piperidinyl]-3-methyl-4-phenylurea,
1-[1-[[2-(2,3-Dimethoxyphenyl)-5-methyl-1H-imidazol-4-yl]methyl]-4-piperidinyl]-3-methyl-1-phenylurea,
1-Benzyl-3-methyl-1-[1-[[5-phenyl-2-[4-(trifluoromethyl)phenyl]-1H-imidazol-4-yl]methyl]-4-piperidinyl]urea,
3-Methyl-1-phenyl-1-[1-[[5-phenyl-2-[4-(trifluoromethyl)phenyl]-1H-imidazol-4-yl]methyl]-4-piperidinyl]urea,
3-Methyl-1-[1-[[5-methyl-2-[4-(trifluoromethyl)phenyl]-1H-imidazol-4-yl]methyl]-4-piperidinyl]-1-phenylthiourea,
1-Benzyl-3-methyl-1-[1-[(5-methyl-1H-imidazol-4-yl)methyl]-4-piperidinyl]urea,
1-Benzyl-1-[1-[(2-iodo-5-methyl-1-imidazol-4-yl)methyl]-4-piperidinyl]-3-methylurea,
1-Allyl-1-[1-[[5-methyl-2-[4-(trifluoromethyl)phenyl]-1H-imidazol-4-yl]methyl]-4-piperidinyl]-3-(4-nitrobenzyl)urea,
1-[1-[(2-Benzoyl-5-methyl-1H-imidazol-4-yl)methyl]-4-piperidinyl]-1-benzyl-3-methylurea,
1-Benzyl-1-[1-[[2-[(RS)-(hydroxy)(phenyl)methyl]-5-methyl-1H-imidazol-4-yl]methyl]-4-piperidinyl]-3-methylurea,
1-Benzyl-1-[1-[[1-benzyl-5-methyl-2-[4-(trifluoromethyl)phenyl]-1H-imidazol-4-yl]methyl]-4-piperidinyl]-3-methylurea,
1-Benzyl-1-[1-[[3-benzyl-5-methyl-2-[4-(trifluoromethyl)phenyl]-3H-imidazol-4-yl]methyl]-4-piperidinyl]-3-methylurea,
1-[1-[[2-[4-(Trifluoromethyl)phenyl]-5-methyl-1H-imidazol-4-yl]methyl]-4-piperidinyl]-1,3-dimethylurea,
1-Butyl-1-[1-[[2-[4-(trifluoromethyl)phenyl]-5-methyl-1H-imidazol-4-yl]methyl]-4-piperidinyl]-3-methylurea,
1-Cyclohexyl-1-[1-[[2-[4-(trifluoromethyl)phenyl]-5-methyl-1H-imidazol-4yl]methyl]-4-piperidinyl]-3-methylurea,
1-[1-[[2-[4-(Trifluoromethyl)phenyl]-5-methyl-1H-imidazol-4-yl]methyl]-4-piperidinyl]-3-methyl-1-(2-phenethyl)urea,
1-[1-[[2-[4-(Trifluoromethyl)phenyl]-5-methyl-1H-imidazol-4-yl]methyl]-4-piperidinyl]-3-methyl-1-(3-phenylpropyl)urea,
1-[1-[[2-[4-(Trifluoromethyl)phenyl]-5-methyl-1H-imidazol-4-yl]methyl]-4-piperidinyl]-1-(4-methoxybenzyl)-3-methylurea,
1-(4-Chlorobenzyl)-1-[1-[[2-[4-(trifluoromethyl)phenyl]-5-methyl-1H-imidazol-4-yl]methyl]-4-piperidinyl]-3-methylurea,
1-[1-[[2-[4-(Trifluoromethyl)phenyl]-5-methyl-1H-imidazol-4-yl]methyl]-4-piperidinyl]-3-methyl-1-[(4-pyridyl)methyl]urea,
1-Benzyl-3-ethyl-1-[1-[[2-[4-(trifluoromethyl)phenyl]-5-methyl-1H-imidazol-4-yl]methyl]-4-piperidinyl]urea,
1-Benzyl-1-[1-[[2-[4-(trifluoromethyl)phenyl]-5-methyl-1H-imidazol-4-yl]methyl]-4-piperidinyl]-3-propylurea,
1-Benzyl-1-[1-[[2-[4-(trifluoromethyl)phenyl]-5-methyl-1H-imidazol-4-yl]methyl]-4-piperidinyl]-3-phenylurea,
1-Benzyl-1-[1-[[2-[4-trifluoromethyl-phenyl]-5-methyl-1H-imidazol-4-yl]methyl]-4-piperidinyl]-3-(4-methoxyphenyl)urea,
1-Benzyl-3-[4-(trifluoromethyl)phenyl]1-[1-[[2-[4-(trifluoromethyl)phenyl-5methyl-1H-imidazol-4-yl]methyl]-4-piperidinyl]urea,
1,3-Dibenzyl-1-[1-[[2-[4-(trifluoromethyl)phenyl]-5-methyl-1H-imidazol-4-yl]methyl]-4-piperidinyl]urea,
1-Benzyl-3-cyclohexyl-1-[1-[[2-[4-(trifluoromethyl)phenyl]-5-methyl-1H-imidazol-4-yl]methyl]-4-piperidinyl]urea,
1-Benzyl-3-tert.-butyl-1-[1-[[2-[4-(trifluoromethyl)phenyl]-5-methyl-1H-imidazol-4-yl]methyl]-4-piperidinyl]urea,
1-Benzyl-1-[1-[[2-[4-(trifluoromethyl)phenyl]-5-methyl-1H-imidazol-4-yl]methyl]-4-piperidinyl]-3-( 2-phenylethyl)urea,
1-Benzyl-1-[1-[[2-[4-(trifluoromethyl)phenyl]-5-methyl-1H-imidazol-4-yl]methyl]-4-piperidinyl]-3-(3-phenylpropyl)urea,
1-[1-[[2-[4-(Trifluoromethyl)phenyl]-5-methyl-1H-imidazol-4-yl]methyl]-4-piperidinyl]-1-(2,4,6-trimethoxybenzyl)-3-methylurea,
1-Allyl-1-[1-[[1-(2-chlorobenzoyl)-4(R)-phenyl-3(R)-pyrrolidinyl]methyl]-piperidin-4-yl]-3-(4-nitrobenzyl)urea,
1-Benzyl-1-[1-[[2-[4-(trifluoromethyl)phenyl]-5-methyl-1H-imidazol-4-yl]methyl]-4-piperidinyl]-3-(2-methylphenyl)urea,
1-Benzyl-1-[1-[[2-[4-(trifluoromethyl)phenyl]-5-methyl-1H-imidazol-4-yl]methyl]-4-piperidinyl]-3-(3-methylphenyl)urea,
1-Benzyl-1-[1-[[2-[4-(trifluoromethyl)phenyl]-5-methyl-1H-imidazol-4-yl]methyl]-4-piperidinyl]-3-(4-methylphenyl)urea,
1-Benzyl-1-[1-[[2-[4-(trifluoromethyl)phenyl]-5-methyl-1H-imidazol-4-yl]methyl]-4-piperidinyl]-3-(3,4-dimethylphenyl)urea,
1-Benzyl-1-[1-[[2-[4-(trifluoromethyl)phenyl]-5-methyl-1H-imidazol-4-yl]methyl]-4-piperidinyl]-3-(3,5-dimethylphenyl)urea,
1-Benzyl-3-(2-chlorophenyl)-1-[1-[[2-[4-(trifluoromethyl)phenyl]-5-methyl-1H-imidazol-4-yl]methyl]-4-piperidinyl]urea,
1-Benzyl-3-(3-chlorophenyl)-1-[1-[[2-[4-(trifluoromethyl)phenyl]-5-methyl-1H-imidazol-4-yl]methyl]-4-piperidinyl]urea,
1-Benzyl-3-(3,5-dichlorophenyl)-1-[1-[[2-[4-(trifluoromethyl)phenyl]-5-methyl-1H-imidazol-4-yl]methyl]-4-piperidinyl]urea,
1-Benzyl-3-(4-fluorophenyl)-1-[1-[[2-[4-(trifluoromethyl)phenyl]-5-methyl-1H-imidazol-4-yl]methyl]-4-piperidinyl]urea,
1-Benzyl-1-[1-[[2-[4-(trifluoromethyl)phenyl]-5-methyl-1H-imidazol-4-yl]methyl]-4-piperidinyl]-3-[4-(dimethylamino)phenyl]urea,
1-Benzyl-3-(4-cyanophenyl)-1-[1-[[2-[4-(trifluoromethyl)phenyl]-5-methyl-1H-imidazol-4-yl]methyl]-4-piperidinyl]urea,
1-Benzyl-1-[1-[[2-[4-(trifluoromethyl)phenyl]-5-methyl-1H-imidazol-4-yl]methyl]-4-piperidinyl]-3-(4-nitrophenyl)urea,
1-Benzyl-3-(3-bromophenyl)-1-[1-[[2-[4-(trifluoromethyl)phenyl]-5-methyl-1H-imidazol-4-yl]methyl]-4-piperidinyl]urea,
1-Benzyl-3-[3-(trifluoromethyl)phenyl]-1-[1-[[2-[4-(trifluoromethyl)phenyl]-5-methyl-1H-imidazol-4-yl]methyl]-4-piperidinyl]urea,
1-[1-[[2-(2-Methoxyphenyl)-5-methyl-1H-imidazol-4-yl]methyl]-4-piperidinyl]-3-methyl-1-phenylurea,
Methyl 5-[[4-(1-benzyl-3-methylureido)piperidino]methyl]-2-[4-(trifluoromethyl)phenyl]-3H-imidazole-4-carboxylate,
1-Benzyl-1-[1-[5-methyl-2-(4-methylphenyl)-1H-imidazol-4-ylmethyl]-4-piperidinyl]-3-phenylurea,
1-Methyl-3-{1-[5-methyl-2-(4-trifluoromethyl-phenyl)-1H-imidazol-4-ylmethyl]-piperidin-4-yl}-urea,
1-Ethyl-3-methyl-1-{1-[5-methyl-2-(4-trifluoromethyl-phenyl)-1H-imidazol-4-ylmethyl]-piperidin-4-yl}-urea,
3-Methyl-1-{1-[5-methyl-2-(4-trifluoromethyl-phenyl)-1H-imidazol-4-ylmethyl]-piperidin-4-yl}-1-propyl-urea,
1-Isopropyl-3-methyl-1-{1-[5-methyl-2-(4-trifluoromethyl-phenyl)-1H-imidazol-4-ylmethyl]-piperidin-4-yl}-urea,
1-Allyl-3-methyl-1-{1-[5-methyl-2-(4-trifluoromethyl-phenyl)-1H-imidazol-4ylmethyl]-piperidin-4-yl}-urea,
1-Isobutyl-3-methyl-1-{1-[5-methyl-2-(4-trifluoromethyl-phenyl)-1H-imidazol-4ylmethyl]-piperidin-4-yl}-urea,
1-tert.-butyl-3-methyl-1-{1-[5-methyl-2-(4-trifluoromethyl-phenyl)-1H-imidazol-4-ylmethyl]-piperidin-4-yl}-urea,
1-Cyclopropyl-3-methyl-1-{1-[5-methyl-2-(4-trifluoromethyl-phenyl)-1H-imidazol-4-ylmethyl]-piperidin-4-yl}-urea,
1-Cyclopropylmethyl-3-methyl-1-{1-[5-methyl-2-(4-trifluoromethyl-phenyl)-1H-imidazol-4-ylmethyl]-piperidin-4-yl}-urea,
1-Cyclobutylmethyl-3-methyl-1-{1-[5-methyl-2-(4-trifluoromethyl-phenyl)-1H-imidazol-4-ylmethyl]-piperidin-4-yl}-urea,
1-Cyclopentylmethyl-3-methyl-1-{1-[5-methyl-2-(4-trifluoromethyl-phenyl)-1H-imidazol-4-ylmethyl]-piperidin-4-yl}-urea,
1-Cyclohexylmethyl-3-methyl-1-{1-[5-methyl-2-(4-trifluoromethyl-phenyl)-1H-imidazol-4-ylmethyl]-piperidin-4-yl}-urea,
1-(2-Methoxy-phenyl)-3-methyl-1-{1-[5-methyl-2-(4-trifluoromethyl-phenyl)-1H-imidazol-4-ylmethyl]-piperidin-4-yl}-urea,
1-(4-Methoxy-phenyl)-3-methyl-1-{1-[5-methyl-2-(4-trifluoromethyl-phenyl)-1H-imidazol-4-ylmethyl]-piperidin-4-yl}-urea,
1-(2-Chloro-phenyl)-3-methyl-1-{1-[5-methyl-2-(4-trifluoromethyl-phenyl)-1H-imidazol-4-ylmethyl]-piperidin-4-yl}-urea,
1-(4-Chloro-phenyl)-3-methyl-1-{1-[5-methyl-2-(4-trifluoromethyl-phenyl)-1H-imidazol-4-ylmethyl]-piperidin-4-yl}-urea,
3-Methyl-1-{1-[5-methyl-2-(4-trifluoromethyl-phenyl)-1H-imidazol-4-ylmethyl]-piperidin-4-yl}-1-(2-trifluoromethyl-phenyl)-urea,
3-Methyl-1-{1-[5-methyl-2-(4-trifluoromethyl-phenyl)-1H-imidazol-4-ylmethyl]-piperidin-4-yl}-1-(4-trifluoromethyl-phenyl)-urea,
3-Methyl-1-{1-[5-methyl-2-(4-trifluoromethyl-phenyl)-1H-imidazol-4-ylmethyl]-piperidin-4-yl}-1-(4-trifluoromethyl-benzyl)-urea,
3-Methyl-1-{1-[5-methyl-2-(4-trifluoromethyl-phenyl)-1H-imidazol-4-ylmethyl]-piperidin-4-yl}-1-pyridin-4-yl-urea,
3-Methyl-1-{1-[5-methyl-2-(4-trifluoromethyl-phenyl)-1H-imidazol-4-ylmethyl]-piperidin-4-yl}-1-pyridin-3-yl-urea,
3-Methyl-1-{1-[5-methyl-2-(4-trifluoromethyl-phenyl)-1H-imidazol-4-ylmethyl]-piperidin-4-yl}-1-pyridin-3-ylmethyl-urea,
1-Benzyl-3,3-diethyl-1-{1-[5-methyl-2-(4-trifluoromethyl-phenyl)-1H-imidazol-4-ylmethyl]-piperidin-4-yl}-urea,
1-Benzyl-3-(4-chloro-phenyl)-3-methyl-1-{1-[5-methyl-2-(4-trifluoromethyl-phenyl)-1H-imidazol-4-ylmethyl]-piperidin-4-yl}-urea,
1,3-Dibenzyl-3-methyl-1-{1-[5-methyl-2-(4-trifluoromethyl-phenyl)-1H-imidazol-4-ylmethyl]-piperidin-4-yl}-urea,
1-Benzyl-3-cyclopropyl-1-{1-[5-methyl-2-(4-trifluoromethyl-phenyl)-1H-imidazol-4-ylmethyl]-piperidin-4-yl}-urea,
1-Benzyl-1-[1-(2-benzyl-5-methyl-1H-imidazol-4-ylmethyl)-piperidin-4-yl]-3-methyl-urea,
1-Benzyl-3-methyl-1-[1-(5-methyl-2-phenylamino-1H-imidazol-4-ylmethyl)-piperidin-4-yl]-urea,
1-Benzyl-1-{1-[2-(2-methoxy-phenyl)-5-methyl-1H-imidazol-4-ylmethyl]-piperidin-4-yl}-3-methyl-urea,
1-Benzyl-1-{1-[2-(4-tert.-butyl-phenyl)-5-methyl-1H-imidazol-4-ylmethyl]-piperidin-4-yl}-3-methyl-urea,
1-Benzyl-3-(3,4-dichloro-phenyl)-1-{1-[5-methyl-2-(4-trifluoromethyl-phenyl)-1H-imidazol-4-ylmethyl]-piperidin-4-yl}-urea,
3-(4-Amino-phenyl)-1-benzyl-1-{1-[5-methyl-2-(4-trifluoromethyl-phenyl)-1H -imidazol-4-ylmethyl]-piperidin-4-yl}-urea,
4-(3-Benzyl-3-{1-[5-methyl-2-(4-trifluoromethyl-phenyl)-1H-imidazol-4-ylmethyl]-piperidin-4-yl}-ureido)-benzoic acid,
4-(3-Benzyl-3-{1-[5-methyl-2-(4-trifluoromethyl-phenyl)-1H-imidazol-4-ylmethyl]-piperidin-4-yl}-ureido)-benzoic acid methyl ester,
1-Benzyl-1-{1-[5-methyl-2-(4-trifluoromethyl-phenyl)-1H-imidazol-4-ylmethyl]-piperidin-4-yl}-3-pyridin-4-yl-urea,
1-Benzyl-1-{1-[5-methyl-2-(4-trifluoromethyl-phenyl)-1H-imidazol-4-ylmethyl]-piperidin-4-yl}-3-pyridin-3-yl-urea,
1-Benzyl-1-{1-[5-methyl-2-(4-trifluoromethyl-phenyl)-1H-imidazol-4-ylmethyl]-piperidin-4-yl}-3-pyridin-2-yl-urea,
1-Benzyl-1-{1-[5-methyl-2-(4-trifluoromethyl-phenyl)-1H-imidazol-4-ylmethyl]-piperidin-4-yl}-3-pyridazin-3-yl-urea,
1-Benzyl-1-{1-[5-methyl-2-(4-trifluoromethyl-phenyl)-1H-imidazol-4-ylmethyl]-piperidin-4-yl}-3-pyridazin-4-yl-urea,
1-Benzyl-1-{1-[5-methyl-2-(4-trifluoromethyl-phenyl)-1H-imidazol-4-ylmethyl]-piperidin-4-yl}-3-thiophen-2-yl-urea,
1-Benzyl-3-furan-2-yl-1-{1-[5-methyl-2-(4-trifluoromethyl-phenyl)-1H-imidazol-4-ylmethyl]-piperidin-4-yl}-urea,
1-Benzyl-3-(5-methyl-[1,3,4]thiadiazol-2-yl)-1-{1-[5-methyl-2-(4-trifluoromethyl-phenyl)-1H-imidazol-4-ylmethyl]-piperidin-4-yl}-urea,
1-Benzyl-1-{1-[5-methyl-2-(4-trifluoromethyl-phenyl)-1H-imidazol-4-ylmethyl]-piperidin-4-yl}-3-pyridin-4-ylmethyl-urea,
1-Benzyl-1-{1-[5-methyl-2-(4-trifluoromethyl-phenyl)-1H-imidazol-4-ylmethyl]-piperidin-4-yl}-3-pyridin-3-ylmethyl-urea,
1-Benzyl-1-{1-[5-methyl-2-(4-trifluoromethyl-phenyl)-1H-imidazol-4-ylmethyl]-piperidin-4-yl}-3-pyridin-2-ylmethyl-urea,
1-Benzyl-1-{1-[5-methyl-2-(4-trifluoromethyl-phenyl)-1H-imidazol-4-ylmethyl]-piperidin-4-yl}-3-(tetrahydro-pyran-4-yl)-urea,
1-Benzyl-3-(1-formyl-piperidin-4-yl)-1-{1-[5-methyl-2-(4-trifluoromethyl-phenyl)-1H-imidazol-4-ylmethyl]-piperidin-4-yl}-urea,
1-(2,4-Dichloro-benzyl)-1-{1-[5-methyl-2-(4-trifluoromethyl-phenyl)-1H-imidazol-4-ylmethyl]-piperidin-4-yl}-3-phenyl-urea,
1-(2-Chloro-benzyl)-{1-[5-methyl-2-(4-trifluoromethyl-phenyl)-1H-imidazol-4-ylmethyl]-piperidin-4-yl}-3-phenyl-urea,
1-(2-Methoxy-benzyl)-1-{1-[5-methyl-2-(4-trifluoromethyl-phenyl)-1H-imidazol-4-ylmethyl]-piperidin-4-yl}-3-phenyl-urea,
1-(2-Methyl-benzyl)-1-{1-[5-methyl-2-(4-trifluoromethyl-phenyl)-1H-imidazol-4-ylmethyl]-piperidin-4-yl}-3-phenyl-urea,
1-(3,5-Dichloro-benzyl)-1-{1-[5-methyl-2-(4-trifluoromethyl-phenyl)-1H-imidazol-4-ylmethyl]-piperidin-4-yl}-3-phenyl-urea,
1-(3,4-Dichloro-benzyl)-1-{1-[5-methyl-2-(4-trifluoromethyl-phenyl)-1H-imidazol-4-ylmethyl]-piperidin-4-yl}-3-phenyl-urea,
1-(3-Methyl-benzyl)-1-{1-[5-methyl-2-(4-trifluoromethyl-phenyl)-1H-imidazol-4-ylmethyl]-piperidin-4-yl}-3-phenyl-urea,
1-(4-Methyl-benzyl)-1-{1-[5-methyl-2-(4-trifluoromethyl-phenyl)-1H-imidazol-4-ylmethyl]-piperidin-4-yl}-3-phenyl-urea,
1-{1-[5-Methyl-2-(4-trifluoromethyl-phenyl)-1H-imidazol-4-ylmethyl]-piperidin-4yl}-1-(3-nitro-benzyl)-3-phenyl-urea,
1-(4-Dimethylamino-benzyl)-1-{1-[5-methyl-2-(4-trifluoromethyl-phenyl)-1H-imidazol-4-ylmethyl]-piperidin-4-yl}-3-phenyl-urea,
1-{1-[5-Methyl-2-(4-trifluoromethyl-phenyl)-1H-imidazol-4-ylmethyl]-piperidin-4-yl}-1-(4-nitro-benzyl)-3-phenyl-urea,
1-(2,4-Dimethyl-benzyl)-1-{1-[5-methyl-2-(4-trifluoromethyl-phenyl)-1H-imidazol-4-ylmethyl]-piperidin-4-yl}-3-phenyl-urea,
1-(4-Amino-benzyl)-1-{1-[5-methyl-2-(4-trifluoromethyl-phenyl)-1H-imidazol-4-ylmethyl]-piperidin-4-yl}-3-phenyl-urea,
4-(1-{1-[5-Methyl-2-(4-trifluoromethyl-phenyl)-1H-imidazol-4-ylmethyl]-piperidin-4-yl}-3-phenyl-ureidomethyl)-benzoic acid methyl ester,
1-(4-Methanesulfonyl-benzyl)-1-{1-[5-methyl-2-(4-trifluoromethyl-phenyl)-1H-imidazol-4-ylmethyl]-piperidin-4-yl}-3-phenyl-urea,
1-Biphenyl-3-ylmethyl-1-{1-[5-methyl-2-(4-trifluoromethyl-phenyl)-1H-imidazol-4-ylmethyl]-piperidin-4-yl}-3-phenyl-urea,
1-Biphenyl-2-ylmethyl-1-{1-[5-methyl-2-(4-trifluoromethyl-phenyl)-1H-imidazol-4-ylmethyl]-piperidin-4-yl}-3-phenyl-urea,
1-{1-[5-Methyl-2-(4-trifluoromethyl-phenyl)-1H-imidazol-4-ylmethyl]-piperidin-4-yl}-1-(4-phenoxy-benzyl)-3-phenyl-urea,
1-Biphenyl-4-ylmethyl-1-{1-[5-methyl-2-(4-trifluoromethyl-phenyl)-1H-imidazol-4-ylmethyl]-piperidin-4-yl }-3-phenyl-urea,
1-(4-Cyano-benzyl)-1-{1-[5-methyl-2-(4-trifluoromethyl-phenyl)-1H-imidazol-4-ylmethyl]-piperidin-4-yl}-3-phenyl-urea,
1-Benzyl-3-methyl-1-[1-(5-methyl-2-p-tolyl-1H-imidazol-4-ylmethyl)-piperidin-4-yl]-urea,
1-Benzyl-1-{1-[2-(4-methoxy-phenyl)-5-methyl-1H-imidazol-4-ylmethyl]-piperidin-4-yl}-3-methyl-urea,
1-Cyclopentyl-3-methyl-1-{1-[5-methyl-2-(4-trifluoromethyl-phenyl)-1H -imidazol-4-ylmethyl]-piperidin-4-yl}-urea, and
1-Benzyl-3-(4-iodo-phenyl)-1-{1-[5-methyl-2-(4-trifluoromethyl-phenyl)-1H-imidazol-4-ylmethyl]-piperidin-4-yl}-urea.

13. A pharmaceutical composition comprising a pharmaceutically effective amount of a compound according to claim 1 combined with pharmaceutically acceptable excipients.14. A method for treating HIV infection comprising administering a therapeutically effective amount of the pharmaceutical composition according to claim 13.
